(19) United States
(12) Patent Application Publication
(10) Pub. No.: US 2008/0114007 A1
(43) Pub. Date: May 15, 2008

(54) 5-OXO-5,8-DIHYDRO-PYRIDO-PYRIMIDINES AS INHIBITORS OF C-FMS KINASE

(76) Inventors: Mark R. Player, Phoenixville, PA (US); William H. Parsons, Belle Mead, NJ (US); Hui Huang, Monroe Twp., NJ (US); Daniel A. Hutta, Belle Mead, NJ (US); Huaping Hu, Pennington, NJ (US); James Rinker, Reading, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/875,294

(22) Filed: Oct. 19, 2007

Related U.S. Application Data
(60) Provisional application No. 60/855,523, filed on Oct. 31, 2006.

(51) Int. Cl. A61K 31/519; C07D 471/04; C07D 401/02; C07D 401/14; A61P 35/00; A61P 9/00

(52) U.S. Cl. 514/264.11; 544/279

(57) ABSTRACT

The invention addresses the current need for selective and potent protein tyrosine kinase inhibitors by providing potent inhibitors of c-fms kinase. The invention is directed to the novel compounds of Formula I: 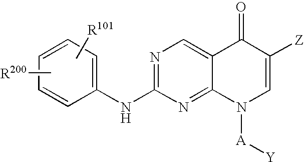
or a salt, stereoisomer, tautomer, crystalline, polymorph, amorphous, solvate, hydrate, ester, prodrug or metabolite form thereof, wherein A, Y, Z, R¹⁰¹ and R²⁰⁰ are described in the specification.

### CROSS REFERENCE TO RELATED APPLICATIONS

This present application claims benefit of U.S. Provisional Patent Application Ser. No. 60/855,523, filed Oct. 31, 2006, which is incorporated herein by reference in its entirety and for all purposes.

### BACKGROUND OF THE INVENTION

The invention relates to novel compounds that function as protein tyrosine kinase inhibitors. The family of 5-oxo-5,8-dihydro-pyrido-pyrimidines has exhibited promising pharmaceutical properties in the past; U.S. Pat. No. 4,556,709, JP 09221424 and DE 19532235 are indicative of recent investigations. More particularly, the invention relates to novel compounds that function as inhibitors of c-fms kinase.

The c-fms kinase is a type III receptor tyrosine kinase selectively expressed on macrophages and their progenitors. The extracellular Ig domain of c-fms binds macrophage colony stimulating factor (M-CSF), also known as colony stimulating factor-1 (CSF-1). Binding of CSF-1 induces receptor dimerization and trans-phosphorylation of the intracellular c-fms kinase domain on Y723 and other tyrosine residues. Once phosphorylated, c-fms efficiently phosphorylates several cytoplasmic signaling molecules that lead to de novo gene expression and proliferation. Small molecule inhibitors of the kinase catalytic site of c-fms are expected to prevent CSF-1 induced cellular responses.

Macrophages are a predominant source of tumor necrosis factor (TNF) and interleukin-1 (IL-1) in the destructive pannus of rheumatoid arthritis. TNF and IL-1 activate stromal expression of hematopoietic factors including CSF-1. In turn, CSF-1 recruits monocytes and promotes macrophage survival, functional activation, and in some settings, proliferation. Thus, TNF and CSF-1 interact in a perpetuating cycle that leads to inflammation and joint destruction. The exclusive receptor for CSF-1 is c-fms, and the disclosed invention is a c-fms inhibitor designed to interrupt this cycle.

Macrophages are abundant at sites of chronic inflammation where they are often the most important source of TNF, IL-1, and other cytokines. Moreover, macrophages can be an important source of factors that function in tissue remodeling such as plasminogen activators, matrix metalloproteases, vascular endothelial growth factor, and transforming growth factor-β (TGF-β). The numbers of macrophages present within target tissues have strongly correlated with disease severity in rheumatoid arthritis (Ann. Rheum. Dis., 53 (1994) pp 39-44), immune nephritis (Kidney Int., 54 (1998) pp 143-151), and graft rejection (Transpl. Int., 7 Suppl 1 (1994) pp 577-579). Macrophage numbers are also elevated in atherosclerotic plaque (Arch. Pathol. Lab. Med., 109 (1985) pp 445-449), adipose tissue in obesity (J. Clin. Invest., 112 (2003) pp 1796-1898), diabetic nephropathy (Kidney Int., 65 (2004) pp 116-128), cardiac hypertrophy (Hypertension, 25 (1999) pp 132-138), and in many solid tumors (Trends in Immunology, 23 (2002) pp 549-555), particularly breast cancer (J. Experimental Medicine, 193 (2001) pp 727-739), where they are thought to contribute to disease progression. Modulation of macrophage function through inhibition of c-fms thus is expected to be useful in treating inflammatory mediated diseases and conditions.

Another example of the invention is the use of any of the compounds described herein in the preparation of a medicament for treating: rheumatoid arthritis, graft rejection, atherosclerosis, obesity, diabetic nephropathy, cardiac hypertrophy and solid tumor cancers, especially breast cancer, in a subject in need of such treatment.

Preclinical data suggest CSF-1/FMS is a particularly viable therapeutic target for rheumatoid arthritis. Recent work has shown that neutralizing antibodies to CSF-1 reduce substantially the severity of collagen-induced arthritis in mice (J. Leukoc. Biol., 68 (2000) pp 144-150). The authors additionally demonstrated that recombinant CSF-1 exacerbated the disease progress in this model. Therefore, a preferred use for the invention is the treatment of rheumatoid arthritis.

### SUMMARY OF THE INVENTION

The invention addresses the current need for selective and potent protein tyrosine kinase inhibitors by providing potent inhibitors of c-fms kinase.

The invention is directed to the novel compounds of Formula I: 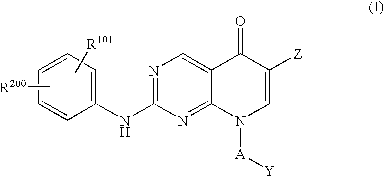
or a form thereof, wherein A, Y, Z, R¹⁰¹ and R²⁰⁰ are as defined herein.

The invention is also directed to a method of using a compound of Formula I for inhibiting protein tyrosine kinase activity comprising administering an effective amount of at least one compound of Formula I.

The invention is directed to a method of inhibiting c-fms kinase activity in a subject in need thereof comprising administering to the subject an effective amount of at least one compound of Formula I.

The invention is also directed to a method of treating or ameliorating a c-fms kinase mediated disorder in a subject in need thereof comprising administering to the subject an effective amount of at least one compound of Formula I.

### DETAILED DESCRIPTION OF THE INVENTION

This invention is directed to a compound of Formula I: 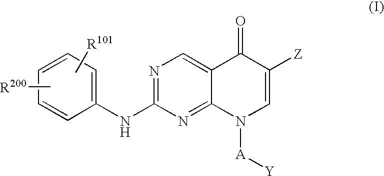
or a form thereof, wherein:
A is absent or C₁₋₈alkyl;
Y is C₃₋₁₄cycloalkyl, aryl, heterocyclyl or heteroaryl each optionally substituted with one, two or three substituents selected from C₁₋₈alkyl, C₂₋₈alkenyl, C₂₋₈alkynyl, C₁₋₈alkoxy, halo-C₁₋₈alkyl, halo-C₁₋₈alkoxy, halogen, hydroxy or amino, wherein heteroaryl is not thiazole;
Z is R¹-amino-carbonyl or heterocyclyl-carbonyl;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₁₋₈alkyl-amino-C₁₋₈alkyl, C₃₋₁₄cycloalkyl-oxy or heteroaryl;
R¹⁰¹ is one or two substituents each selected from hydrogen, halogen or hydroxy;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵—C₃₋₁₄cycloalkyl, R⁵—C₃₋₁₄cycloalkyl-C₁₋₈alkyl, R⁵-aryl, R⁵-aryl-C₁₋₈alkyl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl,
alternatively, R₂₀₀ is a ring selected from heterocyclyl or heteroaryl fused on two adjacent carbon atoms of the phenyl ring of Formula (I) to form an R⁵ substituted bicyclic heterocyclyl or heteroaryl ring;
R² is one, two or three optional substituents each selected from halogen, hydroxy, C₁₋₈alkoxy, carboxy, amino-carbonyl, C₁₋₈alkyl-amino-carbonyl, amino-amino-carbonyl, C₁₋₈alkyl-amino-amino-carbonyl, C₁₋₈alkyl-sulfonyl, amino-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl, R⁵-heterocyclyl-amino-carbonyl or R⁵-heterocyclyl-sulfonyl;
R³ is one, two or three optional substituents each selected from halogen, hydroxy, C₁₋₈alkoxy, amino, C₁₋₈alkyl-amino or R⁵-heterocyclyl;
R⁴ is two substituents each selected from hydrogen, C₁₋₈alkyl, C₁₋₈alkoxy-C₁₋₈alkyl, hydroxy-C₁₋₈alkyl, amino-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or R⁵-heterocyclyl-C₁₋₈alkyl;
R⁵ is one, two, three or four substituents each selected from hydrogen, halogen, hydroxy, oxo, carboxy, R⁶—C₁₋₈alkyl, R⁶—C₁₋₈alkoxy, amino, C₁₋₈alkyl-amino, C₁₋₈alkyl-sulfonyl, amino-sulfonyl, C₁₋₈alkyl-amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, amino, C₁₋₈alkyl-amino, C₁₋₈alkoxy, carboxy, C₁₋₈alkoxy-carbonyl, C₁₋₈alkyl-sulfonyl, C₁₋₈alkyl-sulfonyl-amino-carbonyl, aryl or heterocyclyl, wherein heterocyclyl is optionally substituted with one, two or three substituents each selected from oxo or C₁₋₈alkyl.

An example of the present invention is a compound of Formula I or a form thereof, wherein:
A is absent or C₁₋₈alkyl;
Y is C₃₋₁₄cycloalkyl, aryl or heterocyclyl each optionally substituted with one, two or three substituents each selected from C₁₋₈alkyl, C₂₋₈alkynyl, halo-C₁₋₈alkyl, halo-C₁₋₈alkoxy or halogen;
Z is R¹-amino-carbonyl or heterocyclyl-carbonyl;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₃₋₁₄cycloalkyl-oxy or heteroaryl;
R^{10l} is hydrogen;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-sulfonyl, R⁴-amino-C₁₋₈alkyl-sulfonyl, R⁵-aryl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl;
R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl or R⁵-heterocyclyl-amino-carbonyl;
R³ is one optional substituent selected from C₁₋₈alkoxy or R⁵-heterocyclyl;
R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or R⁵-heterocyclyl-C₁₋₈alkyl;
R⁵ is one, two, three or four substituents each selected from hydrogen, halogen, hydroxy, oxo, carboxy, R⁶—C₁₋₈alkyl, R⁶—C₁₋₈alkoxy, C₁₋₈alkyl-amino, C₁₋₈alkyl-sulfonyl, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or aryl.

An example of the present invention is a compound of Formula I or a form thereof, wherein:
A is absent or C₁₋₈alkyl;
Y is cyclopropyl, cyclopentyl, cyclohexyl, 1H-indenyl, indanyl, phenyl, naphthalenyl or 6,7-dihydro-5H-cyclopenta[b]pyridinyl each optionally substituted with one, two or three substituents each selected from C₁₋₈alkyl, C₂₋₈alkynyl, halo-C₁₋₈alkyl, halo-C₁₋₈alkoxy or halogen;
Z is R¹-amino-carbonyl, morpholinyl-carbonyl or piperidinyl-carbonyl;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, cyclopentyl-oxy or thiazolyl;
R^{10l} is hydrogen;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵-phenyl, R⁵-pyrrolidinyl, R⁵-piperazinyl, R⁵-piperidinyl, R⁵-morpholinyl, R⁵-(1,2,3,6-tetrahydropyridinyl), R⁵-(3,5,11-trioxa-tricyclo[5.3.1.0^{2,6}]undecanyl), R⁵-1H-pyrrolyl, R⁵-1H-pyrazolyl, R⁵-1H-[1,2,4]triazolyl, R⁵-pyrrolidinyl-oxy, R⁵-piperidinyl-oxy, R⁵-pyranyl-oxy, R⁵-(1-azabicyclo[2.2.2]octyl)-oxy, R⁵-pyrrolidinyl-carbonyl, R⁵-piperidinyl-carbonyl, R⁵-morpholinyl-carbonyl, R⁵-piperazinyl-sulfonyl, R⁵-azetidinyl-C₁₋₈alkyl, R⁵-pyrrolidinyl-C₁₋₈alkyl, R⁵-piperazinyl-C₁₋₈alkyl, R⁵-piperidinyl-C₁₋₈alkyl, R⁵-morpholinyl-C₁₋₈alkyl, R⁵-thiomorpholinyl-C₁₋₈alkyl, R⁵-oxazolidinyl-C₁₋₈alkyl, R⁵-(7-aza-bicyclo[2.2.1]heptyl)-C₁₋₈alkyl, 1,5-dioxa-9-aza-spiro[5.5]undec-9-yl-C₁₋₈alkyl, R⁵-(1H-[1,2,4]triazolyl)-C₁₋₈alkyl, R⁵-1H-tetrazol-5-yl-C₁₋₈alkyl, R⁵-imidazolyl-C₁₋₈alkyl or R⁵-pyridinyl-C₁₋₈alkyl,
alternatively, R₂₀₀ is a ring selected from pyrrolidinyl, piperidinyl, 1,4,7,10,13-pentaoxa-cyclopentadecane, 1,4,7,10,13,16-hexaoxa-cyclooctadecane, pyrrolyl, imidazolyl or pyrazolyl fused on two adjacent carbon atoms of the phenyl ring of Formula (I) to form an R⁵ substituted 2,3-dihydro-1H-indolyl, 1,2,3,4-tetrahydroisoquinolinyl, 6,7,9,10,12,13,15,16-octahydro-5,8,11,14,17-pentaoxa-benzocyclopentadecenyl, 6,7,9,10,12,13,15,16,18,19-decahydro-5,8,11,14,17,20-hexaoxa-benzocyclooctadecenyl, 1H-indolyl, 1H-benzimidazolyl or 1H-indazolyl ring;
R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-piperazinyl-carbonyl or R⁵-piperidinyl-amino-carbonyl;
R³ is one optional substituent selected from C₁₋₈alkoxy or R⁵-morpholinyl;
R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, R⁵-adamantanyl, R⁵-bicyclo[2.2.1]heptyl, R⁵-piperidinyl, R⁵-tetrahydro-pyranyl, R⁵-pyrrolidinyl-C₁₋₈alkyl or R⁵-morpholinyl-C₁₋₈alkyl;
R⁵ is one, two, three or four substituents each selected from hydrogen, halogen, hydroxy, oxo, carboxy, R⁶—C₁₋₈alkyl, R⁶—C₁₋₈alkoxy, C₁₋₈alkyl-amino, C₁₋₈alkyl-sulfonyl, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or phenyl.

An example of the present invention is a compound of Formula I or a form thereof, wherein A is absent; Y is C₃₋₁₀cycloalkyl or aryl optionally substituted with C₁₋₈alkyl; Z is R¹-amino-carbonyl; R¹ is C₁₋₈alkoxy; R^{10l} is hydrogen; R²⁰⁰ is R⁵-heterocyclyl; R⁵ is R⁶—C₁₋₈alkyl; and, R⁶ is hydrogen.

An example of the present invention is a compound of Formula I or a form thereof, wherein A is absent; Y is indanyl or phenyl optionally substituted with C₁₋₈alkyl; Z is R¹-amino-carbonyl; R¹ is C₁₋₈alkoxy; R^{10l} is hydrogen; R²⁰⁰ is R⁵-piperidinyl; R⁵ is R⁶—C₁₋₈alkyl; and, R⁶ is hydrogen.

An example of the present invention is a compound of Formula I or a form thereof, wherein A is absent.

An example of the present invention is a compound of Formula I or a form thereof, wherein A is C₁₋₈alkyl.

An example of the present invention is a compound of Formula I or a form thereof, wherein Y is C₃₋₁₄cycloalkyl, aryl or heterocyclyl each optionally substituted with one, two or three substituents each selected from C₁₋₈alkyl, C₂₋₈alkynyl, halo-C₁₋₈alkyl, halo-C₁₋₈alkoxy or halogen.

An example of the present invention is a compound of Formula I or a form thereof, wherein Y is cyclopropyl, cyclopentyl, cyclohexyl, 1H-indenyl, indanyl, phenyl, naphthalenyl or 6,7-dihydro-5H-cyclopenta[b]pyridinyl each optionally substituted with one, two or three substituents each selected from C₁₋₈alkyl, C₂₋₈alkynyl, halo-C₁₋₈alkyl, halo-C₁₋₈alkoxy or halogen.

An example of the present invention is a compound of Formula I or a form thereof, wherein Y is C₃₋₁₀cycloalkyl or aryl each optionally substituted with one or two substituents selected from C₁₋₈alkyl or halo-C₁₋₈alkyl.

An example of the present invention is a compound of Formula I or a form thereof, wherein Y is indanyl or phenyl optionally substituted with C₁₋₈alkyl.

An example of the present invention is a compound of Formula I or a form thereof, wherein Z is R¹-amino-carbonyl.

An example of the present invention is a compound of Formula I or a form thereof, wherein Z is heterocyclyl-carbonyl.

An example of the present invention is a compound of Formula I or a form thereof, wherein Z is morpholinyl-carbonyl or piperidinyl-carbonyl.

An example of the present invention is a compound of Formula I or a form thereof, wherein R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₃₋₁₄cycloalkyl-oxy or heteroaryl.

An example of the present invention is a compound of Formula I or a form thereof, wherein R¹ is one substituent selected from hydrogen, C₁₋₈alkoxy or C₃₋₁₄cycloalkyl-oxy.

An example of the present invention is a compound of Formula I or a form thereof, wherein R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, cyclopentyl-oxy or thiazolyl.

An example of the present invention is a compound of Formula I or a form thereof, wherein R¹ is one substituent selected from hydrogen, C₁₋₈alkoxy or cyclopentyl-oxy.

An example of the present invention is a compound of Formula I or a form thereof, wherein R¹ is hydrogen or C₁₋₈alkoxy.

An example of the present invention is a compound of Formula I or a form thereof, wherein R¹⁰¹ is hydrogen.

An example of the present invention is a compound of Formula I or a form thereof, wherein R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-sulfonyl, R⁴-amino-C₁₋₈alkyl-sulfonyl, R⁵-aryl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl.

An example of the present invention is a compound of Formula I or a form thereof, wherein R²⁰⁰ is one substituent selected from hydrogen, R²—C₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵-phenyl, R⁵-pyrrolidinyl, R⁵-piperazinyl, R⁵-piperidinyl, R⁵-morpholinyl, R⁵-(1,2,3,6-tetrahydropyridinyl), R⁵-(3,5,11-trioxa-tricyclo[5.3.1.0^{2,6}]undecanyl), R⁵-1H-pyrrolyl, R⁵-1H-pyrazolyl, R⁵-1H-[1,2,4]triazolyl, R⁵-pyrrolidinyl-oxy, R⁵-piperidinyl-oxy, R⁵-pyranyl-oxy, R⁵-(1-azabicyclo[2.2.2]octyl)-oxy, R⁵-pyrrolidinyl-carbonyl, R⁵-piperidinyl-carbonyl, R⁵-morpholinyl-carbonyl, R⁵-piperazinyl-sulfonyl, R⁵-azetidinyl-C₁₋₈alkyl, R⁵-pyrrolidinyl-C₁₋₈alkyl, R⁵-piperazinyl-C₁₋₈alkyl, R⁵-piperidinyl-C₁₋₈alkyl, R⁵-morpholinyl-C₁₋₈alkyl, R⁵-thiomorpholinyl-C₁₋₈alkyl, R⁵-oxazolidinyl-C₁₋₈alkyl, R⁵-(7-aza-bicyclo[2.2.1]heptyl)-C₁₋₈alkyl, 1,5-dioxa-9-aza-spiro[5.5]undec-9-yl-C₁₋₈alkyl, R⁵-(1H-[1,2,4]triazolyl)-C₁₋₈alkyl, R⁵-1H-tetrazol-5-yl-C₁₋₈alkyl, R⁵-imidazolyl-C₁₋₈alkyl or R⁵-pyridinyl-C₁₋₈alkyl.

An example of the present invention is a compound of Formula I or a form thereof, wherein, alternatively, R₂₀₀ is a ring selected from pyrrolidinyl, piperidinyl, 1,4,7,10,13-pentaoxa-cyclopentadecane, 1,4,7,10,13,16-hexaoxa-cyclooctadecane, pyrrolyl, imidazolyl or pyrazolyl fused on two adjacent carbon atoms of the phenyl ring of Formula (I) to form an R⁵ substituted 2,3-dihydro-1H-indolyl, 1,2,3,4-tetrahydroisoquinolinyl, 6,7,9,10,12,13,15,16-octahydro-5,8,11,14,17-pentaoxa-benzocyclopentadecenyl, 6,7,9,10,12,13,15,16,18,19-decahydro-5,8,11,14,17,20-hexaoxa-benzocyclooctadecenyl, 1H-indolyl, 1H-benzimidazolyl or 1H-indazolyl ring.

An example of the present invention is a compound of Formula I or a form thereof, wherein R²⁰⁰ is R⁵-heterocyclyl.

An example of the present invention is a compound of Formula I or a form thereof, wherein R²⁰⁰ is R⁵-piperidinyl.

An example of the present invention is a compound of Formula I or a form thereof, wherein
R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl or R⁵-heterocyclyl-amino-carbonyl.

An example of the present invention is a compound of Formula I or a form thereof, wherein R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-piperazinyl-carbonyl or R⁵-piperidinyl-amino-carbonyl.

An example of the present invention is a compound of Formula I or a form thereof, wherein R³ is one optional substituent selected from C₁₋₈alkoxy or R⁵-heterocyclyl.

An example of the present invention is a compound of Formula I or a form thereof, wherein R³ is one optional substituent selected from C₁₋₈alkoxy or R⁵-morpholinyl.

An example of the present invention is a compound of Formula I or a form thereof, wherein R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or R⁵-heterocyclyl-C₁₋₈alkyl.

An example of the present invention is a compound of Formula I or a form thereof, wherein R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, R⁵-adamantanyl, R⁵-bicyclo[2.2.1]heptyl, R⁵-piperidinyl, R⁵-tetrahydro-pyranyl, R⁵-pyrrolidinyl-C₁₋₈alkyl or R⁵-morpholinyl-C₁₋₈alkyl.

An example of the present invention is a compound of Formula I or a form thereof, wherein R⁵ is one, two, three or four substituents each selected from hydrogen, halogen, hydroxy, oxo, carboxy, R⁶—C₁₋₈alkyl, R⁶—C₁₋₈alkoxy, amino, C₁₋₈alkyl-amino, C₁₋₈alkyl-sulfonyl, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl.

An example of the present invention is a compound of Formula I or a form thereof, wherein R⁵ is R⁶—C₁₋₈alkyl.

An example of the present invention is a compound of Formula I or a form thereof, wherein R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or aryl.

An example of the present invention is a compound of Formula I or a form thereof, wherein R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or phenyl.

An example of the present invention is a compound of Formula I or a form thereof, wherein R⁶ is hydrogen.

An example of the present invention is a compound of Formula I selected from the group consisting of: 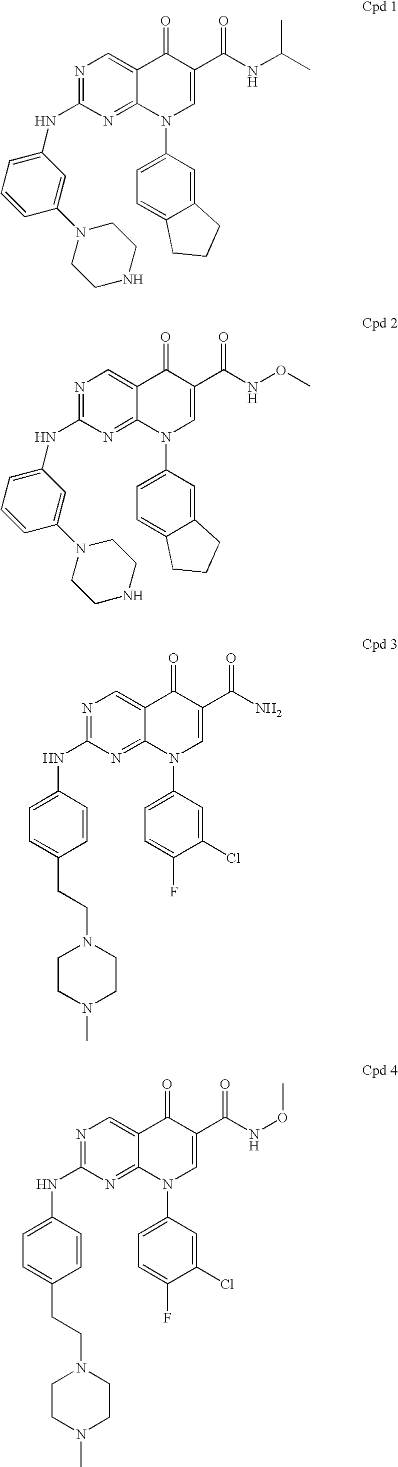 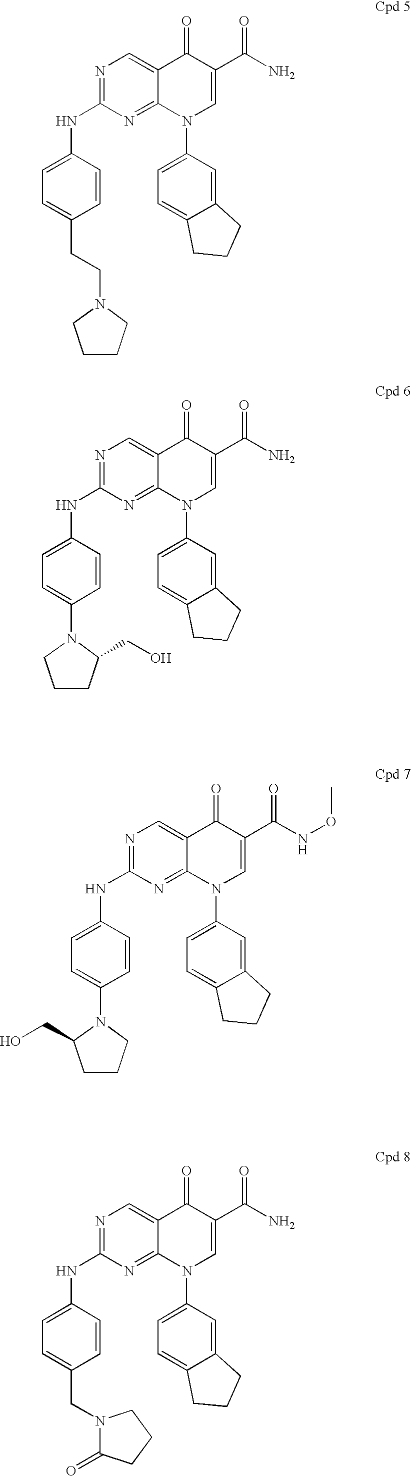 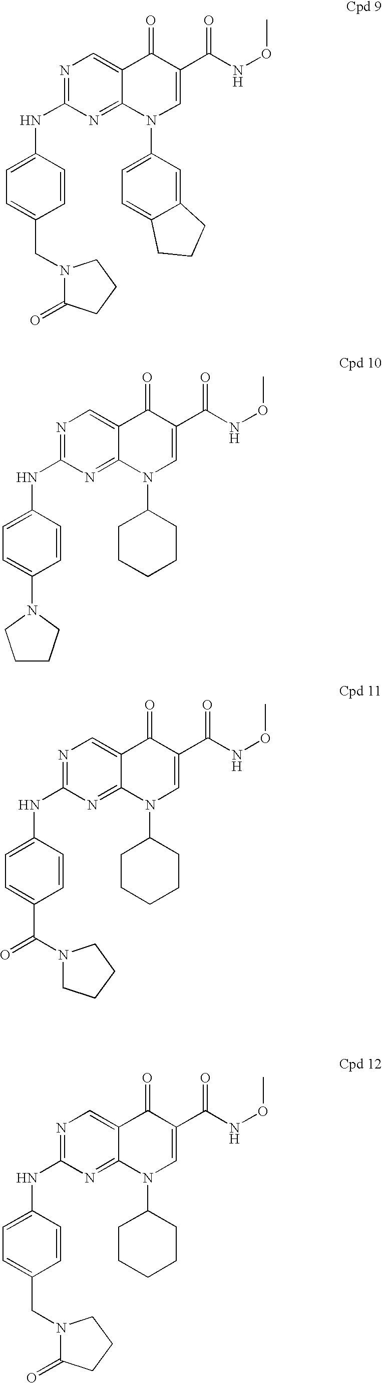 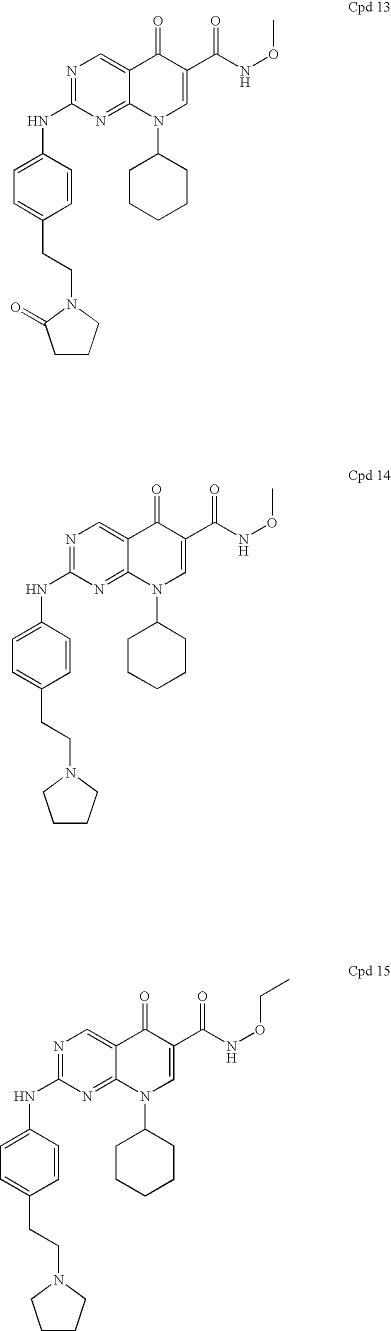 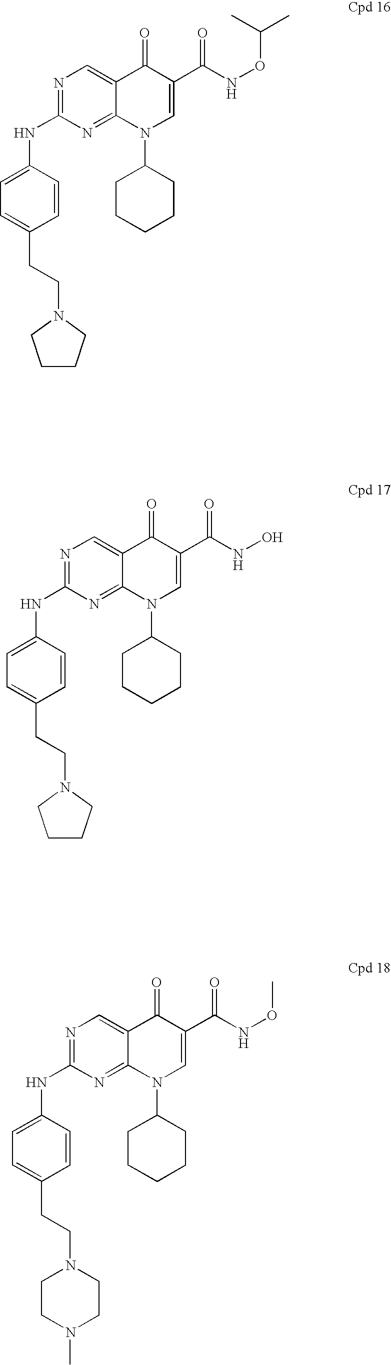 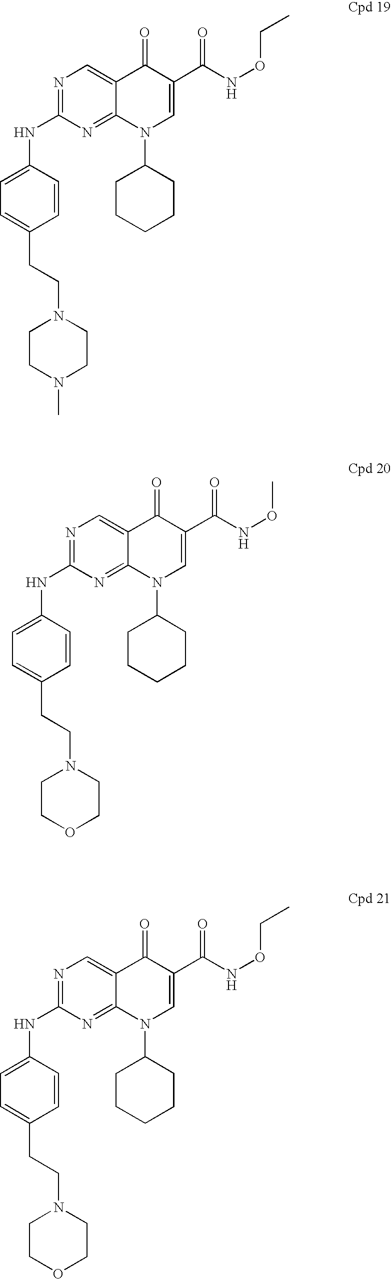 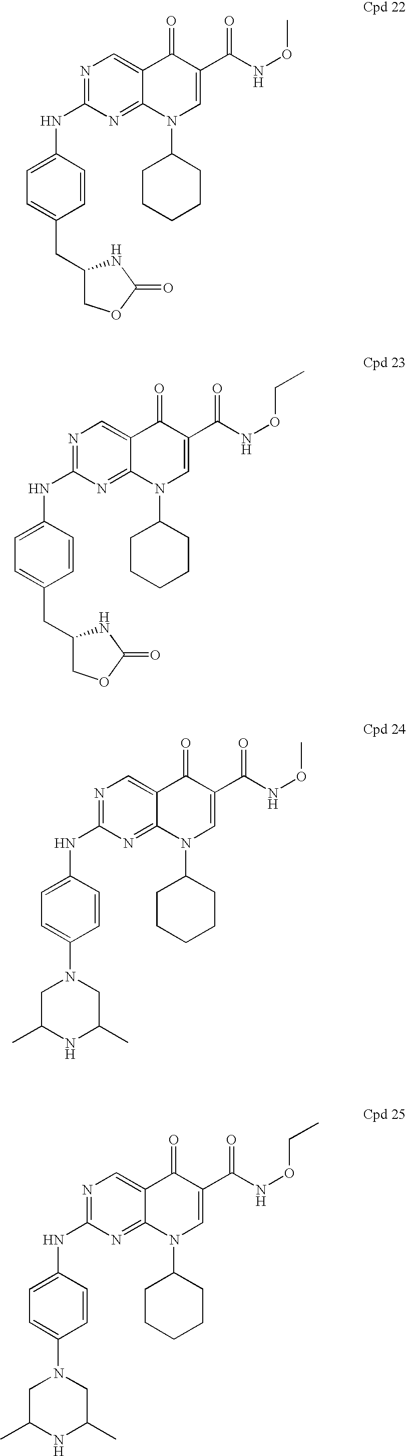 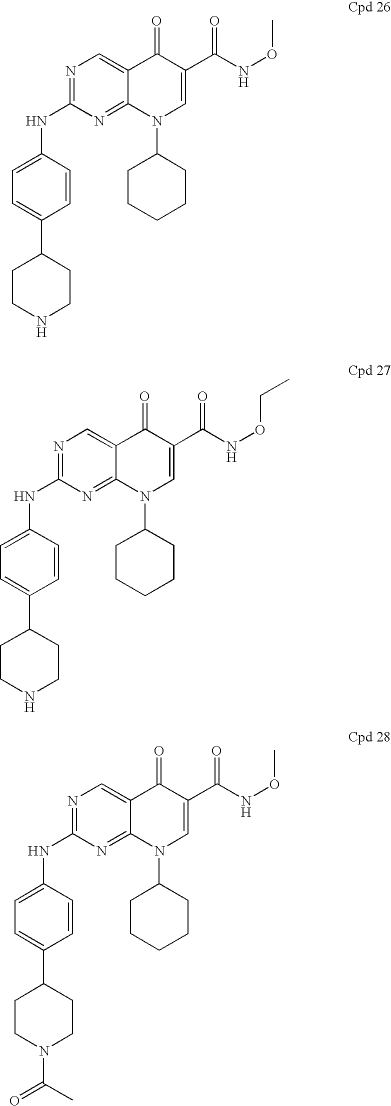 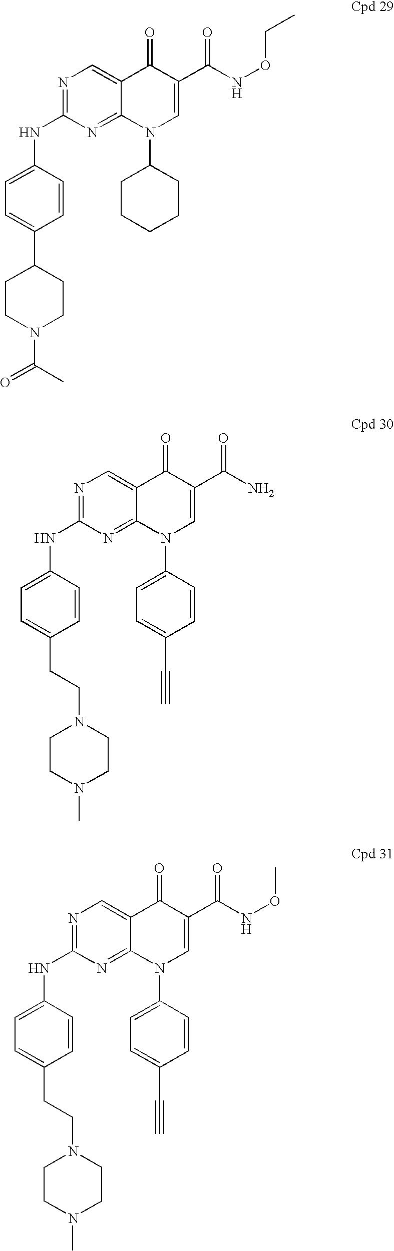 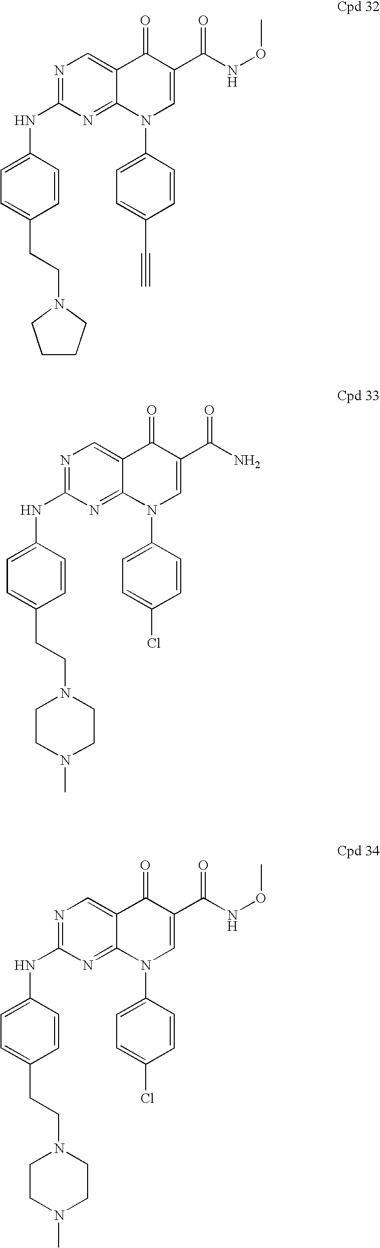 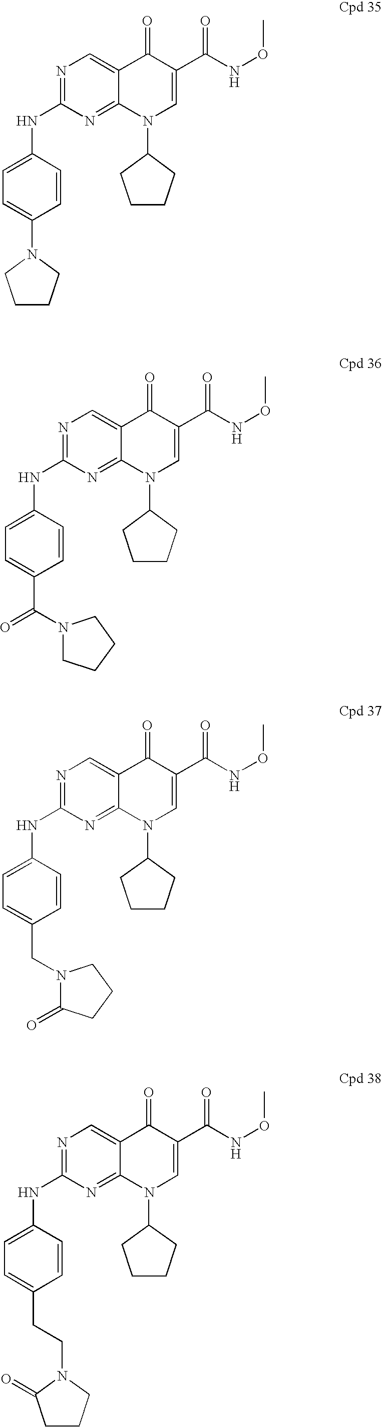 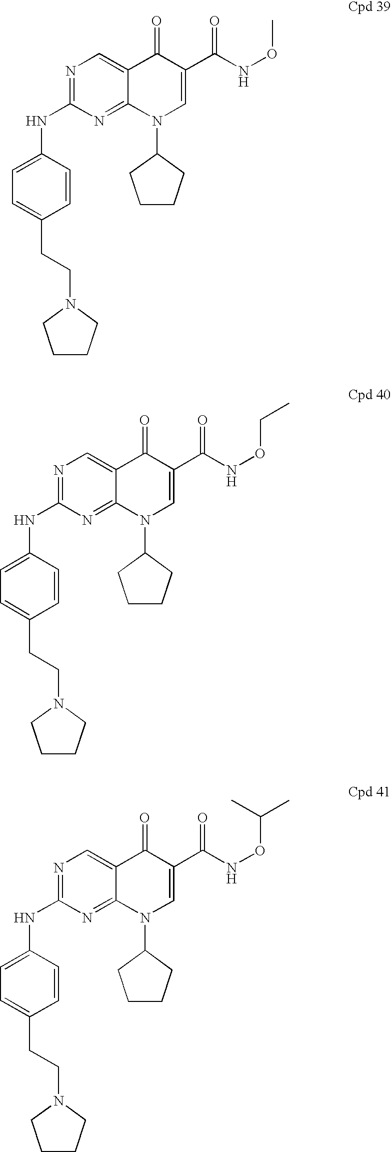 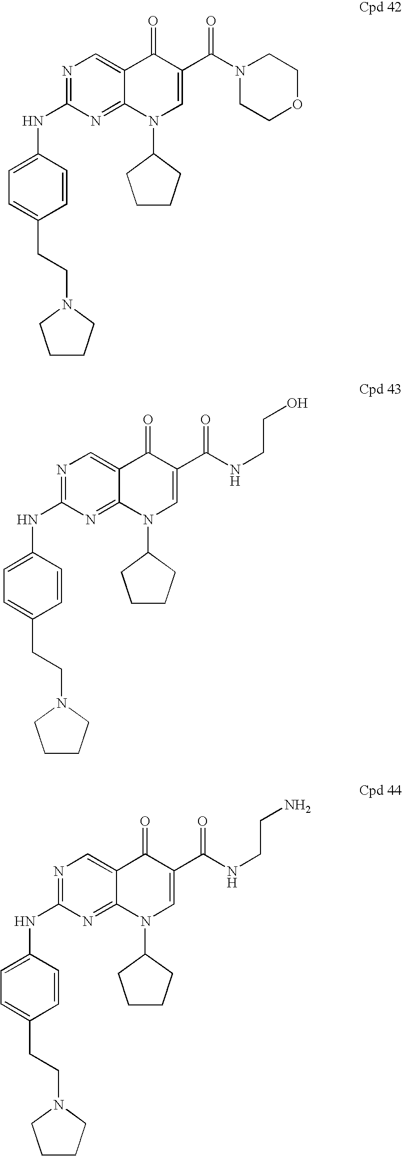 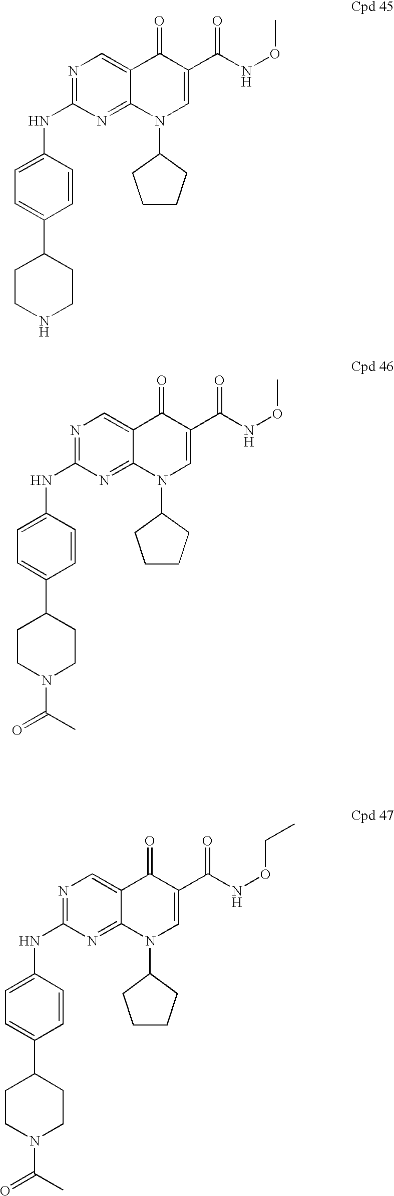 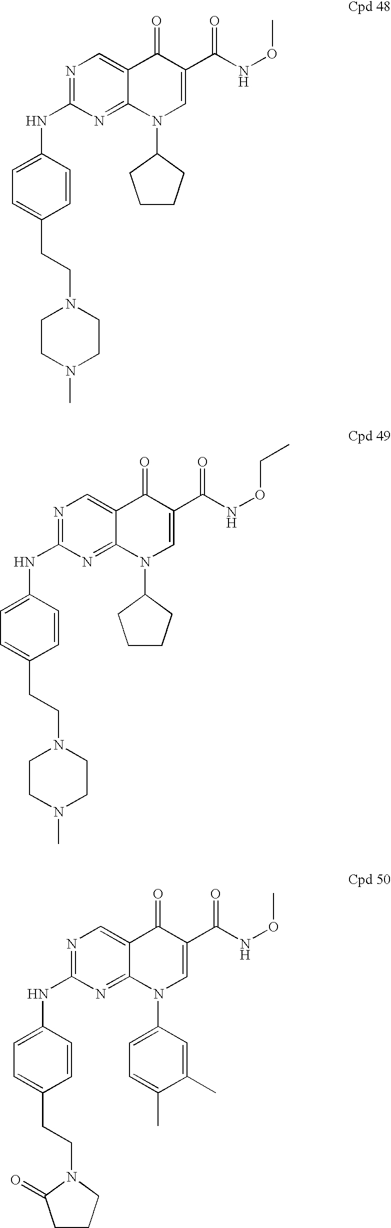 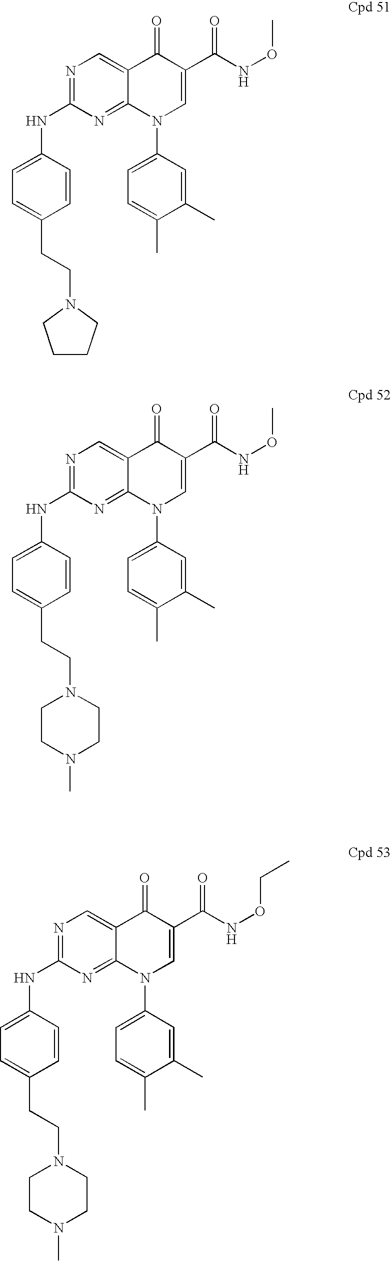 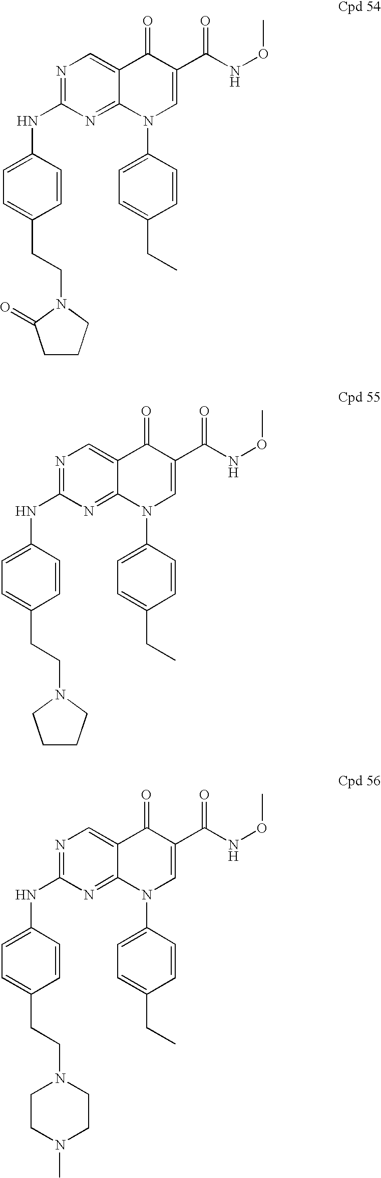 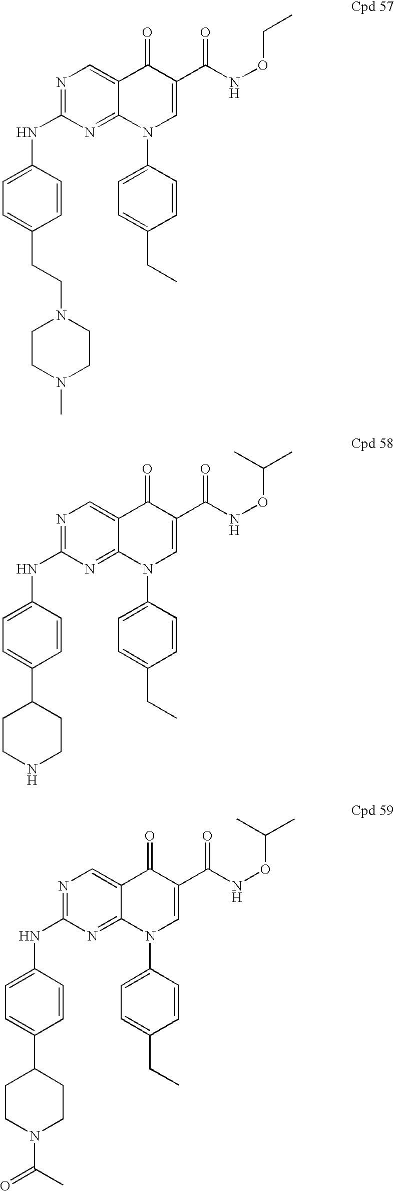 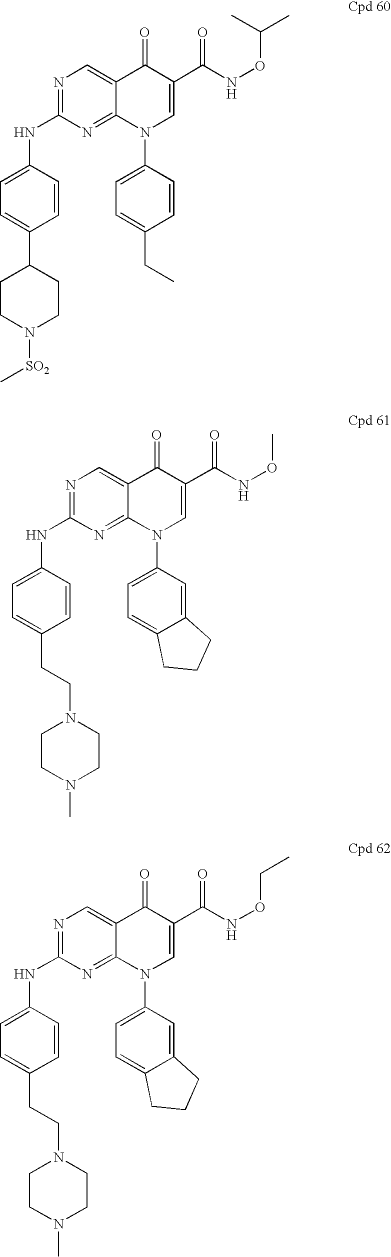 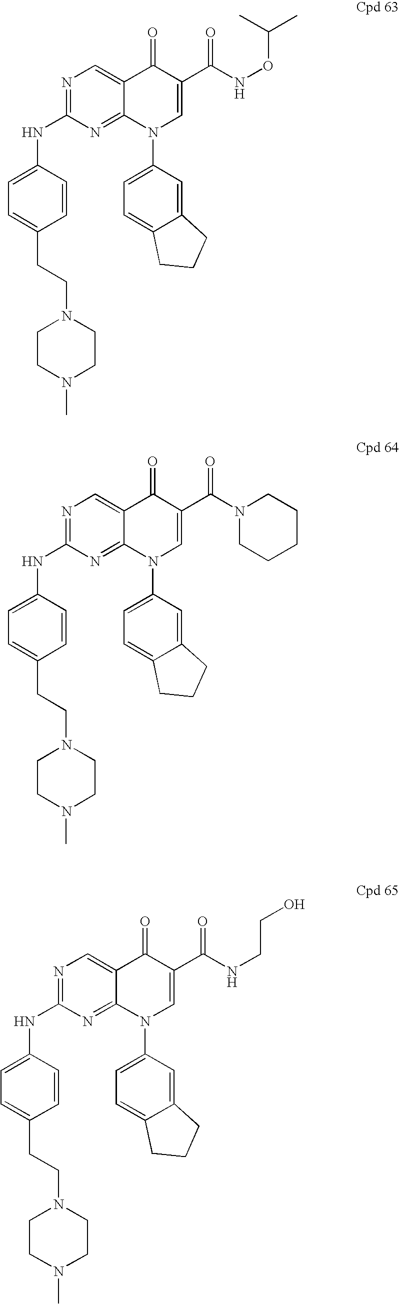 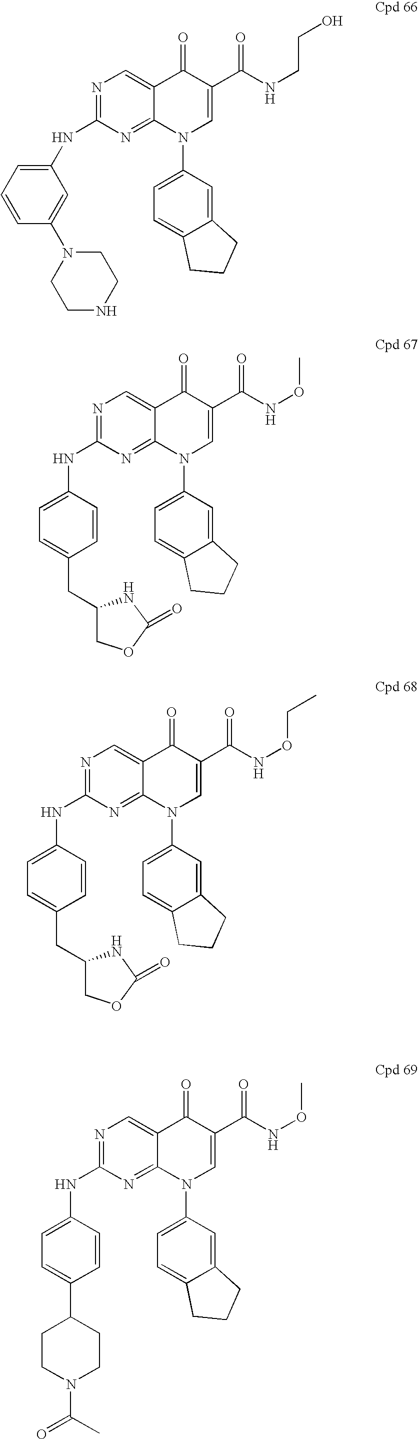 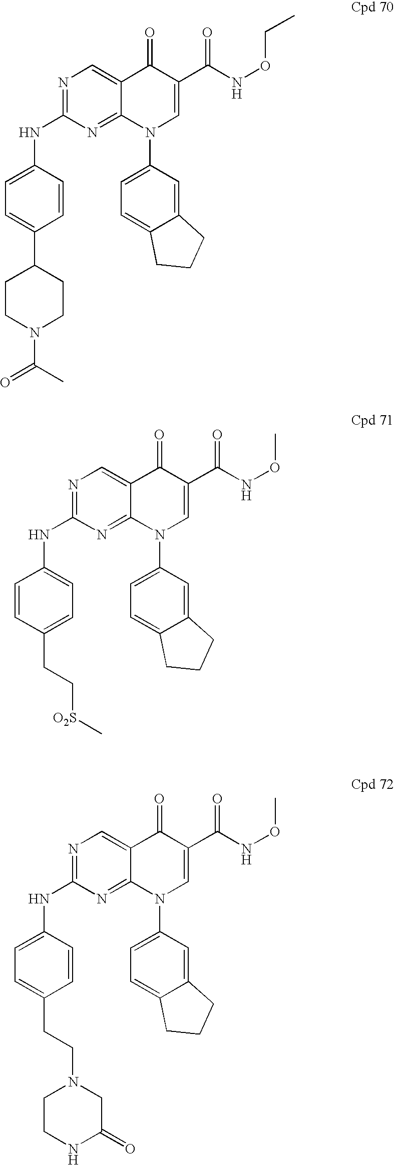 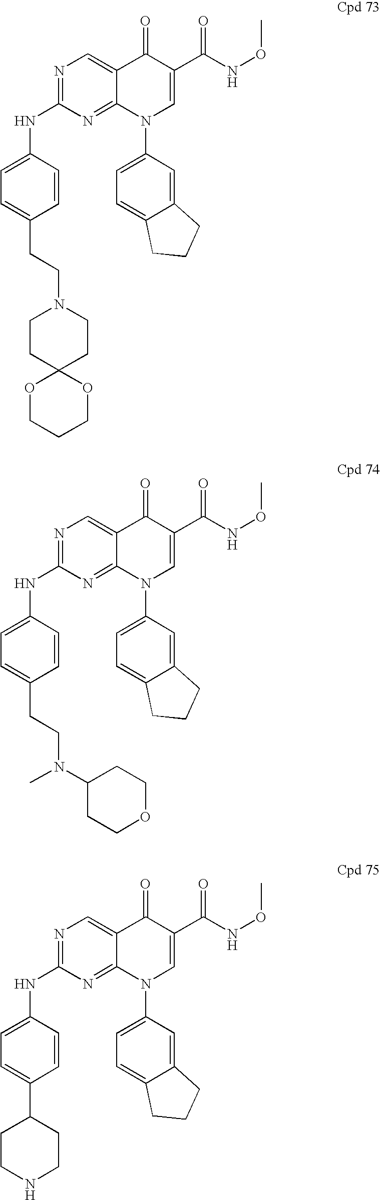 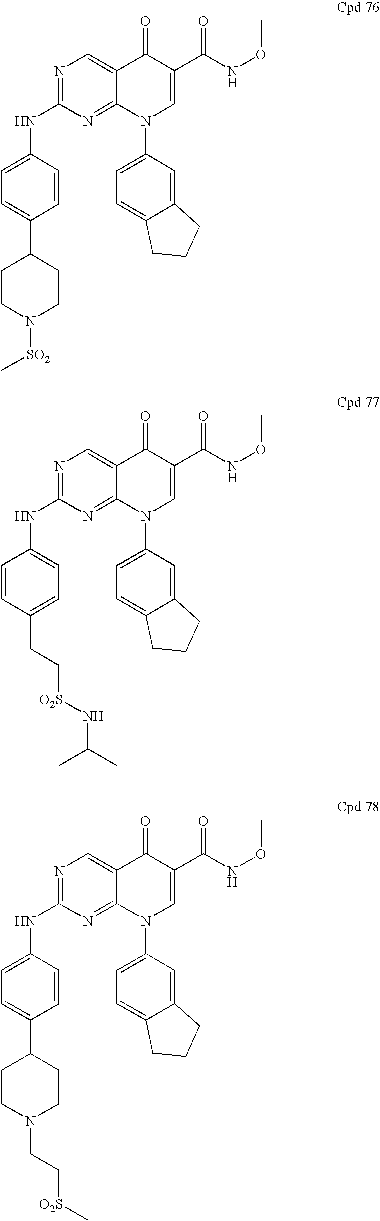 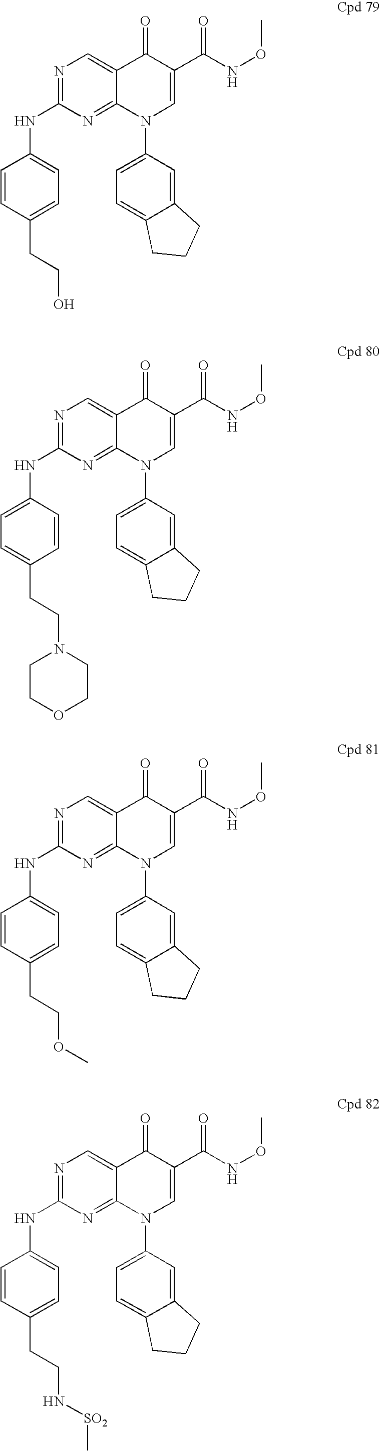 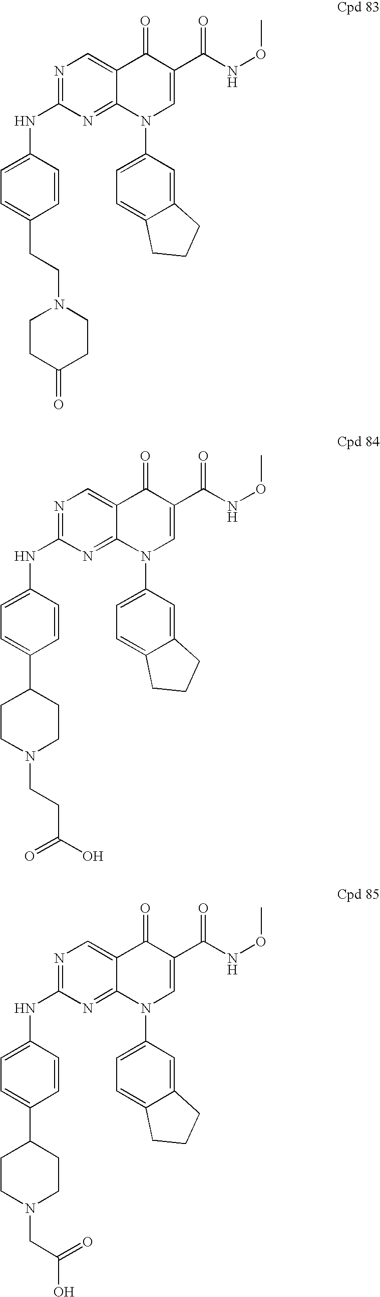 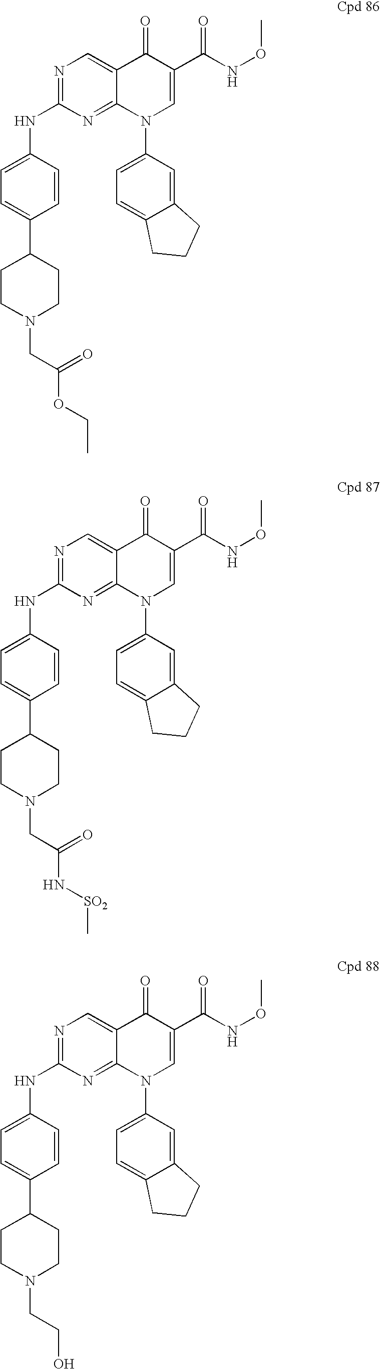 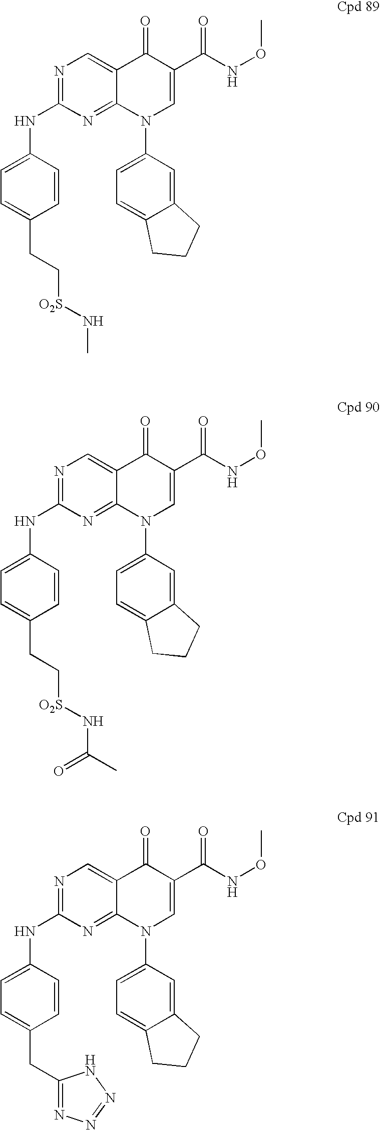 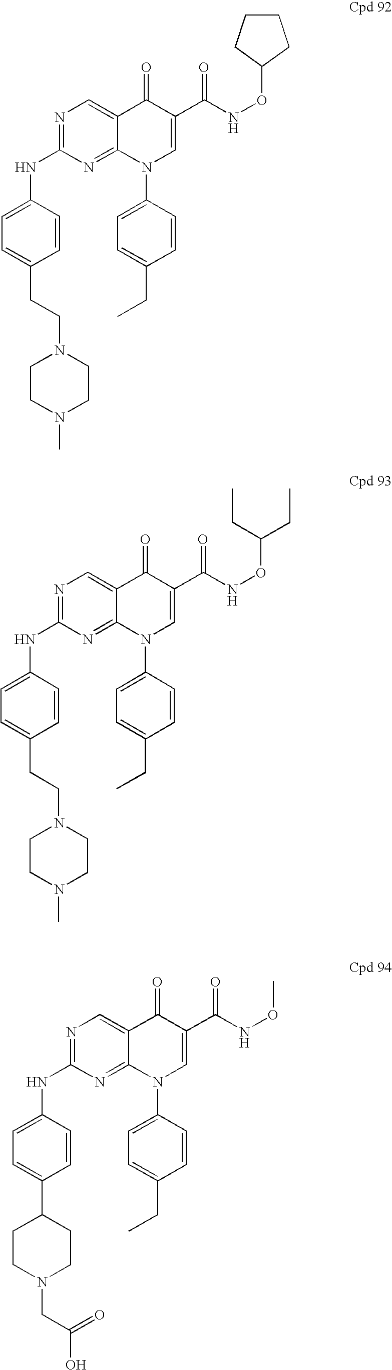 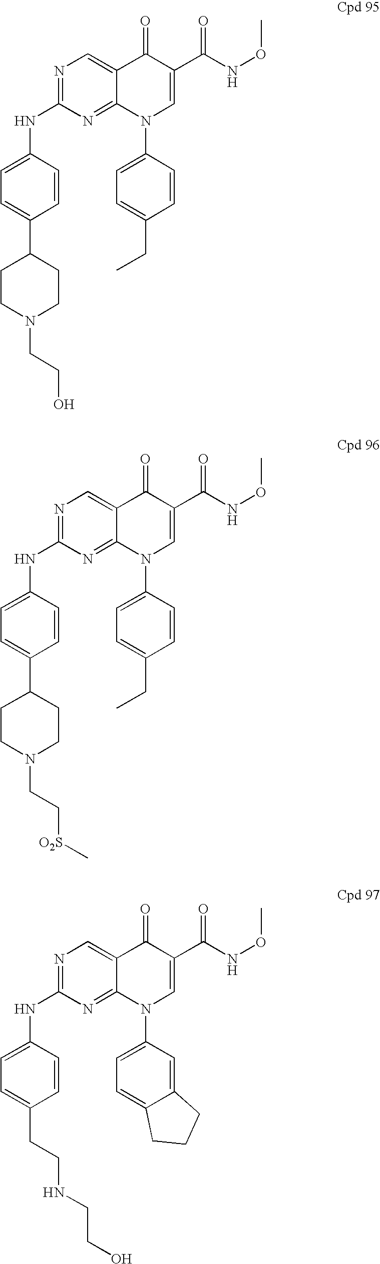 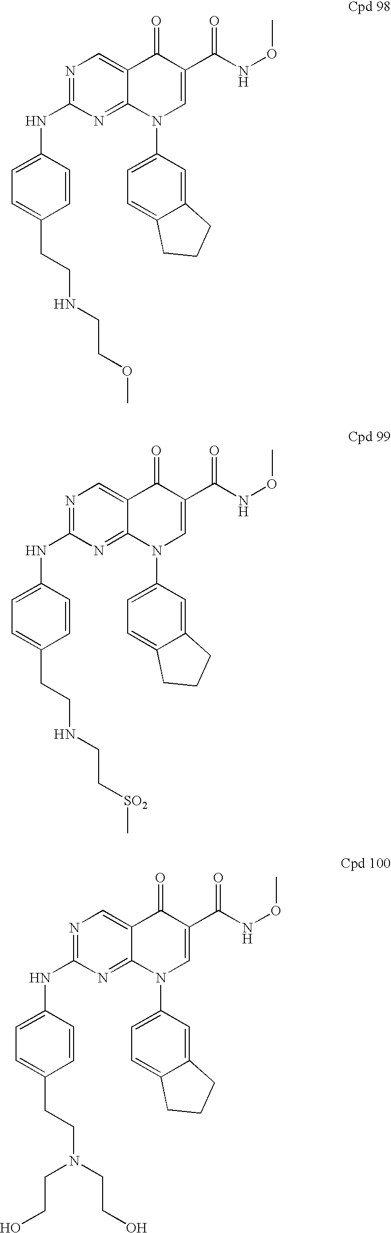 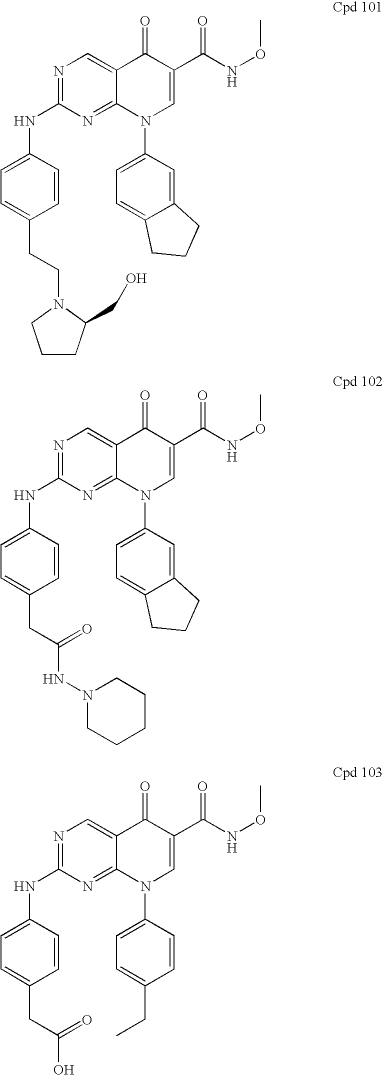 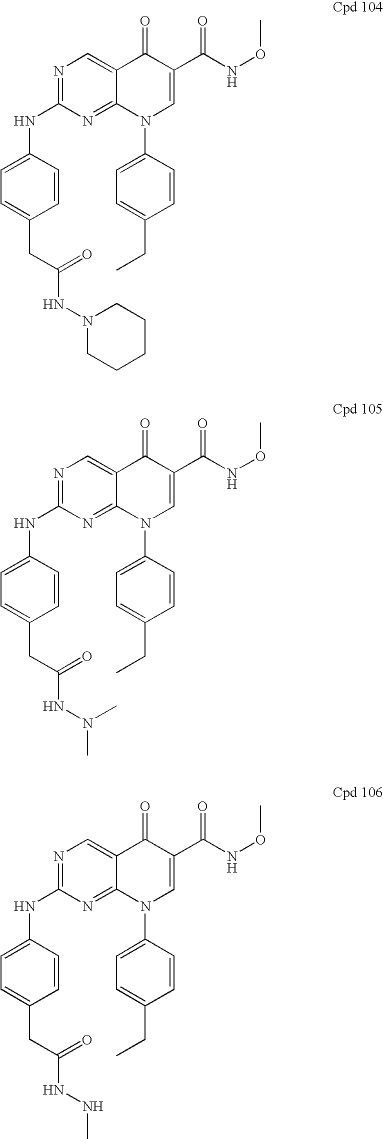 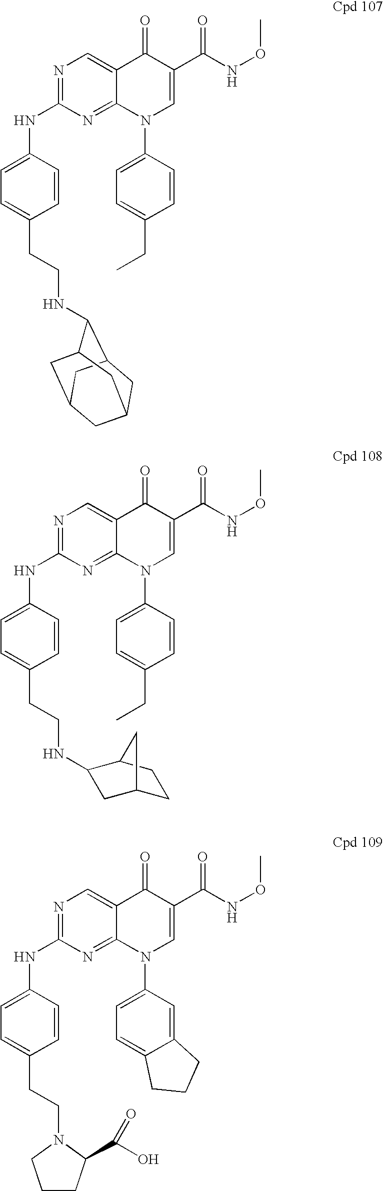 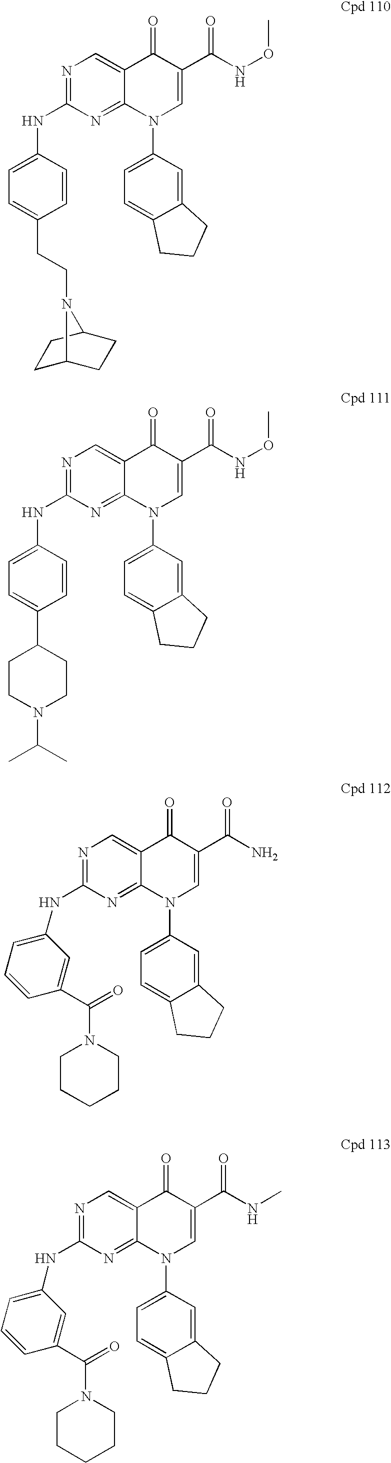 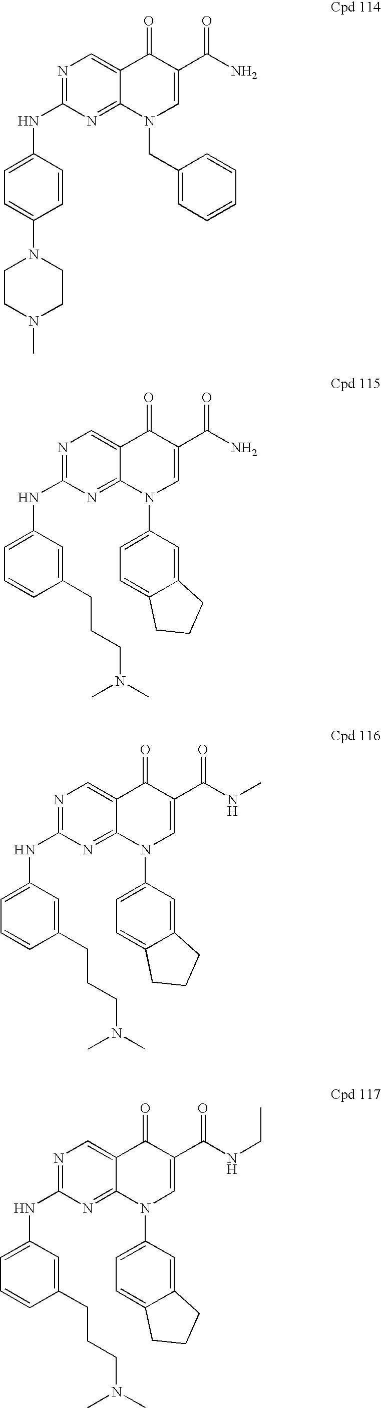 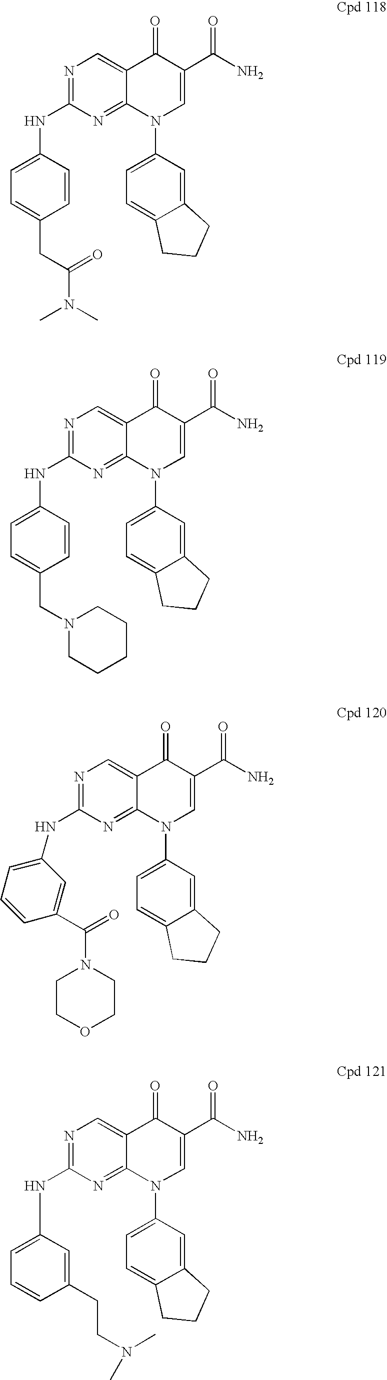 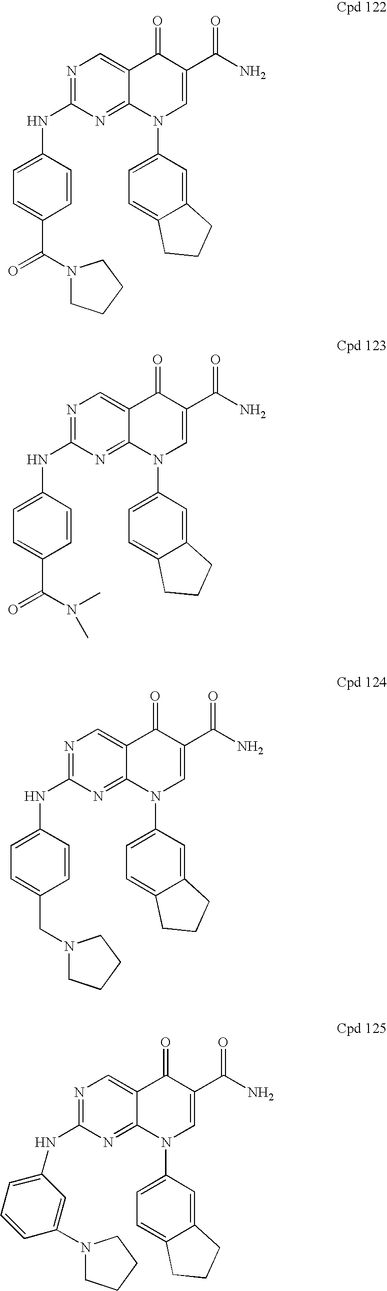 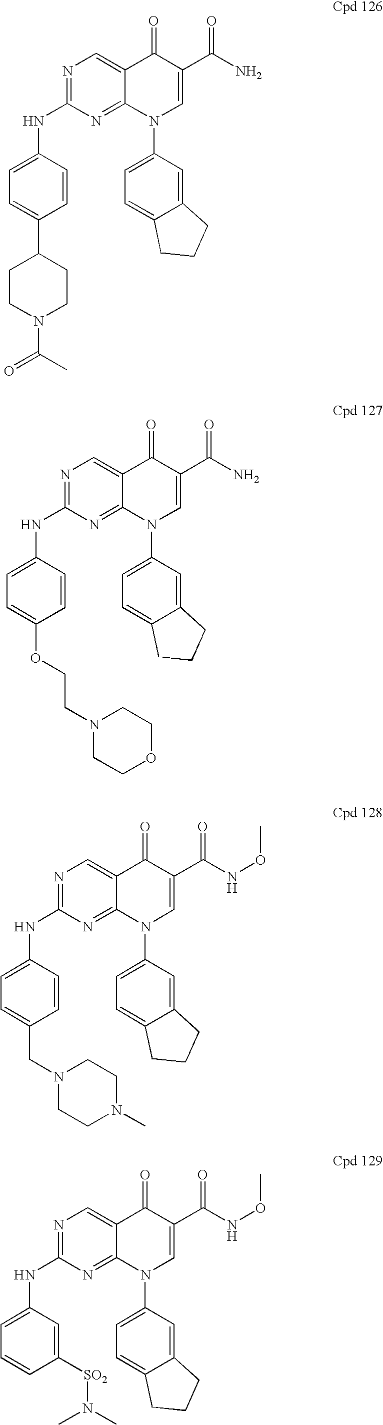 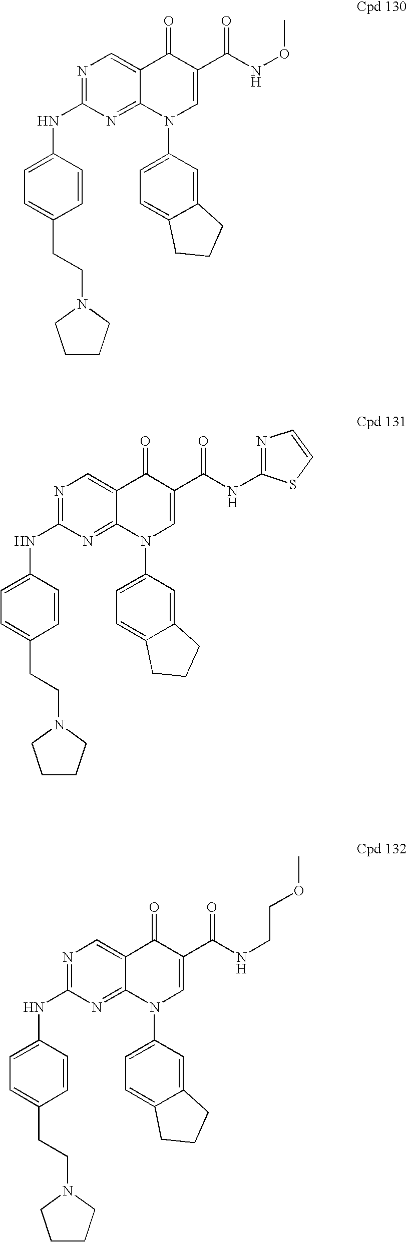 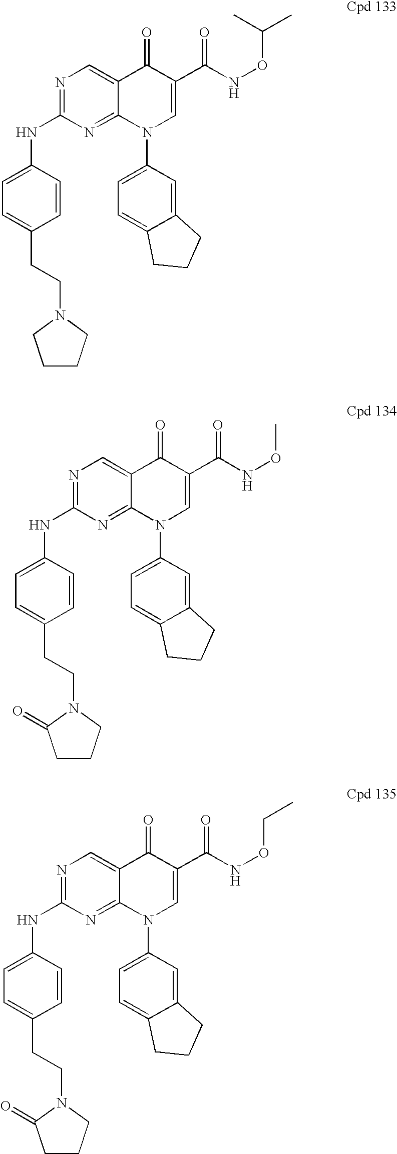 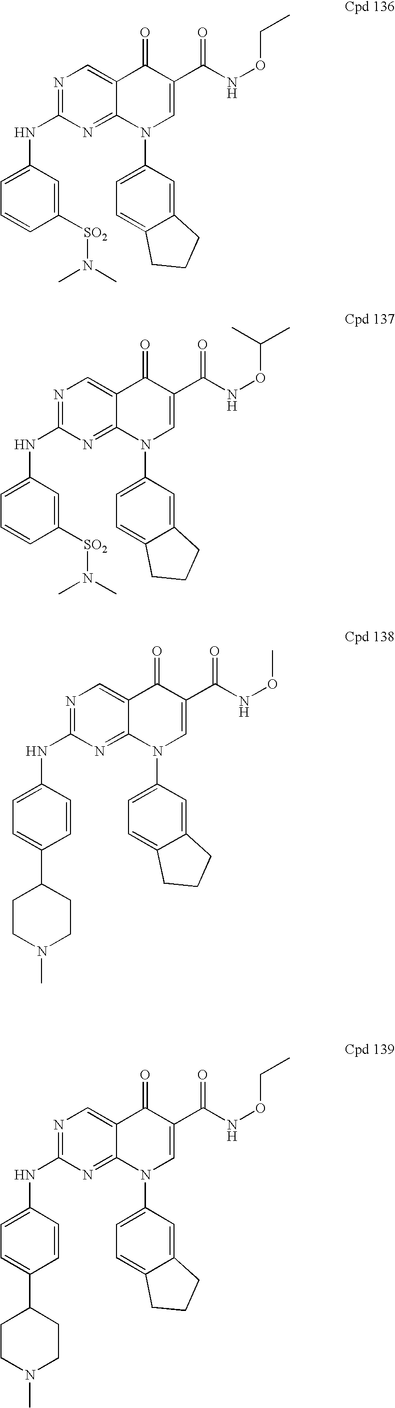 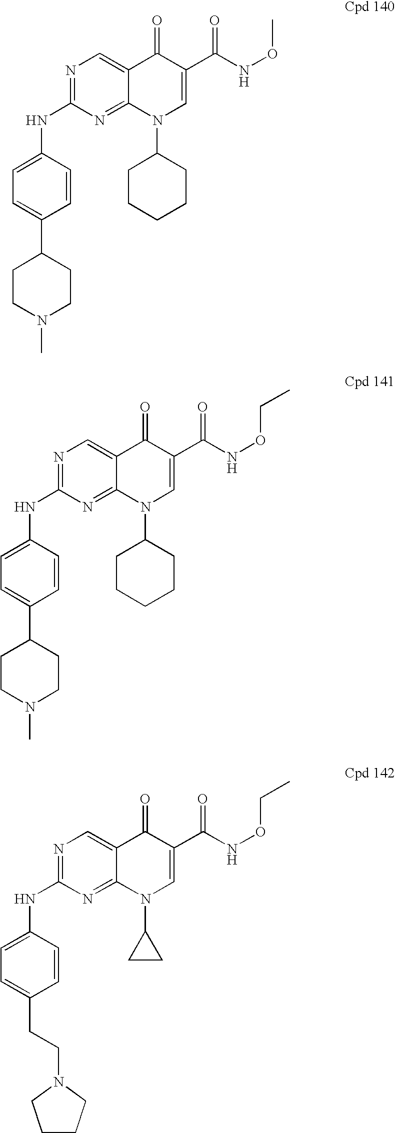 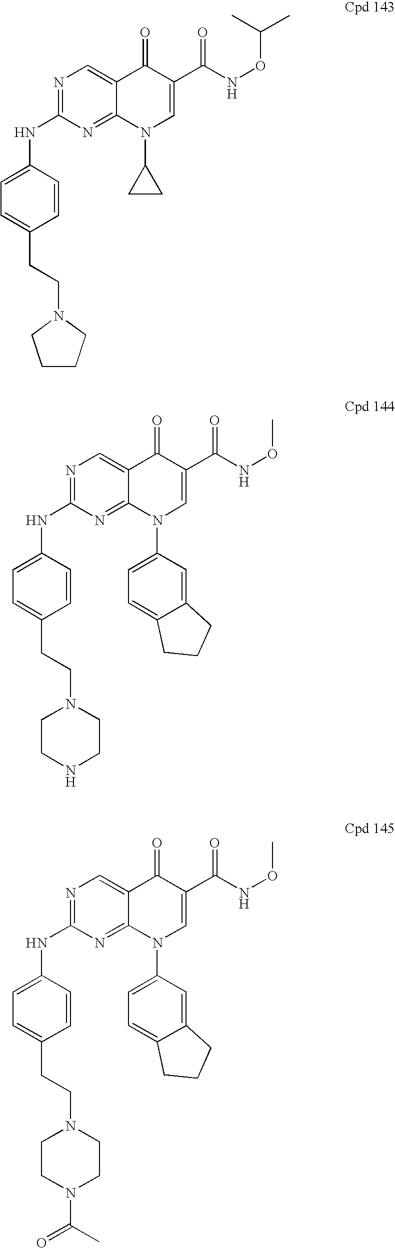 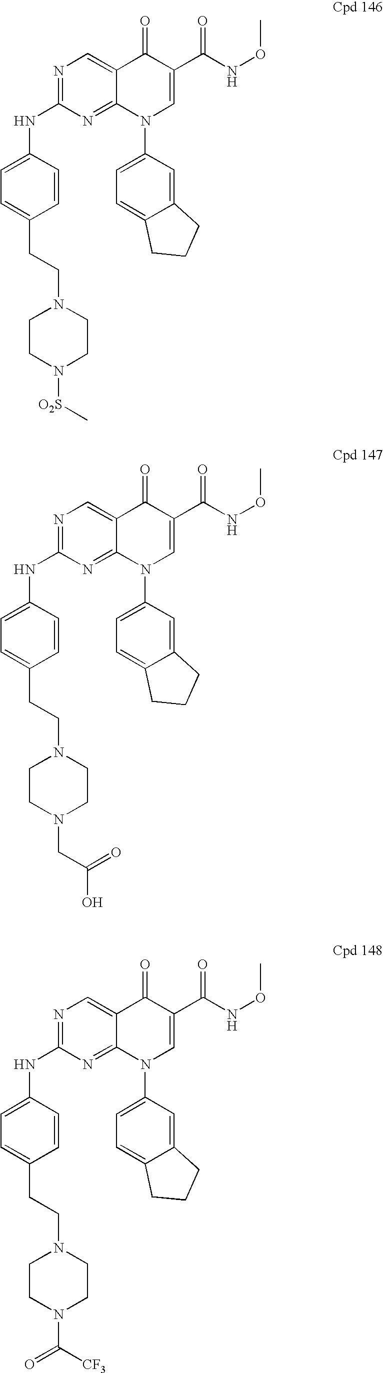 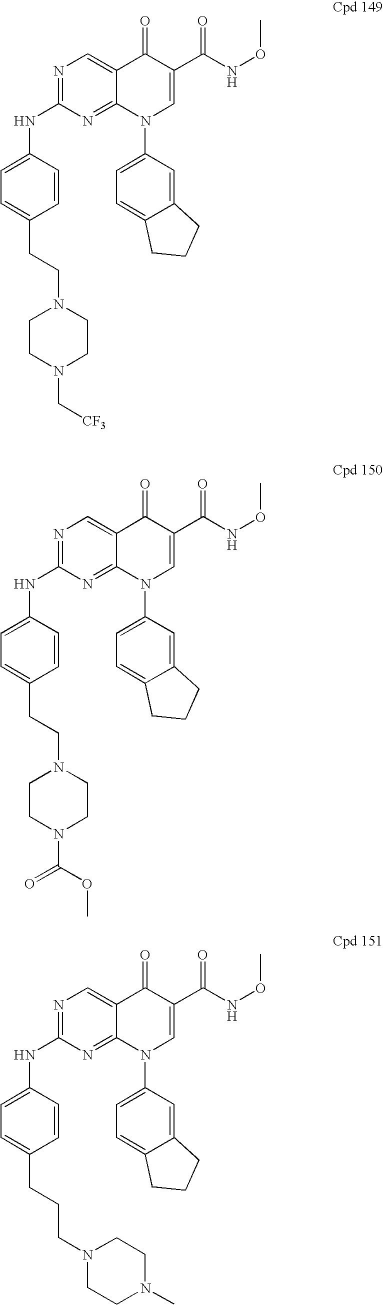 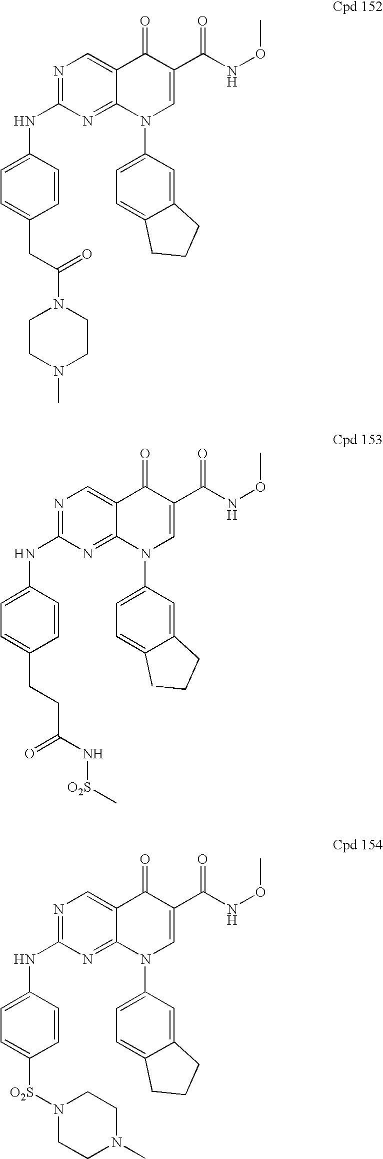 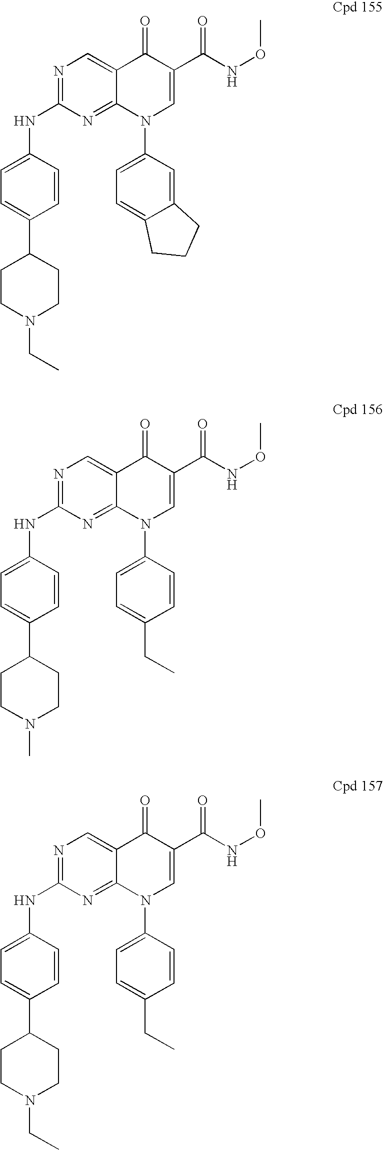 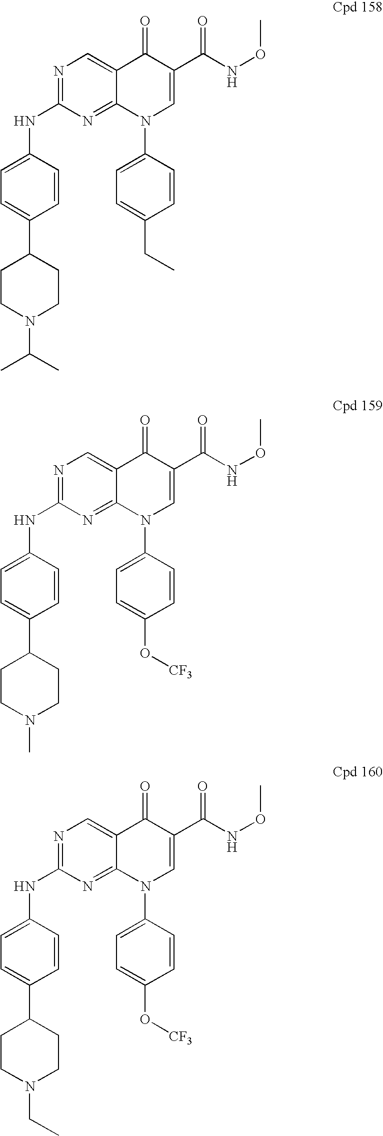 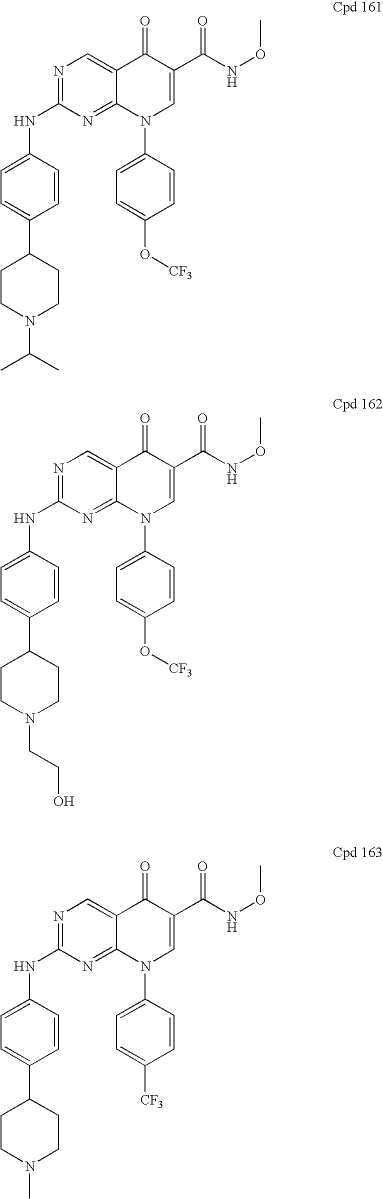 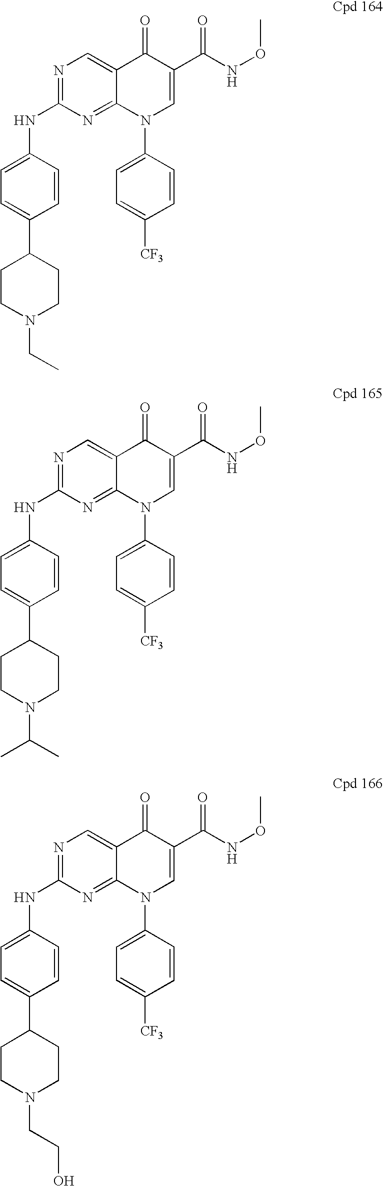 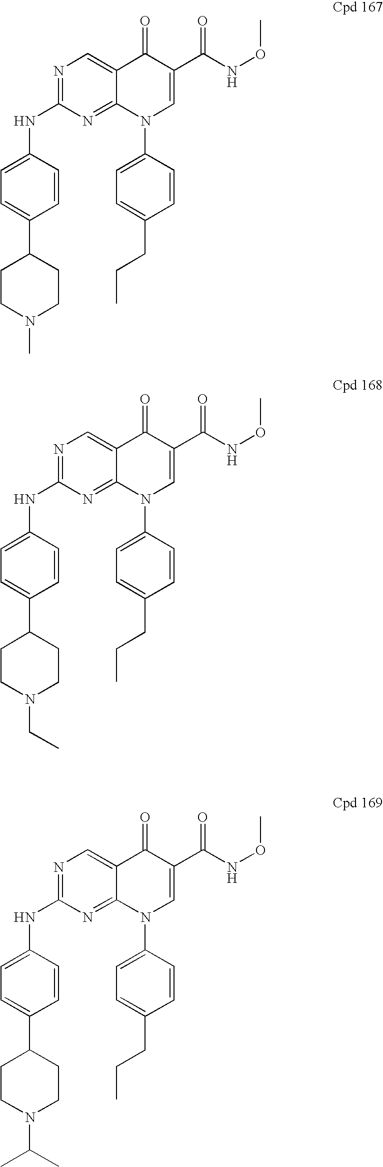 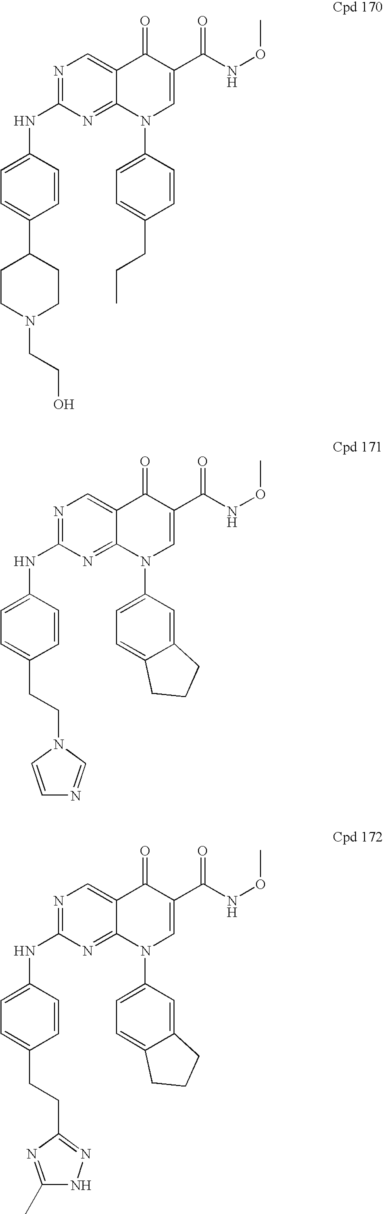 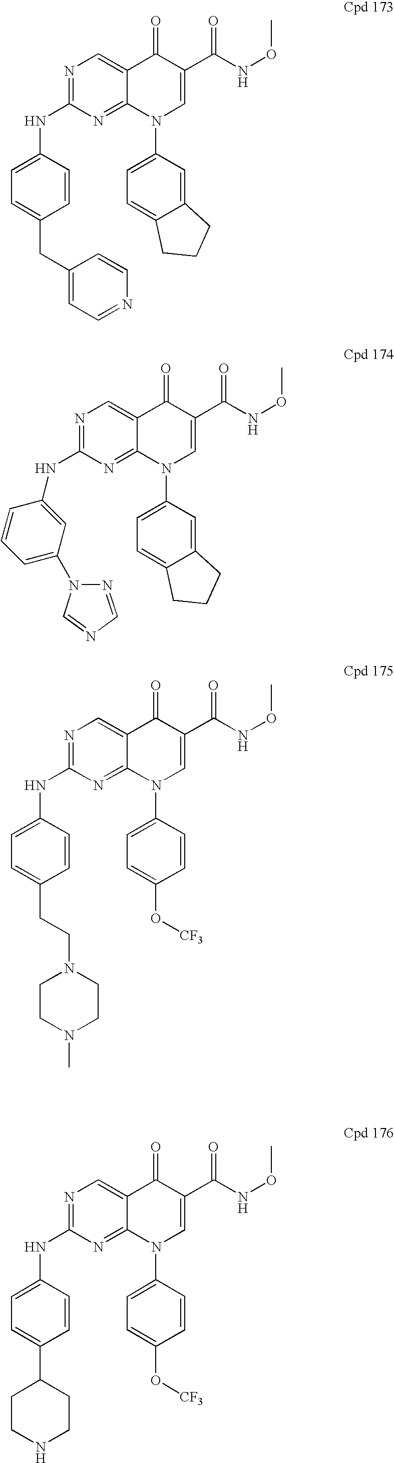 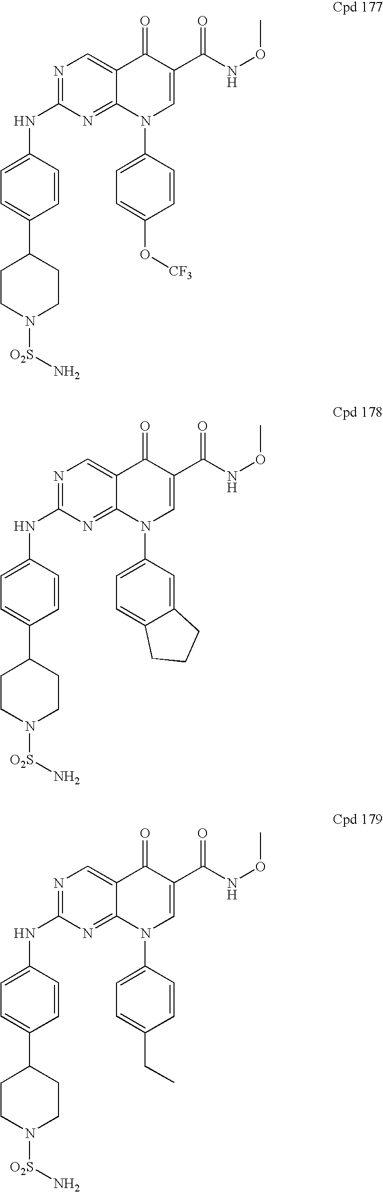 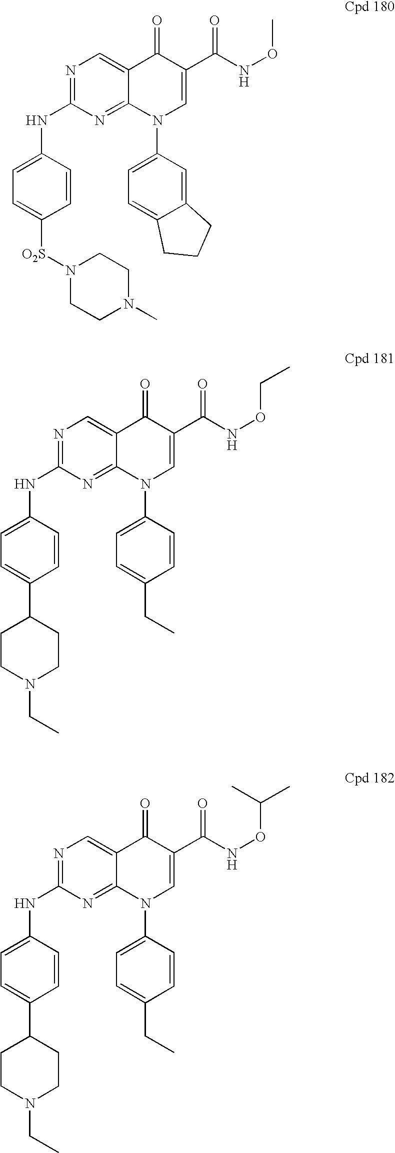 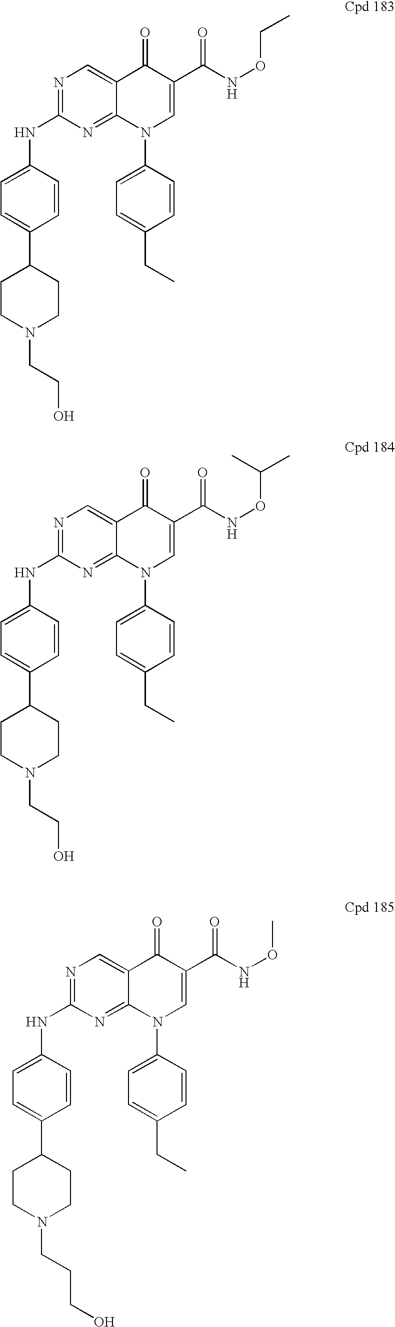 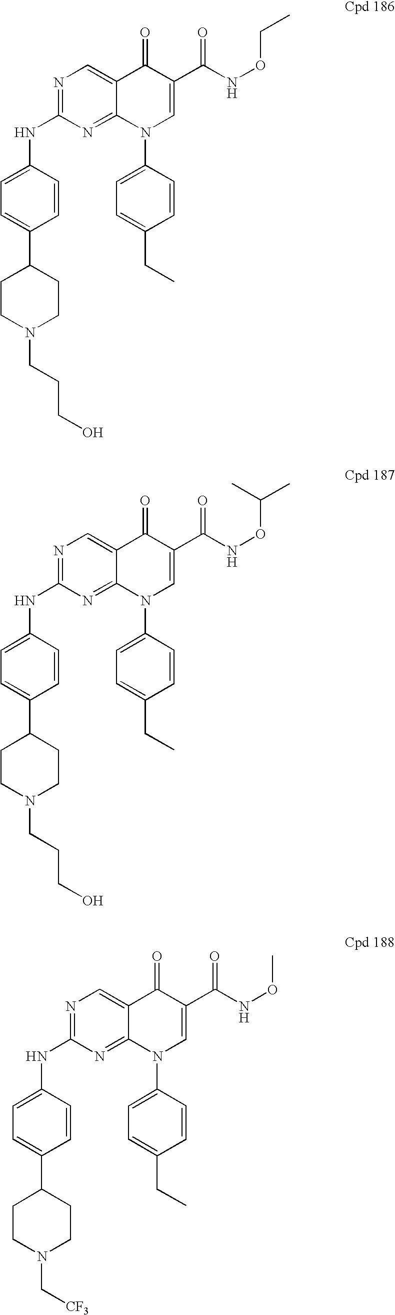 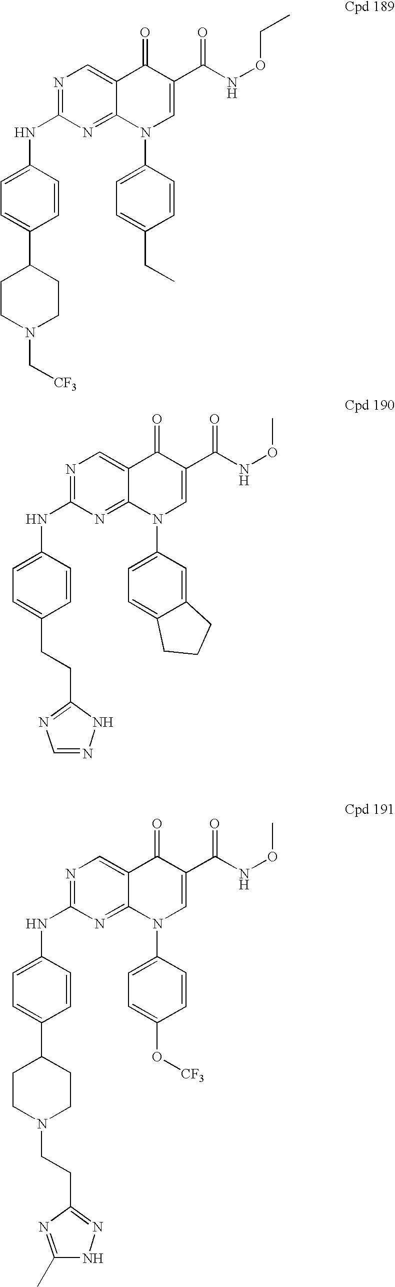 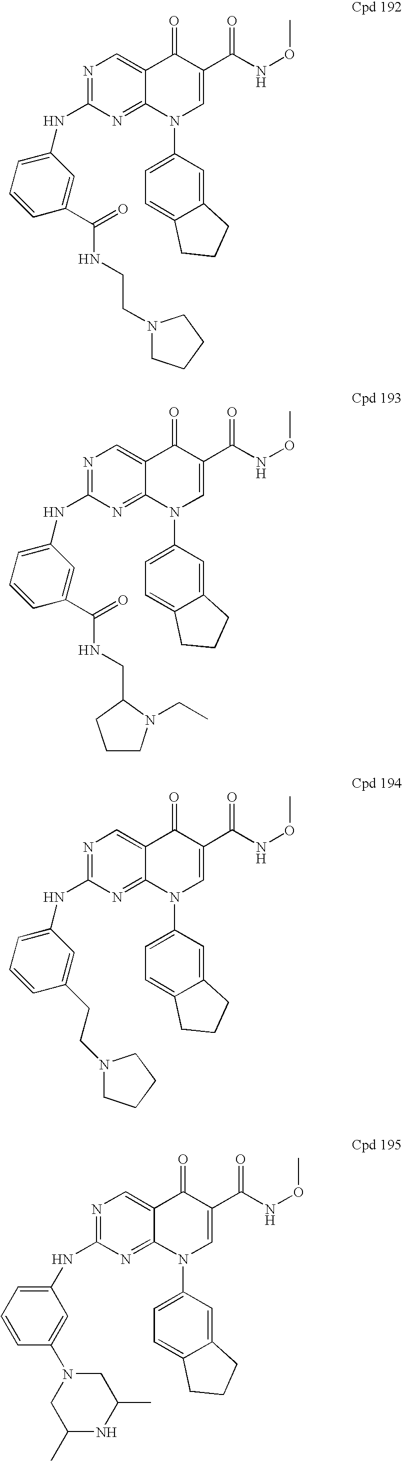 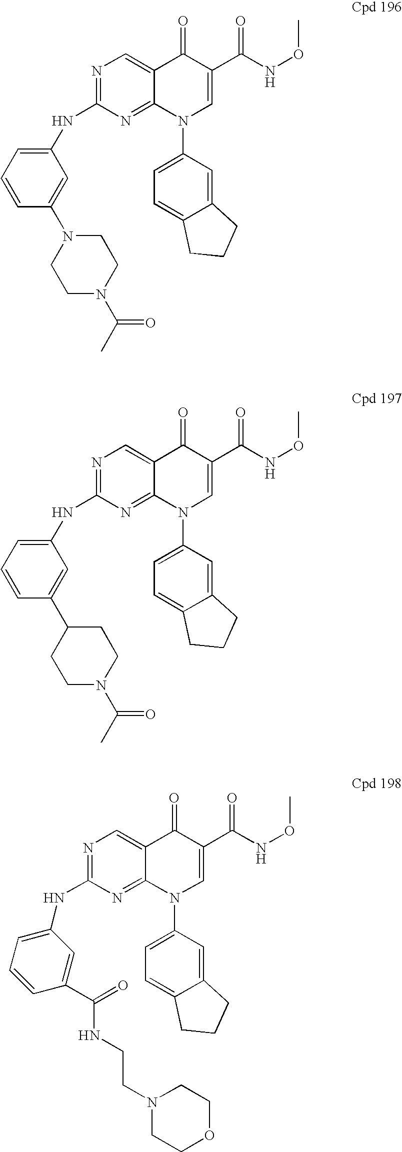 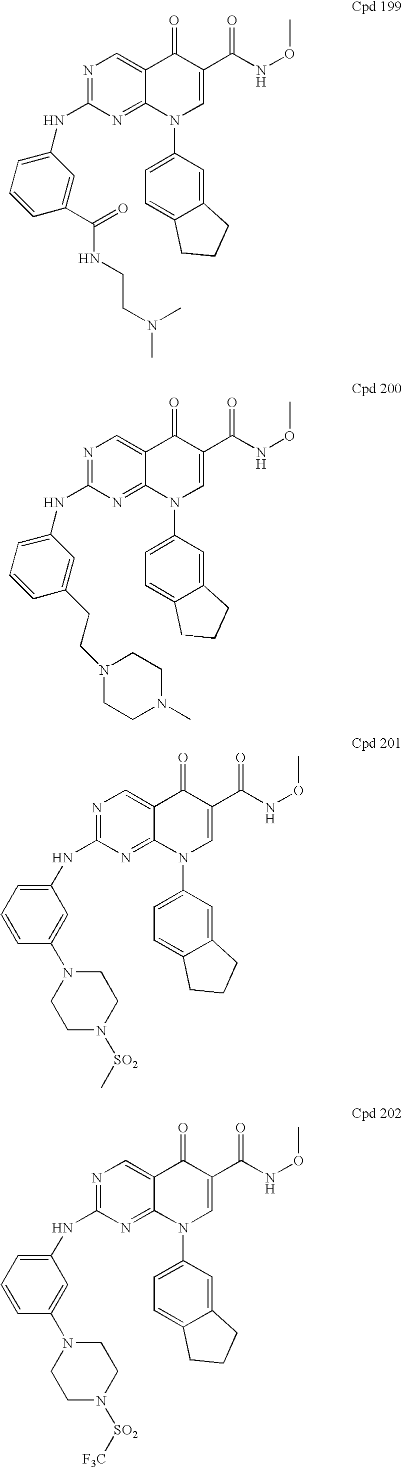 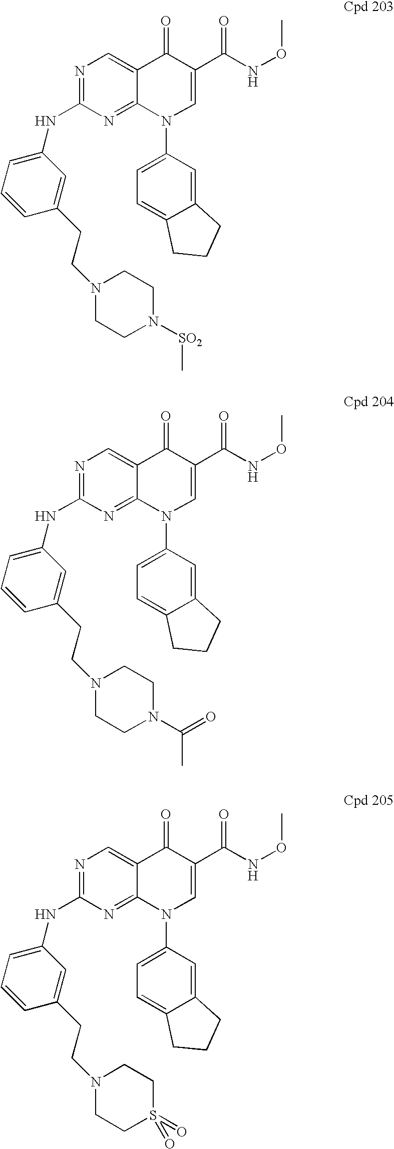 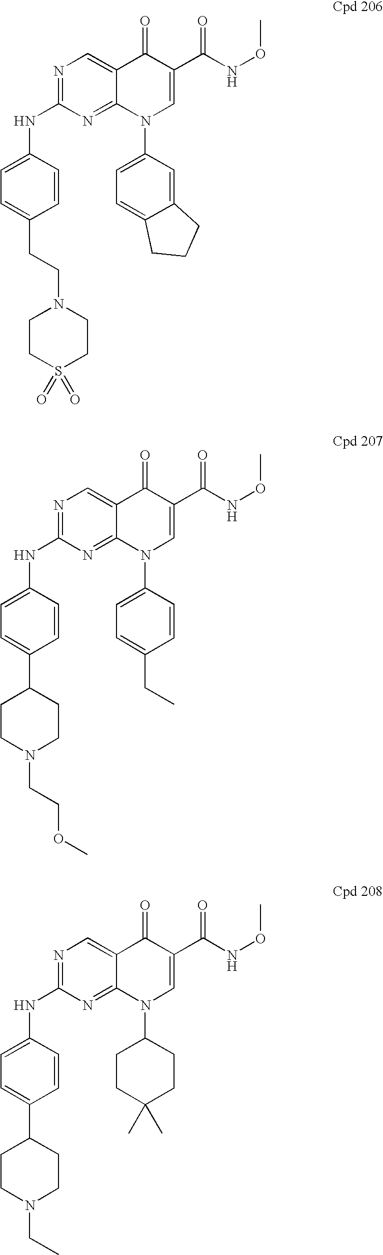 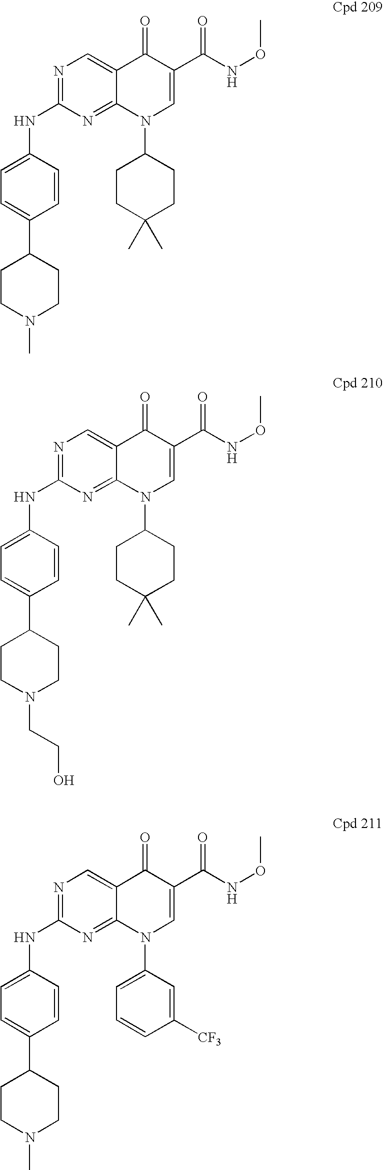 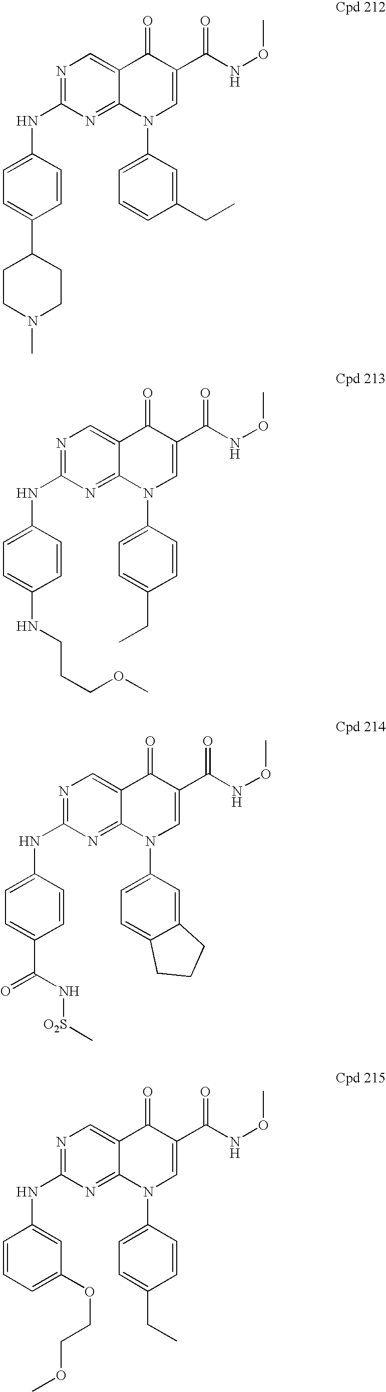 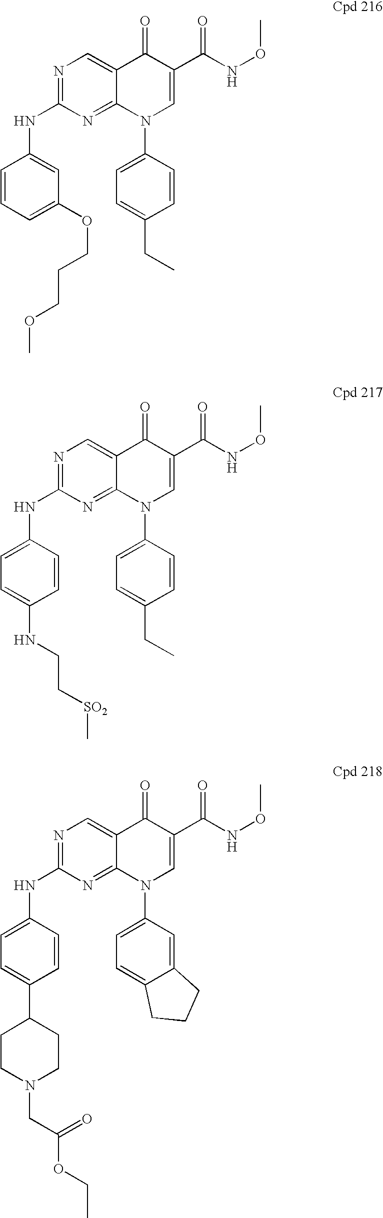 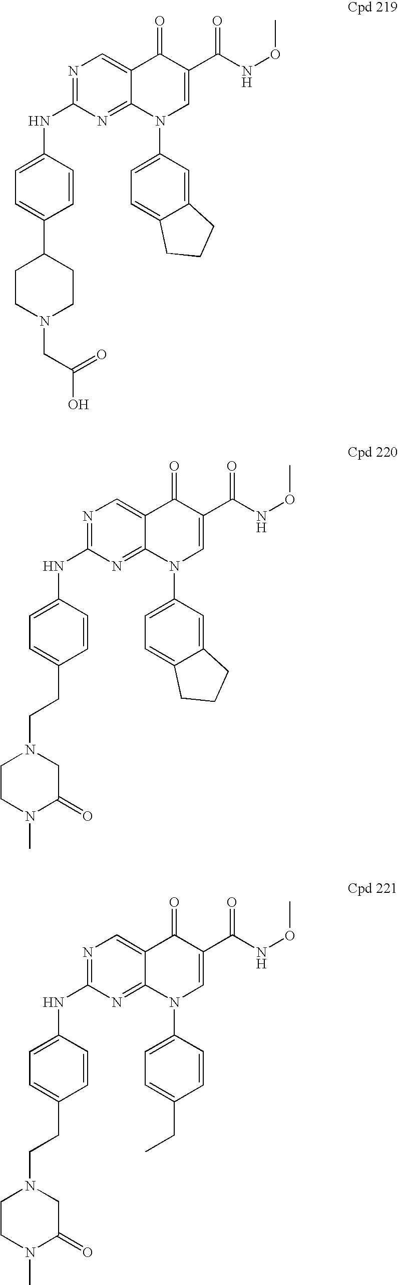 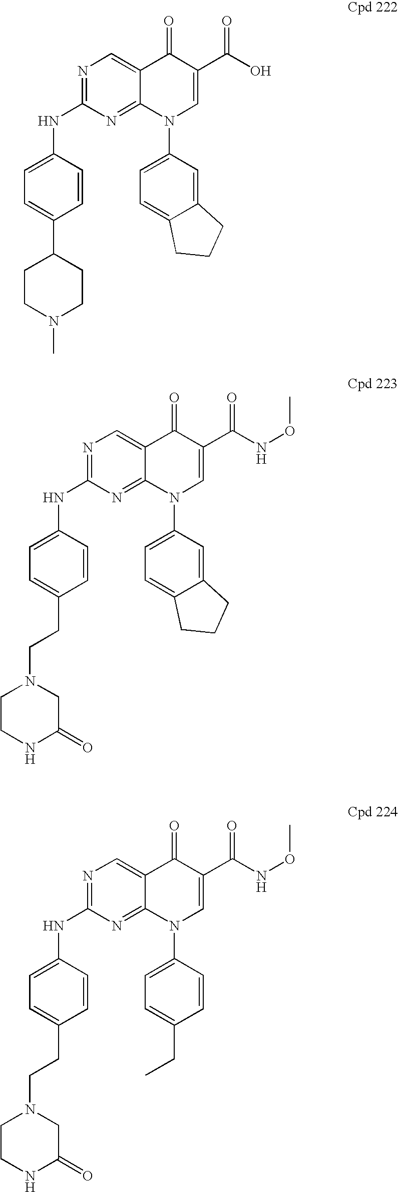 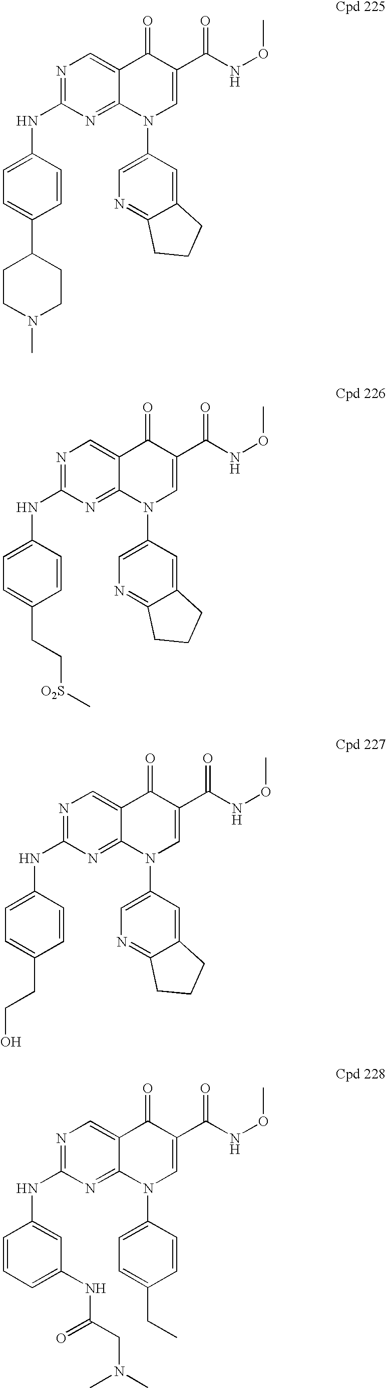 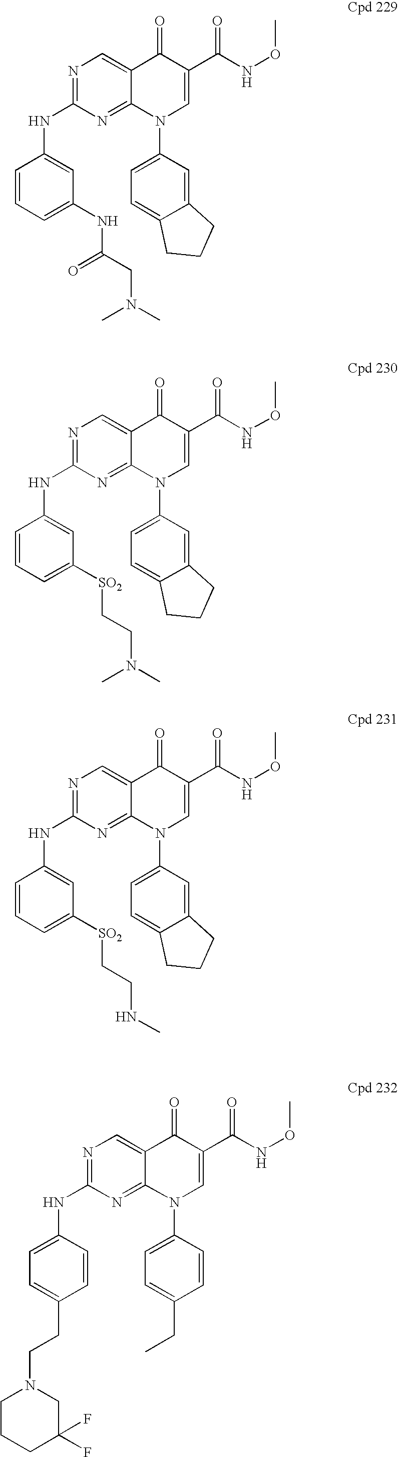 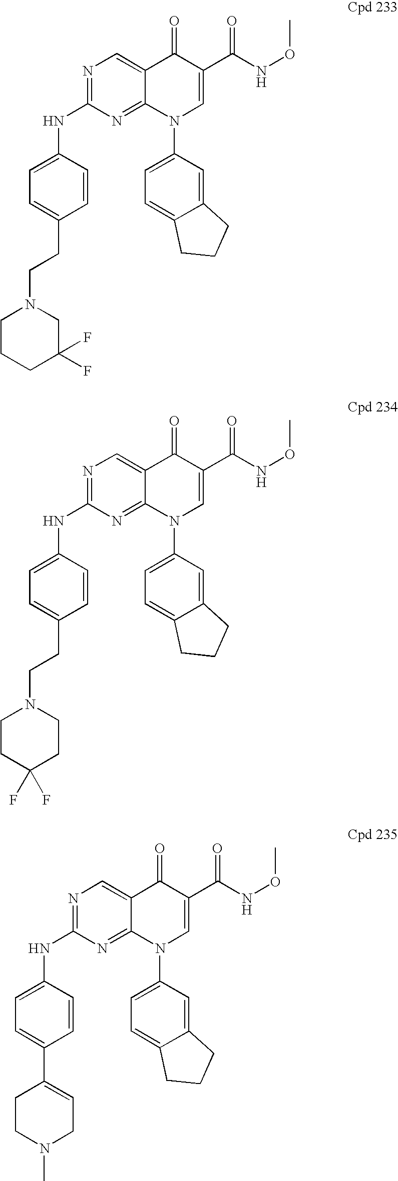 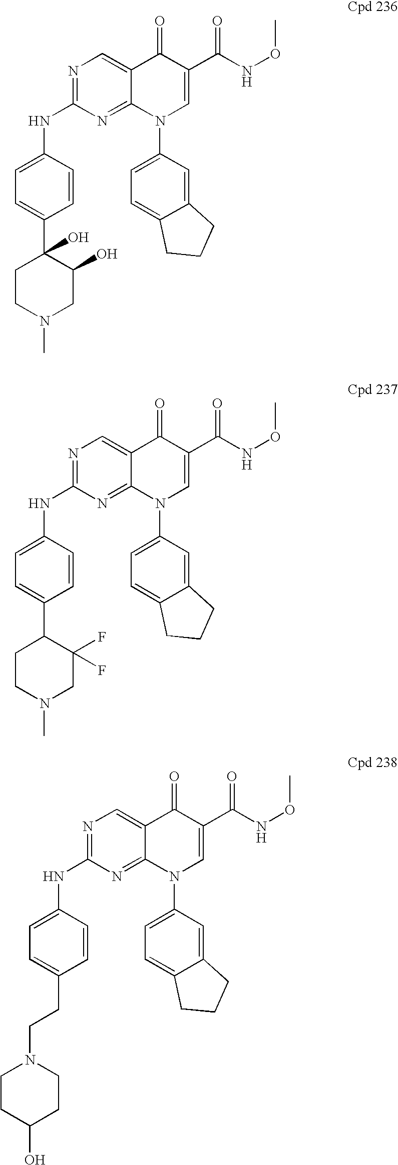 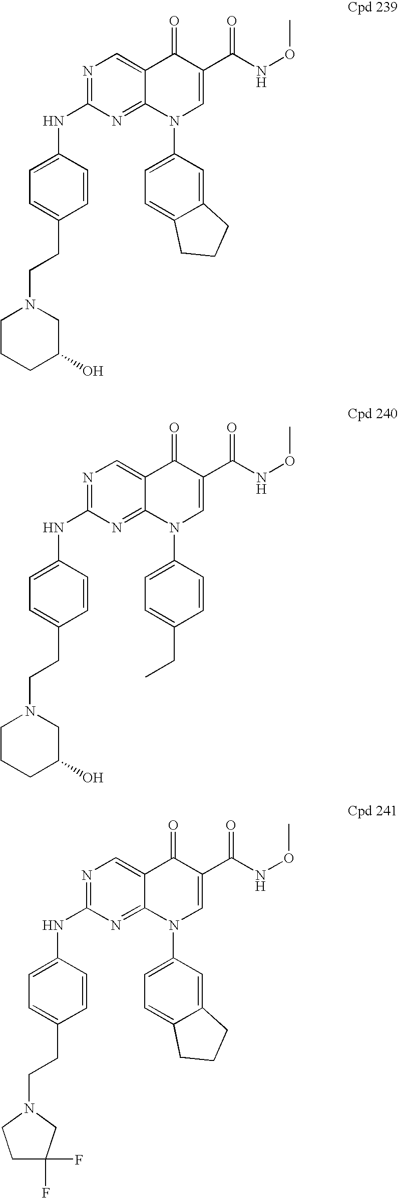 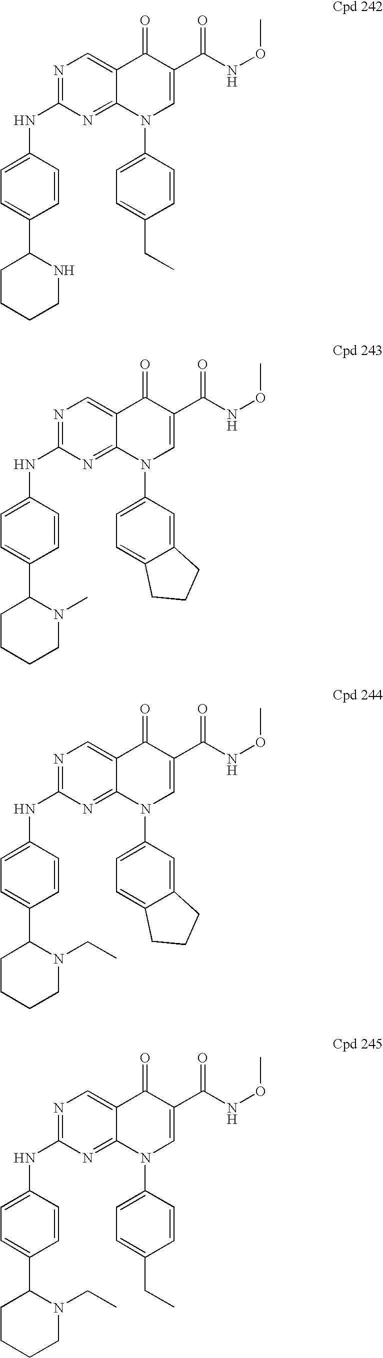 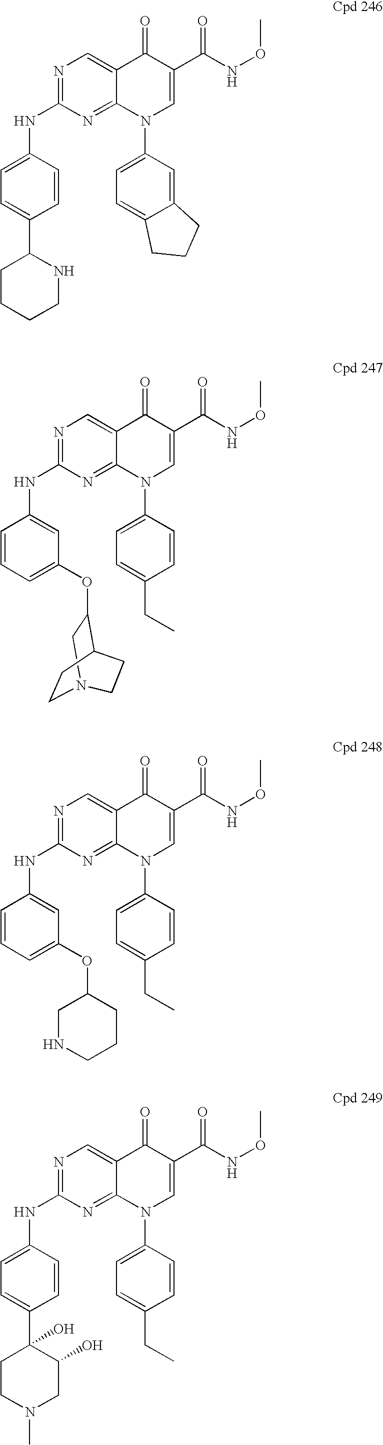 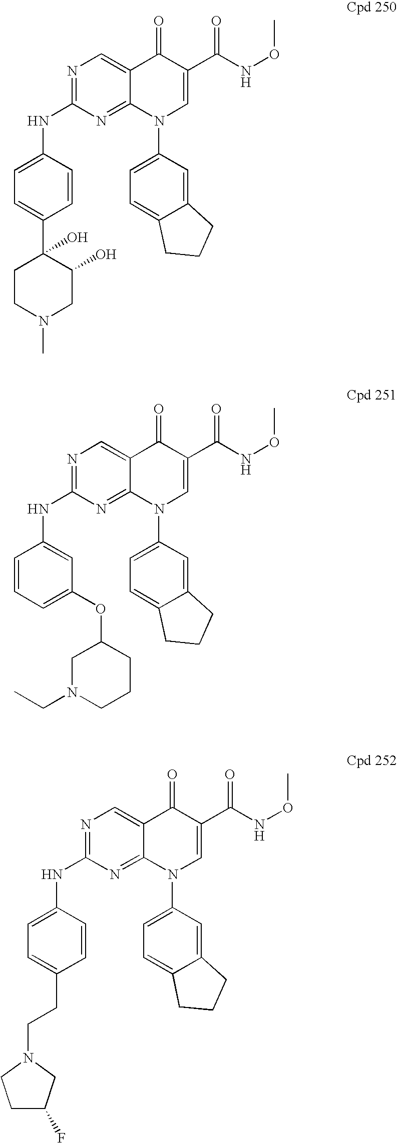 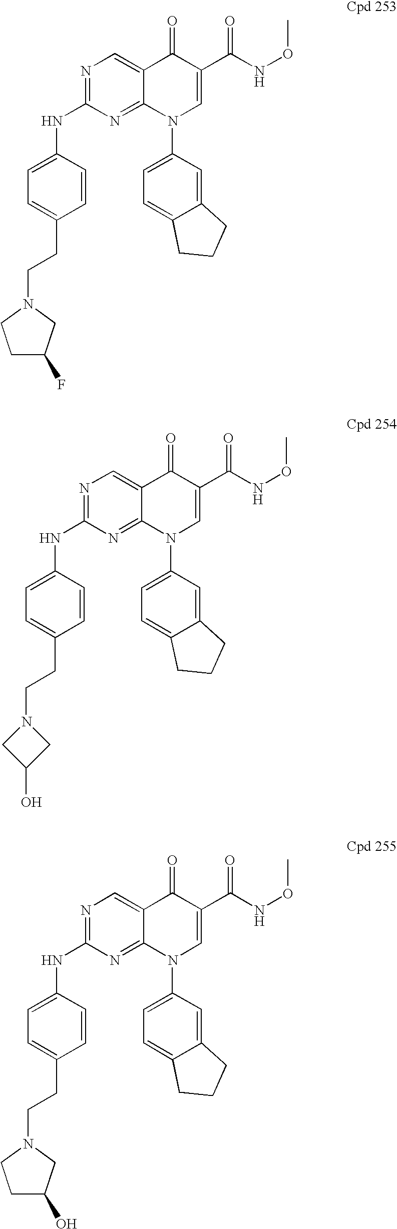 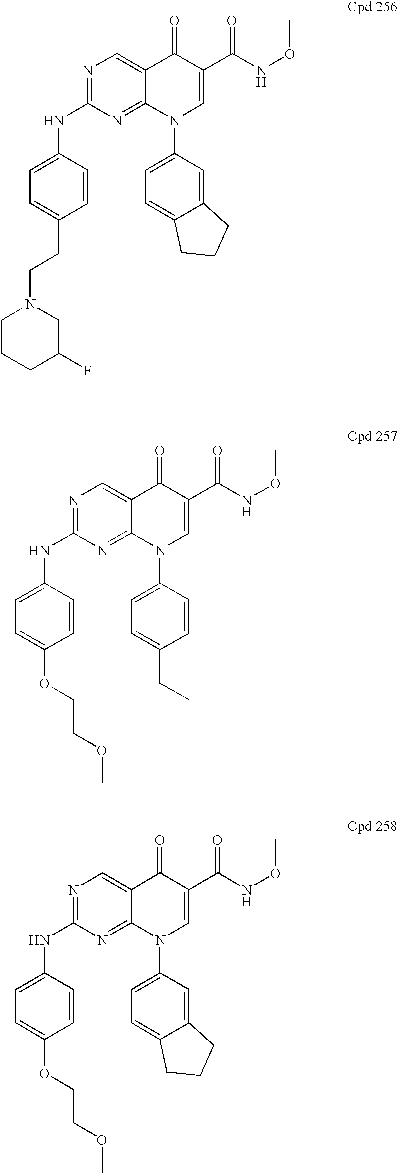 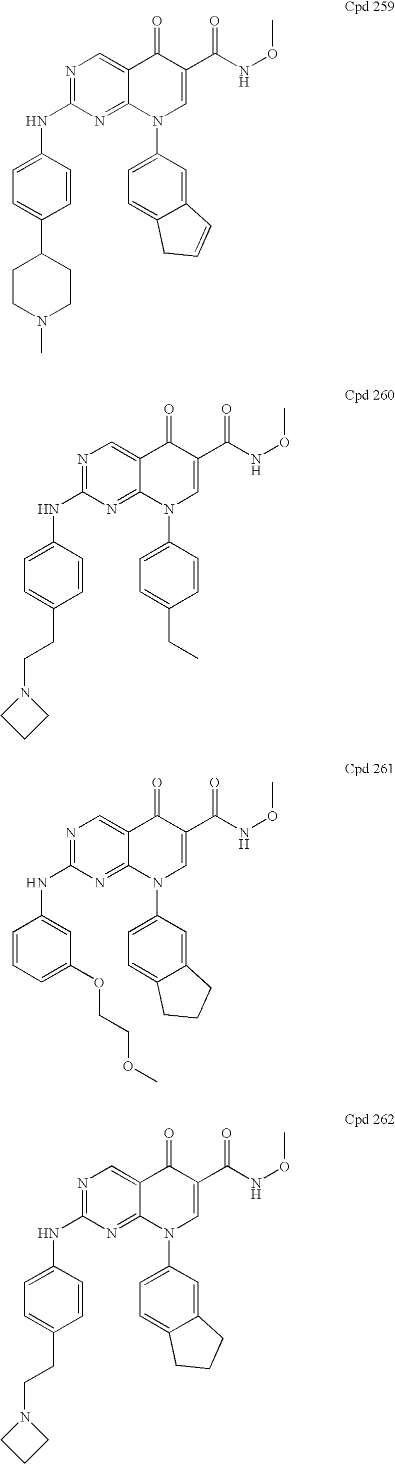 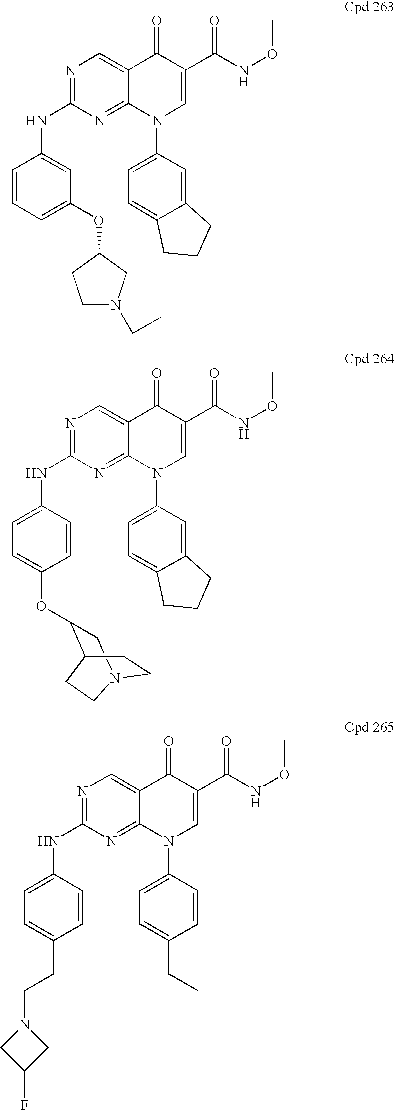 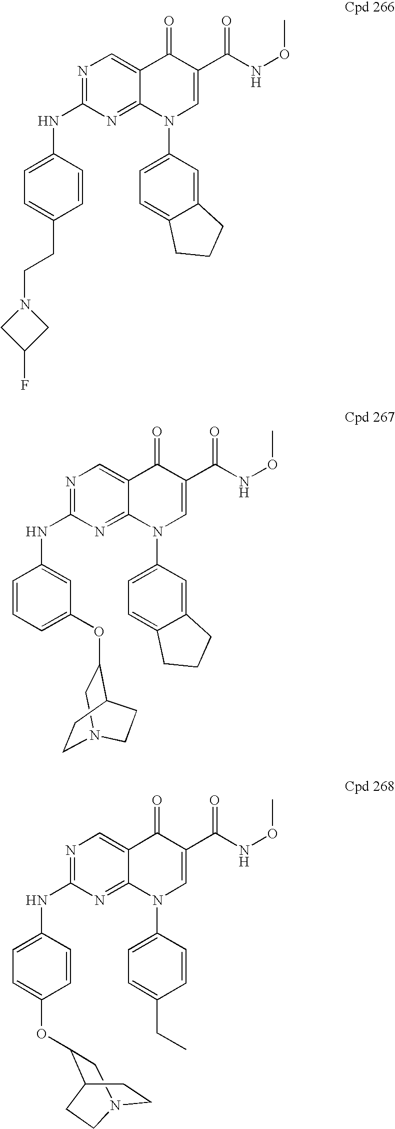 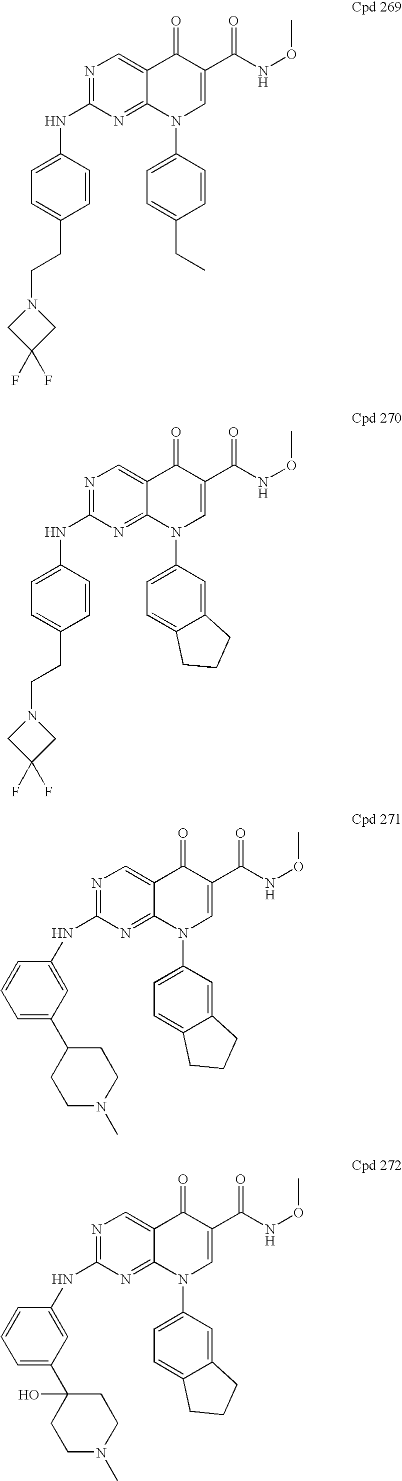 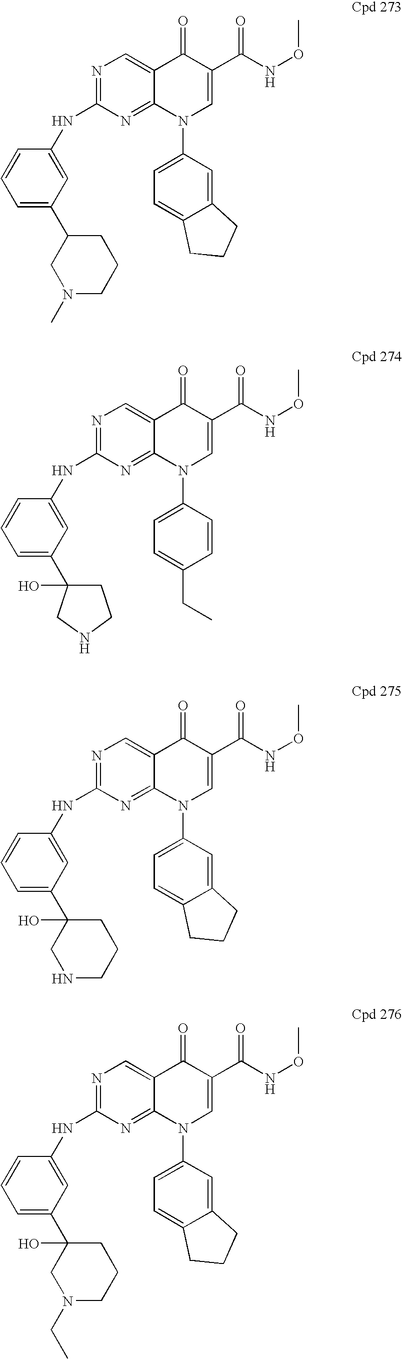 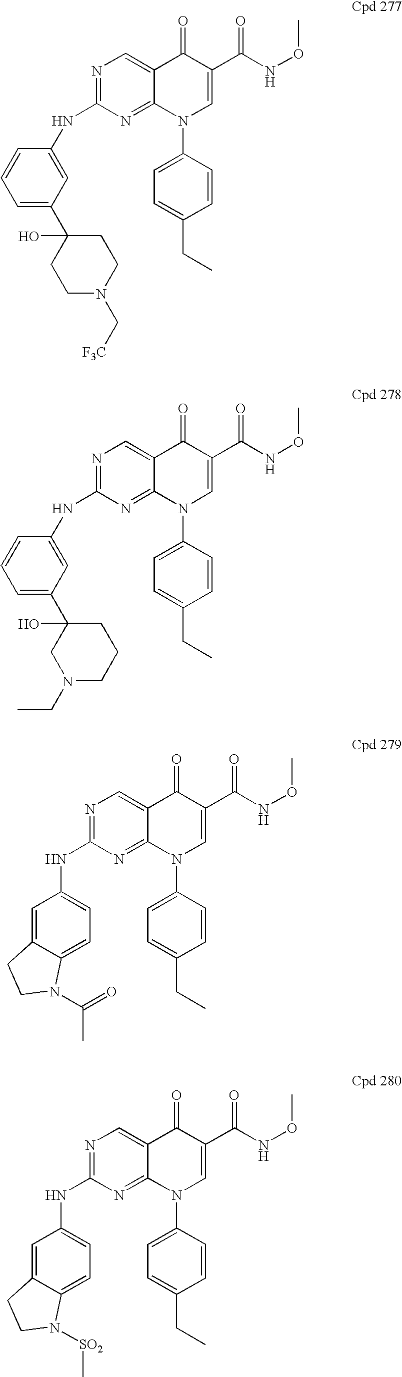 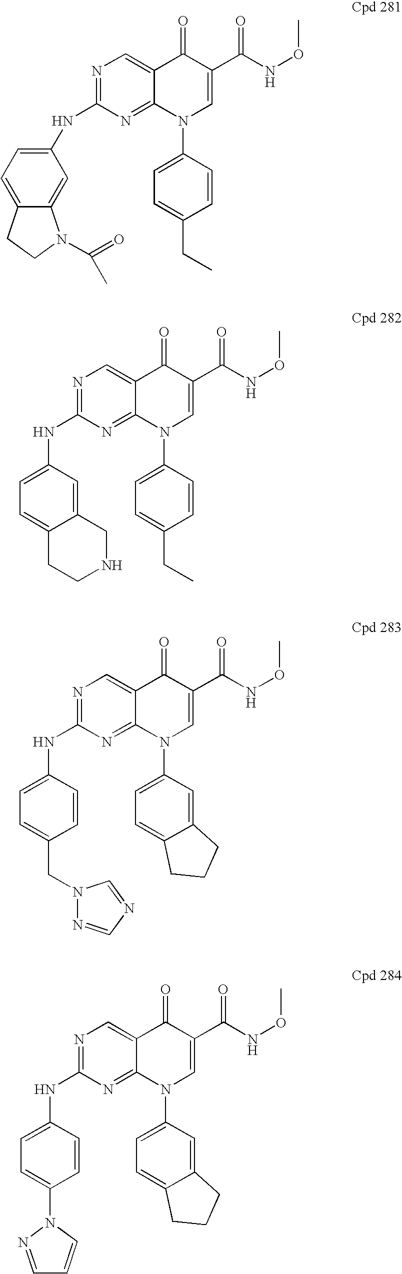 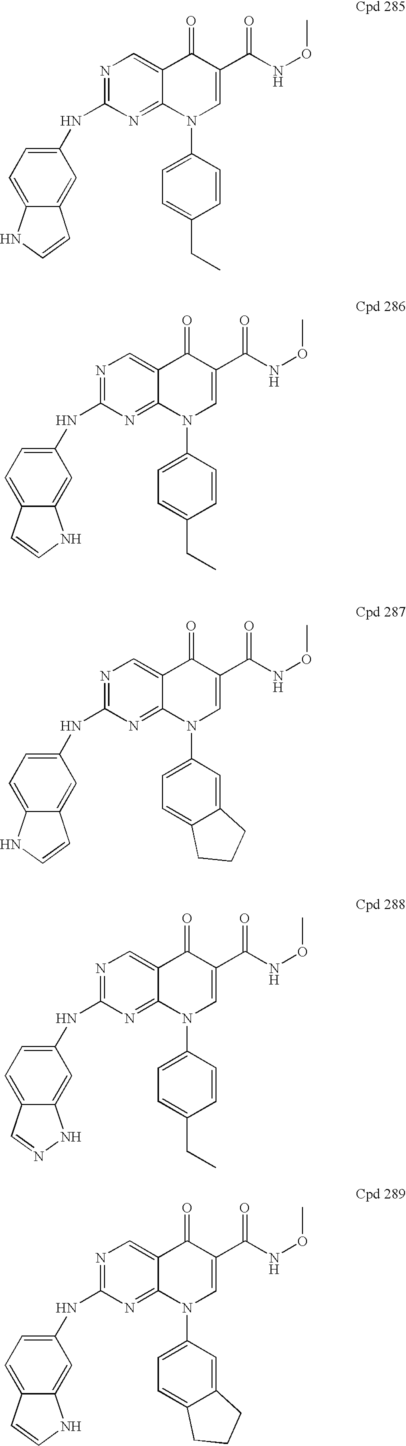 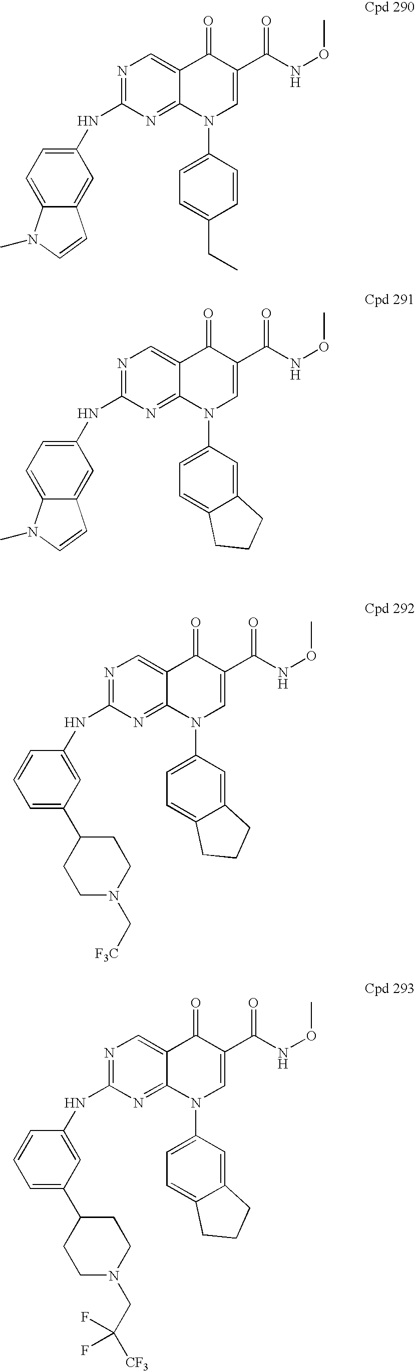 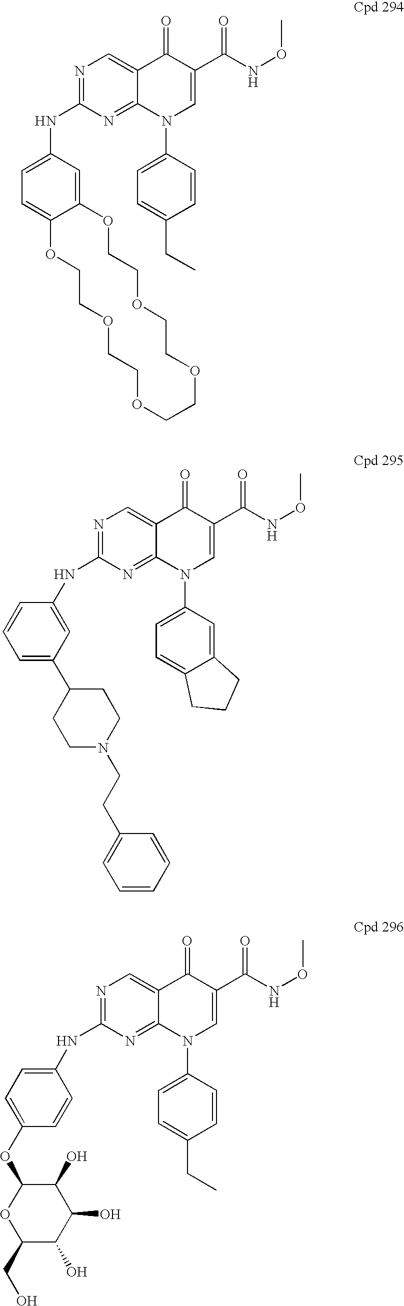 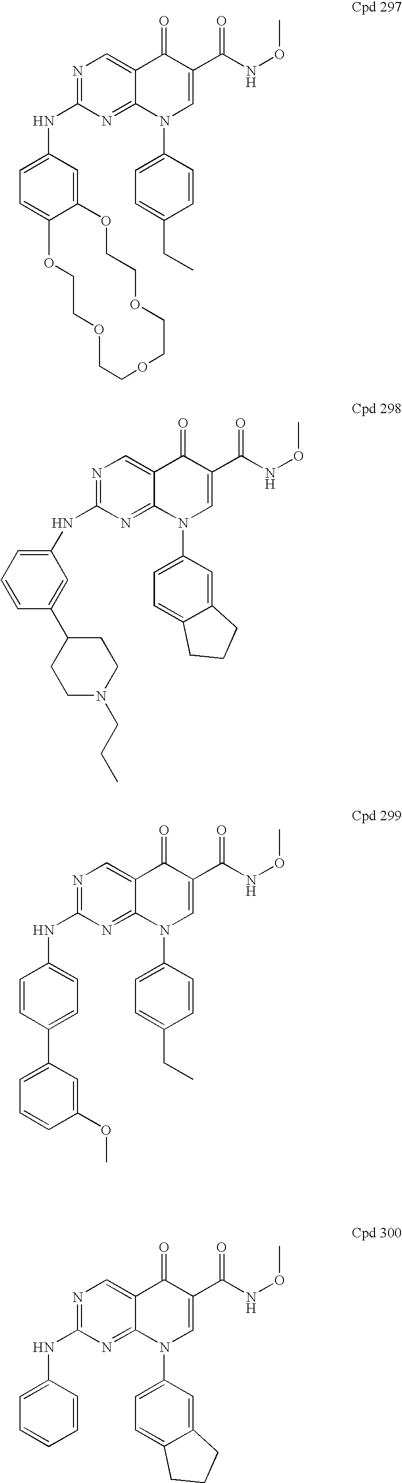 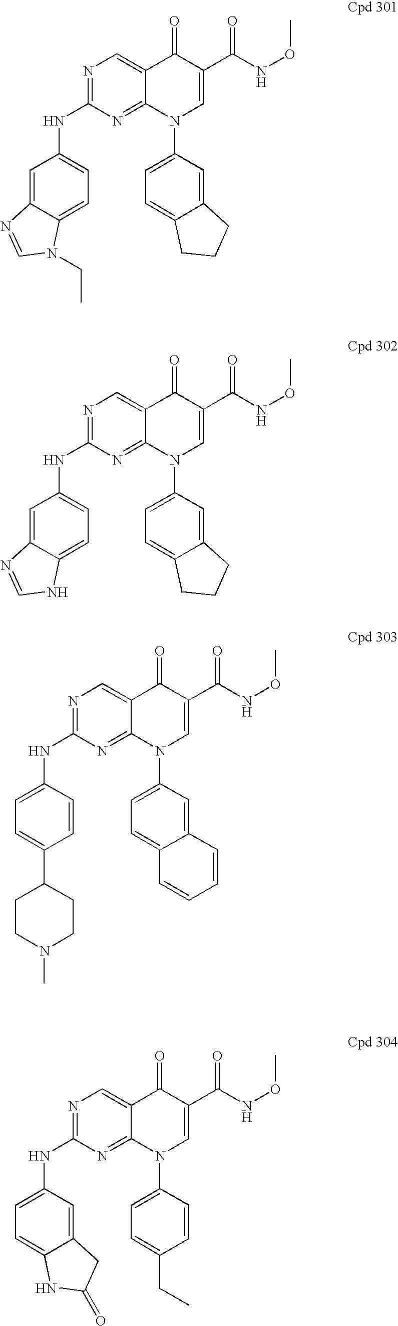 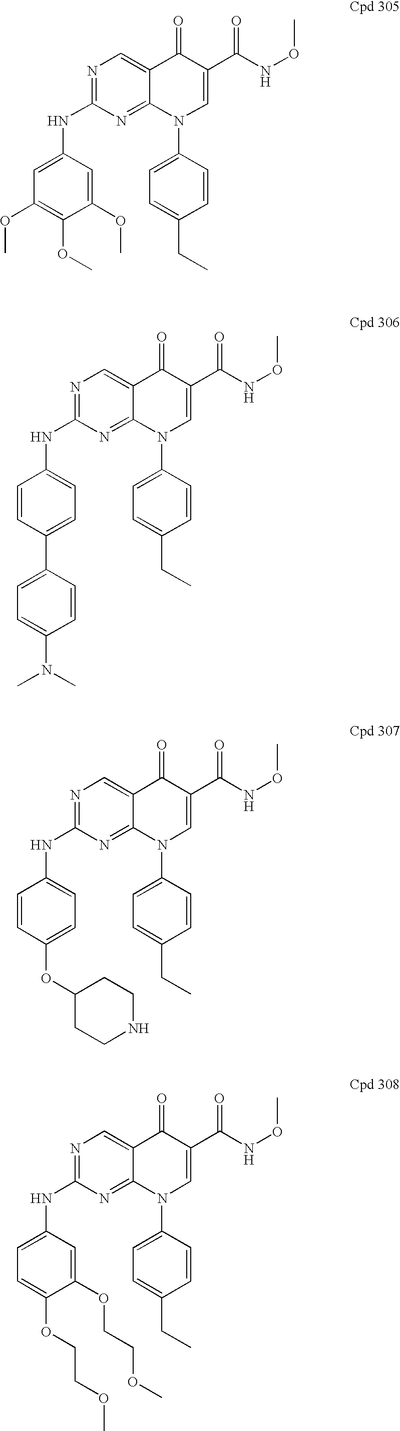 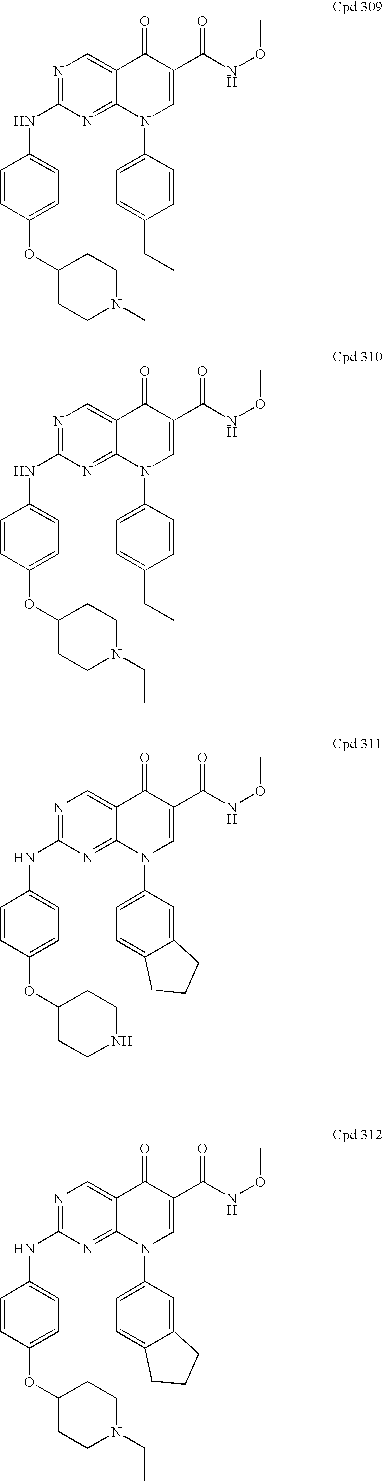 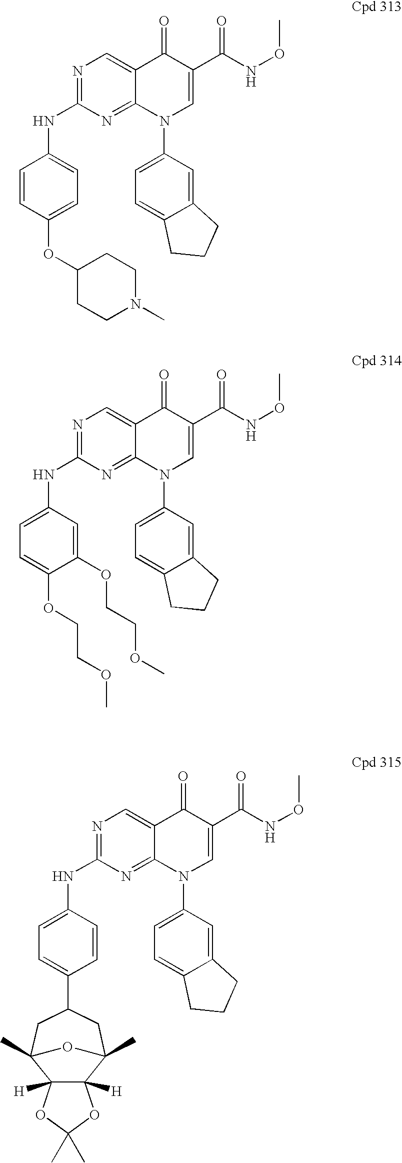 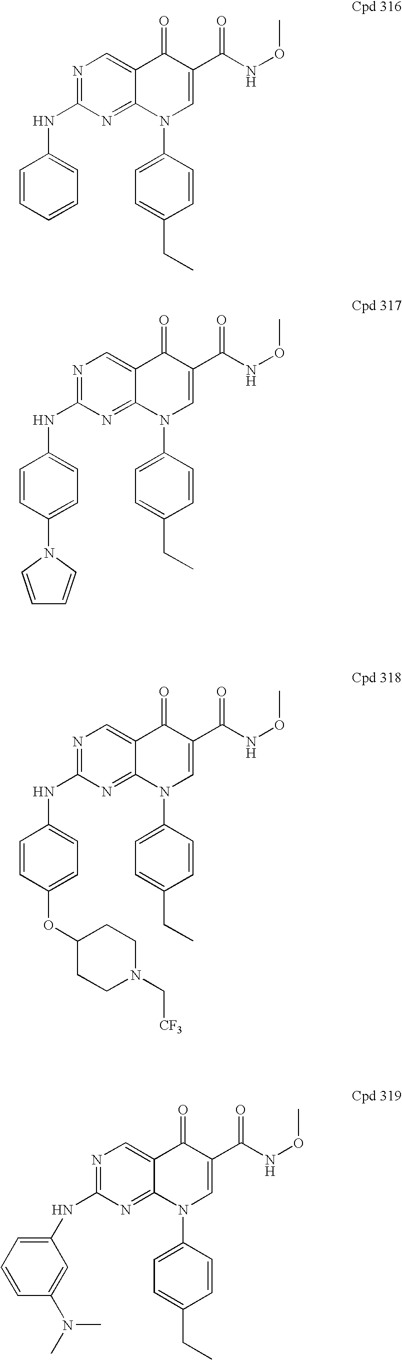 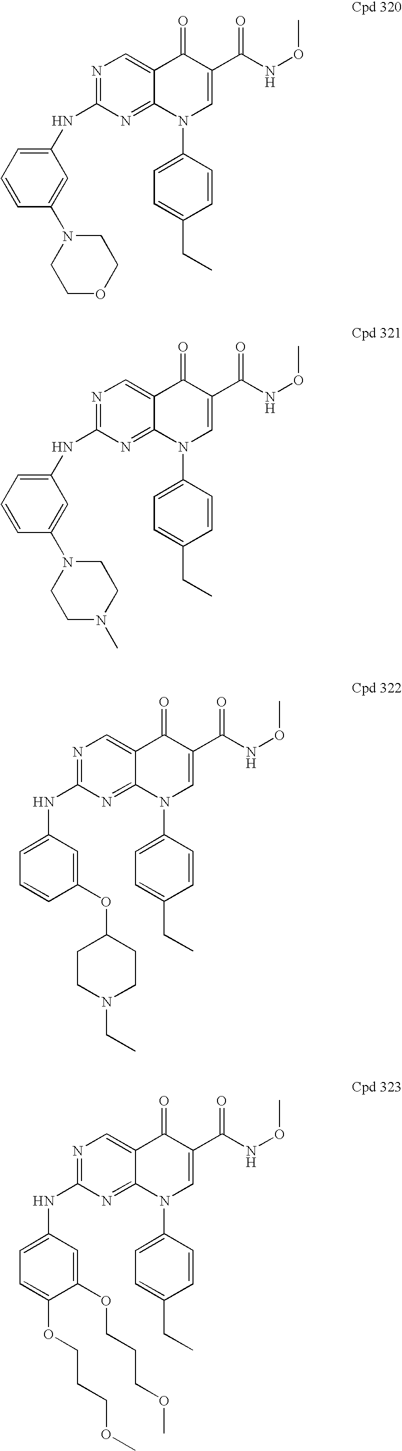 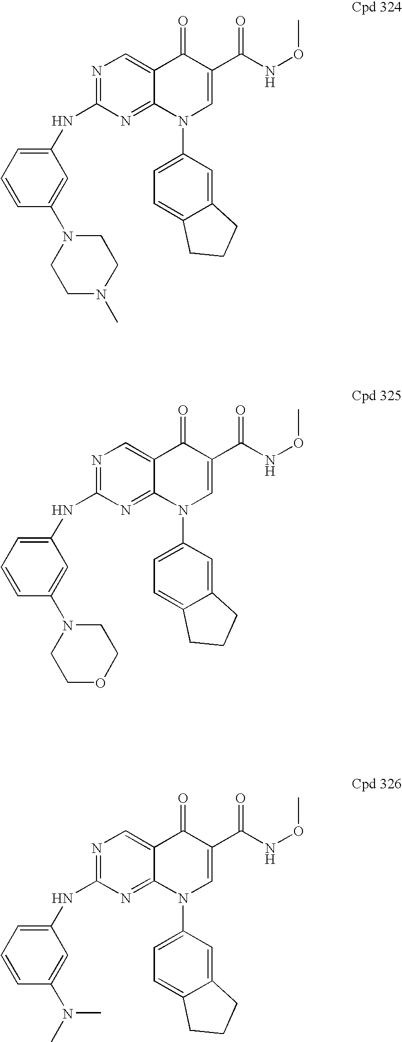
Compound Forms

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

Certain compounds of Formula (I) may exist in various stereoisomeric or tautomeric forms and mixtures thereof. The invention encompasses all such compounds, including active compounds in the form of essentially pure enantiomers, racemic mixtures and tautomers.

The compounds of the present invention may be present in the form of pharmaceutically acceptable salts. For use in medicines, the “pharmaceutically acceptable salts” of the compounds of this invention refer to non-toxic acidic/anionic or basic/cationic salt forms.

Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Furthermore when the compounds of the present invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts. Thus, representative pharmaceutically acceptable salts include the following: acetate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, hydrabamine, hydrobromine, hydrochloride, iodide, isothionate, lactate, malate, maleate, mandelate, mesylate, nitrate, oleate, pamoate, palmitate, phosphate/diphosphate, salicylate, stearate, sulfate, succinate, tartrate, tosylate.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of the isolated species rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule that, in a given configuration, cannot be superimposed on its mirror image. This is in contrast to achiral molecules, which can be superimposed on their mirror images.

The invention is considered to include the tautomeric forms of all compounds of Formula I. In addition, for chiral embodiments of the invention, the invention is considered to include pure enantiomers, racemic mixtures, as well as mixtures of enantiomers having 0.001% to 99.99% enantiomeric excess. In addition, some of the compounds represented by Formula I may be prodrugs, i.e., derivatives of a drug that possess superior delivery capabilities and therapeutic value as compared to the active drug. Prodrugs are transformed into active drugs by in vivo enzymatic or chemical processes.

The two distinct mirror image versions of the chiral molecule are also known as levo (left-handed), abbreviated L, or dextro (right handed), abbreviated D, depending on which way they rotate polarized light. The symbols “R” and “S” represent the configuration of groups around a stereogenic carbon atom(s).

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levorotatory = \frac{( masslevorotatory )}{( massdextrorotatory ) + ( masslevorotatory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( massdextrorotatory )}{( massdextrorotatory ) + ( masslevorotatory )} \times 100$$

“Geometric isomer” means isomers that differ in the orientation of substituent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substituents having higher priority are on opposite sides in relationship to the carbon-carbon double bond. In the “Z” configuration, the substituents having higher priority are oriented on the same side in relationship to the carbon-carbon double bond.

Substituent atoms (other than hydrogen) attached to a ring system may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

The isomeric descriptors (“R,” “S,” “E,” and “Z”) indicate atom configurations relative to a core molecule and are intended to be used as defined in the literature.

Furthermore, compounds of the present invention may have at least one crystalline, polymorph or amorphous form. The plurality of such forms is included in the scope of the invention. In addition, some of the compounds may form solvates with water (i.e., hydrates) or common organic solvents (e.g., organic esters such as ethanolate and the like). The plurality of such solvates is also intended to be encompassed within the scope of this invention.

Chemical Nomenclature and Definitions

Bond lines drawn into a ring system from a substituent variable indicate that the substituent may be attached to any of the substitutable ring atoms.

As used herein, the following terms are intended to have the following meanings (additional definitions are provided where needed throughout the Specification). The definitions herein may specify that a chemical term has an indicated formula. The particular formula provided is not intended to limit the scope of the invention, but is provided as an illustration of the term. The scope of the per se definition of the term is intended to include the plurality of variations expected to be included by one of ordinary skill in the art.

The term “C₁₋₈alkyl” refers to both linear and branched carbon chain radicals of from 1 up to 8 carbon atoms, unless otherwise indicated, and includes, but is not limited to, methyl, ethyl, propyl, isopropyl, butyl, isobutyl, sec-butyl, tert-butyl, pentyl, isopentyl, hexyl, and isohexyl. Examples include C₁₋₆alkyl and C₁₋₄alkyl groups. An alkyl radical may be attached to a core molecule via a terminal carbon atom or via a carbon atom within the chain. Similarly, substituent variables may be attached to an alkyl radical where allowed by available valences.

The term “C₂₋₈alkenyl,” whether used alone or as part of a substituent group, means a straight or branched chain hydrocarbon alkyl radical having at least one carbon-carbon double bond, whereby the double bond is derived by the removal of one hydrogen atom from each of two adjacent carbon atoms of the alkyl radical. Typical alkenyl groups comprising from 2 to 8 carbon atoms, such as, for example, ethenyl, propenyl, allyl (2-propenyl), butenyl, pentenyl, hexenyl and the like. Examples include C₂₋₄alkenyl groups. An alkenyl radical may be attached to a core molecule via a terminal carbon atom or via a carbon atom within the chain. Similarly, substituent variables may be attached to an alkenyl radical where allowed by available valences.

The term “C₂₋₈alkynyl” whether used alone or as part of a substituent group, means a straight or branched chain hydrocarbon alkyl radical having at least one carbon-carbon triple bond, whereby the triple bond is derived by the removal of two hydrogen atoms from each of two adjacent carbon atoms of the alkyl radical. Typical alkynyl groups comprising from 2 to 8 carbon atoms, such as, for example, ethynyl, propynyl, butynyl, pentynyl, hexynyl and the like. Examples include C₂₋₄alkynyl groups. An alkynyl radical may be attached to a core molecule via a terminal carbon atom or via a carbon atom within the chain. Similarly, substituent variables may be attached to an alkynyl radical where allowed by available valences.

The term “aryl” refers to monocyclic or bicyclic aromatic ring systems containing from 6 to 12 carbons in the ring. Alkyl substituents may optionally be present on the ring. Examples include benzene, biphenyl, naphthalene (also referred to as naphthalenyl), azulenyl, anthracenyl and the like. Aryl radicals may be attached to a core molecule and further substituted on any atom when allowed by available valences.

The term “aromatic” refers to a hydrocarbon ring system having an unsaturated, conjugated π electron system.

The term “C₁₋₈alkoxy” refers to a saturated branched or straight chain alcohol radical derived by the removal of the hydrogen atom from the hydroxide oxygen, as in the formula: —O—C₁₋₈alkyl. Examples include methoxy, ethoxy, propoxy, isopropoxy and butoxy. Examples include C₁₋₆alkoxy and C₁₋₄alkoxy groups. An alkoxy radical may be further substituted when allowed by available valences.

The term “C₃₋₁₄cycloalkyl” refers to a saturated or partially unsaturated ring composed of from 3 to 14 carbon atoms. Up to four alkyl substituents may optionally be present on the ring. The term also includes a C₃₋₈cycloalkyl, C₃₋₁₀cycloalkyl, C₅₋₆cycloalkyl, C₅₋₈cycloalkyl, C₅₋₁₂cycloalkyl, C₉₋₁₃cycloalkyl or benzofused C₃₋₁₄cycloalkyl ring system. Examples include 1,1-dimethyl cyclobutyl, 1,2,3-trimethylcyclopentyl, cyclopropyl, cyclobutyl, cyclopentyl, cyclohexyl, cyclohexyl, cyclohexenyl, cycloheptyl, cyclooctyl, 1H-indenyl, indanyl, 9H-fluorenyl, tetrahydro-naphthalenyl, acenaphthenyl, adamantanyl, bicyclo[2.2.1]heptenyl and the like. C₃₋₁₄cycloalkyl radicals may be attached to a core molecule and further substituted on any atom when allowed by available valences.

The term “hetero” used as a prefix for a ring system refers to the replacement of at least one ring carbon atom with one or more heteroatoms independently selected from N, S, or O. Examples include rings wherein 1, 2, 3 or 4 ring members are a nitrogen atom; or, 0, 1, 2 or 3 ring members are nitrogen atoms and 1 member is an oxygen or sulfur atom. When allowed by available valences, up to two adjacent ring members may be heteroatoms; wherein one heteroatom is nitrogen and the other is one heteroatom selected from N, S or O.

The term “heterocyclyl” refers to a nonaromatic (i.e. saturated or partially unsaturated) ring composed of from 3 to 9 carbon atoms and at least one heteroatom selected from N, O or S. Alkyl substituents and/or carbonyl substituents may optionally be present on the ring. Examples include azetidinyl, 2H-pyrrole, 1,3-dioxolanyl, tetrazolyl, tetrazolidinyl, 1,4-dioxanyl, 1,4-dithianyl, 6,7,9,10,12,13,15,16-octahydro-5,8,11,14,17-pentaoxa-benzocyclopentadecenyl, 6,7,9,10,12,13,15,16,18,19-decahydro-5,8,11,14,17,20-hexaoxa-benzocyclooctadecenyl, 1,4,7,10,13-pentaoxa-cyclopentadecane, 1,4,7,10,13,16-hexaoxa-cyclooctadecane, 3,5,11-trioxa-tricyclo[5.3.1.0^{2,6}]undecanyl, 1-azabicyclo[2.2.2]octyl, azetidinyl, azepanyl, hexahydro-1,4-diazepinyl, hexahydro-1,4-oxazepanyl, 6,7-dihydro-5H-cyclopenta[b]pyridinyl, tetrahydro-thienyl, tetrahydro-pyranyl, tetrahydro-pyridazinyl, 1,3-benzodioxolyl (also referred to as benzo[1,3]dioxolyl), 2,3-dihydro-1,4-benzodioxinyl (also referred to as 2,3-dihydro-benzo[1,4]dioxinyl), 2,3-dihydro-1H-indolyl, 1,2,3,4-tetrahydroisoquinolinyl, tetrahydrofuranyl, dihydropyranyl, 1,2,3,6-tetrahydropyridinyl, piperidinyl, 2,5-dimethylpiperidinyl, morpholinyl, piperazinyl, thiomorpholinyl, pyrrolidinyl, pyrrolinyl, pyrazolidinyl, pyrazolinyl, oxazolidinyl, imidazolidinyl, imidazolinyl (also referred to as 4,5-dihydro-1H-imidazolyl) and the like. Heterocyclyl radicals may be attached to a core molecule and further substituted on any atom when allowed by available valences.

The term “heteroaryl” refers to 5- to 7-membered mono- or 8- to 10-membered bicyclic aromatic ring systems, any ring of which may consist of from one to four heteroatoms selected from N, O, S, S(O) or SO₂ where the nitrogen and sulfur atoms can exist in any allowed oxidation state. Examples include benzoimidazolyl, benzothiazolyl, benzothienyl, benzoxazolyl, furanyl, imidazolyl, isothiazolyl, isoxazolyl, oxazolyl, pyrazinyl, pyrazolyl, pyridinyl, pyrimidinyl, pyrrolyl, quinolinyl, thiazolyl, thienyl, oxadiazolyl, triazolyl, 1H-[1,2,4]triazolyl, thiadiazolyl, pyridazinyl, indolizinyl, indolyl, azaindolyl, isoindolyl, benzofuranyl, indazolyl, azaindazolyl, benzoisoxazolyl, benzothiadiazolyl, benzotriazolyl, benzimidazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like. Heteroaryl radicals may be attached to a core molecule and further substituted on any atom when allowed by available valences.

The term “C₁₋₈alkoxy-C₁₋₈alkyl” means a radical of the formula: —C₁₋₈alkyl-O—C₁₋₈alkyl.

The term “C₁₋₈alkoxy-carbonyl” means a radical of the formula: —C(O)—O—C₁₋₈alkyl.

The term “C₁₋₈alkyl-amino” means a radical of the formula: —NH—C₁₋₈alkyl or —N(C₁₋₈alkyl)₂, wherein C₁₋₈alkyl may be further substituted.

The term “C₁₋₈alkyl-amino-C₁₋₈alkyl” means a radical of the formula: —C₁₋₈alkyl-NH—C₁₋₈alkyl or —C₁₋₈alkyl-N(C₁₋₈alkyl)₂, wherein C₁₋₈alkyl may be further substituted.

The term “C₁₋₈alkyl-amino-amino-carbonyl” means a radical of the formula: —C(O)—NH—NH—C₁₋₈alkyl or —C(O)—NH—N(C₁₋₈alkyl)₂, wherein C₁₋₈alkyl or amino may be further substituted where allowed by available valences.

The term “C₁₋₈alkyl-amino-carbonyl” means a radical of the formula: —C(O)—NH—C₁₋₈alkyl or —C(O)—N(C₁₋₈alkyl)₂, wherein C₁₋₈alkyl may be further substituted.

The term “C₁₋₈alkyl-amino-sulfonyl” means a radical of the formula: —SO₂—NH—C₁₋₈alkyl or —SO₂—N(C₁₋₈alkyl)₂, wherein C₁₋₈alkyl may be further substituted.

The term “C₁₋₈alkyl-carbonyl” means a radical of the formula: —C(O)—C₁₋₈alkyl, wherein C₁₋₈alkyl may be further substituted.

The term “C₁₋₈alkyl-carbonyl-amino-sulfonyl” means a radical of the formula: —SO₂—NH—C(O)—C₁₋₈alkyl, wherein C₁₋₈alkyl may be further substituted.

The term “C₁₋₈alkyl-sulfonyl” means a radical of the formula: —SO₂—C₁₋₈alkyl, wherein C₁₋₈alkyl may be further substituted.

The term “C₁₋₈alkyl-sulfonyl-C₁₋₈alkyl” means a radical of the formula: —C₁₋₈alkyl-SO₂—C₁₋₈alkyl, wherein C₁₋₈alkyl may be further substituted.

The term “C₁₋₈alkyl-sulfonyl-amino-carbonyl” means a radical of the formula: —C(O)—NH—SO₂—C₁₋₈alkyl, wherein C₁₋₈alkyl may be further substituted.

The term “amino” means a radical of the formula: —NH₂.

The term “amino-amino-carbonyl” means a radical of the formula: —C(O)—NH—NH₂, wherein amino may be further substituted.

The term “amino-C₁₋₈alkyl” means a radical of the formula: —C₁₋₈alkyl-NH₂, wherein amino may be further substituted.

The term “amino-carbonyl” means a radical of the formula: —C(O)—NH₂, wherein amino may be further substituted.

The term “amino-C₁₋₈alkyl-carbonyl” means a radical of the formula: —C(O)—C₁₋₈alkyl-NH₂, wherein amino may be further substituted.

The term “C₁₋₈alkyl-amino-C₁₋₈alkyl-carbonyl” means a radical of the formula: —C(O)—C₁₋₈alkyl-NH—C₁₋₈alkyl or —C(O)—C₁₋₈alkyl-N(C₁₋₈alkyl)₂, wherein C₁₋₈alkyl or amino may be further substituted where allowed by available valences.

The term “amino-sulfonyl” means a radical of the formula: —SO₂—NH₂, wherein amino may be further substituted.

The term “amino-C₁₋₈alkyl-sulfonyl” means a radical of the formula: —SO₂—C₁₋₈alkyl-NH₂, wherein amino may be further substituted.

The term “aryl-C₁₋₈alkyl” means a radical of the formula: —C₁₋₈alkyl-aryl.

The term “carboxy” means a radical of the formula: —C(O)OH.

The term “C₃₋₁₄cycloalkyl-C₁₋₈alkyl” means a radical of the formula: —C₁₋₈alkyl-C₃₋₁₄cycloalkyl.

The term “C₃₋₁₄cycloalkyl-oxy” means a radical of the formula: —O—C₃₋₁₄cycloalkyl.

The term “heteroaryl-C₁₋₈alkyl” means a radical of the formula: —C₁₋₈alkyl-heteroaryl.

The term “heterocyclyl-C₁₋₈alkyl” means a radical of the formula: —C₁₋₈alkyl-heterocyclyl.

The term “heterocyclyl-amino-carbonyl” means a radical of the formula: —C(O)—NH-heterocyclyl.

The term “heterocyclyl-carbonyl” means a radical of the formula: —C(O)-heterocyclyl.

The term “heterocyclyl-oxy” means a radical of the formula: —O-heterocyclyl.

The term “heterocyclyl-sulfonyl” means a radical of the formula: —SO₂-heterocyclyl.

The term “halogen” or “halo” means the group fluoro, chloro, bromo or iodo.

The term “halo-C₁₋₈alkoxy” means a radical of the formula: —O—C₁₋₈alkyl-(halo)ₙ, wherein one or more halogen atoms may be substituted on C₁₋₈alkyl when allowed by available valences (wherein n represents that amount of available valences based on the number of carbon atoms in the chain), and includes monofluoromethyl, difluoromethyl, trifluoromethyl, trifluoroethyl and the like.

The term “halo-C₁₋₈alkyl” means a radical of the formula: —C₁₋₈alkyl-(halo), wherein one or more halogen atoms may be substituted on C₁₋₈alkyl when allowed by available valences (wherein n represents that amount of available valences based on the number of carbon atoms in the chain), and includes monofluoromethyl, difluoromethyl, trifluoromethyl, trifluoroethyl and the like.

The term “halo-C₁₋₈alkyl-carbonyl” means a radical of the formula: —C(O)—C₁₋₈alkyl-(halo) %, wherein the definition of the C₁₋₈alkyl-(halo) % portion is as described previously herein.

The term “hydroxy-C₁₋₈alkyl” means a radical of the formula: —C₁₋₈alkyl-hydroxy, wherein C₁₋₈alkyl is substituted on one or more available carbon chain atoms with one or more hydroxy radicals when allowed by available valences.

The term “oxo” means a radical of the formula: ═O.

The term “substituted,” refers to a core molecule on which one or more hydrogen atoms have been replaced with one or more functional radical moieties. The number that is allowed by available valences limits the amount of substituents. Substitution is not limited to the core molecule, but may also occur on a substituent radical, whereby the substituent radical becomes a linking group.

The term “independently selected” refers to one or more substituents selected from a group of substituents variable group, wherein the selected substituents may be the same or different.

Therapeutic Uses

The compounds of Formula I represent novel potent inhibitors of protein tyrosine kinases, such as c-fms, and may be useful in the prevention and treatment of a protein tyrosine kinase mediated disorder.

The invention also relates to methods of inhibiting protein tyrosine kinase activity in a mammal by administration of a therapeutically effective amount of at least one compound of Formula I. A preferred tyrosine kinase is c-fms.

The compounds of the present invention are further useful as markers for the c-fms receptor. Compounds of formula (I) when used as markers are for example radio-labeled by for example, substituting at least one hydrogen atom with a tritium atom. Other labeling techniques known in the arts can also be used.

The invention also provides methods of inhibiting a protein tyrosine kinase comprising contacting the protein tyrosine kinase with an effective inhibitory amount of at least one of the compounds of Formula I.

Accordingly, the present invention is directed to a method of using a compound of Formula I for inhibiting protein tyrosine kinase activity comprising administering an effective amount of at least one compound of Formula I.

The present invention is also directed to a method of treating or ameliorating a protein tyrosine kinase mediated disorder in a subject in need thereof comprising administering to the subject an effective amount of at least one compound of Formula I.

An embodiment of the present invention is directed to a method wherein the protein tyrosine kinase is c-fms.

In one embodiment of inhibiting a protein tyrosine kinase, at least one of the compounds of Formula I is combined with a known tyrosine kinase inhibitor.

In various embodiments of the invention, the protein tyrosine kinases inhibited by the compounds of Formula I are located in cells, in a mammal or in vitro. In the case of mammals, which includes humans, a therapeutically effective amount of a pharmaceutically acceptable form of at least one of the compounds of Formula I is administered.

The invention further provides methods of treating cancer in mammals, including humans, by administration of a therapeutically effective amount of a pharmaceutically acceptable composition of least one compound of Formula I.

Examples of solid tumor and blood related cancers include, but are not limited to, ovarian cancer, uterine cancer, breast cancer, colon cancer, stomach cancer, hairy cell leukemia and non-small lung carcinoma.

In one embodiment of the invention, an effective amount of at least one compound of Formula I is administered in combination with an effective amount of a chemotherapeutic agent.

The invention also provides methods of treating cardiovascular and inflammatory diseases in mammals, including humans, by administration of a therapeutically effective amount of a pharmaceutically acceptable form of at least one of the compounds of Formula I.

Examples of diseases that may be effectively treated include glomerulonephritis, immune nephritis, rheumatoid arthritis, inflammatory bowel disease, prosthesis failure, sarcoidosis, congestive obstructive pulmonary disease, idiopathic pulmonary fibrosis, asthma, pancreatitis, HIV infection, psoriasis, graft rejection, diabetes, diabetic retinopathy, diabetic nephropathy, obesity, cardiac hypertrophy, atherosclerosis, restenosis, age-related macular degeneration, tumor related angiogenesis, solid tumor cancers, blood related cancers, multiple sclerosis, schizophrenia or Alzheimer's dementia.

When employed as protein tyrosine kinase inhibitors, the compounds of the invention may be administered in an effective amount within the dosage range of about 0.001 mg/kg to about 10 g, or in a range of between about 0.05 mg/kg to about 5 g, in single or divided daily doses or a dosage of 0.1 mg/kg to about 5 mg/kg, delivered orally.

The dosage administered will be affected by factors such as the route of administration, the health, weight and age of the recipient, the frequency of the treatment and the presence of concurrent and unrelated treatments.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts, which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamine salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

A representative compound of formula (I) or a form thereof includes a compound selected from the group consisting of:
1 8-indan-5-yl-5-oxo-2-(3-piperazin-1-yl-phenylamino)-5,8-dihydro-pyrido[2,3-d]pyrimidine-6-carboxylic acid isopropylamide,
2 8-indan-5-yl-5-oxo-2-(3-piperazin-1-yl-phenylamino)-5,8-dihydro-pyrido[2,3-d]pyrimidine-6-carboxylic acid methoxy-amide,
3 8-(3-chloro-4-fluoro-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
4 8-(3-chloro-4-fluoro-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy amide,
5 8-indan-5-yl-5-oxo-2-[4-(2-pyrrolidin-1-yl-ethyl)-phenylamino]-5,8-dihydro-pyrido[2,3-d]pyrimidine-6-carboxylic acid amide,
6 (S)-2-[4-(2-hydroxymethyl-pyrrolidin-1-yl)-phenylamino]-8-indan-5-yl-5-oxo-5,8-dihydro-pyrido[2,3-d]pyrimidine-6-carboxylic acid amide,
7 (S)-2-[4-(2-hydroxymethyl-pyrrolidin-1-yl)-phenylamino]-8-indan-5-yl-5-oxo-5,8-dihydro-pyrido[2,3-d]pyrimidine-6-carboxylic acid methoxy-amide,
8 8-indan-5-yl-5-oxo-2-[4-(2-oxo-pyrrolidin-1-ylmethyl)-phenylamino]-5,8-dihydro-pyrido[2,3-d]pyrimidine-6-carboxylic acid amide,
9 8-indan-5-yl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
10 8-cyclohexyl-5-oxo-2-(4-pyrrolidin-1-yl-phenylamino)-5,8-dihydro-pyrido[2,3-d]pyrimidine-6-carboxylic acid methoxy-amide,
12 8-cyclohexyl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
13 8-cyclohexyl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
14 8-cyclohexyl-5-oxo-2-[4-(2-pyrrolidin-1-yl-ethyl)-phenylamino]-5,8-dihydro-pyrido[2,3-d]pyrimidine-6-carboxylic acid methoxy-amide,
15 8-cyclohexyl-5-oxo-2-[4-(2-pyrrolidin-1-yl-ethyl)-phenylamino]-5,8-dihydro-pyrido[2,3-d]pyrimidine-6-carboxylic acid ethoxy-amide,
16 8-cyclohexyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
17 8-cyclohexyl-5-oxo-2-[4-(2-pyrrolidin-1-yl-ethyl)-phenylamino]-5,8-dihydro-pyrido[2,3-d]pyrimidine-6-carboxylic acid hydroxyamide,
18 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
19 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
20 8-cyclohexyl-2-[4-(2-morpholin-4-yl-ethyl)-phenylamino]-5-oxo-5,8-dihydro-pyrido[2,3-d]pyrimidine-6-carboxylic acid methoxy-amide,
21 8-cyclohexyl-2-[4-(2-morpholin-4-yl-ethyl)-phenylamino]-5-oxo-5,8-dihydro-pyrido[2,3-d]pyrimidine-6-carboxylic acid ethoxy-amide,
22 (S)-8-cyclohexyl-5-oxo-2-[4-(2-oxo-oxazolidin-4-ylmethyl)-phenylamino]-5,8-dihydro-pyrido[2,3-d]pyrimidine-6-carboxylic acid methoxy-amide,
23 (S)-8-cyclohexyl-5-oxo-2-[4-(2-oxo-oxazolidin-4-ylmethyl)-phenylamino]-5,8-dihydro-pyrido[2,3-d]pyrimidine-6-carboxylic acid ethoxy-amide,
24 8-cyclohexyl-2-[4-(3,5-dimethyl-piperazin-1-yl)-phenylamino]-5-oxo-5,8-dihydro-pyrido[2,3-d]pyrimidine-6-carboxylic acid methoxy-amide,
25 8-cyclohexyl-2-[4-(3,5-dimethyl-piperazin-1-yl)-phenylamino]-5-oxo-5,8-dihydro-pyrido[2,3-d]pyrimidine-6-carboxylic acid ethoxy-amide,
26 8-cyclohexyl-5-oxo-2-(4-piperidin-4-yl-phenylamino)-5,8-dihydro-pyrido[2,3-d]pyrimidine-6-carboxylic acid methoxy-amide,
27 8-cyclohexyl-5-oxo-2-(4-piperidin-4-yl-phenylamino)-5,8-dihydro-pyrido[2,3-d]pyrimidine-6-carboxylic acid ethoxy-amide,
28 2-[4-(1-acetyl-piperidin-4-yl)-phenylamino]-8-cyclohexyl-5-oxo-5,8-dihydro-pyrido[2,3-d]pyrimidine-6-carboxylic acid methoxy-amide,
29 2-[4-(1-acetyl-piperidin-4-yl)-phenylamino]-8-cyclohexyl-5-oxo-5,8-dihydro-pyrido[2,3-d]pyrimidine-6-carboxylic acid ethoxy-amide,
30 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
31 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
32 8-(4-ethynyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
33 8-(4-chloro-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
34 8-(4-chloro-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
35 8-cyclopentyl-5-oxo-2-(4-pyrrolidin-1-yl-phenylamino)-5,8-dihydro-pyrido[2,3-d]pyrimidine-6-carboxylic acid methoxy-amide,
36 8-cyclopentyl-5-oxo-2-[4-(pyrrolidine-1-carbonyl)-phenylamino]-5,8-dihydro-pyrido[2,3-d]pyrimidine-6-carboxylic acid methoxy-amide,
37 8-cyclopentyl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
38 8-cyclopentyl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
39 8-cyclopentyl-5-oxo-2-[4-(2-pyrrolidin-1-yl-ethyl)-phenylamino]-5,8-dihydro-pyrido[2,3-d]pyrimidine-6-carboxylic acid methoxy-amide,
40 8-cyclopentyl-5-oxo-2-[4-(2-pyrrolidin-1-yl-ethyl)-phenylamino]-5,8-dihydro-pyrido[2,3-d]pyrimidine-6-carboxylic acid ethoxy-amide,
41 8-cyclopentyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
42 8-cyclopentyl-6-(morpholine-4-carbonyl)-2-[4-(2-pyrrolidin-1-yl-ethyl)-phenylamino]-8H-pyrido[2,3-d]pyrimidin-5-one,
43 8-cyclopentyl-5-oxo-2-[4-(2-pyrrolidin-1-yl-ethyl)-phenylamino]-5,8-dihydro-pyrido[2,3-d]pyrimidine-6-carboxylic acid (2-hydroxy-ethyl)-amide,
44 8-cyclopentyl-5-oxo-2-[4-(2-pyrrolidin-1-yl-ethyl)-phenylamino]-5,8-dihydro-pyrido[2,3-d]pyrimidine-6-carboxylic acid (2-amino-ethyl)-amide,
45 8-cyclopentyl-5-oxo-2-(4-piperidin-4-yl-phenylamino)-5,8-dihydro-pyrido[2,3-d]pyrimidine-6-carboxylic acid methoxy-amide,
46 2-[4-(1-acetyl-piperidin-4-yl)-phenylamino]-8-cyclopentyl-5-oxo-5,8-dihydro-pyrido[2,3-d]pyrimidine-6-carboxylic acid methoxy-amide,
47 2-[4-(1-acetyl-piperidin-4-yl)-phenylamino]-8-cyclopentyl-5-oxo-5,8-dihydro-pyrido[2,3-d]pyrimidine-6-carboxylic acid ethoxy-amide,
48 8-cyclopentyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
49 8-cyclopentyl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
50 8-(3,4-dimethyl-phenyl)-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
51 8-(3,4-dimethyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
52 8-(3,4-dimethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
53 8-(3,4-dimethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
54 8-(4-ethyl-phenyl)-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
55 8-(4-ethyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
56 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
57 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
58 8-(4-ethyl-phenyl)-5-oxo-2-(4-piperidin-4-yl-phenylamino)-5,8-dihydro-pyrido[2,3-d]pyrimidine-6-carboxylic acid isopropoxy-amide,
59 2-[4-(1-acetyl-piperidin-4-yl)-phenylamino]-8-(4-ethyl-phenyl)-5-oxo-5,8-dihydro-pyrido[2,3-d]pyrimidine-6-carboxylic acid isopropoxy-amide,
60 8-(4-ethyl-phenyl)-2-[4-(1-methanesulfonyl-piperidin-4-yl)-phenylamino]-5-oxo-5,8-dihydro-pyrido[2,3-d]pyrimidine-6-carboxylic acid isopropoxy-amide,
61 8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
62 8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
63 8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid isopropoxy-amide,
64 8-indan-5-yl-2-{4-[2-(4-methyl-piperazin-1-yl)-ethyl]-phenylamino}-6-(piperidine-1-carbonyl)-8H-pyrido[2,3-d]pyrimidin-5-one,
65 8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid (2-hydroxy-ethyl)-amide,
66 8-indan-5-yl-5-oxo-2-(3-piperazin-1-yl-phenylamino)-5,8-dihydro-pyrido[2,3-d]pyrimidine-6-carboxylic acid (2-hydroxy-ethyl)-amide,
67 (S)-8-indan-5-yl-5-oxo-2-[4-(2-oxo-oxazolidin-4-ylmethyl)-phenylamino]-5,8-dihydro-pyrido[2,3-d]pyrimidine-6-carboxylic acid methoxy-amide,
68 (S)-8-indan-5-yl-5-oxo-2-[4-(2-oxo-oxazolidin-4-ylmethyl)-phenylamino]-5,8-dihydro-pyrido[2,3-d]pyrimidine-6-carboxylic acid ethoxy-amide,
69 2-[4-(1-acetyl-piperidin-4-yl)-phenylamino]-8-indan-5-yl-5-oxo-5,8-dihydro-pyrido[2,3-d]pyrimidine-6-carboxylic acid methoxy-amide,
70 2-[4-(1-acetyl-piperidin-4-yl)-phenylamino]-8-indan-5-yl-5-oxo-5,8-dihydro-pyrido[2,3-d]pyrimidine-6-carboxylic acid ethoxy-amide,
71 8-indan-5-yl-2-[4-(2-methanesulfonyl-ethyl)-phenylamino]-5-oxo-5,8-dihydro-pyrido[2,3-d]pyrimidine-6-carboxylic acid methoxy-amide,
72 8-indan-5-yl-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
73 2-{4-[2-(1,5-dioxa-9-aza-spiro[5.5]undec-9-yl)-ethyl]-phenylamino}-8-indan-5-yl-5-oxo-5,8-dihydro-pyrido[2,3-d]pyrimidine-6-carboxylic acid methoxy-amide,
74 8-indan-5-yl-2-(4-{2-[methyl-(tetrahydro-pyran-4-yl)-amino]-ethyl}-phenylamino)-5-oxo-5,8-dihydro-pyrido[2,3-d]pyrimidine-6-carboxylic acid methoxy-amide,
75 8-indan-5-yl-5-oxo-2-(4-piperidin-4-yl-phenylamino)-5,8-dihydro-pyrido[2,3-d]pyrimidine-6-carboxylic acid methoxy-amide,
76 8-indan-5-yl-2-[4-(1-methanesulfonyl-piperidin-4-yl)-phenylamino]-5-oxo-5,8-dihydro-pyrido[2,3-d]pyrimidine-6-carboxylic acid methoxy-amide,
77 8-indan-5-yl-2-[4-(2-isopropylsulfamoyl-ethyl)-phenylamino]-5-oxo-5,8-dihydro-pyrido[2,3-d]pyrimidine-6-carboxylic acid methoxy-amide,
78 8-indan-5-yl-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide,
79 2-[4-(2-hydroxy-ethyl)-phenylamino]-8-indan-5-yl-5-oxo-5,8-dihydro-pyrido[2,3-d]pyrimidine-6-carboxylic acid methoxy-amide,
80 8-indan-5-yl-2-[4-(2-morpholin-4-yl-ethyl)-phenylamino]-5-oxo-5,8-dihydro-pyrido[2,3-d]pyrimidine-6-carboxylic acid methoxy-amide,
81 8-indan-5-yl-2-[4-(2-methoxy-ethyl)-phenylamino]-5-oxo-5,8-dihydro-pyrido[2,3-d]pyrimidine-6-carboxylic acid methoxy-amide,
82 8-indan-5-yl-2-[4-(2-methanesulfonylamino-ethyl)-phenylamino]-5-oxo-5,8-dihydro-pyrido[2,3-d]pyrimidine-6-carboxylic acid methoxy-amide,
83 8-indan-5-yl-5-oxo-2-{4-[2-(4-oxo-piperidin-1-yl)-ethyl]-phenylamino}-5,8-dihydro-pyrido[2,3-d]pyrimidine-6-carboxylic acid methoxy-amide,
84 3-{4-[4-(8-indan-5-yl-6-methoxycarbamoyl-5-oxo-5,8-dihydro-pyrido[2,3-d]pyrimidin-2-ylamino)-phenyl]-piperidin-1-yl}-propionic acid,
85 {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid,
86 {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester,
87 8-indan-5-yl-2-{4-[1-(2-methanesulfonylamino-2-oxo-ethyl)-piperidin-4-yl]-phenylamino}-5-oxo-5,8-dihydro-pyrido[2,3-d]pyrimidine-6-carboxylic acid methoxy-amide,
88 2-{4-[1-(2-hydroxy-ethyl)-piperidin-4-yl]-phenylamino}-8-indan-5-yl-5-oxo-5,8-dihydro-pyrido[2,3-d]pyrimidine-6-carboxylic acid methoxy-amide,
89 8-indan-5-yl-2-[4-(2-methylsulfamoyl-ethyl)-phenylamino]-5-oxo-5,8-dihydro-pyrido[2,3-d]pyrimidine-6-carboxylic acid methoxy-amide,
90 2-[4-(2-acetylsulfamoyl-ethyl)-phenylamino]-8-indan-5-yl-5-oxo-5,8-dihydro-pyrido[2,3-d]pyrimidine-6-carboxylic acid methoxy-amide,
91 8-indan-5-yl-5-oxo-2-[4-(1H-tetrazol-5-ylmethyl)-phenylamino]-5,8-dihydro-pyrido[2,3-d]pyrimidine-6-carboxylic acid methoxy-amide,
92 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid cyclopentyloxy-amide,
93 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid (1-ethyl-propoxy)-amide,
94 (4-{4-[8-(4-ethyl-phenyl)-6-methoxycarbamoyl-5-oxo-5,8-dihydro-pyrido[2,3-d]pyrimidin-2-ylamino]-phenyl}-piperidin-1-yl)-acetic acid,
95 8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide,
96 8-(4-ethyl-phenyl)-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide,
97 2-{4-[2-(2-hydroxy-ethylamino)-ethyl]-phenylamino}-8-indan-5-yl-5-oxo-5,8-dihydro-pyrido[2,3-d]pyrimidine-6-carboxylic acid methoxy-amide,
98 8-indan-5-yl-2-{4-[2-(2-methoxy-ethylamino)-ethyl]-phenylamino}-5-oxo-5,8-dihydro-pyrido[2,3-d]pyrimidine-6-carboxylic acid methoxy-amide,
99 8-indan-5-yl-2-{4-[2-(2-methanesulfonyl-ethylamino)-ethyl]-phenylamino}-5-oxo-5,8-dihydro-pyrido[2,3-d]pyrimidine-6-carboxylic acid methoxy-amide,
100 2-(4-{2-[bis-(2-hydroxy-ethyl)-amino]-ethyl}-phenylamino)-8-indan-5-yl-5-oxo-5,8-dihydro-pyrido[2,3-d]pyrimidine-6-carboxylic acid methoxy-amide,
101 (R)-2-{4-[2-(2-hydroxymethyl-pyrrolidin-1-yl)-ethyl]-phenylamino}-8-indan-5-yl-5-oxo-5,8-dihydro-pyrido[2,3-d]pyrimidine-6-carboxylic acid methoxy-amide,
102 8-indan-5-yl-5-oxo-2-[4-(piperidin-1-ylcarbamoylmethyl)-phenylamino]-5,8-dihydro-pyrido[2,3-d]pyrimidine-6-carboxylic acid methoxy-amide,
103 {4-[8-(4-ethyl-phenyl)-6-methoxycarbamoyl-5-oxo-5,8-dihydro-pyrido[2,3-d]pyrimidin-2-ylamino]-phenyl}-acetic acid,
104 8-(4-ethyl-phenyl)-5-oxo-2-[4-(piperidin-1-ylcarbamoylmethyl)-phenylamino]-5,8-dihydro-pyrido[2,3-d]pyrimidine-6-carboxylic acid methoxy-amide,
105 2-[4-(N′,N′-dimethyl-hydrazinocarbonylmethyl)-phenylamino]-8-(4-ethyl-phenyl)-5-oxo-5,8-dihydro-pyrido[2,3-d]pyrimidine-6-carboxylic acid methoxy-amide,
106 8-(4-ethyl-phenyl)-2-[4-(N′-methyl-hydrazinocarbonylmethyl)-phenylamino]-5-oxo-5,8-dihydro-pyrido[2,3-d]pyrimidine-6-carboxylic acid methoxy-amide,
107 2-{4-[2-(adamantan-2-ylamino)-ethyl]-phenylamino}-8-indan-5-yl-5-oxo-5,8-dihydro-pyrido[2,3-d]pyrimidine-6-carboxylic acid methoxy-amide,
108 2-{4-[2-(bicyclo[2.2.1]hept-2-ylamino)-ethyl]-phenylamino}-8-indan-5-yl-5-oxo-5,8-dihydro-pyrido[2,3-d]pyrimidine-6-carboxylic acid methoxy-amide,
109 (R)-1-{2-[4-(8-indan-5-yl-6-methoxycarbamoyl-5-oxo-5,8-dihydro-pyrido[2,3-d]pyrimidin-2-ylamino)-phenyl]-ethyl}-pyrrolidine-2-carboxylic acid,
110 2-{4-[2-(7-aza-bicyclo[2.2.1]hept-7-yl)-ethyl]-phenylamino}-8-indan-5-yl-5-oxo-5,8-dihydro-pyrido[2,3-d]pyrimidine-6-carboxylic acid methoxy-amide,
111 8-indan-5-yl-2-[4-(1-isopropyl-piperidin-4-yl)-phenylamino]-5-oxo-5,8-dihydro-pyrido[2,3-d]pyrimidine-6-carboxylic acid methoxy-amide,
112 8-indan-5-yl-5-oxo-2-[3-(piperidine-1-carbonyl)-phenylamino]-5,8-dihydro-pyrido[2,3-d]pyrimidine-6-carboxylic acid amide,
114 8-benzyl-2-[4-(4-methyl-piperazin-1-yl)-phenylamino]-5-oxo-5,8-dihydro-pyrido[2,3-d]pyrimidine-6-carboxylic acid amide,
115 2-[3-(3-dimethylamino-propyl)-phenylamino]-8-indan-5-yl-5-oxo-5,8-dihydro-pyrido[2,3-d]pyrimidine-6-carboxylic acid amide,
116 2-[3-(3-dimethylamino-propyl)-phenylamino]-8-indan-5-yl-5-oxo-5,8-dihydro-pyrido[2,3-d]pyrimidine-6-carboxylic acid methylamide,
117 2-[3-(3-dimethylamino-propyl)-phenylamino]-8-indan-5-yl-5-oxo-5,8-dihydro-pyrido[2,3-d]pyrimidine-6-carboxylic acid ethylamide,
118 2-(4-dimethylcarbamoylmethyl-phenylamino)-8-indan-5-yl-5-oxo-5,8-dihydro-pyrido[2,3-d]pyrimidine-6-carboxylic acid amide,
120 8-indan-5-yl-2-[3-(morpholine-4-carbonyl)-phenylamino]-5-oxo-5,8-dihydro-pyrido[2,3-d]pyrimidine-6-carboxylic acid amide,
121 2-[4-(2-dimethylamino-ethyl)-phenylamino]-8-indan-5-yl-5-oxo-5,8-dihydro-pyrido[2,3d]pyrimidine-6-carboxylic acid amide,
122 8-indan-5-yl-5-oxo-2-[4-(pyrrolidine-1-carbonyl)-phenylamino]-5,8-dihydro-pyrido[2,3-d]pyrimidine-6-carboxylic acid amide,
123 2-(4-dimethylcarbamoyl-phenylamino)-8-indan-5-yl-5-oxo-5,8-dihydro-pyrido[2,3-d]pyrimidine-6-carboxylic acid amide,
124 8-indan-5-yl-5-oxo-2-(4-pyrrolidin-1-ylmethyl-phenylamino)-5,8-dihydro-pyrido[2,3-d]pyrimidine-6-carboxylic acid amide,
125 8-indan-5-yl-5-oxo-2-(3-pyrrolidin-1-yl-phenylamino)-5,8-dihydro-pyrido[2,3-d]pyrimidine-6-carboxylic acid amide,
126 2-[4-(1-acetyl-piperidin-4-yl)-phenylamino]-8-indan-5-yl-5-oxo-5,8-dihydro-pyrido[2,3-d]pyrimidine-6-carboxylic acid amide,
128 8-indan-5-yl-2-[4-(4-methyl-piperazin-1-ylmethyl)-phenylamino]-5-oxo-5,8-dihydro-pyrido[2,3-d]pyrimidine-6-carboxylic acid methoxy-amide,
129 2-(3-dimethylsulfamoyl-phenylamino)-8-indan-5-yl-5-oxo-5,8-dihydro-pyrido[2,3-d]pyrimidine-6-carboxylic acid methoxy-amide,
130 8-indan-5-yl-5-oxo-2-[4-(2-pyrrolidin-1-yl-ethyl)-phenylamino]-5,8-dihydro-pyrido[2,3-d]pyrimidine-6-carboxylic acid methoxy-amide,
132 8-indan-5-yl-5-oxo-2-[4-(2-pyrrolidin-1-yl-ethyl)-phenylamino]-5,8-dihydro-pyrido[2,3-d]pyrimidine-6-carboxylic acid (2-methoxy-ethyl)-amide,
133 8-indan-5-yl-5-oxo-2-[4-(2-pyrrolidin-1-yl-ethyl)-phenylamino]-5,8-dihydro-pyrido[2,3-d]pyrimidine-6-carboxylic acid isopropoxy-amide,
134 8-indan-5-yl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
135 8-indan-5-yl-5-oxo-2-{4-[2-(2-oxo-pyrrolidin-1-yl)-ethyl]-phenylamino}-5,8-dihydro-pyrido[2,3-d]pyrimidine-6-carboxylic acid ethoxy-amide,
136 2-(4-dimethylsulfamoyl-phenylamino)-8-indan-5-yl-5-oxo-5,8-dihydro-pyrido[2,3-d]pyrimidine-6-carboxylic acid ethoxy-amide,
137 2-(4-dimethylsulfamoyl-phenylamino)-8-indan-5-yl-5-oxo-5,8-dihydro-pyrido[2,3-d]pyrimidine-6-carboxylic acid isopropoxy-amide,
138 8-indan-5-yl-2-[4-(1-methyl-piperidin-4-yl)-phenylamino]-5-oxo-5,8-dihydro-pyrido[2,3-d]pyrimidine-6-carboxylic acid methoxy-amide,
139 8-indan-5-yl-2-[4-(1-methyl-piperidin-4-yl)-phenylamino]-5-oxo-5,8-dihydro-pyrido[2,3-d]pyrimidine-6-carboxylic acid ethoxy-amide,
140 8-cyclohexyl-2-[4-(1-methyl-piperidin-4-yl)-phenylamino]-5-oxo-5,8-dihydro-pyrido[2,3-d]pyrimidine-6-carboxylic acid methoxy-amide,
141 8-cyclohexyl-2-[4-(1-methyl-piperidin-4-yl)-phenylamino]-5-oxo-5,8-dihydro-pyrido[2,3-d]pyrimidine-6-carboxylic acid ethoxy-amide,
142 8-cyclopropyl-5-oxo-2-[4-(2-pyrrolidin-1-yl-ethyl)-phenylamino]-5,8-dihydro-pyrido[2,3-d]pyrimidine-6-carboxylic acid ethoxy-amide,
143 8-cyclopropyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
144 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide,
145 2-{4-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide,
146 8-indan-5-yl-2-{4-[2-(4-methanesulfonyl-piperazin-1-yl)-ethyl]-phenylamino}-5-oxo-5,8-dihydro-pyrido[2,3-d]pyrimidine-6-carboxylic acid methoxy-amide,
147 (4-{2-[4-(8-indan-5-yl-6-methoxycarbamoyl-5-oxo-5,8-dihydro-pyrido[2,3-d]pyrimidin-2-ylamino)-phenyl]-ethyl}-piperazin-1-yl)-acetic acid,
148 8-indan-5-yl-5-oxo-2-(4-{2-[4-(2,2,2-trifluoro-acetyl)-piperazin-1-yl]-ethyl}-phenylamino)-5,8-dihydro-pyrido[2,3-d]pyrimidine-6-carboxylic acid methoxy-amide,
149 8-indan-5-yl-5-oxo-2-(4-{2-[4-(2,2,2-trifluoro-ethyl)-piperazin-1-yl]-ethyl}-phenylamino)-5,8-dihydro-pyrido[2,3-d]pyrimidine-6-carboxylic acid methoxy-amide,
150 4-{2-[4-(8-indan-5-yl-6-methoxycarbamoyl-5-oxo-5,8-dihydro-pyrido[2,3-d]pyrimidin-2-ylamino)-phenyl]-ethyl}-piperazine-1-carboxylic acid methyl ester,
151 8-indan-5-yl-2-{4-[3-(4-methylpiperazin-1-yl)-propyl]-phenylamino}-5-oxo-5,8-dihydro-pyrido[2,3-d]pyrimidine-6-carboxylic acid methoxy-amide,
152 8-indan-5-yl-2-{4-[2-(4-methylpiperazin-1-yl)-2-oxo-ethyl]-phenylamino}-5-oxo-5,8-dihydro-pyrido[2,3-d]pyrimidine-6-carboxylic acid methoxy-amide,
153 8-indan-5-yl-2-[4-(3-methanesulfonylamino-3-oxo-propyl)-phenylamino]-5-oxo-5,8-dihydro-pyrido[2,3-d]pyrimidine-6-carboxylic acid methoxy-amide,
154 8-indan-5-yl-2-[4-(4-methyl-piperazine-1-sulfonyl)-phenylamino]-5-oxo-5,8-dihydro-pyrido[2,3-d]pyrimidine-6-carboxylic acid methoxy-amide,
155 2-[4-(1-ethyl-piperidin-4-yl)-phenylamino]-8-indan-5-yl-5-oxo-5,8-dihydro-pyrido[2,3-d]pyrimidine-6-carboxylic acid methoxy-amide,
156 8-(4-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide,
157 8-(4-ethyl-phenyl)-2-[4-(1-ethyl-piperidin-4-yl)-phenylamino]-5-oxo-5,8-dihydro-pyrido[2,3-d]pyrimidine-6-carboxylic acid methoxy-amide,
159 2-[4-(1-methylpiperidin-4-yl)-phenylamino]-5-oxo-8-(4-trifluoromethoxyphenyl)-5,8-dihydropyrido[2,3-d]pyrimidine-6-carboxylic acid methoxy-amide,
162 2-{4-[1-(2-hydroxyethyl)-piperidin-4-yl]-phenylamino}-5-oxo-8-(4-trifluoromethoxy-phenyl)-5,8-dihydropyrido[2,3-d]pyrimidine-6-carboxylic acid methoxy-amide,
171 2-[4-(2-imidazol-1-yl-ethyl)-phenylamino]-8-indan-5-yl-5-oxo-5,8-dihydro-pyrido[2,3-d]pyrimidine-6-carboxylic acid methoxy-amide,
172 8-indan-5-yl-2-{4-[2-(5-methyl-1H-[1,2,4]triazol-3-yl)-ethyl]-phenylamino}-5-oxo-5,8-dihydro-pyrido[2,3-d]pyrimidine-6-carboxylic acid methoxy-amide,
173 8-indan-5-yl-5-oxo-2-(4-pyridin-4-ylmethyl-phenylamino)-5,8-dihydro-pyrido[2,3-d]pyrimidine-6-carboxylic acid methoxy-amide,
174 8-indan-5-yl-5-oxo-2-(3-[1,2,4]triazol-1-yl-phenylamino)-5,8-dihydro-pyrido[2,3-d]pyrimidine-6-carboxylic acid methoxy-amide,
175 2-{4-[2-(4-methylpiperazin-1-yl)-ethyl]-phenylamino}-5-oxo-8-(4-trifluoromethoxy-phenyl)-5,8-dihydropyrido[2,3-d]pyrimidine-6-carboxylic acid methoxy-amide,
176 5-oxo-2-(4-piperidin-4-yl-phenylamino)-8-(4-trifluoromethoxyphenyl)-5,8-dihydro-pyrido[2,3-d]pyrimidine-6-carboxylic acid methoxy-amide,
177 5-oxo-2-[4-(1-sulfamoyl-piperidin-4-yl)-phenylamino]-8-(4-trifluoromethoxyphenyl)-5,8-dihydropyrido[2,3-d]pyrimidine-6-carboxylic acid methoxy-amide,
178 8-indan-5-yl-5-oxo-2-[4-(1-sulfamoyl-piperidin-4-yl)-phenylamino]-5,8-dihydro-pyrido[2,3-d]pyrimidine-6-carboxylic acid methoxy-amide,
179 8-(4-ethyl-phenyl)-5-oxo-2-[4-(1-sulfamoyl-piperidin-4-yl)-phenylamino]-5,8-dihydro-pyrido[2,3-d]pyrimidine-6-carboxylic acid methoxy-amide,
181 8-(4-ethyl-phenyl)-2-[4-(1-ethyl-piperidin-4-yl)-phenylamino]-5-oxo-5,8-dihydro-pyrido[2,3-d]pyrimidine-6-carboxylic acid ethoxy-amide,
182 8-(4-ethyl-phenyl)-2-[4-(1-ethyl-piperidin-4-yl)-phenylamino]-5-oxo-5,8-dihydro-pyrido[2,3-d]pyrimidine-6-carboxylic acid isopropoxy-amide,
183 8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid ethoxy-amide,
184 8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid isopropoxy-amide,
185 8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid methoxy-amide,
186 8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid ethoxy-amide,
187 8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid isopropoxy-amide,
188 8-(4-ethyl-phenyl)-5-oxo-2-{4-[1-(2,2,2-trifluoro-ethyl)-piperidin-4-yl]-phenylamino}-5,8-dihydro-pyrido[2,3-d]pyrimidine-6-carboxylic acid methoxy-amide,
189 8-(4-ethyl-phenyl)-5-oxo-2-{4-[1-(2,2,2-trifluoro-ethyl)-piperidin-4-yl]-phenylamino}-5,8-dihydro-pyrido[2,3-d]pyrimidine-6-carboxylic acid ethoxy-amide,
190 8-indan-5-yl-5-oxo-2-{4-[2-(1H-[1,2,4]triazol-3-yl)-ethyl]-phenylamino}-5,8-dihydro-pyrido[2,3-d]pyrimidine-6-carboxylic acid methoxy-amide,
191 2-{4-[2-(5-methyl-1H-[1,2,4]triazol-3-yl)-ethyl]-phenylamino}-5-oxo-8-(4-trifluoromethoxyphenyl)-5,8-dihydropyrido[2,3-d]pyrimidine-6-carboxylic acid methoxy-amide,
192 8-indan-5-yl-5-oxo-2-[3-(2-pyrrolidin-1-yl-ethylcarbamoyl)-phenylamino]-5,8-dihydro-pyrido[2,3-d]pyrimidine-6-carboxylic acid methoxy-amide,
193 2-{3-[(1-ethyl-pyrrolidin-2-ylmethyl)-carbamoyl]-phenylamino}-8-indan-5-yl-5-oxo-5,8-dihydro-pyrido[2,3-d]pyrimidine-6-carboxylic acid methoxy-amide,
194 8-indan-5-yl-5-oxo-2-[3-(2-pyrrolidin-1-yl-ethyl)-phenylamino]-5,8-dihydro-pyrido[2,3-d]pyrimidine-6-carboxylic acid methoxy-amide,
195 2-[3-(3,5-dimethyl-piperazin-1-yl)-phenylamino]-8-indan-5-yl-5-oxo-5,8-dihydro-pyrido[2,3-d]pyrimidine-6-carboxylic acid methoxy-amide,
196 2-[3-(4-acetyl-piperazin-1-yl)-phenylamino]-8-indan-5-yl-5-oxo-5,8-dihydro pyrido[2,3-d]pyrimidine-6-carboxylic acid methoxy-amide,
197 2-[3-(1-acetyl-piperidin-4-yl)-phenylamino]-8-indan-5-yl-5-oxo-5,8-dihydro-pyrido[2,3-d]pyrimidine-6-carboxylic acid methoxy-amide,
198 8-indan-5-yl-2-[3-(2-morpholin-4-yl-ethylcarbamoyl)-phenylamino]-5-oxo-5,8-dihydro-pyrido[2,3-d]pyrimidine-6-carboxylic acid methoxy-amide,
199 2-[3-(2-dimethylamino-ethylcarbamoyl)-phenylamino]-8-indan-5-yl-5-oxo-5,8-dihydro-pyrido[2,3-d]pyrimidine-6-carboxylic acid methoxy-amide,
200 8-indan-5-yl-2-{3-[2-(4-methyl-piperazin-1-yl)-ethyl]-phenylamino}-5-oxo-5,8-dihydro-pyrido[2,3-d]pyrimidine-6-carboxylic acid methoxy-amide,
201 8-indan-5-yl-2-[3-(4-methanesulfonyl-piperazin-1-yl)-phenylamino]-5-oxo-5,8-dihydro-pyrido[2,3-d]pyrimidine-6-carboxylic acid methoxy-amide,
202 8-indan-5-yl-5-oxo-2-[3-(4-trifluoromethanesulfonyl-piperazin-1-yl)-phenylamino]-5,8-dihydro-pyrido[2,3-d]pyrimidine-6-carboxylic acid methoxy-amide,
203 8-indan-5-yl-2-{3-[2-(4-methanesulfonyl-piperazin-1-yl)-ethyl]-phenylamino}-5-oxo-5,8-dihydro-pyrido[2,3-d]pyrimidine-6-carboxylic acid methoxy-amide,
204 2-{3-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide,
205 2-{3-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide,
206 2-{4-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide,
207 8-(4-ethyl-phenyl)-2-{4-[1-(2-methoxy-ethyl)-piperidin-4-yl]-phenylamino}-5-oxo-5,8-dihydro-pyrido[2,3-d]pyrimidine-6-carboxylic acid methoxy-amide,
208 8-(4,4-dimethyl-cyclohexyl)-2-[4-(1-ethyl-piperidin-4-yl)-phenylamino]-5-oxo-5,8-dihydro-pyrido[2,3-d]pyrimidine-6-carboxylic acid methoxy-amide,
209 8-(4,4-dimethyl-cyclohexyl)-2-[4-(1-methyl-piperidin-4-yl)-phenylamino]-5-oxo-5,8-dihydro-pyrido[2,3-d]pyrimidine-6-carboxylic acid methoxy-amide,
210 8-(4,4-dimethyl-cyclohexyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide,
211 8-(4-ethyl-phenyl)-2-{4-[1-(2-methoxy-ethyl)-piperidin-4-yl]-phenylamino}-5-oxo-5,8-dihydro-pyrido[2,3-d]pyrimidine-6-carboxylic acid methoxy-amide,
212 8-(3-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide,
213 8-(4-ethyl-phenyl)-2-[4-(3-methoxy-propylamino)-phenylamino]-5-oxo-5,8-dihydro-pyrido[2,3-d]pyrimidine-6-carboxylic acid methoxy-amide,
214 8-indan-5-yl-2-(4-methanesulfonylaminocarbonyl-phenylamino)-5-oxo-5,8-dihydro-pyrido[2,3-d]pyrimidine-6-carboxylic acid methoxy-amide,
215 8-(4-ethyl-phenyl)-2-[3-(2-methoxy-ethoxy)-phenylamino]-5-oxo-5,8-dihydro-pyrido[2,3-d]pyrimidine-6-carboxylic acid methoxy-amide,
216 8-(4-ethyl-phenyl)-2-[3-(3-methoxy-propoxy)-phenylamino]-5-oxo-5,8-dihydro-pyrido[2,3-d]pyrimidine-6-carboxylic acid methoxy-amide,
217 8-(4-ethyl-phenyl)-2-[4-(2-methanesulfonyl-ethylamino)-phenylamino]-5-oxo-5,8-dihydro-pyrido[2,3-d]pyrimidine-6-carboxylic acid methoxy-amide,
218 {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester,
219 {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid,
220 8-indan-5-yl-2-{4-[2-(4-methyl-3-oxo-piperazin-1-yl)-ethyl]-phenylamino}-5-oxo-5,8-dihydro-pyrido[2,3-d]pyrimidine-6-carboxylic acid methoxy-amide,
221 8-(4-ethyl-phenyl)-2-{4-[2-(4-methyl-3-oxo-piperazin-1-yl)-ethyl]-phenylamino}-5-oxo-5,8-dihydro-pyrido[2,3-d]pyrimidine-6-carboxylic acid methoxy-amide,
222 8-indan-5-yl-2-[4-(1-methyl-piperidin-4-yl)-phenylamino]-5-oxo-5,8-dihydro-pyrido[2,3-d]pyrimidine-6-carboxylic acid,
223 8-indan-5-yl-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
224 8-(4-ethyl-phenyl)-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
225 8-(6,7-dihydro-5H-[1]pyrindin-3-yl)-2-[4-(1-methyl-piperidin-4-yl)-phenylamino]-5-oxo-5,8-dihydro-pyrido[2,3-d]pyrimidine-6-carboxylic acid methoxy-amide,
226 8-(6,7-dihydro-5H-[1]pyrindin-3-yl)-2-[4-(2-methanesulfonyl-ethyl)-phenylamino]-5-oxo-5,8-dihydro-pyrido[2,3-d]pyrimidine-6-carboxylic acid methoxy-amide,
227 8-(6,7-dihydro-5H-[1]pyrindin-3-yl)-2-[4-(2-hydroxy-ethyl)-phenylamino]-5-oxo-5,8-dihydro-pyrido[2,3-d]pyrimidine-6-carboxylic acid methoxy-amide,
228 2-[3-(2-dimethylamino-acetylamino)-phenylamino]-8-(4-ethyl-phenyl)-5-oxo-5,8-dihydro-pyrido[2,3-d]pyrimidine-6-carboxylic acid methoxy-amide,
229 2-[3-(2-dimethylamino-acetylamino)-phenylamino]-8-indan-5-yl-5-oxo-5,8-dihydro-pyrido[2,3-d]pyrimidine-6-carboxylic acid methoxy-amide,
230 2-[3-(2-dimethylamino-ethanesulfonyl)-phenylamino]-8-indan-5-yl-5-oxo-5,8-dihydro-pyrido[2,3-d]pyrimidine-6-carboxylic acid methoxy-amide,
231 8-indan-5-yl-2-[3-(2-methylamino-ethanesulfonyl)-phenylamino]-5-oxo-5,8-dihydro-pyrido[2,3-d]pyrimidine-6-carboxylic acid methoxy-amide,
232 2-{4-[2-(3,3-difluoro-piperidin-1-yl)-ethyl]-phenylamino}-8-(4-ethyl-phenyl)-5-oxo-5,8-dihydro-pyrido[2,3-d]pyrimidine-6-carboxylic acid methoxy-amide,
233 2-{4-[2-(3,3-difluoro-piperidin-1-yl)-ethyl]-phenylamino}-8-(4-ethyl-phenyl)-5-oxo-5,8-dihydro-pyrido[2,3-d]pyrimidine-6-carboxylic acid methoxy-amide,
234 2-{4-[2-(4,4-difluoro-piperidin-1-yl)-ethyl]-phenylamino}-8-indan-5-yl-5-oxo-5,8-dihydro-pyrido[2,3-d]pyrimidine-6-carboxylic acid methoxy-amide,
235 8-indan-5-yl-2-[4-(1-methyl-1,2,3,6-tetrahydro-pyridin-4-yl)-phenylamino]-5-oxo-5,8-dihydro-pyrido[2,3-d]pyrimidine-6-carboxylic acid methoxy-amide,
236 2-{4-[(3S,4S)-(3,4-dihydroxy-1-methyl-piperidin-4-yl)]-phenylamino}-8-indan-5-yl-5-oxo-5,8-dihydro-pyrido[2,3-d]pyrimidine-6-carboxylic acid methoxy-amide,
237 2-[4-(3,3-difluoro-1-methyl-piperidin-4-yl)-phenylamino]-8-indan-5-yl-5-oxo-5,8-dihydro-pyrido[2,3-d]pyrimidine-6-carboxylic acid methoxy-amide,
238 2-{4-[2-(4-hydroxy-piperidin-1-yl)-ethyl]-phenylamino}-8-indan-5-yl-5-oxo-5,8-dihydro-pyrido[2,3-d]pyrimidine-6-carboxylic acid methoxy-amide,
239 2-{4-{2-[(3R)-(3-hydroxy-piperidin-1-yl)]-ethyl}-phenylamino}-8-indan-5-yl-5-oxo-5,8-dihydro-pyrido[2,3-d]pyrimidine-6-carboxylic acid methoxy-amide,
240 8-(4-ethyl-phenyl)-2-(4-{2-[(3R)-(3-hydroxy-piperidin-1-yl)]-ethyl}-phenylamino)-5-oxo-5,8-dihydro-pyrido[2,3-d]pyrimidine-6-carboxylic acid methoxy-amide,
241 2-{4-[2-(3,3-difluoro-pyrrolidin-1-yl)-ethyl]-phenylamino}-8-indan-5-yl-5-oxo-5,8-dihydro-pyrido[2,3-d]pyrimidine-6-carboxylic acid methoxy-amide,
242 8-(4-ethyl-phenyl)-5-oxo-2-(4-piperidin-2-yl-phenylamino)-5,8-dihydro-pyrido[2,3-d]pyrimidine-6-carboxylic acid methoxy-amide,
243 8-indan-5-yl-2-[4-(1-methyl-piperidin-2-yl)-phenylamino]-5-oxo-5,8-dihydro-pyrido[2,3-d]pyrimidine-6-carboxylic acid methoxy-amide,
244 2-[4-(1-ethyl-piperidin-2-yl)-phenylamino]-8-indan-5-yl-5-oxo-5,8-dihydro-pyrido[2,3-d]pyrimidine-6-carboxylic acid methoxy-amide,
245 8-(4-ethyl-phenyl)-2-[4-(1-ethyl-piperidin-2-yl)-phenylamino]-5-oxo-5,8-dihydro-pyrido[2,3-d]pyrimidine-6-carboxylic acid methoxy-amide,
246 8-indan-5-yl-5-oxo-2-(4-piperidin-2-yl-phenylamino)-5,8-dihydro-pyrido[2,3-d]pyrimidine-6-carboxylic acid methoxy-amide,
247 2-[3-(1-aza-bicyclo[2.2.2]oct-3-yloxy)-phenylamino]-8-(4-ethyl-phenyl)-5-oxo-5,8-dihydro-pyrido[2,3-d]pyrimidine-6-carboxylic acid methoxy-amide,
248 8-(4-ethyl-phenyl)-5-oxo-2-[3-(piperidin-3-yloxy)-phenylamino]-5,8-dihydro-pyrido[2,3-d]pyrimidine-6-carboxylic acid methoxy-amide,
249 2-{4-[(3R,4R)-(3,4-dihydroxy-1-methyl-piperidin-4-yl)]-phenylamino}-8-(4-ethyl-phenyl)-5-oxo-5,8-dihydro-pyrido[2,3-d]pyrimidine-6-carboxylic acid methoxy-amide,
250 2-{4-[(3R,4R)-(3,4-dihydroxy-1-methyl-piperidin-4-yl)]-phenylamino}-8-indan-5-yl-5-oxo-5,8-dihydro-pyrido[2,3-d]pyrimidine-6-carboxylic acid methoxy-amide,
251 2-[3-(1-ethyl-piperidin-3-yloxy)-phenylamino]-8-indan-5-yl-5-oxo-5,8-dihydro-pyrido[2,3-d]pyrimidine-6-carboxylic acid methoxy-amide,
252 2-(4-{2-[(3R)-(3-fluoro-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
253 2-(4-{2-[(3S)-(3-fluoro-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
254 2-{4-[2-(3-hydroxy-azetidin-1-yl)-ethyl]-phenylamino}-8-indan-5-yl-5-oxo-5,8-dihydro-pyrido[2,3-d]pyrimidine-6-carboxylic acid methoxy-amide,
255 2-(4-{2-[(3S)-(3-hydroxy-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
256 2-{4-[2-(3-fluoro-piperidin-1-yl)-ethyl]-phenylamino}-8-indan-5-yl-5-oxo-5,8-dihydro-pyrido[2,3-d]pyrimidine-6-carboxylic acid methoxy-amide,
257 8-(4-ethyl-phenyl)-2-[4-(2-methoxy-ethoxy)-phenylamino]-5-oxo-5,8-dihydro-pyrido[2,3-d]pyrimidine-6-carboxylic acid methoxy-amide,
258 8-indan-5-yl-2-[4-(2-methoxy-ethoxy)-phenylamino]-5-oxo-5,8-dihydro-pyrido[2,3-d]pyrimidine-6-carboxylic acid methoxy-amide,
259 8-(1H-inden-5-yl)-2-[4-(1-methyl-piperidin-4-yl)-phenylamino]-5-oxo-5,8-dihydro-pyrido[2,3-d]pyrimidine-6-carboxylic acid methoxy-amide,
260 2-[4-(2-azetidin-1-yl-ethyl)-phenylamino]-8-(4-ethyl-phenyl)-5-oxo-5,8-dihydro-pyrido[2,3-d]pyrimidine-6-carboxylic acid methoxy-amide,
261 8-indan-5-yl-2-[3-(2-methoxy-ethoxy)-phenylamino]-5-oxo-5,8-dihydro-pyrido[2,3-d]pyrimidine-6-carboxylic acid methoxy-amide,
262 2-[4-(2-azetidin-1-yl-ethyl)-phenylamino]-8-indan-5-yl-5-oxo-5,8-dihydro-pyrido[2,3-d]pyrimidine-6-carboxylic acid methoxy-amide,
263 2-{3-[(3S)-(1-ethyl-pyrrolidin-3-yloxy)]-phenylamino}-8-indan-5-yl-5-oxo-5,8-dihydro-pyrido[2,3-d]pyrimidine-6-carboxylic acid methoxy-amide,
264 2-[4-(1-aza-bicyclo[2.2.2]oct-3-yloxy)-phenylamino]-8-indan-5-yl-5-oxo-5,8-dihydro-pyrido[2,3-d]pyrimidine-6-carboxylic acid methoxy-amide,
265 8-(4-ethyl-phenyl)-2-{4-[2-(3-fluoro-azetidin-1-yl)-ethyl]-phenylamino}-5-oxo-5,8-dihydro-pyrido[2,3-d]pyrimidine-6-carboxylic acid methoxy-amide,
266 2-{4-[2-(3-fluoro-azetidin-1-yl)-ethyl]-phenylamino}-8-indan-5-yl-5-oxo-5,8-dihydro-pyrido[2,3-d]pyrimidine-6-carboxylic acid methoxy-amide,
267 2-[3-(1-aza-bicyclo[2.2.2]oct-3-yloxy)-phenylamino]-8-indan-5-yl-5-oxo-5,8-dihydro-pyrido[2,3-d]pyrimidine-6-carboxylic acid methoxy-amide,
268 2-[4-(1-aza-bicyclo[2.2.2]oct-3-yloxy)-phenylamino]-8-(4-ethyl-phenyl)-5-oxo-5,8-dihydro-pyrido[2,3-d]pyrimidine-6-carboxylic acid methoxy-amide,
269 2-{4-[2-(3,3-difluoro-azetidin-1-yl)-ethyl]-phenylamino}-8-(4-ethyl-phenyl)-5-oxo-5,8-dihydro-pyrido[2,3-d]pyrimidine-6-carboxylic acid methoxy-amide,
270 2-{4-[2-(3,3-difluoro-azetidin-1-yl)-ethyl]-phenylamino}-8-indan-5-yl-5-oxo-5,8-dihydro-pyrido[2,3-d]pyrimidine-6-carboxylic acid methoxy-amide,
271 8-indan-5-yl-2-[3-(1-methyl-piperidin-3-yl)-phenylamino]-5-oxo-5,8-dihydro-pyrido[2,3-d]pyrimidine-6-carboxylic acid methoxy-amide,
272 2-[3-(4-hydroxy-1-methyl-piperidin-4-yl)-phenylamino]-8-indan-5-yl-5-oxo-5,8-dihydro-pyrido[2,3-d]pyrimidine-6-carboxylic acid methoxy-amide,
273 8-indan-5-yl-2-[3-(1-methyl-piperidin-4-yl)-phenylamino]-5-oxo-5,8-dihydro-pyrido[2,3-d]pyrimidine-6-carboxylic acid methoxy-amide,
274 2-[3-(3-hydroxy-pyrrolidin-3-yl)-phenylamino]-8-indan-5-yl-5-oxo-5,8-dihydro-pyrido[2,3-d]pyrimidine-6-carboxylic acid methoxy-amide,
275 2-[3-(3-hydroxy-piperidin-3-yl)-phenylamino]-8-indan-5-yl-5-oxo-5,8-dihydro-pyrido[2,3-d]pyrimidine-6-carboxylic acid methoxy-amide,
276 2-[3-(1-ethyl-3-hydroxy-piperidin-3-yl)-phenylamino]-8-indan-5-yl-5-oxo-5,8-dihydro-pyrido[2,3-d]pyrimidine-6-carboxylic acid methoxy-amide,
277 2-{3-[4-hydroxy-1-(2,2,2-trifluoro-ethyl)-piperidin-4-yl]-phenylamino}-8-indan-5-yl-5-oxo-5,8-dihydro-pyrido[2,3-d]pyrimidine-6-carboxylic acid methoxy-amide,
278 2-[3-(1-ethyl-3-hydroxy-piperidin-3-yl)-phenylamino]-8-(4-ethyl-phenyl)-5-oxo-5,8-dihydro-pyrido[2,3-d]pyrimidine-6-carboxylic acid methoxy-amide,
279 2-(1-acetyl-2,3-dihydro-1H-indol-5-ylamino)-8-(4-ethyl-phenyl)-5-oxo-5,8-dihydro-pyrido[2,3-d]pyrimidine-6-carboxylic acid methoxy-amide,
280 8-(4-ethyl-phenyl)-2-(1-methanesulfonyl-2,3-dihydro-1H-indol-5-ylamino)-5-oxo-5,8-dihydro-pyrido[2,3-d]pyrimidine-6-carboxylic acid methoxy-amide,
281 2-(1-acetyl-2,3-dihydro-1H-indol-6-ylamino)-8-(4-ethyl-phenyl)-5-oxo-5,8-dihydro-pyrido[2,3-d]pyrimidine-6-carboxylic acid methoxy-amide,
282 8-(4-ethyl-phenyl)-5-oxo-2-(1,2,3,4-tetrahydro-isoquinolin-7-ylamino)-5,8-dihydro-pyrido[2,3-d]pyrimidine-6-carboxylic acid methoxy-amide,
283 8-indan-5-yl-5-oxo-2-(4-[1,2,4]triazol-1-ylmethyl-phenylamino)-5,8-dihydro-pyrido[2,3-d]pyrimidine-6-carboxylic acid methoxy-amide,
284 8-indan-5-yl-5-oxo-2-(4-pyrazol-1-yl-phenylamino)-5,8-dihydro-pyrido[2,3-d]pyrimidine-6-carboxylic acid methoxy-amide,
285 8-(4-ethyl-phenyl)-2-(1H-indol-5-ylamino)-5-oxo-5,8-dihydro-pyrido[2,3-d]pyrimidine-6-carboxylic acid methoxy-amide,
286 8-(4-ethyl-phenyl)-2-(1H-indol-6-ylamino)-5-oxo-5,8-dihydro-pyrido[2,3-d]pyrimidine-6-carboxylic acid methoxy-amide,
287 8-indan-5-yl-2-(1H-indol-5-ylamino)-5-oxo-5,8-dihydro-pyrido[2,3-d]pyrimidine-6-carboxylic acid methoxy-amide,
288 8-(4-ethyl-phenyl)-2-(1H-indazol-6-ylamino)-5-oxo-5,8-dihydro-pyrido[2,3-d]pyrimidine-6-carboxylic acid methoxy-amide,
289 8-indan-5-yl-2-(1H-indol-6-ylamino)-5-oxo-5,8-dihydro-pyrido[2,3-d]pyrimidine-6-carboxylic acid methoxy-amide,
290 8-(4-ethyl-phenyl)-2-(1-methyl-1H-indol-5-ylamino)-5-oxo-5,8-dihydro-pyrido[2,3-d]pyrimidine-6-carboxylic acid methoxy-amide,
291 8-indan-5-yl-2-(1-methyl-1H-indol-5-ylamino)-5-oxo-5,8-dihydro-pyrido[2,3-d]pyrimidine-6-carboxylic acid methoxy-amide,
292 8-indan-5-yl-5-oxo-2-{4-[1-(2,2,2-trifluoro-ethyl)-piperidin-4-yl]-phenylamino}-5,8-dihydro-pyrido[2,3-d]pyrimidine-6-carboxylic acid methoxy-amide,
293 8-indan-5-yl-5-oxo-2-{4-[1-(2,2,3,3,3-pentafluoro-propyl)-piperidin-4-yl]-phenylamino}-5,8-dihydro-pyrido[2,3-d]pyrimidine-6-carboxylic acid methoxy-amide,
294 2-(6,7,9,10,12,13,15,16,18,19-decahydro-5,8,11,14,17,20-hexaoxa-benzocyclooctadecen-2-ylamino)-8-(4-ethyl-phenyl)-5-oxo-5,8-dihydro-pyrido[2,3-d]pyrimidine-6-carboxylic acid methoxy-amide,
295 8-(4-ethyl-phenyl)-5-oxo-2-[4-(1-phenethyl-piperidin-4-yl)-phenylamino]-5,8-dihydro-pyrido[2,3-d]pyrimidine-6-carboxylic acid methoxy-amide,
296 8-(4-ethyl-phenyl)-5-oxo-2-{4-[(2S,3S,4S,5S,6R)(3,4,5-trihydroxy-6-hydroxymethyl-tetrahydro-pyran-2-yloxy)]-phenylamino}-5,8-dihydro-pyrido[2,3-d]pyrimidine-6-carboxylic acid methoxy-amide,
297 8-(4-ethyl-phenyl)-2-(6,7,9,10,12,13,15,16-octahydro-5,8,11,14,17-pentaoxa-benzocyclopentadecen-2-ylamino)-5-oxo-5,8-dihydro-pyrido[2,3-d]pyrimidine-6-carboxylic acid methoxy-amide,
298 8-indan-5-yl-5-oxo-2-[4-(1-propyl-piperidin-4-yl)-phenylamino]-5,8-dihydro-pyrido[2,3-d]pyrimidine-6-carboxylic acid methoxy-amide,
299 8-(4-ethyl-phenyl)-2-(3′-methoxy-biphenyl-4-ylamino)-5-oxo-5,8-dihydro-pyrido[2,3-d]pyrimidine-6-carboxylic acid methoxy-amide,
300 8-indan-5-yl-5-oxo-2-phenylamino-5,8-dihydro-pyrido[2,3-d]pyrimidine-6-carboxylic acid methoxy-amide,
301 2-(1-ethyl-1H-benzoimidazol-5-ylamino)-8-indan-5-yl-5-oxo-5,8-dihydro-pyrido[2,3-d]pyrimidine-6-carboxylic acid methoxy-amide,
302 2-(1H-benzoimidazol-5-ylamino)-8-indan-5-yl-5-oxo-5,8-dihydro-pyrido[2,3-d]pyrimidine-6-carboxylic acid methoxy-amide,
303 2-[4-(1-methyl-piperidin-4-yl)-phenylamino]-8-naphthalen-2-yl-5-oxo-5,8-dihydro-pyrido[2,3-d]pyrimidine-6-carboxylic acid methoxy-amide,
304 8-(4-ethyl-phenyl)-5-oxo-2-(2-oxo-2,3-dihydro-1H-indol-5-ylamino)-5,8-dihydro-pyrido[2,3-d]pyrimidine-6-carboxylic acid methoxy-amide,
305 8-(4-ethyl-phenyl)-5-oxo-2-(3,4,5-trimethoxy-phenylamino)-5,8-dihydro-pyrido[2,3-d]pyrimidine-6-carboxylic acid methoxy-amide,
306 2-(4′-dimethylamino-biphenyl-4-ylamino)-8-(4-ethyl-phenyl)-5-oxo-5,8-dihydro-pyrido[2,3-d]pyrimidine-6-carboxylic acid methoxy-amide,
307 8-(4-ethyl-phenyl)-5-oxo-2-[4-(piperidin-4-yloxy)-phenylamino]-5,8-dihydro-pyrido[2,3-d]pyrimidine-6-carboxylic acid methoxy-amide,
308 2-[3,4-bis-(2-methoxy-ethoxy)-phenylamino]-8-(4-ethyl-phenyl)-5-oxo-5,8-dihydro-pyrido[2,3-d]pyrimidine-6-carboxylic acid methoxy-amide,
309 8-(4-ethyl-phenyl)-2-[4-(1-methyl-piperidin-4-yloxy)-phenylamino]-5-oxo-5,8-dihydro-pyrido[2,3-d]pyrimidine-6-carboxylic acid methoxy-amide,
310 8-(4-ethyl-phenyl)-2-[4-(1-ethyl-piperidin-4-yloxy)-phenylamino]-5-oxo-5,8-dihydro-pyrido[2,3-d]pyrimidine-6-carboxylic acid methoxy-amide,
311 8-indan-5-yl-5-oxo-2-[4-(piperidin-4-yloxy)-phenylamino]-5,8-dihydro-pyrido[2,3-d]pyrimidine-6-carboxylic acid methoxy-amide,
312 2-[4-(1-ethyl-piperidin-4-yloxy)-phenylamino]-8-indan-5-yl-5-oxo-5,8-dihydro-pyrido[2,3-d]pyrimidine-6-carboxylic acid methoxy-amide,
313 8-indan-5-yl-2-[4-(1-methyl-piperidin-4-yloxy)-phenylamino]-5-oxo-5,8-dihydro-pyrido[2,3-d]pyrimidine-6-carboxylic acid methoxy-amide,
314 2-[3,4-bis-(2-methoxy-ethoxy)-phenylamino]-8-indan-5-yl-5-oxo-5,8-dihydro-pyrido[2,3-d]pyrimidine-6-carboxylic acid methoxy-amide,
315 8-indan-5-yl-5-oxo-2-[4-(2aR,1R,7S,6aS)-(1,4,4,7-tetramethyl-3,5,11-trioxa-tricyclo[5.3.1.0^{2,6}]undec-9-yl)-phenylamino]-5,8-dihydro-pyrido[2,3-d]pyrimidine-6-carboxylic acid methoxy-amide,
316 8-(4-ethyl-phenyl)-5-oxo-2-phenylamino-5,8-dihydro-pyrido[2,3-d]pyrimidine-6-carboxylic acid methoxy-amide,
317 8-(4-ethyl-phenyl)-5-oxo-2-(4-pyrrol-1-yl-phenylamino)-5,8-dihydro-pyrido[2,3-d]pyrimidine-6-carboxylic acid methoxy-amide,
318 8-(4-ethyl-phenyl)-5-oxo-2-{4-[1-(2,2,2-trifluoro-ethyl)-piperidin-4-yloxy]-phenylamino}-5,8-dihydro-pyrido[2,3-d]pyrimidine-6-carboxylic acid methoxy-amide,
319 2-(3-dimethylamino-phenylamino)-8-(4-ethyl-phenyl)-5-oxo-5,8-dihydro-pyrido[2,3-d]pyrimidine-6-carboxylic acid methoxy-amide,
320 8-(4-ethyl-phenyl)-2-(3-morpholin-4-yl-phenylamino)-5-oxo-5,8-dihydro-pyrido[2,3-d]pyrimidine-6-carboxylic acid methoxy-amide,
321 8-(4-ethyl-phenyl)-2-[3-(4-methyl-piperazin-1-yl)-phenylamino]-5-oxo-5,8-dihydro-pyrido[2,3-d]pyrimidine-6-carboxylic acid methoxy-amide,
322 8-(4-ethyl-phenyl)-2-[3-(1-ethyl-piperidin-4-yloxy)-phenylamino]-5-oxo-5,8-dihydro-pyrido[2,3-d]pyrimidine-6-carboxylic acid methoxy-amide,
323 2-[3,4-bis-(3-methoxy-propoxy)-phenylamino]-8-(4-ethyl-phenyl)-5-oxo-5,8-dihydro-pyrido[2,3-d]pyrimidine-6-carboxylic acid methoxy-amide,
324 8-indan-5-yl-2-[3-(4-methyl-piperazin-1-yl)-phenylamino]-5-oxo-5,8-dihydro-pyrido[2,3-d]pyrimidine-6-carboxylic acid methoxy-amide,
325 8-indan-5-yl-2-(3-morpholin-4-yl-phenylamino)-5-oxo-5,8-dihydro-pyrido[2,3-d]pyrimidine-6-carboxylic acid methoxy-amide, and
326 2-(3-dimethylamino-phenylamino)-8-indan-5-yl-5-oxo-5,8-dihydro-pyrido[2,3-d]pyrimidine-6-carboxylic acid methoxy-amide.
   General Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

The terms used in describing the invention are commonly used and known to those skilled in the art. As used herein, the following abbreviations and formulas have the indicated meanings:

| Abbreviation | Meaning |
|---|---|
| Cpd | compound |
| DCM | dichloromethane |
| DIC | 2-dimethylaminoisopropyl chloride |
| DMF | N,N-dimethylformamide |
| DMSO | dimethyl sulfoxide |
| HOBT | 1-hydroxybenzotriazole hydrate |
| min | minute(s) |
| h/hr/hrs | hour(s) |
| TEA or Et₃N | triethylamine |
| THF | tetrahydrofuran |

The following general reaction schemes display various methods of preparing the compounds of Formula I. It is recognized by those skilled in the art that some compounds of Formula I may be further derivatized to provide additional embodiments of the invention. Representative compounds prepared by such derivatizations appear in Schemes I, II, and V.

A typical preparation of compounds of the present invention is shown in Scheme I. An amine is reacted with ethyl 3-chloropropionate at elevated temperature in the presence of an inorganic base and a catalytic amount of tetrabutylammonium bromide to afford the aminopropionate ester 1-1.

The amine is reacted with ethyl 4-chloro-2-methylthio-5-pyrimidine carboxylate to provide the corresponding 4-substituted aminopyrimidine 1-2. Cyclization of this diester under Dieckmann conditions affords the bicyclic compound 1-3.

Subsequent halogenation with bromine followed by dehydrohalogenation gives the unsaturated 1-4 (Eur J Med Chem 9 (2000) pp 585-590). The methylthio group is oxidized to the sulfone 1-5, which is subsequently replaced with a substituted amine (wherein Ph is phenyl further optionally substituted with R¹⁰¹) by nucleophilic substitution.

The resulting carboxylic ester 1-6 is converted to the carboxylic acid 1-7 via basic hydrolysis. Decarboxylation to give 1-8 occurs when the carboxylic acid is heated in DMSO in the presence of sodium cyanide (Tet Lett 35 (1994) pp 8303-8306).

The nitrile 1-8 is reacted with an amine under normal coupling conditions to form the corresponding amide 1-9. The amide 1-9 may also be prepared directly from the ester 1-6 when the amine R₁—NH₂ is ammonia, or an alkylamine.

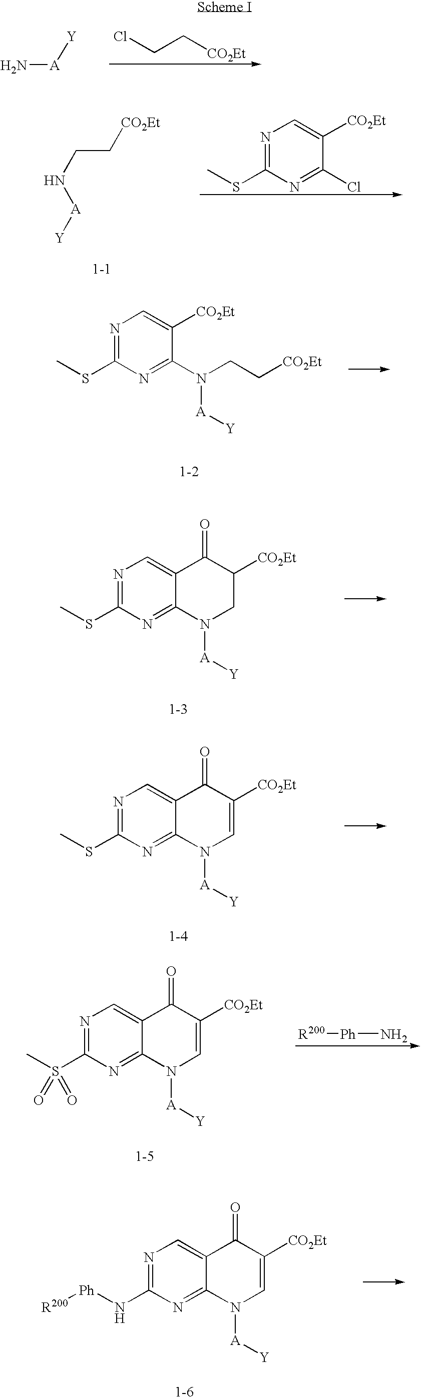

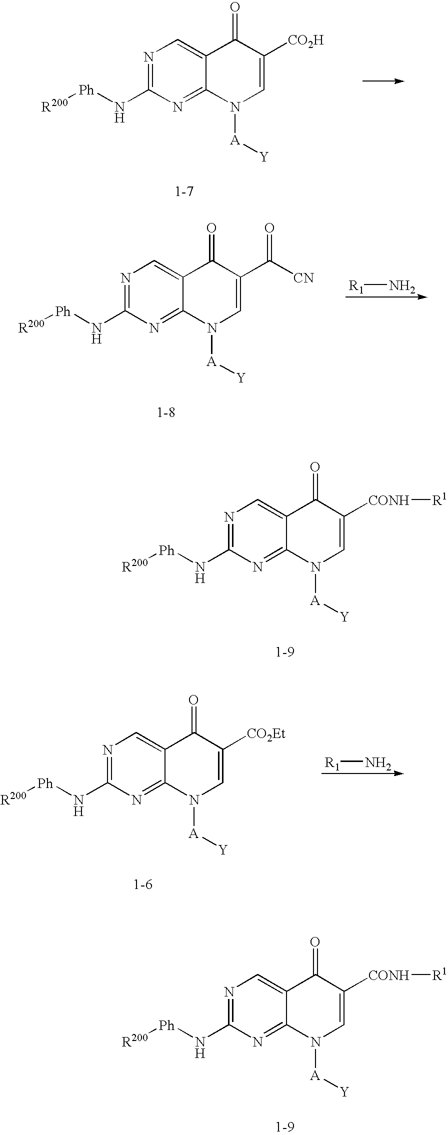

The synthesis is further extended to include the preparation of compounds with a carbonitrile functional group at the C₆ position, as shown in Scheme II. The method of preparation is identical with that used for preparing the esters (Scheme I) except that suitably 3-substituted aminopropionitriles 2-1 are used in the first step. Hydrolysis of 2-5 (wherein Ph is phenyl optionally substituted with R¹⁰¹) under basic conditions provided the corresponding primary amide 2-6.

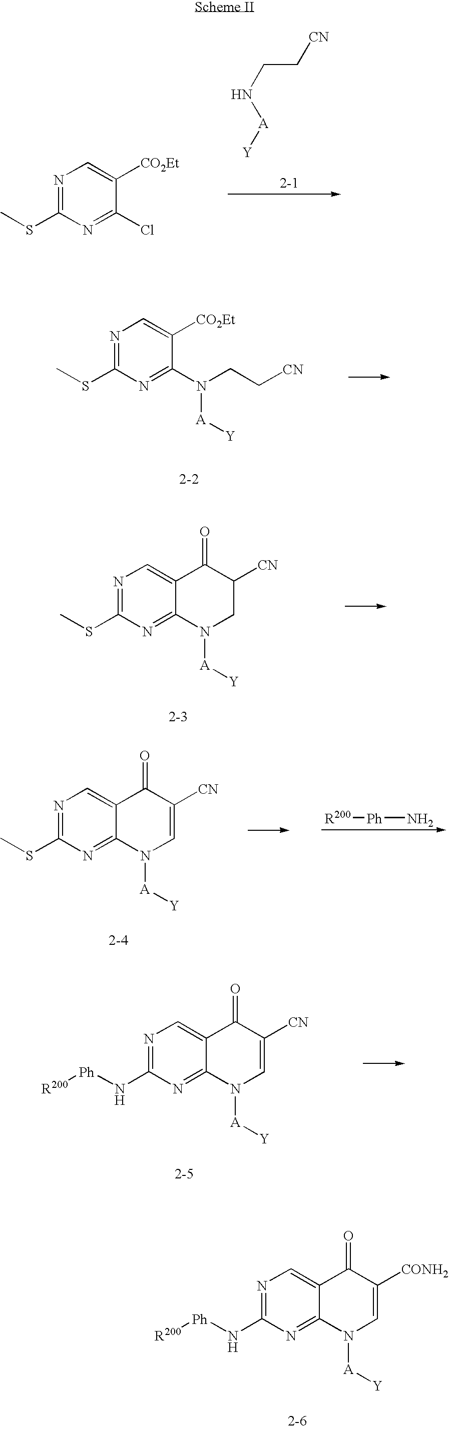

As shown in Scheme III, when a 6-amide is the desired product, the intermediate 3-1 is converted to the primary amide 3-2 using liquid ammonia in a pressure bottle. Subsequent oxidation to methyl sulfone and nucleophilic substitution by a substituted amine (wherein Ph is phenyl further optionally substituted with R¹⁰¹) provides the desired 6-amide analogs 3-3.

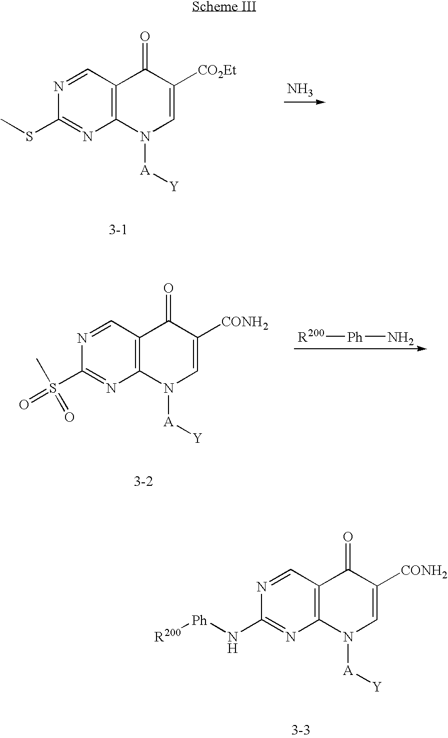

When R²⁰⁰ is a R⁵-heterocyclyl, R³—C₁₋₈alkoxy or R⁴-amino, anilines of the form R²⁰⁰-phenyl-NH₂ (wherein phenyl is further optionally substituted with R¹⁰¹) are prepared using SNAr reactions as shown in Scheme IV (Step A) followed by hydrogenation to convert the nitro group to the amino group.

Where R²⁰⁰ is R²—C₁₋₈alkyl, anilines of the form R²—C₁₋₈alkyl-phenyl-NH₂ (wherein n is an integer from 1 to 8 and phenyl is further optionally substituted with R¹⁰¹) are prepared using SN₂ reactions as shown in Scheme IV (Step B) followed by hydrogenation to convert the nitro group to the amino group.

Where R²⁰⁰ is R⁴-amino-C₁₋₈alkyl-carbonyl, preparation of the aniline R²⁰⁰-phenyl-NH₂ (wherein phenyl is further optionally substituted with R¹⁰¹) may be accomplished using SN₂ reactions as shown in Scheme IV (Step C) followed by hydrogenation to convert the nitro group to the amino group. It is recognized by those skilled in the art that where n is 0, when R²⁰⁰ is R⁴-amino-carbonyl, the desired product may be obtained from nitrobezoic acid, nitrobenzoyl chloride and other starting materials.

Alternatively, anilines where R²⁰⁰ is piperidinyl substituted with an R⁵ substituent R⁶—C₁₋₈alkyl-carbonyl, wherein R⁶ is amino or C₁₋₈alkyl-amino, may be obtained according to Scheme IV (Steps D and E).

As shown in Step D, ketones of formula 4-1 may be converted to a vinyl triflate of formula 4-2 by treatment with a non-nucleophilic base such as LDA and then trapping the resulting enolate with a triflating reagent such as trifluoromethanesulfonic anhydride or preferably N-phenyltrifluoromethanesulfonimide. Suzuki coupling of boronic acids or boronate esters of formula 4-3 (prepared by palladium catalyzed borylation, see for example J. Org. Chem., 60: 7508 (1995)) to vinyl triflates of formula 4-2 provides compounds of formula 4-4 (see, for example, Synthesis, 993 (1991)). Reduction of the olefin with hydrogen over palladium on carbon gives the aniline 4-5.

N-Boc protected anilines of formula 4-6 may be converted to amides of formula 4-7 through normal amide formation reactions (Scheme V, Step E). Anilines of formula 4-8 are obtained by acidic deprotection of the Boc group. It is recognized by those skilled in the art that the same procedure described for Scheme IV (Step E) can also be used to generate ureas wherein the R²⁰⁰ piperidine is substituted with R⁶—C₁₋₈alkyl-carbonyl and R⁶ is amino or C₁₋₈alkyl-amino.

When R²⁰⁰ is R²—C₁₋₈alkyl, anilines of the form R²—C₁₋₈alkyl-phenyl-NH₂ (wherein R² is C₁₋₈alkyl-sulfonyl, amino-sulfonyl, C₁₋₈alkyl-amino-sulfonyl or C₁₋₈alkyl-carbonyl-amino-sulfonyl and phenyl is further optionally substituted with R¹⁰¹) are prepared as described in Scheme IV (Steps F1 and F2).

The thioacetate of formula 4-9 is obtained from nucleophilic replacement of bromide with potassium thioacetate. Hydrolysis followed by treatment with thionyl chloride affords the sulfonyl chloride of formula 4-10, which is subsequently converted to sulfonamides of formula 4-11 when treated with various amines (wherein R^{a} is two substituents each selected from hydrogen, C₁₋₈alkyl or C₁₋₈alkyl-carbonyl). The final nitro reduction provides the anilines of formula 4-12.

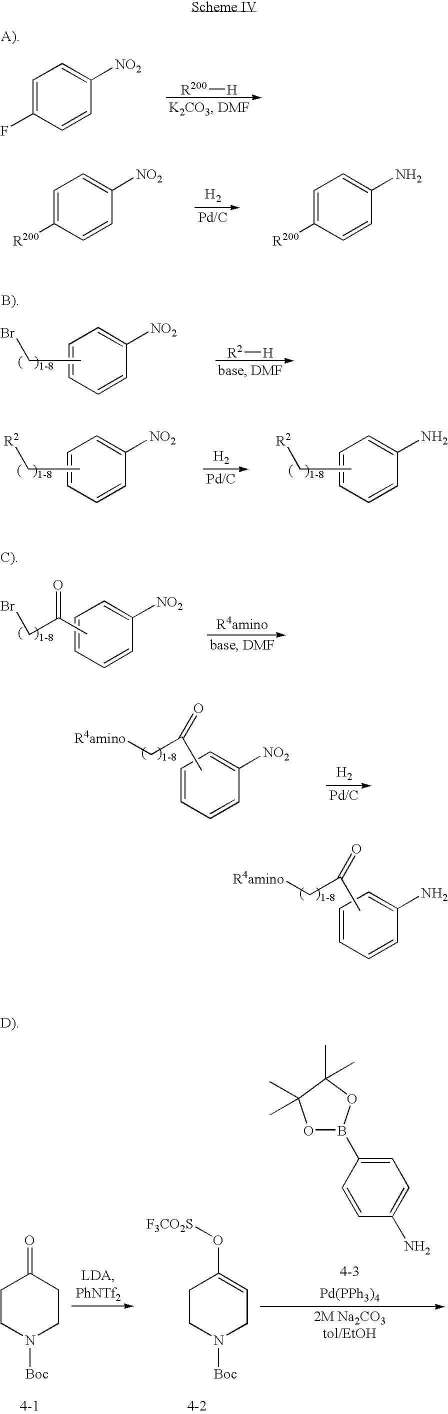

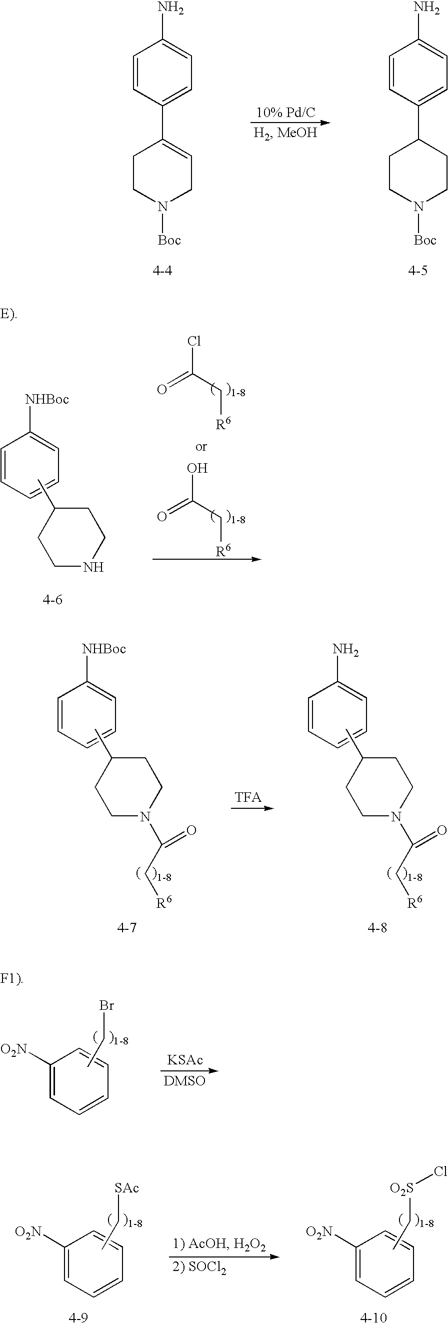

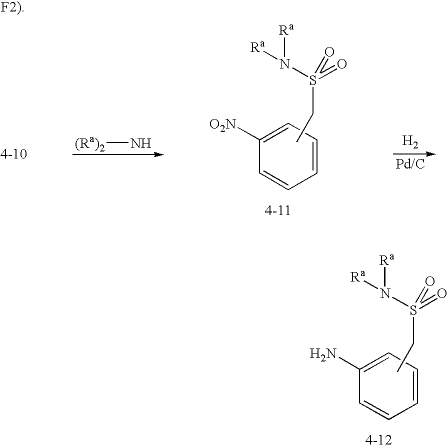

An alternative synthetic sequence to produce compounds of the present invention is described in Scheme V. Reaction of ethyl 4-chloro-2-methylthio-5-pyrimidinecarboxylate with sodium hydroxide in methanol converts the ester group to the corresponding acid group and replaces the chloride group with a methoxy group. The carboxylic acid is then converted to the corresponding acid chloride 5-1 using oxalyl chloride and a drop of DMF in a solvent such as methylene chloride. Reaction of mono-ethylmalonate with a base such as methyl magnesium bromide in a solvent such as ether gives the enolate anion, to which is then added a suspension of acid chloride 5-1 in a solvent such as THF to give ketoester 5-2.

Reaction of 5-2 with triethylorthoformate in acetic anhydride with heating, followed by addition of a substituted amine (wherein A and Y are as defined herein) gives an enamine 5-3. Cyclization of 5-3 with potassium carbonate in a solvent such as THF affords a pyrimidinopyridine 5-4. To further derivatize the ester group, ester 5-4 is hydrolyzed in high yield by heating with a strong acid (such as 1N HCl) in a solvent (such as dioxane and the like) to give a carboxylic acid 5-5. Acid 5-5 can be converted to a variety of amides or alkyl hydroxamides using functional transformations known to those skilled in the art.

One such method is to convert 5-5 to its acid chloride with thionyl chloride or oxalyl chloride. The acid chloride is then reacted with substituted amines to give amide 5-6. Oxidation with mCPBA affords methylsulfone 5-7. Displacement of the methylsulfone with substituted anilines provides 1-9.

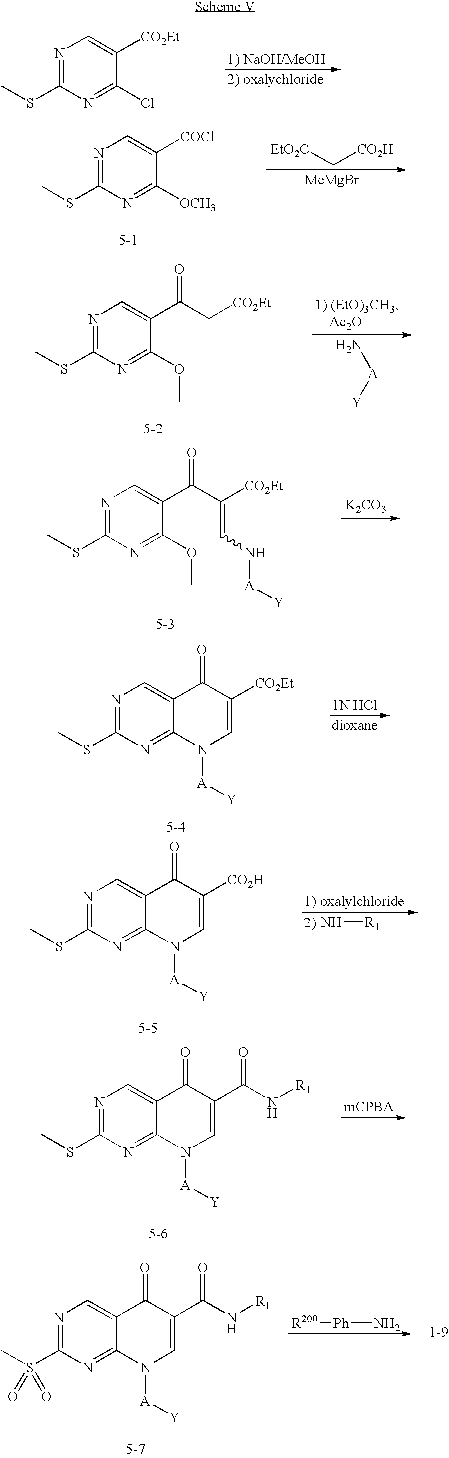

### Example 1

### 8-indan-5-yl-55-oxo-2-(3-piperazin-1-yl-phenylamino)-5,8- dihydro-pyrido[2,3-d]pyrimidine-6-carboxylic acid methoxy-amide (Cpd 2)

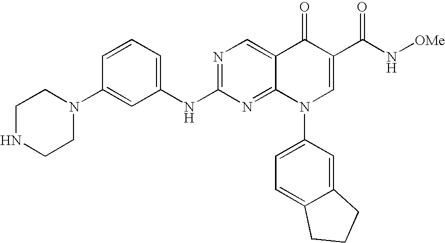

### A. 3-(indan-5-ylamino)-propionic acid ethyl ester

A mixture of tetrabutylammonium bromide (200 mg), 5-aminoindane (5 g, 37.6 mmol), ethyl 3-chloropropionate (4.7 mL, 37.6 mmol) and potassium carbonate (5.2 g, 37.6 mmol) was stirred at 100° C. for 16 hours. After cooling to room temperature (rt), the reaction mixture was extracted with ethyl acetate (EtOAc). The organic fractions were washed with water and brine, and then dried with sodium sulfate (Na₂SO₄) and filtered. The filtrate was concentrated under reduced pressure and the residue was purified by chromatography (silica, EtOAc/hexanes, 1:20-1:10, v/v), to give 6.2 g (71%) of the title compound. ¹H NMR (300 MHz, CDCl₃) δ (ppm): 7.03 (d, J=7.6 Hz, 1H), 6.55 (s, 1H), 6.43 (d, J=7.6 Hz, 1H), 4.15 (q, 2H), 3.86 (br, 1H), 3.43 (t, 2H), 2.82 (m, 4H), 2.60 (t, 2H), 2.06 (m, 2H), 1.27 (t, 3H).

### B. 4-[(2-ethoxycarbonyl-ethyl)-indan-5-yl-amino]-2-methylsulfanyl-pyrimidine-5-carboxylic acid ethyl ester

A solution of 3-(indan-5-ylamino)-propionic acid ethyl ester (4.63 g, 19.9 mmol), ethyl 4-chloro-2-methylthio-5-pyrimidinecarboxylate (4.62 g, 19.9 mmol) and triethylamine (3 g, 29.8 mmol) in 40 mL of n-butanol was stirred at rt for 2 days. The reaction mixture was concentrated under reduced pressure. The residue was dissolved in EtOAc, and the solution was washed with water and brine, dried (Na₂SO₄) and filtered. The filtrate was concentrated, and the residue was purified by chromatography (silica, EtOAc/hexanes, 1:10-1:6, v/v), to give 8.2 g (90%) of the title compound as a white solid. ¹H NMR (300 MHz, CDCl₃) δ (ppm): 8.22 (s, 1H), 7.16 (d, J=7.6 Hz, 1H), 6.95 (s, 1H), 6.87 (d, J=7.6 Hz, 1H), 4.35 (t, 2H), 4.06 (q, 2H), 3.55 (q, 2H), 2.82 (m, 4H), 2.69 (t, 2H), 2.58 (s, 3H), 2.06 (m, 2H), 1.20 (t, 3H), 1.02 (t, 3H).

### C. 8-indan-5-yl-2-methylsulfanyl-5-oxo-5,6,7,8-tetrahydro-pyrido[2,3-d]pyrimidine-6-carboxylic acid ethyl ester

A mixture of sodium (25 wt % dispersion in paraffin wax, 1.6 g, 16.9 mmol) and t-butanol (30 mL) was stirred under N₂. After 10 minutes, a solution of 4-[(2-ethoxycarbonyl-ethyl)-indan-5-yl-amino]-2-methylsulfanyl-pyrimidine-5-carboxylic acid ethyl ester (6.6 g, 15.4 mmol) in 40 mL of toluene was added to the sodium t-butoxide solution, and the reaction mixture was heated at 90° C. for 30 minutes. The solution was cooled and poured onto crushed ice. The mixture was adjusted to pH 7 with HCl. The reaction mixture was extracted twice with EtOAc. The organic fractions were concentrated under vacuum and the title compound (bright yellow solid, 4 g, 62%) was recrystallized from isopropanol.

### D. 8-indan-5-yl-2-methylsulfanyl-5-oxo-5,8-dihydro-pyrido[2,3-d]pyrimidine-6-carboxylic acid ethyl ester

To a solution of 8-indan-5-yl-2-methylsulfanyl-5-oxo-5,6,7,8-tetrahydro-pyrido[2,3-d]pyrimidine-6-carboxylic acid ethyl ester (0.32 g, 0.84 mmol) in 5 mL of methylene chloride (CH₂Cl₂) under N₂ was slowly added bromine (43 μL, 0.84 mmol). The reaction was stirred at room temperature for 2 hours (or to completion). The solvent was removed under reduced pressure without heating. The residue was dissolved in 2 mL of CH₂Cl₂, then triethylamine (234 μL, 1.68 mmol) in 1 mL of CH₂Cl₂ was added, and the reaction was stirred at rt for 4 hours. The progress of the reaction was monitored by LC-MS. The reaction was concentrated under reduced pressure, and the residue was purified by chromatography (silica gel, EtOAc/hexanes, 1:5-1:2.5, v/v) to give the title compound as a white solid (0.30 g, 94%). ¹H NMR (300 MHz, CDCl₃) δ (ppm): 9.42 (s, 1H), 8.59 (s, 1H), 7.37 (d, J=7.8 Hz, 1H), 7.24 (s, 1H), 7.16 (d, J=7.8 Hz, 1H), 4.40 (q, 2H), 3.00 (m, 4H), 2.35 (s, 3H), 2.10 (m, 2H), 1.40 (t, 3H).

### E. 8-indan-5-yl-2-methanesulfonyl-5-oxo-5,8-dihydro-pyrido[2,3-d]pyrimidine-6-carboxylic acid ethyl ester

To a solution of 8-indan-5-yl-2-methylsulfanyl-5-oxo-5,8-dihydro-pyrido[2,3-d]pyrimidine-6-carboxylic acid ethyl ester (0.3 g, 0.79 mmol) in 5 mL of CH₂Cl₂, was added portionwise 3-chloroperoxybenzoic acid (m-CPBA, 69.5%, 431 mg, 1.73 mmol). The reaction was stirred at room temperature for 3 hours. To the reaction was added an aqueous solution of 10% sodium thiosulfate to quench the reaction. After 30 minutes, saturated sodium bicarbonate solution was added, and the mixture was extracted with CH₂Cl₂. The combined organic fractions were washed with brine, dried over Na₂SO₄ and filtered. The filtrate was concentrated under reduced pressure and the residue was purified by chromatography (silica, EtOAc/hexanes, 1:3-1:1.6, v/v) to give the title compound (0.22 g) as an off-white solid. ¹H NMR (300 MHz, CDCl₃) δ (ppm): 9.75 (s, 1H), 8.70 (s, 1H), 7.39 (d, J=7.8 Hz, 1H), 7.24 (s, 1H), 7.16 (d, J=7.8 Hz, 1H), 4.38 (q, 2H), 3.19 (s, 3H), 3.00 (m, 4H), 2.10 (m, 2H), 1.40 (t, 3H).

### F. 4-(3-amino-phenyl)-piperazine-1-carboxylic acid tert-butyl ester

A mixture of 1-fluoro-3-nitrobenzene (Ig, 7 mmol), potassium carbonate (Ig, 7 mmol), and 1-Boc-piperazine (1.9 g, 10 mmol) in DMSO (10 mL) was stirred at 122° C. for 24 hours. After cooling, the mixture was diluted with water (100 mL) and extracted with EtOAc (3×100 mL). The combined organic fractions were washed with brine, dried (Na₂SO₄), and filtered. The filtrate was concentrated in vacuo to afford an orange oil. Recrystallization from EtOAc/hexanes gave 4-(3-nitro-phenyl)-piperazine-1-carboxylic acid tert-butyl ester, which was reduced via hydrogenation to give the title compound (beige solid, 1.9 g). ¹H NMR (400 MHz, CDCl₃) δ (ppm): 7.03 (t, J=8.0 Hz, 1H), 6.34 (d, J=8.2 Hz, 1H), 6.23 (m, 2H), 3.62 (br, 2H), 3.53 (m, 4H), 3.07 (m, 2H), 1.47 (s, 9H).

### G. 8-indan-5-yl-5-oxo-2-(3-piperazin-1-yl-phenylamino)-5,8-dihydro-pyrido[2,3-d]pyrimidine-6-carboxylic acid ethyl ester

A solution of 4-(3-amino-phenyl)-piperazine-1-carboxylic acid tert-butyl ester (54 mg, 0.19 mmol) and 8-indan-5-yl-2-methanesulfonyl-5-oxo-5,8-dihydro-pyrido[2,3-d]pyrimidine-6-carboxylic acid ethyl ester (80 mg, 0.19 mmol) in iPrOH was heated at 90° C. for 1 h. The reaction mixture was concentrated, and the residue was purified by chromatography (HPLC, C-18 YMC ODS-A 5 m 30×100 mm, 120 A column, 32 mL/min 5-100% MeCN/H₂O gradient over 10 min) to obtain a yellow solid (60 mg, 62%). The yellow solid was stirred for 1 hour in a solution of TFA and CH₂Cl₂ (1:1 v/v) and concentrated to give the title compound. ¹H NMR (400 MHz, CDCl₃/CD₃OD 10:1 v/v) δ (ppm): 9.26 (br, 1H), 8.49 (s, 1H), 7.35 (d, J=7.9 Hz, 1H), 7.20 (s, 1H), 7.11 (m, 2H), 6.78 (br, 2H), 6.54 (m, 1H), 4.24 (q, J=7.1 Hz, 2H), 3.98 (m, 4H), 2.29 (m, 4H), 2.96 (m, 4H), 2.14 (m, 2H), 1.27 (t, J=7.1 Hz, 3H).

### H. 8-indan-5-yl-5-oxo-2-(3-piperazin-1-yl-phenylamino)-5,8-dihydro-pyrido[2,3-d]pyrimidine-6-carboxylic acid methoxy-amide

A mixture of 8-indan-5-yl-5-oxo-2-(3-piperazin-1-yl-phenylamino)-5,8-dihydro-pyrido[2,3-d]pyrimidine-6-carboxylic acid ethyl ester (23 mg, 0.045 mmol), 141 mg of methoxyamine hydrochloride, 1 mL of triethylamine in 0.5 mL of methanol was heated in a sealed vial for 12 h at 100° C. After cooling, 20 mL of water was added and the mixture was extracted with 30 mL of CH₂Cl₂. The organic fraction was concentrated under reduced pressure. After purification (HPLC, C-18 YMC ODS-A 5 m 30×100 mm, 120 A column, 32 mL/min, 5-80% MeCN/H₂O (0.1% TFA v/v) gradient over 12 min), the title compound was obtained as a yellow solid was (TFA salt, 6.1 mg, 26%). ¹H NMR (400 MHz, CDCl₃/CD₃OD 10:1 v/v) δ (ppm): 9.30 (s, 1H), 8.73 (s, 1H), 7.37 (d, 1H), 7.23 (s, 1H), 7.17 (br, 1H), 7.10 (d, 1H), 6.85 (br, 1H), 6.54 (br, 2H), 3.82 (s, 3H), 3.23 (br, 8H), 2.97 (t, 2H), 2.91 (t, 2H), 2.18 (m, 2H). Mass Spectrum (LCMS, ESI pos.) Calcd. For C₂₈H₂₉N₇O₃: 512.23 (M+H), Found 512.2.

Using the procedures described in Example 1, and reagents, starting materials and conditions known to those skilled in the art, the following compounds representative of the present invention were prepared:

| Cpd | Name and Data |
|---|---|
| 1 | 8-indan-5-yl-5-oxo-2-(3-piperazin-1-yl-phenylamino)-5,8-dihydro-pyrido[2,3- |
| | d]pyrimidine-6-carboxylic acid isopropylamide |
| | ¹H NMR (400 MHz, CDCl₃/CD₃OD 10:1 v/v) δ (ppm): 9.65 (d, 1H), 9.36 (s, 1H), |
| | 8.80 (s, 1H), 7.39 (d, J = 7.9 Hz, 1H), 7.23 (s, 1H), 7.17 (br, 1H), 7.15 (d, J = 7.9 Hz, 1H), |
| | 6.90 (br, 1H), 6.60 (br, 1H), 6.57 (m, 1H), 4.23 (m, 1H), 3.27 (m, 8H), 3.02 (t, 2H), |
| | 2.95 (t, 2H), 2.20 (m, 2H), 1.25 (d, 6H). Mass Spectrum (LCMS, ESI pos.) Calcd. For |
| | C₃₀H₃₃N₇O₂: 524.27 (M + H), Found 524.2. |
| 3 | 8-(3-chloro-4-fluoro-phenyl)-2-{4-[2-(4-methyl-piperazin-1-yl)-ethyl]-phenylamino}-5- |
| | oxo-5,8-dihydro-pyrido[2,3-d]pyrimidine-6-carboxylic acid amide |
| | Preparation of 4-[2-(4-methyl-piperazin-1-yl)-ethyl]-phenylamine: A mixture of 1-(2- |
| | bromo-ethyl)-4-nitro-benzene (0.9 g, 3.9 mmol), 1-methyl-piperazine (520 μL, 4.7 mmol) |
| | and potassium carbonate (1 g, 4.7 mmol) in 5 mL of DMSO was stirred at 100° C. |
| | for 2 hours. After cooling,, the mixture was extracted into ethyl acetate. The organic |
| | fractions were washed with water and brine, dried (Na₂SO₄) and filtered. The filtrate |
| | was concentrated, and the residue was purified by chromatography (silica, |
| | EtOAc/CH₃OH/NH₄OH, 10:1:0.1, v/v), to give 850 mg of 1-methyl-4-[2-(4-nitro- |
| | phenyl)-ethyl]-piperazine, which was converted under normal hydrogenation conditions |
| | to give the title compound as a yellow solid (780 mg, 91%). ¹H NMR (400 MHz, |
| | CDCl₃) δ (ppm): 6.98 (d, J = 8.3 Hz, 2H), 6.61 (d, J = 8.3 Hz, 2H), 3.57 (br, 2H), |
| | 2.71 (br, 12H), 2.43 (br, 3H). ¹H NMR (300 MHz, CDCl₃) δ (ppm): 9.28 (s, 1H), 8.70 (s, |
| | 1H), 7.54 (br, 2H), 7.34 (m, 2H), 7.19 (m, 1H), 6.95 (br, 2H), 2.60-2.90 (m, 15H). Mass |
| | Spectrum (LCMS, ESI pos.) Calcd. For C₂₇H₂₇ClFN₇O₂: 536.19 (M + H), Found 536.2. |
| 4 | 8-(3-chloro-4-fluoro-phenyl)-2-{4-[2-(4-methyl-piperazin-1-yl)-ethyl]-phenylamino}-5- |
| | oxo-5,8-dihydro-pyrido[2,3-d]pyrimidine-6-carboxylic acid methoxy amide |
| | ¹H NMR (400 MHz, CDCl₃) δ (ppm): 11.82 (s, 1H), 9.30 (s, 1H), 8.70 (s, 1H), 7.52 (br, |
| | 2H), 7.34 (m, 2H), 7.19 (m, 1H), 6.95 (br, 2H), 3.83 (s, 3H), 2.60-2.90 (m, 15H). Mass |
| | Spectrum (LCMS, ESI pos.) Calcd. For C₂₈H₂₉ClFN₇O₃: 566.20 (M + H), Found 566.2. |
| 5 | 8-indan-5-yl-5-oxo-2-[4-(2-pyrrolidin-1-yl-ethyl)-phenylamino]-5,8-dihydro-pyrido[2,3- |
| | d]pyrimidine-6-carboxylic acid amide |
| | ¹H NMR (400 MHz, CDCl₃) δ (ppm): 9.28 (s, 1H), 8.78 (s, 1H), 7.60 (br, 1H), 7.40 (d, J = |
| | 7.9 Hz, 1H), 7.21 (br, 3H), 7.12 (d, J = 7.9 Hz, 1H), 6.86 (br, 2H), 3.33 (m, 2H), |
| | 3.05 (t, J = 7.4 Hz, 2H), 2.93 (t, J = 7.4 Hz, 2H), 2.80 (m, 6H), 2.19 (m, 2H), 1.80 (m, 4H). |
| | Mass Spectrum (LCMS, ESI pos.) Calcd. For C₂₉H₃₀N₆O₂: 495.24 (M + H), Found |
| | 495.3. |
| 6 | (S)-2-[4-(2-hydroxymethyl-pyrrolidin-1-yl)-phenylamino]-8-indan-5-yl-5-oxo-5,8- |
| | dihydro-pyrido[2,3-d]pyrimidine-6-carboxylic acid amide |
| | (S)-4-(2-hydroxymethyl-pyrrolidin-1-yl)-phenylamine was prepared as follows: a |
| | mixture of potassium carbonate (1.9 g, 14.2 mmol), 1-fluoro-4-nitrobenzene (1 g, 7.1 mmol) |
| | and (S)-pyrrolidin-2-yl-methanol (0.85 g, 8.5 mmol) in methyl sulfoxide (DMSO, |
| | 5 mL) was stirred at 80° C. for 3 hours. After cooling, the reaction was extracted with |
| | EtOAc. The organic fractions were washed with water and brine, dried (Na₂SO₄) and |
| | filtered. The filtrate was concentrated in vacuo to give an orange solid. A mixture of |
| | the solid in 25 mL of methanol and palladium on carbon (10% Pd/C, 50 mg) was stirred |
| | at rt for 16 hours under hydrogen. The reaction mixture was filtered through a pad of |
| | celite and the filtrate was concentrated to give the title compound (0.91 g). ¹H NMR |
| | (400 MHz, CDCl₃) δ (ppm): 9.48 (br, 1H), 9.20 (s, 1H), 8.78 (s, 1H), 7.60 (br, 1H), |
| | 7.35 (d, J = 7.9 Hz, 1H), 7.10 (m, 4H), 6.33 (d, J = 8.2 Hz, 1H), 3.50 (m, 2H), 3.34 (m, 3H), |
| | 2.98 (m, 4H), 2.12 (m, 2H), 1.80 (m, 4H). Mass Spectrum (LCMS, ESI pos.) Calcd. For |
| | C₂₈H₂₈N₆O₃: 497.22 (M + H), Found 497.2. |
| 7 | (S)-2-[4-(2-hydroxymethyl-pyrrolidin-1-yl)-phenylamino]-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ (ppm): 11.95 (s, 1H), 9.25 (s, 1H), 8.78 (s, 1H), 7.60 (br, |
| | 1H), 7.37 (d, J = 7.9 Hz, 1H), 7.10 (m, 4H), 6.35 (m, 2H), 3.80 (s, 3H), 3.40-3.80 (m, |
| | 5H), 2.98 (m, 4H), 2.18 (m, 2H), 1.93 (br, 4H). Mass Spectrum (LCMS, ESI pos.) |
| | Calcd. For C₂₉H₃₀N₆O₄: 527.24 (M + H), Found 527.2. |
| 8 | 8-indan-5-yl-5-oxo-2-[4-(2-oxo-pyrrolidin-1-ylmethyl)-phenylamino]-5,8-dihydro- |
| | pyrido[2,3-d]pyrimidine-6-carboxylic acid amide |
| | 4-(2-oxo-pyrrolidin-1-ylmethyl)-phenylamine was prepared as follows: a mixture of p- |
| | nitrobenzyl bromide (2.16 g, 10.0 mmol), pyrrolidin-2-one (1.02 g, 12.0 mmol) and |
| | potassium carbonate (2.0 g, 15.0 mmol) in 1 mL of DMSO was stirred at 120° C. for 16 |
| | hours. After cooling, the mixture was extracted with ethyl acetate. The organic |
| | fractions were washed with water and brine, dried (Na₂SO₄) and filtered. The filtrate |
| | was concentrated and the residue was purified by chromatography (silica, |
| | EtOAc/CH₃OH/NH₄OH, 10:1:0.1, v/v), to give 200 mg of 1-(4-nitro-benzyl)-pyrrolidin- |
| | 2-one, which was converted to 3-(4-methyl-piperazin-1-ylmethyl)-phenylamine under |
| | hydrogenation conditions and obtained as a yellow solid (51 mg). ¹H NMR (400 MHz, |
| | CDCl₃) δ (ppm): 9.45 (br, 1H), 9.33 (s, 1H), 8.80 (s, 1H), 7.37 (d, J = 7.9 Hz, 1H), |
| | 7.23 (m, 3H), 7.15 (d, J = 7.9 Hz, 1H), 6.90 (br, 2H), 5.89 (br, 1H), 4.40 (s, 2H), 3.18 (t, 2H), |
| | 3.05 (t, 2H), 2.95 (t, 2H), 2.38 (t, 2H), 2.12 (m, 2H), 1.82 (m, 2H). Mass Spectrum |
| | (LCMS, ESI pos.) Calcd. For C₂₈H₂₆N₆O₃: 495.21 (M + H), Found 495.2. |
| 9 | 8-indan-5-yl-5-oxo-2-[4-(2-oxo-pyrrolidin-1-ylmethyl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 11.88 (s, 1H), 8.78 (s, 1H), 7.37 (d, J = 7.9 Hz, |
| | 1H), 7.23 (br, 3H), 7.10 (d, J = 7.9 Hz, 1H), 6.90 (br, 2H), 4.30 (s, 2H), 3.83 (s, 3H), |
| | 3.18 (t, 2H), 3.02 (t, 2H), 2.95 (t, 2H), 2.38 (t, 2H), 2.15 (m, 2H), 1.90 (m, 2H). Mass |
| | Spectrum (LCMS, ESI pos.) Calcd. For C₂₉H₂₈N₆O₄: 525.22 (M + H), Found 525.2. |
| 10 | 8-cyclohexyl-5-oxo-2-(4-pyrrolidin-1-yl-phenylamino)-5,8-dihydro-pyrido[2,3- |
| | d]pyrimidine-6-carboxylic acid methoxy-amide |
| | ¹H NMR (400 MHz, CDCl₃) δ (ppm): 9.26 (br, 1H), 8.70 (s, 1H), 7.47 (br, 2H), 6.62 (br, |
| | 2H), 5.10 (m, 1H), 3.83 (s, 3H), 3.32 (br, 4H), 2.03 (m, 4H), 1.36-1.87 (m, 6H). Mass |
| | Spectrum (LCMS, ESI pos.) Calcd. For C₂₅H₃₀N₆O₃: 463.24 (M + H), Found 463.3. |
| 11 | 8-cyclohexyl-5-oxo-2-[4-(pyrrolidine-1-carbonyl)-phenylamino]-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.79 (s, 1H), |
| | 7.70 (dd, 2H), 6.53 (dd, 2H), 5.10 (m, 1H), 3.83 (s, 3H), 3.58 (t, 2H), 3.42 (t, 2H), 2.03 (m, |
| | 4H), 1.30-2.10 (m, 14H). Mass Spectrum (LCMS, ESI pos.) Calcd. For C₂₆H₃₀N₆O₄: |
| | 491.23 (M + H), Found 491.3. |
| 12 | 8-cyclohexyl-5-oxo-2-[4-(2-oxo-pyrrolidin-1-ylmethyl)-phenylamino]-5,8-dihydro- |
| | pyrido[2,3-d]pyrimidine-6-carboxylic acid methoxy-amide |
| | 4-(2-oxo-pyrrolidin-1-ylmethyl)-phenylamine was prepared as described for Cpd 8. ¹H |
| | NMR (400 MHz, CDCl₃/CD₃OD 10:1 v/v) δ (ppm): 9.35 (s, 1H), 8.80 (s, 1H), 7.62 (d, |
| | 2H), 7.23 (d, 2H), 5.12 (m, 1H), 3.83 (s, 3H), 3.25 (t, 2H), 2.40 (t, 2H), 2.03 (m, 4H), |
| | 1.30-1.90 (m, 8H). Mass Spectrum (LCMS, ESI pos.) Calcd. For C₂₆H₃₀N₆O₄: |
| | 491.23 (M + H), Found 491.3. |
| 13 | 8-cyclohexyl-5-oxo-2-{4-[2-(2-oxo-pyrrolidin-1-yl)-ethyl]-phenylamino}-5,8-dihydro- |
| | pyrido[2,3-d]pyrimidine-6-carboxylic acid methoxy-amide |
| | 1-[2-(4-amino-phenyl)-ethyl]-pyrrolidin-2-one was prepared using the procedure for |
| | preparation of 4-[2-(4-methyl-piperazin-1-yl)-ethyl]-phenylamine. ¹H NMR (400 MHz, |
| | CDCl₃) δ (ppm): 11.95 (s, 1H), 9.30 (s, 1H), 8.78 (s, 1H), 7.58 (d, 2H), 7.23 (d, 2H), |
| | 5.08 (m, 1H), 3.81 (s, 3H), 3.45 (t, 2H), 3.25 (t, 2H), 2.80 (t, 2H), 2.30 (t, 2H), 1.95 (m, |
| | 4H), 1.20-1.80 (m, 6H), 1.25 (t, 2H). Mass Spectrum (LCMS, ESI pos.) Calcd. For |
| | C₂₇H₃₂N₆O₄: 505.25 (M + H), Found 505.3. |
| 14 | 8-cyclohexyl-5-oxo-2-[4-(2-pyrrolidin-1-yl-ethyl)-phenylamino]-5,8-dihydro- |
| | pyrido[2,3-d]pyrimidine-6-carboxylic acid methoxy-amide |
| | 4-(2-pyrrolidin-1-yl-ethyl)-phenylamine was prepared using the procedure for |
| | preparation of 4-[2-(4-methyl-piperazin-1-yl)-ethyl]-phenylamine. ¹H NMR (400 MHz, |
| | CDCl₃) δ (ppm): 11.95 (s, 1H), 9.30 (s, 1H), 8.75 (s, 1H), 7.75 (br, 1H), 7.55 (d, 2H), |
| | 7.20 (d, 2H), 5.08 (m, 1H), 3.81 (s, 3H), 2.83 (t, 2H), 2.70 (t, 2H), 2.60 (br, 4H), |
| | 1.20-2.00 (m, 14H). Mass Spectrum (LCMS, ESI pos.) Calcd. For C₂₇H₃₄N₆O₃: 491.27 (M + H), |
| | Found 491.3. |
| 15 | 8-cyclohexyl-5-oxo-2-[4-(2-pyrrolidin-1-yl-ethyl)-phenylamino]-5,8-dihydro- |
| | pyrido[2,3-d]pyrimidine-6-carboxylic acid ethoxy-amide |
| | ¹H NMR (400 MHz, CDCl₃) δ (ppm): 11.87 (s, 1H), 9.30 (s, 1H), 8.75 (s, 1H), 7.55 (d, |
| | 2H), 7.20 (d, 2H), 5.08 (m, 1H), 4.02 (q, 2H), 2.90 (m, 2H), 1.20-2.00 (m, 20H), 1.25 (t, |
| | 3H). Mass Spectrum (LCMS, ESI pos.) Calcd. For C₂₈H₃₆N₆O₃: 505.28 (M + H), Found |
| | 505.4. |
| 16 | 8-cyclohexyl-5-oxo-2-[4-(2-pyrrolidin-1-yl-ethyl)-phenylamino]-5,8-dihydro- |
| | pyrido[2,3-d]pyrimidine-6-carboxylic acid isopropoxy-amide |
| | ¹H NMR (400 MHz, CDCl₃) δ (ppm): 11.85 (s, 1H), 9.38 (s, 1H), 8.80 (s, 1H), 7.68 (br, |
| | 1H), 7.59 (d, 2H), 7.23 (d, 2H), 5.12 (m, 1H), 4.25 (m, 1H), 2.95 (m, 2H), 2.80 (m, 2H), |
| | 2.70 (br, 4H), 2.03 (m, 4H), 1.20-1.90 (m, 10H), 1.32 (s, 3H), 1.30 (s, 3H). Mass |
| | Spectrum (LCMS, ESI pos.) Calcd. For C₂₉H₃₈N₆O₃: 519.30 (M + H), Found 519.4. |
| 17 | 8-cyclohexyl-5-oxo-2-[4-(2-pyrrolidin-1-yl-ethyl)-phenylamino]-5,8-dihydro- |
| | pyrido[2,3-d]pyrimidine-6-carboxylic acid hydroxyamide |
| | ¹H NMR (300 MHz, CDCl₃) δ (ppm): ¹H NMR (400 MHz, CDCl₃) δ (ppm): 11.95 (s, |
| | 1H), 9.30 (s, 1H), 8.75 (s, 1H), 7.75 (br, 1H), 7.55 (d, 2H), 7.20 (d, 2H), 5.08 (m, 1H), |
| | 2.83 (t, 2H), 2.70 (t, 2H), 2.60 (br, 4H), 1.20-2.00 (m, 14H). Mass Spectrum (LCMS, |
| | ESI pos.) Calcd. For C₂₆H₃₂N₆O₃: 477.25 (M + H), Found 477.3. |
| 61 | 8-indan-5-yl-2-{4-[2-(4-methyl-piperazin-1-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 11.98 (s, 1H), 9.25 (s, 1H), 8.81 (s, 1H), 7.50 (br, |
| | 1H), 7.36 (d, 1H), 7.25 (br, 2H), 7.20 (s, 1H), 7.10 (d, 1H), 6.90 (br, 2H), 3.85 (s, 3H), |
| | 3.05 (br, 2H), 2.95 (t, 2H), 2.70 (m, 2H), 2.50 (m, 10H), 2.30 (s, 3H), 2.20 (m, 2H). |
| | Mass Spectrum (LCMS, ESI pos.) Calcd. For C₃₁H₃₅N₇O₃: 554.28 (M + H), Found |
| | 554.1. |
| 112 | 8-indan-5-yl-5-oxo-2-[3-(piperidine-1-carbonyl)-phenylamino]-5,8-dihydro-pyrido[2,3- |
| | d]pyrimidine-6-carboxylic acid amide |
| | ¹H NMR (TFA salt) (400 MHz, CDCl₃) δ (ppm): 9.34 (s, 1H), 8.78 (s, 1H), 7.54 (d, J = 7.18 Hz, |
| | 1H), 7.33 (d, J = 7.90 Hz, 2H), 7.11 (dd, J = 7.89, 1.95 Hz, 2H), 6.95 (br s, |
| | 2H), 3.76-3.50 (m, 2H), 3.34-3.12 (m, 2H), 2.99 (t, J = 7.40, 2H), 2.92 (t, J = 7.39 2H), |
| | 2.24-2.03 (m, 2H), 1.57 (d, J = 16.31 Hz, 4H), 1.42 (dd, J = 2.03, 1.14 Hz, 2H); Mass |
| | Spectrum (LCMS, APCI pos.) Calcd. For C₂₉H₂₈N₆O₃: 508.22; Found: 509.2 (M + H). |
| 113 | 8-indan-5-yl-5-oxo-2-[3-(piperidine-1-carbonyl)-phenylamino]-5,8-dihydro-pyrido[2,3- |
| | d]pyrimidine-6-carboxylic acid methylamide |
| | ¹H NMR (TFA salt) (400 MHz, CDCl₃) δ (ppm): 9.32 (s, 1H), 8.76 (s, 1H), |
| | 7.60-7.43 (m, 2H), 7.40-7.28 (m, 2H), 7.16-7.02 (m, 2H), 7.07-6.86 (m, 3H), 3.74-3.53 (m, 2H), |
| | 3.42-3.12 (m, 2H), 3.07-2.87 (m, 4H), 2.82 (s, 3H), 1.71-1.52 (m, 2H); Mass Spectrum |
| | (LCMS, APCI pos.) Calcd. For C₃₀H₃₀N₆O₃: 522.24; Found: 523.2 (M + H). |
| 114 | 8-benzyl-2-[4-(4-methyl-piperazin-1-yl)-phenylamino]-5-oxo-5,8-dihydro-pyrido[2,3- |
| | d]pyrimidine-6-carboxylic acid amide |
| | Preparation of 4-(4-methyl-piperazin-1-yl)-phenylamine: a mixture of potassium |
| | carbonate (1.9 g, 14.2 mmol), 1-fluoro-4-nitrobenzene (1 g, 7.1 mmol) and 1-methyl- |
| | piperazine (0.94 mL, 8.5 mmol) in methyl sulfoxide (DMSO, 5 mL) was stirred at 80° C. |
| | for 3 hours. After cooling, the reaction mixture was extracted into EtOAc. The organic |
| | fractions were washed with water and brine, dried (Na₂SO₄) and filtered). The residue |
| | was concentrated in vacuo to give an orange solid. A mixture of the product dissolved |
| | in 25 mL of methanol with palladium on carbon (10% Pd/C, 50 mg) was stirred at rt for |
| | 16 hours under hydrogen. The reaction mixture was filtered through a pad of celite, and |
| | the filtrate was concentrated to give the title compound as a dark purple solid (1.3 g, |
| | 80%). ¹H NMR (300 MHz, CD₃OD) δ (ppm): 6.90 (m, 2H), 6.81 (m, 2H), 3.38 (m, 4H), |
| | 3.26 (m, 4H), 2.93 (s, 3H). ¹H NMR (TFA salt) (400 MHz, CDCl₃) δ (ppm): 9.12 (s, |
| | 1H), 8.81 (s, 1H), 7.45 (d, J = 8.72 Hz, 2H), 7.25 (m, 5H), 6.88 (d, J = 8.43 Hz, 2H), |
| | 5.53 (s, 2H), 2.78 (br s, 4H), 1.18 (br s, 4H); Mass Spectrum (LCMS, APCI pos.) Calcd. |
| | For C₂₆H₂₇N₇O₂: 469.22; Found: 470.1 (M + H). |
| 115 | 2-[3-(3-dimethylamino-propyl)-phenylamino]-8-indan-5-yl-5-oxo-5,8-dihydro- |
| | pyrido[2,3-d]pyrimidine-6-carboxylic acid amide |
| | ¹H NMR (TFA salt) (400 MHz, CDCl₃) δ (ppm): 9.26 (s, 1H), 8.44 (s, 1H), 7.38 (d, J = 7.96 Hz, |
| | 2H), 7.13 (d, J = 7.98 Hz, 1H), 7.32-7.23 (m, 2H), 7.07-6.74 (m, 3H), 3.10 (t, J = 7.26, |
| | 2H), 3.00 (t, J = 7.14, 2H), 2.85 (app. D, 6H), 2.64 (t, J = 7.09, 2H), |
| | 2.37-2.19 (m, 2H), 2.08 (m, 2H); Mass Spectrum (LCMS, APCI pos.) Calcd. For C₃₀H₃₄N₆O: |
| | 510.27; Found: 511.3 (M + H). |
| 116 | 2-[3-(3-dimethylamino-propyl)-phenylamino]-8-indan-5-yl-5-oxo-5,8-dihydro- |
| | pyrido[2,3-d]pyrimidine-6-carboxylic acid methylamide |
| | ¹H NMR (TFA salt) (400 MHz, CDCl₃) δ (ppm): 8.84-8.71 (m, 1H), 8.40 (s, 1H), |
| | 7.42-7.32 (m, 4H), 7.17-7.09 (m, 3H), 3.00 (m, 2H), 2.93 (m 8H), 2.72 (m, 9H), 2.56 (s, 2H), |
| | 2.15 (s, 2H); Mass Spectrum (LCMS, APCI pos.) Calcd. For C₂₉H₃₂N₆O₂: 496.26; |
| | Found: 497.3 (M + H). |
| 117 | 2-[3-(3-dimethylamino-propyl)-phenylamino]-8-indan-5-yl-5-oxo-5,8-dihydro- |
| | pyrido[2,3-d]pyrimidine-6-carboxylic acid ethylamide |
| | ¹H NMR (TFA salt) (400 MHz, CDCl₃) δ (ppm): 9.41 (s, 1H), 8.84 (s, 1H), |
| | 7.49-7.39 (m, 2H), 7.22-7.12 (m, 2H), 7.00-6.81 (m, 3H), 3.50 (dd, J = 7.23, 5.61 Hz, 2H), |
| | 3.15-2.92 (m, 6H), 2.79 (app d, 6H), 2.64 (m, 2H), 2.23 (m, 2H), 2.01 (m, 2H), 1.27 (t, J = 7.28, |
| | 3H); Mass Spectrum (LCMS, APCI pos.) Calcd. For C₃₀H₃₄N₆O₂: 510.27; Found: |
| | 511.3 (M + H). |
| 118 | 2-(4-dimethylcarbamoylmethyl-phenylamino)-8-indan-5-yl-5-oxo-5,8-dihydro- |
| | pyrido[2,3-d]pyrimidine-6-carboxylic acid amide |
| | ¹H NMR (TFA salt) (400 MHz, CDCl₃) δ (ppm): 9.38 (s, 1H), 8.85 (s, 1H), |
| | 7.40-7.30 (m, 2H), 7.14-7.07 (m, 2H), 6.96-6.81 (m, 3H), 3.58 (s, 2H), 3.07-2.98 (m, 2H), |
| | 2.90-2.94 (m, 8H), 2.25-2.09 (m, 2H); Mass Spectrum (LCMS, APCI pos.) Calcd. For |
| | C₂₇H₂₆N₆O₃: 482.21; Found: 483.2 (M + H). |
| 119 | 8-indan-5-yl-5-oxo-2-(4-piperidin-1-ylmethyl-phenylamino)-5,8-dihydro-pyrido[2,3- |
| | d]pyrimidine-6-carboxylic acid amide |
| | ¹H NMR (TFA salt) (400 MHz, CDCl₃) δ (ppm): 9.31 (s, 1H), 8.46 (s, 1H), |
| | 7.33-7.19 (m, 2H), 7.23-7.22 (m, 3H), 7.17-6.92 (m, 2H), 3.40 (d, J = 11.6 Hz, 2H), 3.14-253 (m, |
| | 6H), 2.53-2.37 (m, 2H), 2.16-1.88 (m, 2H), 1.77 (m, 4H); Mass Spectrum (LCMS, APCI |
| | pos.) Calcd. For: C₂₉H₃₀N₆O₂: 494.24; Found: 495.2 (M + H). |
| 120 | 8-indan-5-yl-2-[3-(morpholine-4-carbonyl)-phenylamino]-5-oxo-5,8-dihydro- |
| | pyrido[2,3-d]pyrimidine-6-carboxylic acid amide |
| | ¹H NMR (TFA salt) (400 MHz, CDCl₃) δ (ppm): 8.91 (s, 1H), 8.48 (s, 1H), 7.39 (t, J = 7.84, |
| | 2H), 7.32-7.24 (m, 2H), 7.18 (m, 3H), 3.67 (m, J = 4H), 3.12-2.92 (m, 6H), |
| | 2.31-2.18 (m, 2H), 2.21-2.09 (m, 2H). Mass Spectrum (LCMS, APCI pos.) Calcd. For: |
| | C₂₈H₂₆N₆O₄: 510.20; Found: 511.2 (M + H). |
| 121 | 2-[4-(2-dimethylamino-ethyl)-phenylamino]-8-indan-5-yl-5-oxo-5,8-dihydro- |
| | pyrido[2,3d]pyrimidine-6-carboxylic acid amide |
| | ¹H NMR (TFA salt) (400 MHz, CDCl₃) δ (ppm): 9.32 (s, 1H), 8.49 (s, 1H), 7.41 (d, J = 7.94 Hz, |
| | 2H), 7.29-7.25 (m, 3H), 7.17 (s, 2H), 3.14-2.96 (m, 2H), 2.74-2.63 (m, 2H), |
| | 2.61-2.45 (m, 2H), 2.40 (s, 6H), 2.26-2.16 (m, 2H); Mass Spectrum (LCMS, APCI pos.) |
| | Calcd. For: C₂₇H₂₈N₆O₂: 468.23; Found: 469.2 (M + H). |
| 122 | 8-indan-5-yl-5-oxo-2-[4-(pyrrolidine-1-carbonyl)-phenylamino]-5,8-dihydro-pyrido[2,3- |
| | d]pyrimidine-6-carboxylic acid amide |
| | ¹H NMR (TFA salt) (400 MHz, CDCl₃) δ (ppm): 9.34 (s, 1H), 8.78 (s, 1H), 7.37 (d, J = 7.98, |
| | 2H), 7.17-7.08 (m, 3H), 7.31-7.24 (m, 2H), 3.56 (t, J = 7.00 Hz, 2H), |
| | 3.07-2.85 (m, 2H), 2.25-2.03 (m, 4H), 1.95-1.87 (m, 2H), 1.83 (m, 2H), 3.37 (m, 2H); Mass |
| | Spectrum (LCMS, APCI pos.) Calcd. For: C₂₈H₂₆N₆O₃: 494.21; Found: 495.1 (M + H). |
| 123 | 2-(4-dimethylcarbamoyl-phenylamino)-8-indan-5-yl-5-oxo-5,8-dihydro-pyrido[2,3- |
| | d]pyrimidine-6-carboxylic acid amide |
| | ¹H NMR (TFA salt) (400 MHz, CDCl₃) δ (ppm): 9.69 (s, 1H), 8.69 (s, 1H), 7.42 (d, J = 7.95 Hz, |
| | 2H), 7.25-7.27 (m, 3H), 7.31-7.27 (m, 2H), 3.24 (s, 6H), 3.13 (m, 4H), |
| | 2.35-2.11 (m, 2H); Mass Spectrum (LCMS, APCI pos.) Calcd. For: C₂₆H₂₄N₆O₃: 468.19; |
| | Found: 469.2 (M + H). |
| 124 | 8-indan-5-yl-5-oxo-2-(4-pyrrolidin-1-ylmethyl-phenylamino)-5,8-dihydro-pyrido[2,3- |
| | d]pyrimidine-6-carboxylic acid amide |
| | ¹H NMR (TFA salt) (400 MHz, CDCl₃) δ (ppm): 9.33 (s, 1H), 8.52 (s, 1H), |
| | 7.38-7.31 (m, 2H), 7.24-7.18 (m, 3H), 7.15-7.08 (m, 2H), 3.60-3.51 (m, 2H), 3.06-2.82 (m, 4H), |
| | 2.55-2.39 (m, 4H), 2.23-2.11 (m, 2H), 1.75 (br s, 2H); Mass Spectrum (LCMS, APCI |
| | pos.) Calcd. For: C₂₈H₂₆N₆O₃: 494.21; Found: 495.2 (M + H). |
| 125 | 8-indan-5-yl-5-oxo-2-(3-pyrrolidin-1-yl-phenylamino)-5,8-dihydro-pyrido[2,3- |
| | d]pyrimidine-6-carboxylic acid amide |
| | ¹H NMR (TFA salt) (400 MHz, CDCl₃) δ (ppm): 9.35 (s, 1H), 8.75 (s, 1H), 7.35 (d, J = 8.00 Hz, |
| | 2H), 7.20 (dd, J = 0.95, 0.49 Hz, 2H), 7.15-7.06 (m, 3H), 3.59-3.43 (m, 4H), |
| | 2.95 (td, J = 25.60, 7.34 Hz, 4H), 2.25 (br s, 4H), 2.14 (dd, J = 1.82, Hz, 2H); Mass |
| | Spectrum (LCMS, APCI pos.) Calcd. For: C₂₇H₂₆N₆O₂: 466.21; Found: 467.2 (M + H). |
| 126 | 2-[4-(1-acetyl-piperidin-4-yl)-phenylamino]-8-indan-5-yl-5-oxo-5,8-dihydro-pyrido[2,3- |
| | d]pyrimidine-6-carboxylic acid amide |
| | ¹H NMR (TFA salt) (400 MHz, CDCl₃) δ (ppm): 9.35 (s, 1H), 8.75 (s, 1H), 7.35 (d, J = 8.00 Hz, |
| | 2H), 7.24-7.17 (m, 2H), 7.15-7.00 (m, 3H), 3.51 (br s, 6H), 2.95 (td, J = 25.36, |
| | 7.29 Hz, 4H), 2.25 (br s, 4H), 2.14 (t, J = 7.12 Hz, 2H); Mass Spectrum (LCMS, APCI |
| | pos.) Calcd. For: C₃₀H₃₀N₆O₃: 522.24; Found: 523.2 (M + H). |

### Example 2

### 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide (Cpd 30)

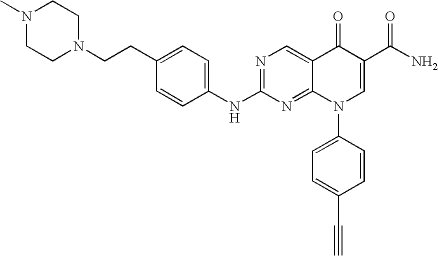

### A. 4-methoxy-2-methylsulfanyl-pyrimidine-5-carbonyl chloride

A suspension of ethyl 4-chloro-2-methylthio-5-pyrimidinecarboxylate (3 g, 12.9 mmol) in 40 mL methanol and 20 mL 1N NaOH solution was heated at 50° C. for 12 hrs. The reaction mixture was poured onto ice water and acidified with conc. HCl. The white creamy mixture was extracted with EtOAc. The organic fractions were dried (Na₂SO₄) filtered and the filtrate was concentrated to give a white solid (2 g). The solid was dissolved in 50 mL of CH₂Cl₂, and cooled under N₂ in an ice-water bath. To the reaction was slowly added oxalyl chloride (2 mL, 19 mmol) and a drop of DMF. The reaction mixture was stirred at rt for 5 h. The reaction mixture was concentrated to give the title compound as a brown solid. This material was used in the next step without further purification.

### B. 3-(4-methoxy-2-methylsulfanyl-pyrimidin-5-yl)-3-oxo-propionic acid ethyl ester

To a solution of 2.8 mL (23.5 mmol) of ethyl hydrogen malonate in 20 mL of THF at 0° C. was added dropwise 15 mL (47 mmol) of MeMgBr (3M in Et₂O). After the mixture was stirred for 20 min, a suspension of 4-methoxy-2-methylsulfanyl-pyrimidine-5-carbonyl chloride (Step A) in THF (25 mL) was slowly added. After stirring at rt for 2 h, the reaction mixture was poured into ice water. The mixture was acidified to pH 5-6 with conc. HCl and extracted with EtOAc. The organic fractions were dried (Na₂SO₄), filtered and the filtrate was concentrated. The residue was purified by chromatography (silica, EtOAc/hexanes, 1:19-1:9, v/v) to give 1.2 g of the title compound as a white solid (34% yield combining three steps).

### C. 8-(4-ethynyl-phenyl)-2-methylsulfanyl-5-oxo-5,8-dihydro-pyrido[2,3-d]pyrimidine-6-carboxylic acid ethyl ester

A solution of 1.2 g of 3-(4-methoxy-2-methylsulfanyl-pyrimidin-5-yl)-3-oxo-propionic acid ethyl ester (4.38 mmol), 1 mL of acetic anhydride (10.95 mmol) and 1.11 mL of triethylorthoformate (6.6 mmol) was heated at reflux for 1 h. After the reaction was concentrated, the residue was dissolved in 12 mL of THF. To this solution was added 102 mg (0.87 mmol) of 4-ethynyl-phenylamine. After the mixture was stirred at rt for 12 h, 200 mg of K₂CO₃ (1.46 mmol) was added. The mixture was stirred at rt for 4 h. After aqueous work up, the reaction mixture was extracted with CH₂Cl₂. The organic fractions were dried (Na₂SO₄), filtered and the filtrate was concentrated. The residue was purified by chromatography (silica, EtOAc/hexanes, 1:4-2:3, v/v) to give 190 mg of the title compound as a white solid (71% yield). ¹H NMR (400 MHz, CDCl₃) δ (ppm): 9.40 (s, 1H), 8.55 (s, 1H), 7.68 (d, 2H), 7.42 (d, 2H), 4.40 (q, 2H), 3.22 (s, 1H), 2.30 (s, 3H), 1.40 (t, 3H).

### D. 8-(4-ethynyl-phenyl)-2-methanesulfonyl-5-oxo-5,8-dihydro-pyrido[2,3-d]pyrimidine-6-carboxylic acid ethyl ester

Using procedure described in Example 1, Step E, the title compound was obtained as an off-white solid (166 mg). ¹H NMR (400 MHz, CDCl₃) δ (ppm): 9.790 (s, 1H), 8.70 (s, 1H), 7.75 (d, 2H), 7.45 (d, 2H), 4.40 (q, 2H), 3.22 (s, 1H), 3.20 (s, 3H), 1.40 (t, 3H).

### E. 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethyl ester

A mixture of and 8-(4-ethynyl-phenyl)-2-methanesulfonyl-5-oxo-5,8-dihydro-pyrido[2,3-d]pyrimidine-6-carboxylic acid ethyl ester (20 mg, 0.05 mmol) (see Example 1, Step E) and 4-[2-(4-methyl-piperazin-1-yl)-ethyl]-phenylamine (20 mg, 0.09 mmol) in 1 mL of isopropanol was heated to 90° C. for 1 hour. The solvent was evaporated and the residue was re-dissolved in a mixture of methanol and CH₂Cl₂ (1:1, v/v) and applied onto a prep-TLC plate (2000 micro). The plate was developed in NH₄OH/MeOH/CH₂Cl₂ (1:9:90, v/v) to provide the title compound as a yellow solid (18 mg, 67%). ¹H NMR (300 MHz, CDCl₃) δ (ppm): 9.38 (s, 1H), 8.52 (s, 1H), 7.44 (m, 2H), 7.28 (s, 1H), 7.19 (m, 3H), 6.66 (m, 2H), 4.40 (q, 2H), 3.00-3.18 (m, 8H), 2.60 (m, 4H), 2.35 (s, 3H), 2.22 (m, 2H), 1.40 (t, 3H). Mass Spectrum (LCMS, ESI pos.) Calcd. For C₃₀H₃₂N₆O₃: 525.25 (M+H), Found: 525.4.

Using the foregoing procedure, 4-[2-(4-methyl-piperazin-1-yl)-ethyl]-phenylamine (20 mg, 0.09 mmol) and 8-(4-ethynyl-phenyl)-2-methanesulfonyl-5-oxo-5,8-dihydro-pyrido[2,3-d]pyrimidine-6-carboxylic acid ethyl ester (20 mg, 0.05 mmol) (see Example 1, Step E) were reacted to provide the title compound as a yellow solid (18 mg, 67%).

### F. 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide

In a pressure bottle (10 mL), ammonia at −78° C. was bubbled for 5 minutes into a solution of 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethyl ester (10 mg, 0.019 mmol) in 1 mL of methanol. The bottle was capped and warmed up to room temperature and stirred for 16 hours. After HPLC purification, the relevant fractions were pooled, made basic (sat. NaHCO₃) and extracted with CH₂Cl₂. The organic fractions were dried (Na₂SO₄) and filtered. The filtrate was concentrated and converted to a hydrochloride salt to provide the title compound as a yellow solid (9 mg, 82%). ¹H NMR (400 MHz, CDCl₃) δ (ppm): 9.28 (s, 1H), 8.75 (s, 1H), 7.65 (d, 2H), 7.35 (d, 2H), 7.16 (br, 2H), 6.90 (br, 2H), 3.38 (s, 1H), 2.40-2.80 (m, 12H), 2.25 (s, 3H). Mass Spectrum (LCMS, ESI pos.) Calcd. For C₂₉H₂₉N₇O₂: 508.24 (M+H), Found 508.3.

Using the foregoing procedure, the title compound was prepared from 8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethyl ester (10 mg, 0.019 mmol). After HPLC purification, the relevant fractions were pooled, made basic (sat. NaHCO₃) and extracted with CH₂Cl₂. The organic fractions were dried (Na₂SO₄) and filtered. The filtrate was concentrated and the product was made the hydrochloride salt. The title compound was obtained as a yellow solid (9 mg, 82%). ¹H NMR (400 MHz, CDCl₃) δ (ppm): 9.28 (s, 1H), 8.75 (s, 1H), 7.65 (d, 2H), 7.35 (d, 2H), 7.16 (br, 2H), 6.90 (br, 2H), 3.38 (s, 1H), 2.40-2.80 (m, 12H), 2.25 (s, 3H). Mass Spectrum (LCMS, ESI pos.) Calcd. For C₂₉H₂₉N₇O₂: 508.24 (M+H), Found 508.3.

Using the procedure of Example 2 and reagents, starting materials and conditions known to those skilled in the art, the following compounds representative of the present invention were prepared:

| Cpd | Name and Data |
|---|---|
| 31 | 8-(4-ethynyl-phenyl)-2-{4-[2-(4-methyl-piperazin-1-yl)-ethyl]-phenylamino}-5-oxo- |
| | 5,8-dihydro-pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.75 (s, 1H), |
| | 7.65 (d, 2H), 7.35 (d, 2H), 7.16 (br, 2H), 6.88 (br, 2H), 3.82 (s, 3H), 3.38 (s, 1H), |
| | 2.50-2.90 (m, 12H), 2.25 (s, 3H). Mass Spectrum (LCMS, ESI pos.) Calcd. For C₃₀H₃₁N₇O₃: |
| | 538.25 (M + H), Found 538.3. |
| 33 | 8-(4-chloro-phenyl)-2-{4-[2-(4-methyl-piperazin-1-yl)-ethyl]-phenylamino}-5-oxo- |
| | 5,8-dihydro-pyrido[2,3-d]pyrimidine-6-carboxylic acid amide |
| | ¹H NMR (400 MHz, CDCl₃/CD₃OD 10:1 v/v) δ (ppm): 9.28 (s, 1H), 8.72 (s, 1H), |
| | 7.65 (d, 2H), 7.35 (d, 2H), 7.16 (br, 2H), 6.90 (br, 2H), 2.40-2.80 (m, 12H), 2.30 (s, 3H). |
| | Mass Spectrum (LCMS, ESI pos.) Calcd. For C₂₇H₂₈ClN₇O₂: 519.20 (M + H), Found |
| | 519.3. |
| 34 | 8-(4-chloro-phenyl)-2-{4-[2-(4-methyl-piperazin-1-yl)-ethyl]-phenylamino}-5-oxo- |
| | 5,8-dihydro-pyrido[2,3-d]pyrimidine-6-carboxylic acid methoxy-amide |
| | ¹H NMR (400 MHz, CDCl₃) δ (ppm): 11.85 (s, 1H), 9.30 (s, 1H), 8.75 (s, 1H), |
| | 7.55 (d, 2H), 7.32 (d, 2H), 7.14 (br, 2H), 6.90 (br, 2H), 3.82 (s, 3H), 2.50-2.80 (m, 12H), |
| | 2.43 (s, 3H). Mass Spectrum (LCMS, ESI pos.) Calcd. For C₂₈H₃₀ClN₇O₃: 548.21 (M + H), |
| | Found 548.2. |
| 35 | 8-cyclopentyl-5-oxo-2-(4-pyrrolidin-1-yl-phenylamino)-5,8-dihydro-pyrido[2,3- |
| | d]pyrimidine-6-carboxylic acid methoxy-amide |
| | ¹H NMR (400 MHz, CDCl₃) δ (ppm): 11.90 (s, 1H), 9.20 (s, 1H), 8.71 (s, 1H), |
| | 7.40 (br, 2H), 6.62 (br, 2H), 5.45 (m, 1H), 3.82 (s, 3H), 3.20-3.50 (m, 4H), 2.20 (m, 2H), |
| | 1.70-2.00 (m, 10H). Mass Spectrum (LCMS, ESI pos.) Calcd. For C₂₄H₂₈N₆O₃: |
| | 449.22 (M + H), Found 449.3. |
| 36 | 8-cyclopentyl-5-oxo-2-[4-(pyrrolidine-1-carbonyl)-phenylamino]-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.76 (s, 1H), |
| | 7.66 (d, 2H), 7.52 (d, 2H), 5.55 (m, 1H), 3.82 (s, 3H), 3.58 (t, 2H), 3.45 (t, 2H), 2.21 (m, 2H), |
| | 1.70-1.96 (m, 10H). Mass Spectrum (LCMS, ESI pos.) Calcd. For C₂₅H₂₈N₆O₄: |
| | 477.22 (M + H), Found 477.3. |
| 37 | 8-cyclopentyl-5-oxo-2-[4-(2-oxo-pyrrolidin-1-ylmethyl)-phenylamino]-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.35 (s, 1H), 8.79 (s, 1H), |
| | 7.60 (d, 2H), 7.21 (d, 2H), 5.55 (m, 1H), 4.40 (s, 2H), 3.84 (s, 3H), 3.30 (t, 2H), 2.43 (t, |
| | 2H), 2.21 (m, 2H), 1.78-2.01 (m, 8H). Mass Spectrum (LCMS, ESI pos.) Calcd. For |
| | C₂₅H₂₈N₆O₄: 477.22 (M + H), Found 477.3. |
| 38 | 8-cyclopentyl-5-oxo-2-{4-[2-(2-oxo-pyrrolidin-1-yl)-ethyl]-phenylamino}-5,8- |
| | dihydro-pyrido[2,3-d]pyrimidine-6-carboxylic acid methoxy-amide |
| | ¹H NMR (400 MHz, CDCl₃) δ (ppm): 12.00 (s, 1H), 9.35 (s, 1H), 8.80 (s, 1H), |
| | 7.78 (br, 1H), 7.60 (d, 2H), 7.22 (d, 2H), 5.58 (m, 1H), 3.89 (s, 3H), 3.55 (t, 2H), 3.30 (t, |
| | 2H), 3.13 (t, 2H), 2.85 (t, 2H), 2.40 (t, 2H), 2.25 (m, 2H), 1.80-2.10 (m, 6H). Mass |
| | Spectrum (LCMS, ESI pos.) Calcd. For C₂₆H₃₀N₆O₄: 491.23 (M + H), Found 491.3. |
| 39 | 8-cyclopentyl-5-oxo-2-[4-(2-pyrrolidin-1-yl-ethyl)-phenylamino]-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.32 (s, 1H), 8.78 (s, 1H), |
| | 7.60 (d, 2H), 7.22 (d, 2H), 5.58 (m, 1H), 3.84 (s, 3H), 3.30 (m, 2H), 3.10 (m, 2H), 2.82 (br, |
| | 4H), 1.80-2.30 (m, 12H). Mass Spectrum (LCMS, ESI pos.) Calcd. For C₂₆H₃₂N₆O₃: |
| | 477.25 (M + H), Found 477.3. |
| 50 | 8-(3,4-dimethyl-phenyl)-5-oxo-2-{4-[2-(2-oxo-pyrrolidin-1-yl)-ethyl]-phenylamino}- |
| | 5,8-dihydro-pyrido[2,3-d]pyrimidine-6-carboxylic acid methoxy-amide |
| | ¹H NMR (400 MHz, CDCl₃) δ (ppm): 12.10 (br, 1H), 11.98 (s, 1H), 8.80 (s, 1H), |
| | 7.35 (d, 1H), 7.30 (br, 2H), 7.20 (m, 3H), 6.94 (br, 2H), 3.89 (s, 3H), 3.45 (t, 2H), 3.30 (t, |
| | 2H), 3.13 (m, 2H), 2.79 (t, 2H), 2.42 (s, 3H), 2.23 (t, 3H), 1.97 (t, 2H). Mass |
| | Spectrum (LCMS, ESI pos.) Calcd. For C₂₉H₃₀N₆O₄: 527.23 (M + H), Found 527.3. |
| 54 | 8-(4-ethyl-phenyl)-5-oxo-2-{4-[2-(2-oxo-pyrrolidin-1-yl)-ethyl]-phenylamino}-5,8- |
| | dihydro-pyrido[2,3-d]pyrimidine-6-carboxylic acid methoxy-amide |
| | ¹H NMR (400 MHz, CDCl₃) δ (ppm): 11.89 (s, 1H), 9.30 (s, 1H), 8.78 (s, 1H), |
| | 7.40 (d, 2H), 7.25 (d, 2H), 7.20 (br, 2H), 6.88 (br, 2H), 3.82 (s, 3H), 3.42 (t, 2H), 3.20 (t, |
| | 2H), 2.78 (m, 2H), 2.70 (t, 2H), 2.35 (t, 2H), 1.90 (q, 2H), 1.32 (t, 3H). Mass |
| | Spectrum (LCMS, ESI pos.) Calcd. For C₂₉H₃₀N₆O₄: 527.23 (M + H), Found 527.3. |

### Example 3

### 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide (Cpd 18)

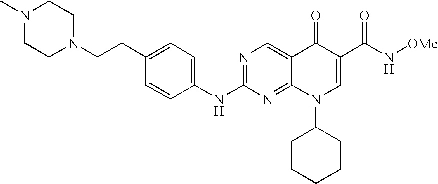

### A. 3-cyclohexylamino-propionic acid ethyl ester

Cyclohexylamine (0.86 g, 8.7 mmol) and 3-chloro-propionic acid ethyl ester (1.18 g, 8.67 mmol) were combined neat and K₂CO₃ (1.2 g, 8.7 mmol) and a catalytic amount of tetrabutylammonium iodide (ca. 5 mg) was added. The mixture was heated at 80° C. overnight, then partitioned between water and DCM. The organic layer was dried (MgSO₄) and concentrated to afford 1.25 g (72%) of the title compound. ¹H-NMR (400 MHz, CDCl₃) δ ppm 4.14 (q, 2H, J=7.2 Hz), 2.90 (t, 2H, J=6.6 Hz), 2.50 (t, 2H, J=6.6 Hz), 1.86-1.89 (m, 2H), 1.70-1.75 (m, 2H), 1.58-1.62 (m, 2H), 1.25 (t, 1H, J=7.2 Hz).

### B. 4-[cyclohexyl-(2-ethoxycarbonyl-ethyl)-amino]-2-methylsulfanyl-pyrimidine-5-carboxylic acid ethyl ester

3-cyclohexylamino-propionic acid ethyl ester (1.0 g, 5.0 mmol) and 4-chloro-2-methylsulfanyl-pyrimidine-5-carboxylic acid ethyl ester (1.17 g, 5.02 mmol) were combined in DCM (15 mL) and diisopropylethylamine (0.81 g, 6.3 mmol) was added. After 16 h, the solution was partitioned between water and DCM and the organic layer was dried (MgSO₄) and concentrated. Chromatography (0-20% EtOAc/hexanes gradient) provided 1.63 g (84%) of the title compound. ¹H-NMR (400 MHz, CDCl₃) δ ppm 8.39 (s, 1H) 5.30 (s, 1H), 4.30 (q, 2H, J=7.1 Hz), 4.14 (q, 1H, J=7.1 Hz), 3.76-3.80 (m, 2H), 2.65-2.69 (m, 2H), 2.49 (s, 3H), 1.81-1.84 (m, 2H), 1.34-1.40 (m, 7H), 1.12-1.27 (m, 7H).

### C. 8-cyclohexyl-2-methylsulfanyl-5-oxo-5,6,7,8-tetrahydro-pyrido[2,3-d]pyrimidine-6-carboxylic acid ethyl ester

Sodium (25 wt % dispersion in paraffin wax, 0.10 g, 3.8 mmol) was added to t-butanol (1.8 mL) at rt. After 10 minutes, a solution of 4-[cyclohexyl-(2-ethoxycarbonyl-ethyl)-amino]-2-methylsulfanyl-pyrimidine-5-carboxylic acid ethyl ester (1.0 g, 2.5 mmol) in 10 mL of toluene was added to the sodium t-butoxide solution and the resulting mixture was heated at 90° C. for 30 minutes. The reaction mixture was cooled and the solution was adjusted to pH 7 using a 1N HCl solution. The solution was extracted with EtOAc (2×20 mL) and the organic layer was dried (MgSO₄) and concentrated to provide 0.55 g, (42%) of the title compound. ¹H NMR indicated the presence of both enol and keto forms in a 1:1.75 ratio. ¹H-NMR (400 MHz, CDCl₃) δ ppm 8.63 (s, 1H), 8.18 (s, 1H), 4.76-4.82 (m), 4.58-4.68 (m), 4.16-4.36 (m), 3.91-3.96 (m), 3.60-3.64 (m), 3.46-3.49 (m), 2.53 (s, 3H), 2.50 (s, 5.25H), 1.86-1.89 (m), 1.71-1.73 (m), 1.32-1.56 (m), 1.26 (t, J=7.2 Hz), 1.10-1.21 (m).

### D. 8-cyclohexyl-2-methylsulfanyl-5-oxo-5,8-dihydro-pyrido[2,3-d]pyrimidine-6-carboxylic acid ethyl ester

Bromine (0.15 g, 0.94 mmol) was added to a solution of 8-cyclohexyl-2-methylsulfanyl-5-oxo-5,6,7,8-tetrahydro-pyrido[2,3-d]pyrimidine-6-carboxylic acid ethyl ester (0.28 g, 0.79 mmol) in DCM (10 mL). After 5 min, the solution was concentrated and the crude residue was redissolved in DCM (10 mL) and diisopropylethylamine (0.42 mL, 2.4 mmol) was added. After 15 h, the reaction mixture was partitioned between water and DCM, the organic layer was separated, dried (MgSO₄) and concentrated to provide 0.28 g (87%) of the title compound. Mass Spectrum (LCMS, ESI pos.) Calcd. For C₁₇H₂₁N₃O₃S: 347.13, found: (M+H) 348.3.

### E. 8-cyclohexyl-2-methanesulfonyl-5-oxo-5,8-dihydro-pyrido[2,3-d]pyrimidine-6-carboxylic acid ethyl ester

m-CPBA (0.33 g, 1.5 mmol of a 70% w/w mixture) was added to a solution of 8-cyclohexyl-2-methylsulfanyl-5-oxo-5,8-dihydro-pyrido[2,3-d]pyrimidine-6-carboxylic acid ethyl ester (0.206 g, 0.59 mmol) in DCM (15 mL). After 2 hours, a 10% solution of Na₂SO₃ (1 mL) was added and the mixture was partitioned between sat. NaHCO₃ and DCM. The organic layer was dried (MgSO₄) and concentrated to provide 0.22 g of the title compound. Mass Spectrum (LCMS, ESI pos.) Calcd. For C₁₇H₂₁N₃O₅S: 379.12, found: (M+H) 380.1.

### F. 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid ethyl ester

A mixture of 4-[2-(4-methyl-piperazin-1-yl)-ethyl]-phenylamine (25 mg, 0.12 mmol) and 8-cyclohexyl-2-methanesulfonyl-5-oxo-5,8-dihydro-pyrido[2,3-d]pyrimidine-6-carboxylic acid ethyl ester (25 mg, 0.065 mmol) in 1 mL of acetic acid was heated at 110° C. for 15 min. The reaction mixture was concentrated, and the residue was purified (HPLC, C-18 YMC ODS-A 5 m 30×100 mm, 120 A column, 32 mL/min, 5-80% MeCN/H₂O (0.1% TFA v/v) gradient over 12 min). The relevant fractions were pooled, made basic (sat. NaHCO₃) and extracted with CH₂Cl₂. The organic fractions were dried (Na₂SO₄) and filtered. The filtrate was concentrated to give the title compound as a yellow solid (15 mg, 44%).

### G. 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide

The title compound was prepared from 8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid ethyl ester and methoxyamine hydrochloride according to procedures described in Example 2, Step F. ¹H NMR (400 MHz, CDCl₃) δ (ppm): 12.00 (s, 1H), 9.37 (s, 1H), 8.80 (s, 1H), 7.70 (br, 1H), 7.58 (d, 2H), 7.24 (d, 2H), 5.15 (m, 1H), 3.90 (s, 3H), 2.85 (m, 2H), 2.60 (m, 6H), 2.38 (s, 3H), 2.06 (m, 4H), 1.23-1.90 (m, 10H). Mass Spectrum (LCMS, ESI pos.) Calcd. For C₂₈H₃₇N₇O₃: 520.30 (M+H), Found 520.3.

Using the procedure of Example 3 and reagents, starting materials and conditions known to those skilled in the art, the following compounds representative of the present invention were prepared:

| Cpd | Name and Data |
|---|---|
| 19 | 8-cyclohexyl-2-{4-[2-(4-methyl-piperazin-1-yl)-ethyl]-phenylamino}-5-oxo-5,8- |
| | dihydro-pyrido[2,3-d]pyrimidine-6-carboxylic acid ethoxy-amide |
| | ¹H NMR (400 MHz, CDCl₃) δ (ppm): 11.95 (s, 1H), 9.37 (s, 1H), 8.80 (s, 1H), 7.60 (br, |
| | 1H), 7.58 (d, 2H), 7.22 (d, 2H), 5.15 (m, 1H), 4.10 (q, 2H), 2.50-2.90 (m, 10H), 2.40 (br, |
| | 3H), 2.06 (m, 4H), 1.23-1.90 (m, 8H), 1.33 (t, 3H). Mass Spectrum (LCMS, ESI pos.) |
| | Calcd. For C₂₉H₃₉N₇O₃: 534.31 (M + H), Found 534.3. |
| 20 | 8-cyclohexyl-2-[4-(2-morpholin-4-yl-ethyl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.30 (s, 1H), 8.78 (s, 1H), 7.57 (br, |
| | 3H), 7.20 (d, 2H), 5.08 (m, 1H), 3.80 (s, 3H), 3.65 (br, 4H), 2.75 (br, 2H), 2.50 (m, 6H), |
| | 1.20-1.98 (m, 10H). Mass Spectrum (LCMS, ESI pos.) Calcd. For C₂₇H₃₄N₆O₄: |
| | 507.26 (M + H), Found 507.2. |
| 21 | 8-cyclohexyl-2-[4-(2-morpholin-4-yl-ethyl)-phenylamino]-5-oxo-5,8-dihydro- |
| | pyrido[2,3-d]pyrimidine-6-carboxylic acid ethoxy-amide |
| | ¹H NMR (400 MHz, CDCl₃) δ (ppm): 11.95 (s, 1H), 9.38 (s, 1H), 8.80 (s, 1H), 7.60 (br, |
| | 3H), 7.25 (d, 2H), 5.15 (m, 1H), 4.10 (q, 2H), 3.80 (br, 4H), 2.50-2.90 (m, 10H), |
| | 2.40 (br, 3H), 2.06 (m, 4H), 2.85 (br, 2H), 2.60 (m, 6H), 2.10 (m, 4H), 1.23-1.90 (m, 6H), |
| | 1.37 (t, 3H). Mass Spectrum (LCMS, ESI pos.) Calcd. For C₂₈H₃₆N₆O₄: 521.28 (M + H), |
| | Found 521.2. |
| 22 | (S)-8-cyclohexyl-5-oxo-2-[4-(2-oxo-oxazolidin-4-ylmethyl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.10 (br, 1H), 8.79 (s, 1H), 8.55 (br, |
| | 1H), 7.65 (d, 2H), 7.10 (d, 2H), 5.10 (m, 1H), 4.55 (m, 1H), 4.15 (m, 2H), 3.90 (s, 3H), |
| | 2.90 (m, 2H), 2.05 (m, 2H), 1.22-1.90 (m, 8H). Mass Spectrum (LCMS, ESI pos.) Calcd. |
| | For C₂₅H₂₈N₆O₅: 493.21 (M + H), Found 493.2. |
| 23 | (S)-8-cyclohexyl-5-oxo-2-[4-(2-oxo-oxazolidin-4-ylmethyl)-phenylamino]-5,8-dihydro- |
| | pyrido[2,3-d]pyrimidine-6-carboxylic acid ethoxy-amide |
| | ¹H NMR (400 MHz, CDCl₃) δ (ppm): 11.84 (s, 1H), 9.19 (br, 1H), 8.79 (s, 1H), 7.60 (d, |
| | 2H), 7.15 (d, 2H), 5.05 (m, 1H), 4.45 (m, 1H), 4.10 (m, 4H), 2.82 (m, 2H), 1.95 (m, |
| | 2H), 1.20-1.90 (m, 8H), 1.25 (t, 3H). Mass Spectrum (LCMS, ESI pos.) Calcd. For |
| | C₂₆H₃₀N₆O₅: 507.23 (M + H), Found 507.2. |
| 24 | 8-cyclohexyl-2-[4-(3,5-dimethyl-piperazin-1-yl)-phenylamino]-5-oxo-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.79 (s, 1H), |
| | 7.60 (br, 2H), 6.95 (d, 2H), 5.10 (m, 1H), 3.85 (s, 3H), 3.58 (d, 2H), 3.45 (br, 2H), 2.84 (t, |
| | 2H), 2.05 (m, 2H), 1.22-1.90 (m, 8H), 1.40 (s, 3H), 1.38 (s, 3H). Mass Spectrum |
| | (LCMS, ESI pos.) Calcd. For C₂₇H₃₅N₇O₃: 506.28 (M + H), Found 506.2. |
| 25 | 8-cyclohexyl-2-[4-(3,5-dimethyl-piperazin-1-yl)-phenylamino]-5-oxo-5,8-dihydro- |
| | pyrido[2,3-d]pyrimidine-6-carboxylic acid ethoxy-amide |
| | ¹H NMR (400 MHz, CDCl₃/CD₃OD 10:1 v/v) δ (ppm): 9.30 (s, 1H), 8.79 (s, 1H), |
| | 7.55 (br, 2H), 6.90 (d, 2H), 5.10 (m, 1H), 4.02 (q, 2H), 3.58 (d, 2H), 3.42 (br, 2H), 2.90 (t, |
| | 2H), 2.00 (m, 4H), 1.22-1.90 (m, 6H), 1.42 (s, 3H), 1.40 (s, 3H), 1.30 (t, 3H). Mass |
| | Spectrum (LCMS, ESI pos.) Calcd. For C₂₈H₃₇N₇O₃: 520.30 (M + H), Found 520.2. |
| 26 | 8-cyclohexyl-5-oxo-2-(4-piperidin-4-yl-phenylamino)-5,8-dihydro-pyrido[2,3- |
| | d]pyrimidine-6-carboxylic acid methoxy-amide |
| | 4-(4-amino-phenyl)-piperidine was prepared as described in Example 4, Step B. |
| | ¹H NMR (400 MHz, CDCl₃/CD₃OD 10:1 v/v) δ (ppm): 9.27 (s, 1H), 8.77 (s, 1H), |
| | 7.58 (d, 2H), 7.19 (d, 2H), 5.10 (m, 1H), 3.81 (s, 3H), 3.50 (d, 2H), 2.95 (br, 2H), 2.70 (br, |
| | 1H), 2.05 (m, 6H), 1.22-1.80 (m, 8H). Mass Spectrum (LCMS, ESI pos.) Calcd. For |
| | C₂₆H₃₂N₆O₃: 477.25 (M + H), Found 477.2. |
| 27 | 8-cyclohexyl-5-oxo-2-(4-piperidin-4-yl-phenylamino)-5,8-dihydro-pyrido[2,3- |
| | d]pyrimidine-6-carboxylic acid ethoxy-amide |
| | ¹H NMR (400 MHz, CDCl₃/CD₃OD 10:1 v/v) δ (ppm): 9.25 (s, 1H), 8.76 (s, 1H), |
| | 7.58 (d, 2H), 7.19 (d, 2H), 5.08 (m, 1H), 4.00 (q, 2H), 3.50 (d, 2H), 2.95 (br, 2H), 2.75 (br, |
| | 1H), 2.00 (m, 6H), 1.22-1.80 (m, 8H), 1.23 (t, 3H). Mass Spectrum (LCMS, ESI pos.) |
| | Calcd. For C₂₇H₃₄N₆O₃: 491.27 (M + H), Found 491.3. |
| 28 | 2-[4-(1-acetyl-piperidin-4-yl)-phenylamino]-8-cyclohexyl-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.38 (s, 1H), 8.80 (s, 1H), 7.65 (br, |
| | 1H), 7.60 (d, 2H), 7.21 (d, 2H), 5.15 (m, 1H), 4.80 (m, 1H), 3.95 (m, 1H), 3.89 (s, 3H), |
| | 3.20 (m, 1H), 2.78 (m, 1H), 2.65 (m, 1H), 2.17 (s, 3H), 2.05 (m, 4H), 1.30-1.98 (m, |
| | 10H). Mass Spectrum (LCMS, ESI pos.) Calcd. For C₂₈H₃₄N₆O₄: 519.26 (M + H), |
| | Found 519.1. |
| 29 | 2-[4-(1-acetyl-piperidin-4-yl)-phenylamino]-8-cyclohexyl-5-oxo-5,8-dihydro- |
| | pyrido[2,3-d]pyrimidine-6-carboxylic acid ethoxy-amide |
| | ¹H NMR (400 MHz, CDCl₃) δ (ppm): 11.96 (s, 1H), 9.38 (s, 1H), 8.80 (s, 1H), 7.60 (d, |
| | 2H), 7.21 (d, 2H), 5.15 (m, 1H), 4.80 (m, 1H), 4.10 (q, 2H), 3.95 (m, 1H), 3.20 (m, 1H), |
| | 2.78 (m, 1H), 2.65 (m, 1H), 2.03 (s, 3H), 2.05 (m, 4H), 1.30-1.98 (m, 10H), 1.35 (t, 3H). |
| | Mass Spectrum (LCMS, ESI pos.) Calcd. For C₂₉H₃₆N₆O₄: 533.28 (M + H), Found |
| | 533.1. |
| 32 | 8-(4-ethynyl-phenyl)-5-oxo-2-[4-(2-pyrrolidin-1-yl-ethyl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 11.87 (s, 1H), 9.30 (s, 1H), 8.75 (s, 1H), 7.65 (d, |
| | 2H), 7.35 (d, 2H), 7.16 (br, 2H), 6.90 (br, 2H), 3.81 (s, 3H), 3.40 (s, 1H), 3.20-3.50 (m, |
| | 4H), 2.98 (m, 2H), 2.77 (m, 2H), 2.10 (m, 2H). Mass Spectrum (LCMS, ESI pos.) Calcd. |
| | For C₂₉H₂₈N₆O₃: 509.22 (M + H), Found 509.3. |
| 40 | 8-cyclopentyl-5-oxo-2-[4-(2-pyrrolidin-1-yl-ethyl)-phenylamino]-5,8-dihydro- |
| | pyrido[2,3-d]pyrimidine-6-carboxylic acid ethoxy-amide |
| | ¹H NMR (300 MHz, CDCl₃) δ (ppm): 11.92 (s, 1H), 9.39 (s, 1H), 8.80 (s, 1H), 7.58 (d, |
| | 2H), 7.25 (d, 2H), 5.60 (m, 1H), 4.10 (q, 2H), 2.90 (m, 8H), 2.25 (m, 2H), 1.50-2.00 (m, |
| | 10H), 1.35 (t, 3H). Mass Spectrum (LCMS, ESI pos.) Calcd. For C₂₇H₃₄N₆O₃: |
| | 491.27 (M + H), Found 491.2. |
| 41 | 8-cyclopentyl-5-oxo-2-[4-(2-pyrrolidin-1-yl-ethyl)-phenylamino]-5,8-dihydro- |
| | pyrido[2,3-d]pyrimidine-6-carboxylic acid isopropoxy-amide |
| | ¹H NMR (300 MHz, CDCl₃) δ (ppm): 11.92 (s, 1H), 9.39 (s, 1H), 8.80 (s, 1H), 7.58 (d, |
| | 2H), 7.25 (d, 2H), 5.60 (m, 1H), 4.25 (m, 1H), 2.70-3.00 (m, 8H), 2.25 (m, 2H), 1.90 (m, |
| | 10H), 1.36 (s, 3H), 1.33 (s, 3H). Mass Spectrum (LCMS, ESI pos.) Calcd. For |
| | C₂₈H₃₆N₆O₃: 505.28 (M + H), Found 505.3. |
| 42 | 8-cyclopentyl-6-(morpholine-4-carbonyl)-2-[4-(2-pyrrolidin-1-yl-ethyl)-phenylamino]- |
| | 8H-pyrido[2,3-d]pyrimidin-5-one |
| | ¹H NMR (300 MHz, CDCl₃) δ (ppm): 10.15 (br, 1H), 9.30 (s, 1H), 8.05 (s, 1H), 7.58 (d, |
| | 2H), 7.22 (d, 2H), 5.57 (m, 1H), 3.80 (m, 4H), 3.62 (m, 2H), 3.42 (m, 2H), 2.70-3.00 (m, |
| | 6H), 2.25 (m, 2H), 1.60-1.98 (m, 12H). Mass Spectrum (LCMS, ESI pos.) Calcd. For |
| | C₂₉H₃₆N₆O₃: 517.28 (M + H), Found 517.3. |
| 43 | 8-cyclopentyl-5-oxo-2-[4-(2-pyrrolidin-1-yl-ethyl)-phenylamino]-5,8-dihydro- |
| | pyrido[2,3-d]pyrimidine-6-carboxylic acid (2-hydroxy-ethyl)-amide |
| | ¹H NMR (300 MHz, CDCl₃) δ (ppm): 10.12 (br, 1H), 9.35 (s, 1H), 8.82 (s, 1H), 7.75 (br, |
| | 1H), 7.56 (d, 2H), 7.22 (d, 2H), 5.59 (m, 1H), 3.81 (t, 2H), 3.62 (m, 2H), 2.86 (m, 2H), |
| | 2.77 (m, 2H), 2.62 (m, 4H), 2.27 (m, 2H), 1.77-2.01 (m, 10H). Mass Spectrum (LCMS, |
| | ESI pos.) Calcd. For C₂₇H₃₄N₆O₃: 491.27 (M + H), Found 491.3. |
| 44 | 8-cyclopentyl-5-oxo-2-[4-(2-pyrrolidin-1-yl-ethyl)-phenylamino]-5,8-dihydro- |
| | pyrido[2,3-d]pyrimidine-6-carboxylic acid (2-amino-ethyl)-amide |
| | ¹H NMR (300 MHz, CDCl₃) δ (ppm): 9.97 (br, 1H), 9.37 (s, 1H), 8.82 (s, 1H), 7.75 (br, |
| | 1H), 7.56 (d, 2H), 7.22 (d, 2H), 5.59 (m, 1H), 3.50 (m, 2H), 2.90 (m, 4H), 2.77 (m, 2H), |
| | 2.62 (m, 4H), 2.27 (m, 2H), 1.77-2.01 (m, 10H). Mass Spectrum (LCMS, ESI pos.) |
| | Calcd. For C₂₇H₃₅N₇O₂: 490.29 (M + H), Found 490.3. |
| 45 | 8-cyclopentyl-5-oxo-2-(4-piperidin-4-yl-phenylamino)-5,8-dihydro-pyrido[2,3- |
| | d]pyrimidine-6-carboxylic acid methoxy-amide |
| | ¹H NMR (400 MHz, CDCl₃/CD₃OD 10:1 v/v) δ (ppm): 9.35 (s, 1H), 8.79 (s, 1H), |
| | 7.60 (d, 2H), 7.22 (d, 2H), 5.56 (m, 1H), 4.80 (m, 1H), 3.84 (s, 3H), 3.50 (d, 2H), 2.95 (br, |
| | 2H), 2.27 (m, 2H), 1.77-2.01 (m, 10H). Mass Spectrum (LCMS, ESI pos.) Calcd. For |
| | C₂₅H₃₀N₆O₃: 463.24 (M + H), Found 463.4. |
| 46 | 2-[4-(1-acetyl-piperidin-4-yl)-phenylamino]-8-cyclopentyl-5-oxo-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.32 (br, 1H), 8.80 (s, 1H), 7.60 (d, |
| | 2H), 7.22 (d, 2H), 5.58 (m, 1H), 4.80 (d, 1H), 3.96 (d, 1H), 3.90 (s, 3H), 3.22 (t, 1H), |
| | 2.80 (t, 1H), 2.70 (t, 1H), 2.27 (m, 2H), 2.20 (s, 3H), 1.60-2.00 (m, 10H). Mass |
| | Spectrum (LCMS, ESI pos.) Calcd. For C₂₇H₃₂N₆O₄: 505.25 (M + H), Found 505.4. |
| 47 | 2-[4-(1-acetyl-piperidin-4-yl)-phenylamino]-8-cyclopentyl-5-oxo-5,8-dihydro- |
| | pyrido[2,3-d]pyrimidine-6-carboxylic acid ethoxy-amide |
| | ¹H NMR (400 MHz, CDCl₃) δ (ppm): 11.98 (s, 1H), 9.35 (br, 1H), 8.80 (s, 1H), 7.60 (d, |
| | 2H), 7.22 (d, 2H), 5.58 (m, 1H), 4.80 (d, 1H), 4.06 (q, 2H), 3.98 (d, 1H), 3.20 (t, 1H), |
| | 2.80 (t, 1H), 2.70 (t, 1H), 2.27 (m, 2H), 2.20 (s, 3H), 1.60-2.00 (m, 10H), 1.35 (t, 3H). |
| | Mass Spectrum (LCMS, ESI pos.) Calcd. For C₂₈H₃₄N₆O₄: 519.26 (M + H), Found |
| | 519.3. |
| 48 | 8-cyclopentyl-2-{4-[2-(4-methyl-piperazin-1-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 12.00 (s, 1H), 9.37 (br, 1H), 8.80 (s, 1H), 7.60 (d, |
| | 2H), 7.21 (d, 2H), 5.55 (m, 1H), 3.85 (s, 3H), 3.60 (br, 4H), 3.27 (m, 2H), 3.02 (m, 2H), |
| | 2.90 (s, 3H), 2.75 (br, 4H), 2.25 (m, 2H), 1.90 (m, 6H). Mass Spectrum (LCMS, ESI |
| | pos.) Calcd. For C₂₇H₃₅N₇O₃: 506.25 (M + H), Found 506.4. |
| 49 | 8-cyclopentyl-2-{4-[2-(4-methyl-piperazin-1-yl)-ethyl]-phenylamino}-5-oxo-5,8- |
| | dihydro-pyrido[2,3-d]pyrimidine-6-carboxylic acid ethoxy-amide |
| | ¹H NMR (400 MHz, CDCl₃/CD₃OD 10:1 v/v) δ (ppm): 9.37 (s, 1H), 8.78 (s, 1H), |
| | 7.60 (d, 2H), 7.20 (d, 2H), 5.55 (m, 1H), 4.02 (q, 2H), 3.45 (m, 6H), 3.10 (br, 4H), 2.95 (m, |
| | 2H), 2.80 (s, 3H), 2.23 (m, 2H), 1.90 (m, 6H), 1.35 (t, 3H). Mass Spectrum (LCMS, ESI |
| | pos.) Calcd. For C₂₈H₃₇N₇O₃: 520.30 (M + H), Found 520.4. |
| 51 | 8-(3,4-dimethyl-phenyl)-5-oxo-2-[4-(2-pyrrolidin-1-yl-ethyl)-phenylamino]-5,8- |
| | dihydro-pyrido[2,3-d]pyrimidine-6-carboxylic acid methoxy-amide |
| | ¹H NMR (400 MHz, CDCl₃) δ (ppm): 12.00 (br, 1H), 11.88 (s, 1H), 9.30 (br, 1H), |
| | 8.78 (s, 1H), 7.35 (d, 1H), 7.30 (br, 2H), 7.20 (m, 3H), 6.90 (br, 2H), 3.82 (s, 3H), 3.23 (m, |
| | 4H), 2.95 (m, 2H), 2.77 (m, 2H), 2.40 (s, 3H), 2.30 (t, 3H), 2.00-2.20 (m, 4H). Mass |
| | Spectrum (LCMS, ESI pos.) Calcd. For C₂₉H₃₂N₆O₃: 513.25 (M + H), Found 513.4. |
| 52 | 8-(3,4-dimethyl-phenyl)-2-{4-[2-(4-methyl-piperazin-1-yl)-ethyl]-phenylamino}-5-oxo- |
| | 5,8-dihydro-pyrido[2,3-d]pyrimidine-6-carboxylic acid methoxy-amide |
| | ¹H NMR (400 MHz, CDCl₃) δ (ppm): 11.98 (s, 1H), 9.39 (s, 1H), 8.81 (s, 1H), 7.78 (br, |
| | 1H), 7.35 (d, 1H), 7.22 (br, 2H), 7.20 (m, 3H), 6.92 (br, 2H), 3.90 (s, 3H), 2.50-2.80 (m, |
| | 12H), 2.42 (s, 6H), 2.38 (s, 3H). Mass Spectrum (LCMS, ESI pos.) Calcd. For |
| | C₃₀H₃₅N₇O₃: 542.28 (M + H), Found 542.2. |
| 53 | 8-(3,4-dimethyl-phenyl)-2-{4-[2-(4-methyl-piperazin-1-yl)-ethyl]-phenylamino}-5-oxo- |
| | 5,8-dihydro-pyrido[2,3-d]pyrimidine-6-carboxylic acid ethoxy-amide |
| | ¹H NMR (400 MHz, CDCl₃) δ (ppm): 11.92 (s, 1H), 9.39 (s, 1H), 8.81 (s, 1H), 7.80 (br, |
| | 1H), 7.35 (d, 1H), 7.22 (br, 2H), 7.20 (m, 3H), 6.92 (br, 2H), 4.10 (q, 2H), 2.50-2.80 (m, |
| | 12H), 2.42 (s, 3H), 2.40 (s, 3H), 2.38 (s, 3H), 1.36 (t, 3H). Mass Spectrum (LCMS, ESI |
| | pos.) Calcd. For C₃₁H₃₇N₇O₃: 556.30 (M + H), Found 556.2. |
| 55 | 8-(4-ethyl-phenyl)-5-oxo-2-[4-(2-pyrrolidin-1-yl-ethyl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 12.15 (br, 1H), 11.88 (s, 1H), 8.78 (s, 1H), |
| | 7.40 (d, 1H), 7.30 (d, 2H), 7.25 (br, 2H), 6.90 (br, 2H), 3.82 (s, 3H), 3.80 (br, 2H), 3.21 (br, |
| | 2H), 2.95 (br, 2H), 2.77 (m, 4H), 2.00-2.20 (m, 4H), 1.30 (t, 3H). Mass Spectrum |
| | (LCMS, ESI pos.) Calcd. For C₂₉H₃₂N₆O₃: 513.25 (M + H), Found 513.3. |
| 56 | 8-(4-ethyl-phenyl)-2-{4-[2-(4-methyl-piperazin-1-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 11.98 (s, 1H), 9.40 (s, 1H), 8.81 (s, 1H), 7.60 (br, |
| | 2H), 7.45 (d, 2H), 7.35 (d, 2H), 6.95 (br, 2H), 3.90 (s, 3H), 2.50-3.00 (m, 17H), 1.35 (t, |
| | 3H). Mass Spectrum (LCMS, ESI pos.) Calcd. For C₃₀H₃₅N₇O₃: 542.24 (M + H), Found |
| | 542.2. |
| 57 | 8-(4-ethyl-phenyl)-2-{4-[2-(4-methyl-piperazin-1-yl)-ethyl]-phenylamino}-5-oxo-5,8- |
| | dihydro-pyrido[2,3-d]pyrimidine-6-carboxylic acid ethoxy-amide |
| | ¹H NMR (400 MHz, CDCl₃) δ (ppm): 11.92 (s, 1H), 9.39 (s, 1H), 8.81 (s, 1H), 7.60 (br, |
| | 1H), 7.44 (d, 2H), 7.35 (d, 2H), 7.22 (br, 2H), 6.92 (br, 2H), 4.10 (q, 2H), 2.60-2.85 (m, |
| | 14H), 2.45 (br, 3H), 1.36 (t, 3H). Mass Spectrum (LCMS, ESI pos.) Calcd. For |
| | C₃₁H₃₇N₇O₃: 556.30 (M + H), Found 556.2. |
| 62 | 8-indan-5-yl-2-{4-[2-(4-methyl-piperazin-1-yl)-ethyl]-phenylamino}-5-oxo-5,8- |
| | dihydro-pyrido[2,3-d]pyrimidine-6-carboxylic acid ethoxy-amide |
| | ¹H NMR (400 MHz, CDCl₃) δ (ppm): 11.83 (s, 1H), 9.30 (s, 1H), 8.78 (s, 1H), 7.58 (br, |
| | 1H), 7.36 (d, 1H), 7.20 (br, 3H), 7.10 (d, 1H), 6.85 (br, 2H), 4.03 (q, 2H), 3.05 (t, 2H), |
| | 2.95 (t, 2H), 2.50-2.80 (m, 12H), 2.38 (s, 3H), 2.20 (m, 2H), 1.30 (t, 3H). Mass |
| | Spectrum (LCMS, ESI pos.) Calcd. For C₃₂H₃₇N₇O₃: 568.30 (M + H), Found 568.4. |
| 63 | 8-indan-5-yl-2-{4-[2-(4-methyl-piperazin-1-yl)-ethyl]-phenylamino}-5-oxo-5,8- |
| | dihydro-pyrido[2,3-d]pyrimidine-6-carboxylic acid isopropoxy-amide |
| | ¹H NMR (400 MHz, CDCl₃) δ (ppm): 11.83 (s, 1H), 9.38 (s, 1H), 8.82 (s, 1H), 7.70 (br, |
| | 1H), 7.42 (d, 1H), 7.25 (br, 3H), 7.15 (d, 1H), 6.90 (br, 2H), 4.30 (q, 2H), 3.05 (t, 2H), |
| | 2.98 (t, 2H), 2.50-2.80 (m, 12H), 2.36 (s, 3H), 2.22 (m, 2H), 1.37 (s, 3H), 1.35 (s, 3H). |
| | Mass Spectrum (LCMS, ESI pos.) Calcd. For C₃₃H₃₉N₇ O₃: 582.31 (M + H), Found |
| | 582.4. |
| 64 | 8-indan-5-yl-2-{4-[2-(4-methyl-piperazin-1-yl)-ethyl]-phenylamino}-6-(piperidine-1- |
| | carbonyl)-8H-pyrido[2,3-d]pyrimidin-5-one |
| | ¹H NMR (400 MHz, CDCl₃/CD₃OD 10:1 v/v) δ (ppm): 9.25 (s, 1H), 7.92 (s, 1H), |
| | 7.35 (d, 1H), 7.22 (m, 3H), 7.15 (d, 1H), 6.90 (br, 2H), 3.65 (br, 2H), 3.37 (br, 2H), 3.02 (t, |
| | 2H), 2.95 (t, 2H), 2.30-2.80 (m, 15H), 2.18 (m, 2H), 1.50 (m, 6H). Mass Spectrum |
| | (LCMS, ESI pos.) Calcd. For C₃₅H₄₁N₇O₂: 592.33 (M + H), Found 592.3. |
| 65 | 8-indan-5-yl-2-{4-[2-(4-methyl-piperazin-1-yl)-ethyl]-phenylamino}-5-oxo-5,8- |
| | dihydro-pyrido[2,3-d]pyrimidine-6-carboxylic acid (2-hydroxy-ethyl)-amide |
| | ¹H NMR (400 MHz, CDCl₃/CD₃OD 10:1 v/v) δ (ppm): 9.30 (s, 1H), 8.75 (s, 1H), |
| | 7.38 (d, 1H), 7.22 (m, 3H), 7.15 (d, 1H), 6.90 (br, 2H), 3.72 (t, 2H), 3.55 (t, 2H), 2.90 (m, |
| | 4H), 2.30-2.80 (m, 15H), 2.18 (m, 2H). Mass Spectrum (LCMS, ESI pos.) Calcd. For |
| | C₃₂H₃₇N₇O₃: 568.30 (M + H), Found 568.3. |
| 66 | 8-indan-5-yl-5-oxo-2-(3-piperazin-1-yl-phenylamino)-5,8-dihydro-pyrido[2,3- |
| | d]pyrimidine-6-carboxylic acid (2-hydroxy-ethyl)-amide |
| | ¹H NMR (400 MHz, CDCl₃/CD₃OD 10:1 v/v) δ (ppm): 9.98 (m, 1H), 9.30 (s, 1H), |
| | 8.75 (s, 1H), 7.39 (d, J = 7.9 Hz, 1H), 7.20 (s, 1H), 7.10 (d, J = 7.9 Hz, 1H), 6.80 (br, 1H), |
| | 6.65 (br, 1H), 6.55 (br, 2H), 3.73 (t, 2H), 3.57 (t, 2H), 3.25 (m, 8H), 3.02 (t, 2H), 2.95 (t, |
| | 2H), 2.20 (m, 2H). Mass Spectrum (LCMS, ESI pos.) Calcd. For C₂₉H₃₁N₇O₃: |
| | 526.25 (M + H), Found 526.3. |
| 67 | (S)-8-indan-5-yl-5-oxo-2-[4-(2-oxo-oxazolidin-4-ylmethyl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.30 (s, 1H), 8.81 (s, 1H), 7.86 (br, |
| | 1H), 7.42 (d, 1H), 7.35 (br, 2H), 7.25 (s, 1H), 7.18 (d, 1H), 6.90 (br, 2H), 5.05 (s, 1H), |
| | 4.49 (t, 1H), 4.16 (m, 1H), 4.04 (m, 1H), 3.90 (s, 3H), 3.10 (t, 2H), 3.00 (t, 2H), 2.80 (m, |
| | 2H), 2.22 (m, 2H). Mass Spectrum (LCMS, ESI pos.) Calcd. For C₂₈H₂₆N₆O₅: |
| | 527.20 (M + H), Found 527.1. |
| 68 | (S)-8-indan-5-yl-5-oxo-2-[4-(2-oxo-oxazolidin-4-ylmethyl)-phenylamino]-5,8-dihydro- |
| | pyrido[2,3-d]pyrimidine-6-carboxylic acid ethoxy-amide |
| | ¹H NMR (400 MHz, CDCl₃/CD₃OD 10:1 v/v) δ (ppm): 9.30 (s, 1H), 8.80 (s, 1H), |
| | 7.42 (d, 1H), 7.35 (br, 2H), 7.25 (s, 1H), 7.18 (d, 1H), 6.87 (br, 2H), 4.42 (t, 1H), 4.04 (m, |
| | 4H), 3.05 (t, 2H), 2.98 (t, 2H), 2.81 (m, 2H), 2.22 (m, 2H), 1.35 (t, 3H). Mass Spectrum |
| | (LCMS, ESI pos.) Calcd. For C₂₉H₂₈N₆O₅: 541.21 (M + H), Found 541.1. |
| 69 | 2-[4-(1-acetyl-piperidin-4-yl)-phenylamino]-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ (ppm): 11.98 (s, 1H), 9.39 (s, 1H), 8.81 (s, 1H), 7.60 (br, |
| | 1H), 7.42 (d, 1H), 7.30 (br, 2H), 7.27 (s, 1H), 7.18 (d, 1H), 6.90 (br, 2H), 4.80 (d, 1H), |
| | 3.95 (d, 1H), 3.92 (s, 3H), 3.18 (t, 1H), 3.10 (t, 2H), 3.00 (t, 2H), 2.62 (m, 2H), 2.22 (m, |
| | 2H), 2.15 (s, 3H), 1.86 (m, 2H), 1.60 (m, 2H). Mass Spectrum (LCMS, ESI pos.) Calcd. |
| | For C₃₁H₃₂N₆O₄: 553.25 (M + H), Found 553.1. |
| 70 | 2-[4-(1-acetyl-piperidin-4-yl)-phenylamino]-8-indan-5-yl-5-oxo-5,8-dihydro-pyrido[2,3- |
| | d]pyrimidine-6-carboxylic acid ethoxy-amide |
| | ¹H NMR (400 MHz, CDCl₃) δ (ppm): 11.90 (s, 1H), 9.39 (s, 1H), 8.81 (s, 1H), 7.60 (br, |
| | 1H), 7.42 (d, 1H), 7.30 (br, 2H), 7.27 (s, 1H), 7.18 (d, 1H), 6.90 (br, 2H), 4.80 (d, 1H), |
| | 4.10 (q, 2H), 3.95 (d, 1H), 3.18 (t, 1H), 3.10 (t, 2H), 3.00 (t, 2H), 2.62 (m, 2H), 2.22 (m, |
| | 2H), 2.15 (s, 3H), 1.86 (m, 2H), 1.60 (m, 2H), 1.35 (t, 3H). Mass Spectrum (LCMS, ESI |
| | pos.) Calcd. For C₃₂H₃₄N₆O₄: 567.26 (M + H), Found 567.1. |
| 127 | 8-indan-5-yl-2-[3-(2-morpholin-4-yl-ethoxy)-phenylamino]-5-oxo-5,8-dihydro- |
| | pyrido[2,3-d]pyrimidine-6-carboxylic acid amide |
| | ¹H NMR (TFA salt) (400 MHz, CDCl₃) δ (ppm): 9.36 (s, 1H), 8.79 (s, 1H), |
| | 7.33-7.32 (m, 2H), 7.22-7.18 (m, 3H), 7.13-7.06 (m, 2H), 4.33-4.17 (m, 2H), 3.92 (m, 2H), |
| | 3.65-3.53 (m, 2H), 3.44-3.35 (m, 2H), 2.87-2.93 (m, 4H), 2.14-2.19 (m, 2H); Mass Spectrum |
| | (LCMS, APCI pos.) Calcd. For: C₂₉H₃₀N₆O₄: 526.23; Found: 527.2 (M + H). |
| 128 | 8-indan-5-yl-2-[4-(4-methyl-piperazin-1-ylmethyl)-phenylamino]-5-oxo-5,8-dihydro- |
| | pyrido[2,3-d]pyrimidine-6-carboxylic acid methoxy-amide |
| | ¹H NMR (TFA salt) (400 MHz, CDCl₃) δ (ppm): 9.25 (s, 1H), 8.68 (s, 1H), |
| | 7.33-7.28 (m, 3H), 7.17-7.10 (m, 4H), 3.41 (s, 3H), 3.70-3.36 (m, 6H), 3.10-3.04 (m, 2H), 2.98 (m, |
| | 2H), 2.76 (s, 3H), 2.32-2.11 (m, 2H); Mass Spectrum (LCMS, APCI pos.) Calcd. For: |
| | C₃₀H₃₃N₇O₃: 539.26; Found: 540.3 (M + H). |
| 129 | 2-(3-dimethylsulfamoyl-phenylamino)-8-indan-5-yl-5-oxo-5,8-dihydro-pyrido[2,3- |
| | d]pyrimidine-6-carboxylic acid methoxy-amide |
| | ¹H NMR (TFA salt) (400 MHz, CDCl₃) δ (ppm): 9.40 (s, 1H), 8.84 (s, 1H), |
| | 7.85-7.79 (m, 2H), 7.41 (dd, J = 4.26, 3.77 Hz, 2H), 7.22-7.04 (m, 3H), 4.23-4.03 (m, 2H), 3.41 (s, |
| | 3H), 3.02 (t, 2H), 2.97 (t, 2H), 2.72 (s, 6H), 2.19-2.22 (m, 2H), 1.40-1.31 (m, 3H); Mass |
| | Spectrum (LCMS, APCI pos.) Calcd. For: C₂₆H₂₆N₆O₅S: 534.17; Found: 535.2 (M + H). |
| 130 | 8-indan-5-yl-5-oxo-2-[4-(2-pyrrolidin-1-yl-ethyl)-phenylamino]-5,8-dihydro-pyrido[2,3- |
| | d]pyrimidine-6-carboxylic acid methoxy-amide |
| | ¹H NMR (TFA salt) (400 MHz, CDCl₃) δ (ppm): 9.41 (s, 1H), 8.88 (s, 1H), |
| | 7.34-7.25 (m, 2H), 7.20-7.13 (m, 3H), 7.03-7.08 (m, 2H), 3.45 (s, 3H), 3.86 (m, 2H), 3.25-3.10 (m, |
| | 2H), 2.94-2.98 (m, 4H), 2.80-2.45 (m, 6H), 2.13 (m, 4H); Mass Spectrum (LCMS, APCI |
| | pos.) Calcd. For: C₃₀H₃₂N₆O₃: 524.25; Found: 525.2 (M + H). |
| 132 | 8-indan-5-yl-5-oxo-2-[4-(2-pyrrolidin-1-yl-ethyl)-phenylamino]-5,8-dihydro-pyrido[2,3- |
| | d]pyrimidine-6-carboxylic acid (2-methoxy-ethyl)-amide |
| | ¹H NMR (TFA salt) (400 MHz, CDCl₃) δ (ppm): 9.35 (s, 1H), 8.76 (s, 1H), |
| | 7.41-7.33 (m, 2H), 7.28-7.21 (m, 3H), 7.13-7.06 (m, 2H), 3.87-3.74 (m, 2H), 3.60 (d, J = 5.48 Hz, |
| | 2H), 3.52 (d, J = 5.13 Hz, 2H), 3.35 (s, 3H), 3.22-3.11 (m, 6H), 2.77-2.63 (m, 2H), |
| | 2.04 (br s 6H); Mass Spectrum (LCMS, APCI pos.) Calcd. For: C₃₂H₃₆N₆O₃: 552.28; Found: |
| | 553.3 (M + H). |
| 133 | 8-indan-5-yl-5-oxo-2-[4-(2-pyrrolidin-1-yl-ethyl)-phenylamino]-5,8-dihydro-pyrido[2,3- |
| | d]pyrimidine-6-carboxylic acid isopropoxy-amide |
| | ¹H NMR (TFA salt) (400 MHz, CDCl₃) δ (ppm): 9.43 (s, 1H), 8.89 (s, 1H), |
| | 7.51-7.37 (m, 2H), 7.37-7.29 (m, 3H), 7.21-7.14 (m, 2H), 4.35-4.23 (m, 1H), 3.96-3.82 (m, 2H), |
| | 3.30-3.16 (m, 2H), 3.16-3.05 (m, 4H), 3.00 (br s, 3H), 2.89-2.71 (m, 2H), 2.48-2.16 (m, |
| | 3H), 2.15-2.01 (m, 2H), 1.34 (app d, J = 6.19 Hz, 6H); Mass Spectrum (LCMS, APCI |
| | pos.) Calcd. For: C₃₂H₃₆N₆O₃: 552.28; Found: 553.3 (M + H). |
| 134 | 8-indan-5-yl-5-oxo-2-{4-[2-(2-oxo-pyrrolidin-1-yl)-ethyl]-phenylamino}-5,8-dihydro- |
| | pyrido[2,3-d]pyrimidine-6-carboxylic acid methoxy-amide |
| | ¹H NMR (TFA salt) (400 MHz, CDCl₃) δ (ppm): 9.28 (s, 1H), 8.76 (s, 1H), 7.36 (d, J = 7.88 Hz, |
| | 2H), 7.22 (br s, 3H), 7.16-7.02 (m, 2H), 3.83 (s, 3H), 3.41 (t, J = 7.45 Hz, 2H), |
| | 3.21 (t, J = 7.01 Hz, 2H), 3.02 (dd, J = 9.66, 5.04 Hz, 2H), 2.93 (t, J = 7.44 Hz, 2H), |
| | 2.69 (dd, J = 14.56, 7.38 Hz, 2H), 2.32 (t, J = 8.12 Hz, 2H), 2.25-2.07 (m, 2H), |
| | 1.91 (dd, J = 15.25, 7.66 Hz, 2H); Mass Spectrum (LCMS, APCI pos.) Calcd. For: |
| | C₃₀H₃₀N₆O₄: 538.23; Found: 539.2 (M + H). |
| 135 | 8-indan-5-yl-5-oxo-2-{4-[2-(2-oxo-pyrrolidin-1-yl)-ethyl]-phenylamino}-5,8-dihydro- |
| | pyrido[2,3-d]pyrimidine-6-carboxylic acid ethoxy-amide |
| | ¹H NMR (TFA salt) (400 MHz, CDCl₃) δ (ppm): 9.22 (s, 1H), 8.44 (s, 1H), |
| | 7.39-7.31 (m, 2H), 7.30-7.22 (m, 3H), 7.13 (dd, J = 7.79, 1.95 Hz, 2H), 4.41-4.24 (m, 2H), |
| | 3.50-3.38 (m, 2H), 3.27-3.13 (m, 2H), 3.00 (t, J = 7.35 Hz, 2H), 2.93 (t, J = 7.42 Hz, 2H), |
| | 2.69 (dd, J = 10.03, 4.89 Hz, 2H), 2.31 (t, J = 8.11 Hz, 2H), 2.14-2.17 (m, 2H), |
| | 1.98-1.80 (m, 2H), 1.30 (t, J = 7.15 Hz, 3H); Mass Spectrum (LCMS, APCI pos.) Calcd. For: |
| | C₃₁H₃₂N₆O₄: 552.25; Found: 553.2 (M + H). |
| 136 | 2-(4-dimethylsulfamoyl-phenylamino)-8-indan-5-yl-5-oxo-5,8-dihydro-pyrido[2,3- |
| | d]pyrimidine-6-carboxylic acid ethoxy-amide |
| | ¹H NMR (TFA salt) (400 MHz, CDCl₃) δ (ppm): 9.48 (s, 1H), 8.94 (s, 1H), |
| | 7.57-7.40 (m, 7H), 4.17-4.05 (m, 2H), 3.17 (s, 6H), 3.07-2.94 (m, 2H), 2.67 (d, J = 2.38 Hz, 2H), |
| | 2.34-2.14 (m, 2H), 1.37 (t, 3H); Mass Spectrum (LCMS, APCI pos.) Calcd. For: |
| | C₂₇H₂₈N₆O₅S: 548.18; Found: 549.2 (M + H). |
| 137 | 2-(4-dimethylsulfamoyl-phenylamino)-8-indan-5-yl-5-oxo-5,8-dihydro-pyrido[2,3- |
| | d]pyrimidine-6-carboxylic acid isopropoxy-amide |
| | ¹H NMR (TFA salt) (400 MHz, CDCl₃) δ (ppm): 9.42 (s, 1H), 8.63 (s, 1H), |
| | 7.59-7.41 (m, 6H), 7.28 (br s, 1H), 3.93 (d, 1H), 3.09 (t, J = 7.60 Hz, 2H), 3.00 (t, J = 7.31 Hz, |
| | 2H), 2.68 (app d, J = 4.50 Hz, 6H), 2.35-2.18 (m, 2H); Mass Spectrum (LCMS, APCI |
| | pos.) Calcd. For: C2₈H₃₀N₆O₅S: 562.20; Found: 563.2 (M + H). |
| 141 | 8-cyclohexyl-2-[4-(1-methyl-piperidin-4-yl)-phenylamino]-5-oxo-5,8-dihydro- |
| | pyrido[2,3-d]pyrimidine-6-carboxylic acid ethoxy-amide |
| | ¹H NMR (TFA salt) (400 MHz, CDCl₃) δ (ppm): 9.44 (s, 1H), 8.90 (s, 1H), |
| | 7.49-7.42 (m, 2H), 7.21-7.14 (m, 2H), 3.92 (q, J = 7.04 Hz, 2H), 3.08 (s, 3H), 3.00 (d, J = 7.59 Hz, |
| | 2H), 2.80 (br s, 8H), 2.65 (br s, 2H), 2.61-2.38 (m, 2H), 2.23 (s, 1H), 2.13-2.03 (m, |
| | 2H), 1.36 (t, J = 7.02 Hz, 3H); Mass Spectrum (LCMS, APCI pos.) Calcd. For: |
| | C₂₈H₃₆N₆O₃: 504.28; Found: 505.3 (M + H). |
| 142 | 8-cyclopropyl-5-oxo-2-[4-(2-pyrrolidin-1-yl-ethyl)-phenylamino]-5,8-dihydro- |
| | pyrido[2,3-d]pyrimidine-6-carboxylic acid ethoxy-amide |
| | ¹H NMR (TFA salt) (400 MHz, CDCl₃) δ (ppm): 9.31 (s, 1H), 8.79 (s, 1H), |
| | 7.86-7.67 (m, 4H), 4.08 (q, J = 7.04 Hz, 2H), 2.87-2.72 (m, 6H), 2.27-2.16 (m, 4H), 2.16-2.04 (m, |
| | 4H), 1.22 (t, J = 7.00 Hz, 3H); Mass Spectrum (LCMS, APCI pos.) Calcd. For: |
| | C₂₅H₃₀N₆O₃: 462.24; Found: 463.2 (M + H). |
| 143 | 8-cyclopropyl-5-oxo-2-[4-(2-pyrrolidin-1-yl-ethyl)-phenylamino]-5,8-dihydro- |
| | pyrido[2,3-d]pyrimidine-6-carboxylic acid isopropoxy-amide |
| | ¹H NMR (TFA salt) (400 MHz, CDCl₃) δ (ppm): 9.30 (s, 1H), 8.80 (s, 1H), |
| | 7.89-7.71 (m, 2H), 7.34-7.25 (m, 2H), 4.32-4.19 (m, 1H), 3.99-3.81 (m, 2H), 3.65-3.51 (m, 2H), |
| | 3.40-3.25 (m, 2H), 3.23-3.11 (m, 2H), 2.89-2.68 (m, 2H), 2.09 (br s 5H), 1.33 (app d, J = 6.18 Hz, |
| | 6H), 1.22-1.09 (m, 2H); Mass Spectrum (LCMS, APCI pos.) Calcd. For: |
| | C₂₆H₃₂N₆O₃: 476.25; Found: 477.2 (M + H). |

### Example 4

### 8-(4-ethyl-phenyl)-5-oxo-2-(4-piperidin-4-yl-phenylamino)-5,8-dihydro-pyrido[2,3-d]pyrimidine-6-carboxylic acid isopropoxy-amide (Cpd 58)

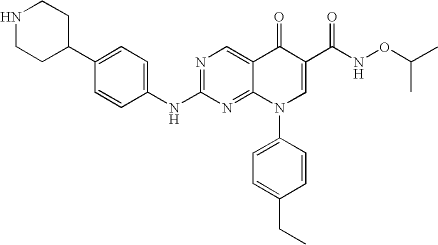

### A. 4-(4-amino-phenyl)-3,6-dihydro-2H-pyridine-1-carboxylic acid tert-butyl ester

The title compound was prepared by Suzuki coupling of 4-(4,4,5,5-tetramethyl-[1,3,2]dioxaborolan-2-yl)-phenylamine with 4-trifluoromethanesulfonyloxy-3,6-dihydro-2H-pyridine-1-carboxylic acid tert-butyl ester (Synthesis, 993, (1991)). Mass spectrum (ESI, m/z): Calcd. for C₁₆H₂₂N₂O₂, 275.2 (M+H), found 275.1.

### B. 4-(4-amino-phenyl)-piperidine-1-carboxylic acid tert-butyl ester

A solution of 4-(4-amino-phenyl)-3,6-dihydro-2H-pyridine-1-carboxylic acid tert-butyl ester (0.35 g, 1.2 mmol) in methanol was hydrogenated over 10% Pd/C at 20 psi for 1 h. The solution was filtered and concentrated to give 0.35 g (100%) of the title compound as a yellow solid. ¹H NMR (400 MHz, DMSO-d₆) δ (ppm): 6.85 (d, J=8.3 Hz, 2H), 6.50 (d, J=8.3 Hz, 2H), 4.81 (s, 2H), 4.012 (m, 2H), 3.85 (br, 2H), 2.44 (m, 1H), 2.66 (m, 2H), 1.42 (m, 11H).

### C. 8-(4-ethyl-phenyl)-5-oxo-2-(4-piperidin-4-yl-phenylamino)-5,8-dihydro-pyrido[2,3-d]pyrimidine-6-carboxylic acid ethyl ester

Using procedures described in Example 3, Step F, the title compound was prepared from 4-(4-amino-phenyl)-piperidine-1-carboxylic acid tert-butyl ester (140 mg, 0.5 mmol) and 8-(4-ethyl-phenyl)-2-methanesulfonyl-5-oxo-5,8-dihydro-pyrido[2,3-d]pyrimidine-6-carboxylic acid ethyl ester (100 mg, 0.25 mmol). The Boc group was removed using TFA before HPLC purification. The title compound was obtained as a yellow solid (70 mg, 56

### D. 8-(4-ethyl-phenyl)-5-oxo-2-(4-piperidin-4-yl-phenylamino)-5,8-dihydro-pyrido[2,3-d]pyrimidine-6-carboxylic acid isopropoxy-amide

The title compound was prepared according to procedures described in Example 1. ¹H NMR (400 MHz, CDCl₃/CD₃OD 10:1 v/v) δ (ppm): 9.30 (s, 1H), 8.72 (s, 1H), 7.40 (d, 2H), 7.30 (d, 2H), 7.25 (br, 2H), 6.87 (br, 2H), 4.20 (m, 1H), 3.45 (d, 2H), 2.90 (m, 2H), 2.80 (m, 2H), 2.65 (br, 1H), 1.90 (m, 4H), 1.32 (t, 3H), 1.26 (d, 6H). Mass Spectrum (LCMS, APCI pos.) Calcd. For C₃₀H₃₄N₆O₃: 527.27 (M+H), Found 527.1.

### Example 5

### 2-[4-(1-acetyl-piperidin-4-yl)-phenylamino]-8-(4-ethyl-phenyl)-5-oxo-5,8-dihydro-pyrido[2,3-d]pyrimidine-6-carboxylic acid isopropoxy-amide (Cpd 59)

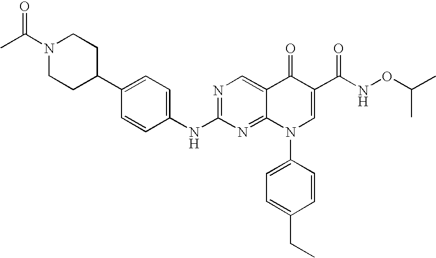

### A. 2-[4-(1-acetyl-piperidin-4-yl)-phenylamino]-8-(4-ethyl-phenyl)-5-oxo-5,8-dihydro-pyrido[2,3-d]pyrimidine-6-carboxylic acid ethyl ester

To a solution of 8-(4-ethyl-phenyl)-5-oxo-2-(4-piperidin-4-yl-phenylamino)-5,8-dihydro-pyrido[2,3-d]pyrimidine-6-carboxylic acid ethyl ester (Example 4, Step C, 20 mg, 0.04 mmol) in 2 mL of pyridine was added acetic anhydride (0.019 mL, 0.2 mmol). After 2 h, water was added to quench the reaction. The reaction mixture was extracted with CH₂Cl₂. The organic fractions were dried (Na₂SO₄), filtered and the filtrate was concentrated. The residue was purified by chromatography (HPLC (32 mL/min, 15-100% MeCN/H₂O (0.1% TFA v/v) gradient over 12 min). The relevant HPLC fractions were pooled, made basic (sat. NaHCO₃) and extracted with CH₂Cl₂. The organic fractions were dried (Na₂SO₄) and filtered. The filtrate was concentrated to provide the title compound as a white solid (20 mg, 93%).

### B. 2-[4-(1-acetyl-piperidin-4-yl)-phenylamino]-8-(4-ethyl-phenyl)-5-oxo-5,8-dihydro-pyrido[2,3-d]pyrimidine-6-carboxylic acid isopropoxy-amide

The title compound was prepared according to procedures described in Example 1. ¹H NMR (400 MHz, CDCl₃/CD₃OD 10:1 v/v) δ (ppm): 9.30 (s, 1H), 8.72 (s, 1H), 7.38 (d, 2H), 7.25 (d, 2H), 7.20 (br, 2H), 6.84 (br, 2H), 4.63 (m, 1H), 4.20 (m, 1H), 3.85 (m, 1H), 3.10 (m, 1H), 2.80 (m, 2H), 2.60 (m, 2H), 2.08 (s, 3H), 1.80 (m, 2H), 1.46 (m, 2H), 1.32 (t, 3H), 1.26 (d, 6H). Mass Spectrum (LCMS, APCI pos.) Calcd. For C₃₂H₃₆N₆O₄: 569.28 (M+H), Found 569.2.

### Example 6

### 8-(4-ethyl-phenyl)-2-[4-(1-methanesulfonyl-piperidin-4-yl)-phenylamino]-5-oxo-5,8-dihydro-pyrido[2,3-d]pyrimidine-6-carboxylic acid isopropoxy-amide (Cpd 60)

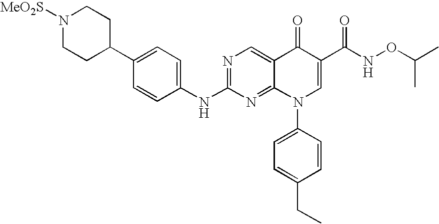

### A. 8-(4-ethyl-phenyl)-2-[4-(1-methanesulfonyl-piperidin-4-yl)-phenylamino]-5-oxo-5,8-dihydro-pyrido[2,3-d]pyrimidine-6-carboxylic acid ethyl ester

To mixture of 8-(4-ethyl-phenyl)-5-oxo-2-(4-piperidin-4-yl-phenylamino)-5,8-dihydro-pyrido[2,3-d]pyrimidine-6-carboxylic acid ethyl ester (Example 4, Step C, 20 mg, 0.04 mmol) in 2 mL of CH₂Cl₂, was added methanesulfonyl chloride (0.008 mL, 0.1 mmol). After stirring for 12 h at rt, water was added to quench the reaction. The aqueous solution was extracted with CH₂Cl₂. The organic fractions were dried (Na₂SO₄), filtered and the filtrate was evaporated. The residue was purified by HPLC (32 mL/min, 15-100% MeCN/H₂O (0.1% TFA v/v) gradient over 12 min). The relevant HPLC fractions were pooled, made basic (sat. NaHCO₃) and extracted with CH₂Cl₂. The organic fractions were dried (Na₂SO₄) and filtered. The filtrate was concentrated to leave a white solid (14 mg, 61%).

### B. 8-(4-ethyl-phenyl)-2-[4-(1-methanesulfonyl-piperidin-4-yl)-phenylamino]-5-oxo-5,8-dihydro-pyrido[2,3-d]pyrimidine-6-carboxylic acid isopropoxy-amide

The tile compound was prepared according to procedures described in Example 1. ¹H NMR (400 MHz, CDCl₃) δ (ppm): 11.78 (s, 1H), 9.34 (s, 1H), 8.75 (s, 1H), 7.55 (br, 1H), 7.38 (d, 2H), 7.25 (d, 2H), 7.20 (br, 2H), 6.84 (br, 2H), 4.22 (m, 1H), 3.88 (m, 2H), 2.80 (m, 5H), 2.70 (m, 2H), 2.48 (m, 1H), 1.85 (m, 2H), 1.70 (m, 2H), 1.35 (t, 3H), 1.30 (d, 6H). Mass Spectrum (LCMS, APCI pos.) Calcd. For C₃₁H₃₆N₆O₅S: 605.25 (M+H), Found 605.1.

### Example 7

### 8-indan-5-yl-2-[4-(1-methanesulfonyl-piperidin-4-yl)-phenylamino]-5-oxo-5,8-dihydro-pyrido[2,3-d]pyrimidine-6-carboxylic acid methoxy-amide (Cpd 76)

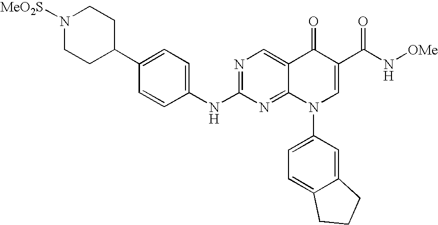

The title compound was prepared from 8-indan-5-yl-5-oxo-2-(4-piperidin-4-yl-phenylamino)-5,8-dihydro-pyrido[2,3-d]pyrimidine-6-carboxylic acid methoxy-amide Cpd 75 (Example 13) according to procedures described in Example 6, Step A. ¹H NMR (400 MHz, CDCl₃) δ (ppm): 11.97 (s, 1H), 9.40 (s, 1H), 8.82 (s, 1H), 7.62 (br, 1H), 7.42 (d, 1H), 7.22 (br, 3H), 7.18 (d, 1H), 6.92 (br, 2H), 3.95 (d, 1H), 3.90 (s, 3H), 3.10 (m, 2H), 3.00 (t, 2H), 2.82 (s, 3H), 2.75 (t, 2H), 2.54 (m, 1H), 2.22 (m, 2H), 1.90 (m, 2H), 1.75 (m, 2H). Mass Spectrum (LCMS, APCI pos.) Calcd. For C₃₀H₃₂N₆O₅S: 589.22 (M+H), Found 589.1.

### Example 8

### 8-indan-5-yl-2-[4-(2-methanesulfonyl-ethyl)-phenylamino]-5-oxo-5,8-dihydro-pyrido[2,3-d]pyrimidine-6-carboxylic acid methoxy-amide (Cpd 71)

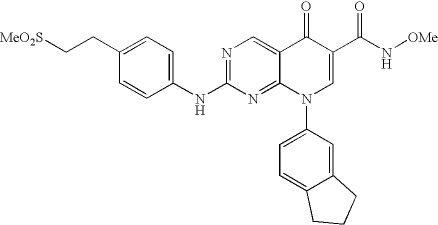

### A. 4-(2-methanesulfonyl-ethyl)-phenylamine

To a solution of 1-(2-bromo-ethyl)-4-nitro-benzene (3 g, 13 mmol) in EtOH (70 mL) was added sodium thiomethoxide (1 g, 14 mmol). After stirring at rt for 12 h, the reaction mixture was concentrated in vacuo. The residue was diluted with EtOAc and washed with water. The organic fraction was dried (Na₂SO₄), filtered and the filtrate was concentrated to give a white solid. The sulfide was diluted with CH₂Cl₂ (100 mL) and to it was added portionwise mCPBA (8 g). After stirring at rt for 5 h, the reaction mixture was washed with 1N NaOH solution (2×100 mL). The organic fraction was dried (Na₂SO₄), filtered and the filtrate was concentrated to give a white solid. The title compound (500 mg) was hydrogenated over Pd/C (10%) in methanol under atmospheric pressure. The reaction mixture was filtered, and the filtrate was concentrated to give the title compound as a white solid (450 mg).

### B. 8-indan-5-yl-2-methanesulfonyl-5-oxo-5,8-dihydro-pyrido[2,3-d]pyrimidine-6-carboxylic acid methoxy-amide

A solution of 8-indan-5-yl-2-methylsulfanyl-5-oxo-5,8-dihydro-pyrido[2,3-d]pyrimidine-6-carboxylic acid ethyl ester (4 g, 10.5 mmol) in 120 mL of 1N HCl (aq.) and 100 mL of 1,4-dioxane was heated at reflux for 4 h. The reaction mixture was cooled, resulting in precipitation of product. The product was isolated by filtration, washed with water and dried in vacuo. To a solution of the acid (3.8 g) in 30 mL of CH₂Cl₂ at 0° C. was added 1.4 mL of oxalyl chloride (16.1 mmol) and 20 μL of DMF. The solution was stirred at room temperature for 1 h. The solvent and excess oxalyl chloride was removed in vacuo. To the residue in 100 mL of CH₂Cl₂ at 0° C. was added MeONH₂.HCl (1.8 g, 21.6 mmol) followed by slow addition of Et₃N (6 mL, 43 mmol). The reaction mixture was stirred at 0° C. for 10 min and at rt for 12 h. Water was added and the reaction mixture was extracted with CH₂Cl₂ (200 mL×2). The combined organic fractions were washed with brine, dried (Na₂SO₄), and filtered. The filtrate was concentrated in vacuo to give an off-white solid (2 g). The sulfide was converted to the sulfone using the procedure described in Example 1, Step E. The title compound was obtained as yellow solid (2.3 g, 53%).

### C. 8-indan-5-yl-2-[4-(2-methanesulfonyl-ethyl)-phenylamino]-5-oxo-5,8-dihydro-pyrido[2,3-d]pyrimidine-6-carboxylic acid ethyl ester

Using procedures described in Example 3, Step F, the title compound was prepared from 4-(2-methanesulfonyl-ethyl)-phenylamine (24 mg, 0.12 mmol) and 8-indan-5-yl-2-methanesulfonyl-5-oxo-5,8-dihydro-pyrido[2,3-d]pyrimidine-6-carboxylic acid methoxy-amide (28 mg, 0.068 mmol). The title compound was obtained as a yellow solid (15.8 mg, 43%). ¹H NMR (400 MHz, CDCl₃/CD₃OD 10:1 v/v) δ (ppm): 9.30 (s, 1H), 8.77 (s, 1H), 7.40 (d, 1H), 7.30 (br, 2H), 7.23 (s, 1H), 7.15 (d, 1H), 6.90 (br, 2H), 3.85 (s, 3H), 3.20 (m, 2H), 3.05 (m, 4H), 2.98 (t, 2H), 2.80 (s, 3H), 2.22 (m, 2H). Mass Spectrum (LCMS, ESI pos.) Calcd. For C₂₇H₂₇N₅O₅S: 534.17 (M+H), Found 534.0.

Using the procedure of Example 8 and reagents, starting materials and conditions known to those skilled in the art, the following compounds representative of the present invention were prepared:

| Cpd | Name and Data |
|---|---|
| 72 | 8-indan-5-yl-5-oxo-2-{4-[2-(3-oxo-piperazin-1-yl)-ethyl]-phenylamino}-5,8-dihydro- |
| | pyrido[2,3-d]pyrimidine-6-carboxylic acid methoxy-amide |
| | ¹H NMR (400 MHz, CDCl₃) δ (ppm): 11.90 (s, 1H), 9.30 (s, 1H), 8.77 (s, 1H), 7.72 (br, |
| | 1H), 7.38 (d, 1H), 7.22 (br, 3H), 7.15 (d, 1H), 6.90 (br, 2H), 6.00 (br, 1H), 3.82 (s, 3H), |
| | 3.35 (s, 2H), 3.20 (s, 2H), 3.00 (t, 2H), 2.96 (t, 2H), 2.60 (m, 6H), 2.18 (m, 2H). Mass |
| | Spectrum (LCMS, APCI pos.) Calcd. For C₃₀H₃₁N₇O₄: 554.24 (M + H), Found 554.1. |
| 73 | 2-{4-[2-(1,5-dioxa-9-aza-spiro[5.5]undec-9-yl)-ethyl]-phenylamino}-8-indan-5-yl-5- |
| | oxo-5,8-dihydro-pyrido[2,3-d]pyrimidine-6-carboxylic acid methoxy-amide |
| | ¹H NMR (400 MHz, CDCl₃) δ (ppm): 11.98 (s, 1H), 9.39 (s, 1H), 8.81 (s, 1H), 7.76 (br, |
| | 1H), 7.42 (d, 1H), 7.25 (br, 3H), 7.17 (d, 1H), 6.95 (br, 2H), 3.90 (m, 7H), 3.08 (t, 2H), |
| | 3.00 (t, 2H), 2.70-3.00 (m, 6H), 2.15 (m, 2H), 2.10 (m, 4H), 1.75-1.90 (m, 4H). Mass |
| | Spectrum (LCMS, APCI pos.) Calcd. For C₃₄H₃₈N₆O₅: 611.29 (M + H), Found 611.2. |
| 74 | 8-indan-5-yl-2-(4-{2-[methyl-(tetrahydro-pyran-4-yl)-amino]-ethyl}-phenylamino)-5- |
| | oxo-5,8-dihydro-pyrido[2,3-d]pyrimidine-6-carboxylic acid methoxy-amide |
| | ¹H NMR (400 MHz, CDCl₃) δ (ppm): 11.90 (s, 1H), 9.30 (s, 1H), 8.77 (s, 1H), 7.72 (br, |
| | 1H), 7.38 (d, 1H), 7.22 (br, 3H), 7.15 (d, 1H), 6.90 (br, 2H), 4.00 (m, 2H), 3.80 (s, 3H), |
| | 3.35 (t, 2H), 3.00 (m, 2H), 2.96 (t, 2H), 2.30-2.65 (m, 6H), 2.18 (m, 2H), 1.40-1.80 (m, |
| | 6H). Mass Spectrum (LCMS, APCI pos.) Calcd. For C₃₂H₃₆N₆O₄: 569.28 (M + H), |
| | Found 569.2. |
| 77 | 8-indan-5-yl-2-[4-(2-isopropylsulfamoyl-ethyl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 11.90 (s, 1H), 9.30 (s, 1H), 8.77 (s, 1H), 7.58 (br, |
| | 1H), 7.38 (d, 1H), 7.22 (br, 3H), 7.10 (d, 1H), 6.90 (br, 2H), 3.82 (s, 3H), 3.58 (m, 1H), |
| | 3.15 (m, 2H), 2.97 (m, 6H), 2.18 (m, 2H), 1.15 (s, 3H), 1.13 (s, 3H). Mass Spectrum |
| | (LCMS, APCI pos.) Calcd. For C₂₉H₃₂N₆O₅S: 577.22 (M + H), Found 577.1. |
| 79 | 2-[4-(2-hydroxy-ethyl)-phenylamino]-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ (ppm): 11.90 (s, 1H), 9.30 (s, 1H), 8.77 (s, 1H), 7.58 (br, |
| | 1H), 7.38 (d, 1H), 7.22 (br, 3H), 7.10 (d, 1H), 6.90 (br, 2H), 3.82 (s, 3H), 3.78 (m, 2H), |
| | 3.00 (t, 2H), 2.95 (t, 2H), 2.78 (m, 2H), 2.15 (m, 2H). Mass Spectrum (LCMS, APCI |
| | pos.) Calcd. For C₂₆H₂₅N₅O₄: 472.19 (M + H), Found 472.1. |
| 80 | 8-indan-5-yl-2-[4-(2-morpholin-4-yl-ethyl)-phenylamino]-5-oxo-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.77 (s, 1H), |
| | 7.40 (d, 1H), 7.30 (br, 2H), 7.20 (s, 1H), 7.15 (d, 1H), 6.93 (br, 2H), 4.15 (t, 2H), 3.95 (d, |
| | 2H), 3.82 (s, 3H), 3.48 (d, 2H), 2.98 (m, 10H), 2.20 (m, 2H). Mass Spectrum (LCMS, |
| | APCI pos.) Calcd. For C₃₀H₃₂N₆O₄: 541.24 (M + H), Found 541.2. |
| 83 | 8-indan-5-yl-5-oxo-2-{4-[2-(4-oxo-piperidin-1-yl)-ethyl]-phenylamino}-5,8-dihydro- |
| | pyrido[2,3-d]pyrimidine-6-carboxylic acid methoxy-amide |
| | ¹H NMR (400 MHz, CDCl₃) δ (ppm): 11.90 (s, 1H), 9.30 (s, 1H), 8.77 (s, 1H), 7.50 (br, |
| | 1H), 7.38 (d, 1H), 7.20 (br, 3H), 7.12 (d, 1H), 6.90 (br, 2H), 3.82 (d, 2H), 3.02 (m, 2H), |
| | 2.98 (t, 2H), 2.70 (m, 6H), 2.42 (m, 6H), 2.20 (m, 2H). Mass Spectrum (LCMS, APCI |
| | pos.) Calcd. For C₃₁H₃₂N₆O₄: 553.25 (M + H), Found 553.2. |
| 89 | 8-indan-5-yl-2-[4-(2-methylsulfamoyl-ethyl)-phenylamino]-5-oxo-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.32 (s, 1H), 8.77 (s, 1H), |
| | 7.40 (d, 1H), 7.30 (br, 2H), 7.25 (s, 1H), 7.15 (d, 1H), 6.90 (br, 2H), 3.82 (s, 3H), 3.20 (m, |
| | 2H), 3.00 (m, 6H), 2.67 (s, 3H), 2.20 (m, 2H). Mass Spectrum (LCMS, ESI pos.) Calcd. |
| | For C₂₇H₂₈N₆O₅S: 549.18 (M + H), Found 549.2. |
| 94 | (4-{4-[8-(4-ethyl-phenyl)-6-methoxycarbamoyl-5-oxo-5,8-dihydro-pyrido[2,3- |
| | d]pyrimidin-2-ylamino]-phenyl}-piperidin-1-yl)-acetic acid |
| | ¹H NMR (400 MHz, CD₃OD) δ (ppm): 9.30 (s, 1H), 8.66 (s, 1H), 7.50 (d, 2H), 7.45 (d, |
| | 2H), 7.37 (br, 2H), 6.95 (br, 2H), 4.10 (br, 2H), 3.83 (s, 3H), 3.78 (br, 2H), 3.22 (m, 2H), |
| | 2.84 (m, 3H), 2.00 (m, 3H), 1.40 (m, 3H). Mass Spectrum (LCMS, ESI pos.) Calcd. For |
| | C₃₀H₃₂N₆O₅: 557.24 (M + H), Found 557.3. |
| 96 | 8-(4-ethyl-phenyl)-2-{4-[1-(2-methanesulfonyl-ethyl)-piperidin-4-yl]-phenylamino}-5- |
| | oxo-5,8-dihydro-pyrido[2,3-d]pyrimidine-6-carboxylic acid methoxy-amide |
| | ¹H NMR (400 MHz, CDCl₃) δ (ppm): 11.98 (s, 1H), 9.40 (s, 1H), 8.82 (s, 1H), 7.62 (br, |
| | 1H), 7.42 (d, 2H), 7.35 (d, 2H), 7.22 (br, 2H), 6.94 (br, 2H), 3.90 (s, 3H), 3.22 (m, 2H), |
| | 3.06 (s, 3H), 2.94 (br, 2H), 2.82 (q, 2H), 2.44 (m, 1H), 2.20 (m, 2H), 1.83 (m, 2H), |
| | 1.66 (br, 4H), 1.38 (t, 3H). Mass Spectrum (LCMS, ESI pos.) Calcd. For C₃₁H₃₆N₆O₅S: |
| | 605.25 (M + H), Found 605.3. |
| 107 | 2-{4-[2-(adamantan-2-ylamino)-ethyl]-phenylamino}-8-indan-5-yl-5-oxo-5,8-dihydro- |
| | pyrido[2,3-d]pyrimidine-6-carboxylic acid methoxy-amide |
| | adamantan-2-yl-[2-(4-amino-phenyl)-ethyl]-amine was prepared using the procedure for |
| | preparation of 4-[2-(4-methyl-piperazin-1-yl)-ethyl]-phenylamine. ¹H NMR (400 MHz, |
| | CDCl₃) δ (ppm): 11.90 (s, 1H), 9.30 (s, 1H), 8.78 (s, 1H), 7.57 (br, 1H), 7.36 (d, 1H), |
| | 7.20 (br, 3H), 7.10 (d, 1H), 6.90 (br, 2H), 3.85 (s, 3H), 3.01 (t, 2H), 2.95 (t, 2H), |
| | 2.83 (m, 5H), 2.20 (m, 2H), 1.40-1.90 (m, 14H). Mass Spectrum (LCMS, ESI pos.) Calcd. |
| | For C₃₆H₄₀N₆O₃: 605.32 (M + H), Found 605.6. |
| 108 | 2-{4-[2-(bicyclo[2.2.1]hept-2-ylamino)-ethyl]-phenylamino}-8-indan-5-yl-5-oxo-5,8- |
| | dihydro-pyrido[2,3-d]pyrimidine-6-carboxylic acid methoxy-amide |
| | [2-(4-amino-phenyl)-ethyl]-bicyclo[2.2.1]hept-2-yl-amine was prepared using the |
| | procedure for preparation of 4-[2-(4-methyl-piperazin-1-yl)-ethyl]-phenylamine. ¹H |
| | NMR (400 MHz, CDCl₃) δ (ppm): 11.90 (s, 1H), 9.30 (s, 1H), 8.78 (s, 1H), 7.57 (br, |
| | 1H), 7.36 (d, 1H), 7.20 (br, 3H), 7.10 (d, 1H), 6.90 (br, 2H), 3.81 (s, 3H), 3.00 (m, 2H), |
| | 2.95 (t, 2H), 2.70 (m, 3H), 2.20 (m, 4H), 1.40-1.90 (m, 10H). Mass Spectrum (LCMS, |
| | ESI pos.) Calcd. For C₃₃H₃₆N₆O₃: 565.28 (M + H), Found 565.6. |
| 109 | (R)-1-{2-[4-(8-indan-5-yl-6-methoxycarbamoyl-5-oxo-5,8-dihydro-pyrido[2,3- |
| | d]pyrimidin-2-ylamino)-phenyl]-ethyl}-pyrrolidine-2-carboxylic acid |
| | (R)-1-[2-(4-amino-phenyl)-ethyl]-pyrrolidine-2-carboxylic acid was prepared using the |
| | procedure for preparation of 4-[2-(4-methyl-piperazin-1-yl)-ethyl]-phenylamine. ¹H |
| | NMR (400 MHz, CDCl₃/CD₃OD 10:1 v/v) δ (ppm): 9.30 (s, 1H), 9.38 (s, 1H), 8.77 (s, |
| | 1H), 7.40 (d, J = 7.9 Hz, 1H), 7.23 (br, 2H), 7.20 (s, 1H), 7.12 (d, J = 7.9 Hz, 1H), |
| | 6.90 (br, 2H), 4.10 (br, 1H), 3.82 (s, 3H), 3.00 (m, 8H), 2.43 (m, 2H), 2.20 (m, 6H). Mass |
| | Spectrum (LCMS, ESI pos.) Calcd. For C₃₁H₃₂N₆O₅: 569.24 (M + H), Found 569.5. |
| 110 | 2-{4-[2-(7-aza-bicyclo[2.2.1]hept-7-yl)-ethyl]-phenylamino}-8-indan-5-yl-5-oxo-5,8- |
| | dihydro-pyrido[2,3-d]pyrimidine-6-carboxylic acid methoxy-amide |
| | 4-[2-(7-aza-bicyclo[2.2.1]hept-7-yl)-ethyl]-phenylamine was prepared using the |
| | procedure for preparation of 4-[2-(4-methyl-piperazin-1-yl)-ethyl]-phenylamine. ¹H |
| | NMR (400 MHz, CDCl₃) δ (ppm): 11.98 (s, 1H), 9.39 (s, 1H), 8.80 (s, 1H), 7.59 (br, |
| | 1H), 7.43 (d, 1H), 7.23 (br, 3H), 7.18 (d, 1H), 6.95 (br, 2H), 3.90 (s, 3H), 3.42 (br, 2H), |
| | 3.06 (t, 2H), 3.00 (t, 2H), 2.80 (m, 2H), 2.60 (br, 2H), 2.22 (m, 2H), 1.40-1.90 (m, 8H). |
| | Mass Spectrum (LCMS, ESI pos.) Calcd. For C₃₂H₃₄N₆O₃: 551.27 (M + H), Found |
| | 551.5. |
| 171 | 2-[4-(2-imidazol-1-yl-ethyl)-phenylamino]-8-indan-5-yl-5-oxo-5,8-dihydro-pyrido [2,3- |
| | d]pyrimidine-6-carboxylic acid methoxy-amide |
| | 4-(2-imidazol-1-yl-ethyl)-phenylamine was prepared using the procedure for preparation |
| | of 4-[2-(4-methyl-piperazin-1-yl)-ethyl]-phenylamine. ¹H NMR (400 MHz, CDCl₃) δ |
| | (ppm): 11.9 (s, 1H), 9.30 (s, 1H), 8.76 (s, 1H), 7.70 (s, 1H), 7.34 (d, 1H), 7.19 (s, 1H), |
| | 7.11 (d, 1H), 6.97 (s, 1H), 6.74 (s, 1H), 6.66 (br, 4H), 4.05 (t, 2H), 3.83 (s, 3H), |
| | 2.99-2.87 (m, 6H), 2.15 (m, 2H). Mass Spectrum (LCMS, ESI pos.) Calcd. For C₂₉H₂₇N₇O₃: |
| | 522.2 (M + H), Found: 522.3. |
| 172 | 8-indan-5-yl-2-{4-[2-(5-methyl-1H-[1,2,4]triazol-3-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 9.26 (s, 1H), 8.72 (s, 1H), 7.37 (d, 1H), 7.20 (br, |
| | 3H), 7.10 (d, 1H), 6.87 (br, 2H), 3.82 (s, 3H), 3.02-2.80 (m, 6H), 2.35 (s, 3H), 2.10 (m, |
| | 2H). Mass Spectrum (LCMS, ESI pos.) Calcd. For C₂₉H₂₈N₈O₃: 537.23 (M + H), Found: |
| | 537.6. |
| 173 | 8-indan-5-yl-5-oxo-2-(4-pyridin-4-ylmethyl-phenylamino)-5,8-dihydro-pyrido[2,3- |
| | d]pyrimidine-6-carboxylic acid methoxy-amide |
| | ¹H NMR (TFA salt) (400 MHz, CDCl₃) δ (ppm): 9.31 (s, 1H), 8.78 (s, 1H), 8.71 (br s, |
| | 3H), 7.65 (br s, 3H), 7.30 (br s, 3H), 7.18 (d, 2H), 3.90 (s, 3H) 2.85 (m, 4H), 2.10 (t, |
| | 2H). Mass Spectrum (LCMS, APCI pos.) Calcd. For: C₃₀H₂₆N₆O₃: 518.21; Found: |
| | 519.5 (M + H). |
| 174 | 8-indan-5-yl-5-oxo-2-(3-[1,2,4]triazol-1-yl-phenylamino)-5,8-dihydro-pyrido[2,3- |
| | d]pyrimidine-6-carboxylic acid methoxy-amide |
| | ¹H NMR (TFA salt) (400 MHz, CDCl₃) δ (ppm): 9.36 (s, 1H), 8.75 (s, 1H), 8.95 (s, |
| | 1H), 1.07 (s, 1H), 7.22-7.35 (m, 4H), 7.02-7.15 (m, 3H), 3.92 (s, 3H), 2.90 (m, 4H), |
| | 2.08 (m 2H). Mass Spectrum (LCMS, APCI pos.) Calcd. For: C₂₆H₂₂N₈O₃: 494.18; Found: |
| | 495.1 (M + H). |

### Example 9

### 8-indan-5-yl-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide (Cpd 78)

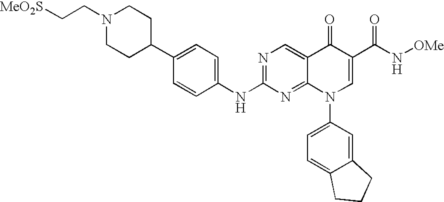

### A. ethanesulfonic acid 2-methanesulfonyl-ethyl ester

To a solution of 2-methanesulfonylethanol (540 mg, 4.35 mmol), methanesulfonyl chloride (370 μL, 4.8 mmol) in CH₂Cl₂ (10 mL) at 0° C. was added triethylamine (660 μL, 4.8 mmol). After stirring at rt for 2 h, the reaction mixture was concentrated in vacuo to give the title compound as a colorless oil, which was used in the next step without further purification.

### B. 4-[1-(2-methanesulfonyl-ethyl)-piperidin-4-yl]-phenylamine

To a solution of 4-(4-nitro-phenyl)-piperidine (140 mg, 0.68 mmol) and methanesulfonic acid 2-methanesulfonyl-ethyl ester (132 mg, 0.65 mmol) in CH₂Cl₂ (10 mL) was added diisopropylethylamine (120 μL, 0.68 mmol). The reaction mixture was stirred at rt for 12 h. After an aqueous work-up, the reaction mixture was extracted with CH₂Cl₂. The combined organic fractions were dried over Na₂SO₄, filtered and the filtrate was concentrated. The residue was purified by chromatography (silica, 5% methanol in CH₂Cl₂) to give 155 mg of the nitro analog as a white solid. The nitro analog was hydrogenated over Pd/C (10%) in methanol under atmosphere pressure. The reaction mixture was filtered and the filtrate was concentrated to give the title compound as a white solid (132 mg).

### C. 8-indan-5-yl-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide

The title compound was prepared by reacting 4-[1-(2-methanesulfonyl-ethyl)-piperidin-4-yl]-phenylamine (15 mg, 0.053 mmol) with 8-indan-5-yl-2-methanesulfonyl-5-oxo-5,8-dihydro-pyrido[2,3-d]pyrimidine-6-carboxylic acid methoxy-amide (20 mg, 0.048 mmol). ¹H NMR (400 MHz, CDCl₃) δ (ppm): 11.98 (s, 1H), 9.40 (s, 1H), 8.82 (s, 1H), 7.58 (br, 1H), 7.42 (d, 1H), 7.25 (br, 3H), 7.20 (d, 1H), 6.94 (br, 2H), 3.89 (s, 3H), 3.20 (m, 2H), 3.02 (m, 9H), 2.92 (m, 2H), 2.25 (m, 1H), 2.20 (m, 4H), 1.83 (m, 2H), 1.60 (m, 4H). Mass Spectrum (LCMS, APCI pos.) Calcd. For C₃₂H₃₆N₆O₅S: 617.25 (M+H), Found 617.2.

### Example 10

### 8-indan-5-yl-2-[4-(2-methoxy-ethyl)-phenylamino]-5-oxo-5,8-dihydro-pyrido[2,3-d]pyrimidine-6-carboxylic acid methoxy-amide (Cpd 81)

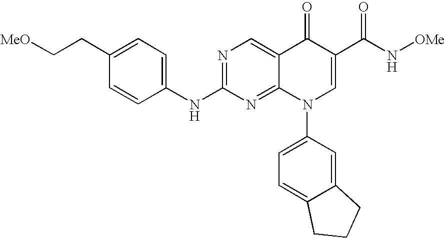

### A. 4-(2-methoxy-ethyl)-phenylamine

To a solution of 2-(4-nitro-phenyl)-ethanol (1 g, 6 mmol) in 50 mL of acetone was added dimethyl sulfate (1.13 g, 9 mmol) and K₂CO₃ (1.24 g, 9 mmol). The mixture was heated at reflux for 24 h. After an aqueous work-up, the reaction mixture was extracted with CH₂Cl₂. The combined organic fractions were dried over Na₂SO₄, filtered and the filtrate was concentrated. The residue was purified by chromatography (silica, EtOAc/hexanes 1:4-1:1 v/v). The nitro analog was hydrogenated over Pd/C (10%) in methanol under atmosphere pressure. The reaction mixture was filtered and the filtrate was concentrated to give the title compound as brown oil.

### B. 8-indan-5-yl-2-[4-(2-methoxy-ethyl)-phenylamino]-5-oxo-5,8-dihydro-pyrido[2,3-d]pyrimidine-6-carboxylic acid methoxy-amide

The title compound was prepared by reacting 4-(2-methoxy-ethyl)-phenylamine (10 mg, 0.066 mmol) with 8-indan-5-yl-2-methanesulfonyl-5-oxo-5,8-dihydro-pyrido[2,3-d]pyrimidine-6-carboxylic acid methoxy-amide (20 mg, 0.048 mmol). ¹H NMR (400 MHz, CDCl₃) δ (ppm): 11.96 (s, 1H), 9.39 (s, 1H), 8.82 (s, 1H), 7.60 (br, 1H), 7.40 (d, 1H), 7.27 (br, 3H), 7.19 (d, 1H), 6.96 (br, 2H), 4.30 (t, 2H), 3.90 (s, 3H), 3.78 (s, 3H), 3.10 (t, 2H), 3.02 (t, 2H), 2.95 (t, 2H), 2.22 (m, 2H). Mass Spectrum (LCMS, APCI pos.) Calcd. For C₂₇H₂₇N₅O₄: 486.21 (M+H), Found 486.2.

### Example 11

### 8-indan-5-yl-2-[4-(2-methanesulfonylamino-ethyl)-phenylamino]-5-oxo-5,8-dihydro-pyrido[2,3-d]pyrimidine-6-carboxylic acid methoxy-amide (Cpd 82)

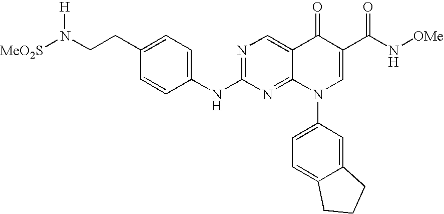

### A. N-[2-(4-amino-phenyl)-ethyl]-methanesulfonamide

To a solution of 2-(4-nitro-phenyl)-ethylamine (290 mg, 1.44 mmol) in 20 mL of CH₂Cl₂ at 0° C. was added methanesulfonyl chloride (133 μL, 1.73 mmol) and triethylamine (440 μL, 3.17 mmol). The mixture was stirred at rt for 4 h. After an aqueous work-up, the reaction mixture was extracted with CH₂Cl₂. The combined organic fractions were dried over Na₂SO₄, filtered and the filtrate was concentrated. The residue was purified by chromatography (silica, 2% methanol in CH₂Cl₂). The resulting nitro compound was hydrogenated over Pd/C (10%) in methanol under atmosphere pressure. The reaction mixture was filtered and the filtrate was concentrated to give the title compound as a yellow solid.

### B. 8-indan-5-yl-2-[4-(2-methanesulfonylamino-ethyl)-phenylamino]-5-oxo-5,8-dihydro-pyrido[2,3-d]pyrimidine-6-carboxylic acid methoxy-amide

The title compound was prepared by reacting N-[2-(4-amino-phenyl)-ethyl]-methanesulfonamide (10 mg, 0.047 mmol) with 8-indan-5-yl-2-methanesulfonyl-5-oxo-5,8-dihydro-pyrido[2,3-d]pyrimidine-6-carboxylic acid methoxy-amide (20 mg, 0.048 mmol). ¹H NMR (400 MHz, CDCl₃) δ (ppm): 11.90 (s, 1H), 9.30 (s, 1H), 8.78 (s, 1H), 7.60 (br, 1H), 7.40 (d, 1H), 7.27 (br, 3H), 7.15 (d, 1H), 6.90 (br, 2H), 4.16 (m, 1H), 3.80 (s, 3H), 3.28 (m, 2H), 3.02 (t, 2H), 2.95 (t, 2H), 2.80 (s, 3H), 2.75 (m, 2H), 2.18 (m, 2H). Mass Spectrum (LCMS, APCI pos.) Calcd. For C₂₇H₂₈N₆O₅S: 549.18 (M+H), Found 549.1.

### Example 12

### 3-{4-[4-(8-indan-5-yl-6-methoxycarbamoyl-5-oxo-5,8-dihydro-pyrido[2,3-d]pyrimidin-2-ylamino)-phenyl]-piperidin-1-yl}-propionic acid (Cpd 84)

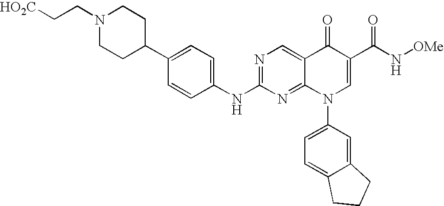

To a solution of 8-indan-5-yl-5-oxo-2-(4-piperidin-4-yl-phenylamino)-5,8-dihydro-pyrido[2,3-d]pyrimidine-6-carboxylic acid methoxy-amide Cpd 75 (Example 13) (10 mg, 0.02 mmol) in DMF (1 mL) was added 3-bromopropionic acid (3.4 mg, 0.022 mmol) and triethylamine (3 μL, 0.022 mmol). The reaction mixture was stirred at rt for 12 h. The reaction mixture was concentrated and the residue was purified (HPLC, 32 mL/min, 5-80% MeCN/H₂O (0.1% TFA v/v) gradient over 12 min) to give the title compound as a yellow solid (4.5 mg, 39%). ¹H NMR (400 MHz, CDCl₃/CD₃OD 10:1 v/v) δ (ppm): 9.30 (s, 1H), 8.75 (s, 1H), 7.38 (d, 1H), 7.22 (br, 2H), 7.20 (s, 1H), 7.12 (d, 1H), 6.88 (br, 2H), 3.80 (s, 3H), 3.45 (m, 2H), 3.12 (br, 1H), 3.05 (m, 2H), 2.96 (t, 2H), 2.70 (m, 6H), 2.18 (m, 2H), 1.98 (m, 4H). Mass Spectrum (LCMS, ESI pos.) Calcd. For C₃₂H₃₄N₆O₅: 583.26 (M+H), Found 583.3.

### Example 13

### 8-indan-5-yl-5-oxo-2-(4-piperidin-4-yl-phenylamino)-5,8-dihydro-pyrido[2,3-d]pyrimidine-6-carboxylic acid methoxy-amide (Cpd 75)

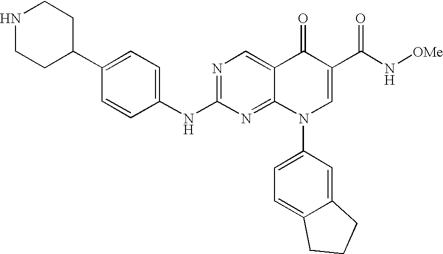

The title compound was prepared from 4-(4-amino-phenyl)-piperidine-1-carboxylic acid tert-butyl ester (Example 4, Step B) and 8-indan-5-yl-2-methanesulfonyl-5-oxo-5,8-dihydro-pyrido[2,3-d]pyrimidine-6-carboxylic acid methoxy-amide according to procedures described in Example 4. ¹H NMR (400 MHz, CDCl₃) δ (ppm): 11.90 (s, 1H), 9.30 (s, 1H), 8.77 (s, 1H), 7.58 (br, 1H), 7.38 (d, 1H), 7.22 (br, 3H), 7.15 (d, 1H), 6.90 (br, 2H), 3.80 (s, 3H), 3.20 (d, 2H), 3.00 (m, 2H), 2.96 (t, 2H), 2.70 (t, 2H), 2.50 (m, 1H), 2.18 (m, 2H), 1.65 (m, 4H). Mass Spectrum (LCMS, APCI pos.) Calcd. For C₂₉H₃₀N₆O₃: 511.24 (M+H), Found 511.1.

Using the procedure of Example 13 and reagents, starting materials and conditions known to those skilled in the art, the following compounds representative of the present invention were prepared:

| Cpd | Name and Data |
|---|---|
| 155 | 2-[4-(1-ethyl-piperidin-4-yl)-phenylamino]-8-indan-5-yl-5-oxo-5,8-dihydro-pyrido[2,3- |
| | d]pyrimidine-6-carboxylic acid methoxy-amide |
| | To a solution of 8-indan-5-yl-5-oxo-2-(4-piperidin-4-yl-phenylamino)-5,8-dihydro- |
| | pyrido[2,3-d]pyrimidine-6-carboxylic acid methoxy-amide Cpd 75 (50 mg, 0.098 mmol) |
| | in DMSO (1 mL) was added iodoethane (15 mg, 0.098 mmol) and triethylamine (13 μL, |
| | 0.098 mmol). The reaction mixture was stirred for 32 hrs at rt. Water was added and the |
| | reaction mixture was extracted with CH₂Cl₂, then dried (MgSO₄) and filtered. The filtrate |
| | was concentrated and purified (HPLC, 32 mL/min, 5-80% MeCN/H₂O (0.1% TFA v/v) |
| | gradient over 12 min) to give the title compound as a solid (8.9 mg). ¹H NMR (400 MHz, |
| | CDCL₃) δ (ppm): 11.92 (s, 1H), 9.38 (s, 1H), 8.83 (s, 1H), 7.41 (d, 1H), 7.31 (br, 3H), |
| | 7.17 (s, 2H), 3.92 (s, 3H), 3.76 (d, 2H), 2.89-3.21 (m, 6H), 2.61-2.78 (m, 4H), |
| | 2.08-2.32 (m, 2H), 1.92 (d, 2H), 1.41 (t, 3H). Mass Spectrum (LCMS, APCI pos.) Calcd. For |
| | C₃₁H₃₄N₆O₃: 538.27 (M + H), Found 539.3. |

### Example 14

### {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid (Cpd 85)

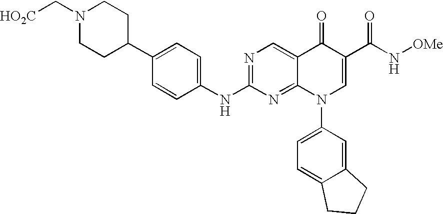

To a solution of 8-indan-5-yl-5-oxo-2-(4-piperidin-4-yl-phenylamino)-5,8-dihydro-pyrido[2,3-d]pyrimidine-6-carboxylic acid methoxy-amide Cpd 75 (10 mg, 0.02 mmol) in DMF (1 mL) was added bromoacetic acid (3 mg, 0.022 mmol) and triethylamine (3 μL, 0.022 mmol). The reaction mixture was stirred for 12 h at rt. The reaction mixture was concentrated and the residue was purified (HPLC, 32 mL/min, 5-80% MeCN/H₂O (0.1% TFA v/v) gradient over 12 min) to give the title compound as a yellow solid (6.8 mg, 61%). ¹H NMR (300 MHz, CDCl₃/CD₃OD 10:1 v/v) δ (ppm): 9.20 (s, 1H), 8.65 (s, 1H), 7.35 (d, 1H), 7.22 (br, 2H), 7.17 (s, 1H), 7.05 (d, 1H), 6.80 (br, 2H), 3.75 (s, 3H), 3.58 (d, 2H), 3.45 (s, 2H), 2.90 (m, 6H), 2.63 (br, 1H), 2.10 (m, 2H), 1.95 (m, 4H). Mass Spectrum (LCMS, APCI pos.) Calcd. For C₃₁H₃₂N₆O₅: 569.24 (M+H), Found 569.1.

### Example 15

### {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester (Cpd 86)

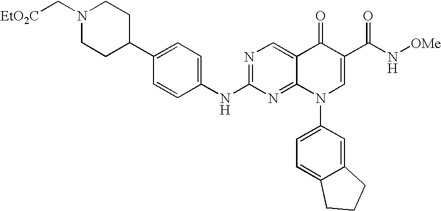

### A. {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetyl chloride

To a solution of {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid Cpd 85 (10 mg, 0.017 mmol) in CH₂Cl₂ (1 mL) was added oxalyl chloride (3 μL, 0.034 mmol) and a catalytic amount of DMF. The solution was stirred for 2 hrs at rt. The reaction mixture was concentrated in vacuo to give the title compound as a brown oil.

### B. {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester

To the acid chloride from Step A (5 mg) was added ethanol (0.5 mL). After 1 h at rt, the reaction mixture was concentrated in vacuo. The residue was purified (HPLC, 32 mL/min, 5-80% MeCN/H₂O (0.1% TFA v/v) gradient over 12 min) to give the title compound as a white solid (3.1 mg, 61%). ¹H NMR (400 MHz, CDCl₃) δ (ppm): 11.90 (s, 1H), 9.30 (s, 1H), 8.77 (s, 1H), 7.50 (br, 1H), 7.35 (d, 1H), 7.20 (br, 3H), 7.10 (d, 1H), 6.90 (br, 2H), 4.15 (m, 2H), 3.80 (s, 3H), 3.58 (m, 2H), 3.45 (s, 2H), 2.90 (m, 6H), 2.63 (br, 1H), 2.20 (m, 2H), 1.77 (m, 2H), 1.50 (m, 2H), 1.22 (t, 3H). Mass Spectrum (LCMS, ESI pos.) Calcd. For C₃₃H₃₆N₆O₅: 597.27 (M+H), Found 597.4.

### Example 16

### 8-indan-5-yl-2-{4-[1-(2-methanesulfonylamino-2-oxo-ethyl)-piperidin-4-yl]-phenylamino}-5-oxo-5,8-dihydro-pyrido[2,3-d]pyrimidine-6-carboxylic acid methoxy-amide (Cpd 87)

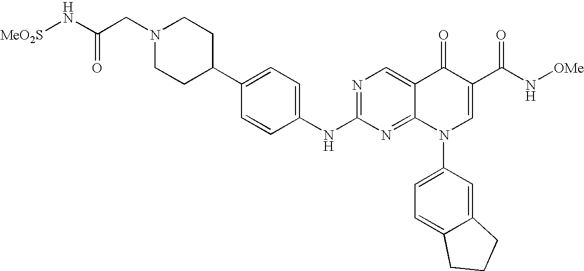

To a solution of {4-[4-(8-indan-5-yl-6-methoxycarbamoyl-5-oxo-5,8-dihydro-pyrido[2,3-d]pyrimidin-2-ylamino)-phenyl]-piperidin-1-yl}-acetyl chloride (Example 15, Step A, 5 mg) in CH₂Cl₂ (0.5 mL) was added methanesulfonamide (1.5 mg) and triethylamine (2 μL). The reaction mixture was stirred at rt for 12 h. The reaction mixture was concentrated in vacuo, and the residue was purified (HPLC, 32 mL/min, 5-80% MeCN/H₂O (0.1% TFA v/v) gradient over 12 min) to give the title compound as a white solid (1 mg). ¹H NMR (400 MHz, CDCl₃/CD₃OD 10:1 v/v) δ (ppm): 9.35 (s, 1H), 8.80 (s, 1H), 7.42 (d, 1H), 7.25 (br, 3H), 7.15 (d, 1H), 6.92 (br, 2H), 3.90 (s, 3H), 3.58 (m, 2H), 3.42 (s, 3H), 3.30 (m, 4H), 3.10 (m, 2H), 3.00 (t, 2H), 2.65 (br, 1H), 2.20 (m, 2H), 1.95 (m, 4H). Mass Spectrum (LCMS, ESI pos.) Calcd. For C₃₂H₃₅N₇O₆S: 646.24 (M+H), Found 646.3.

### Example 17

### 2-[4-(2-acetylsulfamoyl-ethyl)-phenylamino]-8-indan-5-yl-5-oxo-5,8-dihydro-pyrido[2,3-d]pyrimidine-6-carboxylic acid methoxy-amide (Cpd 90)

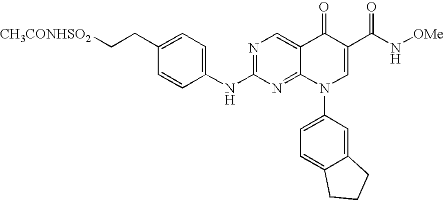

### A. 2-(4-nitro-phenyl)-ethanesulfonyl chloride

1-(2-bromo-ethyl)-4-nitro-benzene (3 g, 13 mmol) and potassium thioacetate (3 g, 26 mmol) in DMSO (10 mL) were stirred at r.t. for 3 hours. EtOAc was used to dilute the reaction mixture. The organic layer was washed with water twice (2×100 mL), then with brine and dried over Na₂SO₄. The solvent was evaporated in vacuo to give a brown solid (˜3 g), which was taken up in 50 mL of acetic acid. To the stirring solution was added 20 mL of hydrogen peroxide (30% in water). The resulting yellow solution was stirred at r.t. overnight. Water (50 mL) was added and the solvent was evaporated in vacuo with minimal heating. The yellow residue was dried in vacuo for two days, suspended in thionyl chloride (18 mL) and heated at reflux (80° C.) for 6 hours. The volatiles were evaporated to give the title compound as a yellow solid, which was used in the next step without further purification.

### B. 2-(4-amino-phenyl)-ethanesulfonic acid acetyl-amide

A mixture of 2-(4-nitro-phenyl)-ethanesulfonyl chloride (200 mg, 0.8 mmol) and ammonium hydroxide (5 mL) in CH₂Cl₂ (10 mL) was stirred for 2 h at rt. To the reaction mixture was added CH₂Cl₂ (100 mL) and water (100 mL). The organic fraction was dried over Na₂SO₄, filtered, and the filtrate was concentrated in vacuo. To a solution of the residue in CH₂Cl₂ (10 mL) was added pyridine (0.3 mL) and acetic anhydride (75 μL), and the reaction mixture was stirred at rt for 3 days. After an aqueous work-up, the reaction mixture was extracted with CH₂Cl₂. The combined organic fractions were dried over Na₂SO₄, filtered and the filtrate was concentrated. The residue was purified by chromatography (silica, 5% methanol in CH₂Cl₂). The resulting nitro compound was hydrogenated over Pd/C (10%) in methanol under atmosphere pressure. The reaction mixture was filtered and the filtrate was concentrated to give the title compound as a pink solid (66 mg).

### C. 2-[4-(2-acetylsulfamoyl-ethyl)-phenylamino]-8-indan-5-yl-5-oxo-5,8-dihydro-pyrido[2,3-d]pyrimidine-6-carboxylic acid methoxy-amide

The title compound was prepared by reacting 2-(4-amino-phenyl)-ethanesulfonic acid acetyl-amide (15 mg, 0.062 mmol) with 8-indan-5-yl-2-methanesulfonyl-5-oxo-5,8-dihydro-pyrido[2,3-d]pyrimidine-6-carboxylic acid methoxy-amide (20 mg, 0.048 mmol). ¹H NMR (400 MHz, CDCl₃/CD₃OD 10:1 v/v) δ (ppm): 9.32 (s, 1H), 8.77 (s, 1H), 7.40 (d, 1H), 7.30 (br, 2H), 7.25 (s, 1H), 7.15 (d, 1H), 6.90 (br, 2H), 3.85 (s, 3H), 3.58 (m, 2H), 3.00 (m, 6H), 2.20 (m, 2H), 1.95 (s, 3H). Mass Spectrum (LCMS, ESI pos.) Calcd. For C₂₈H₂₈N₆O₆S: 577.18 (M+H), Found 577.2.

### Example 18

### 8-indan-5-yl-5-oxo-2-[4-(1H-tetrazol-5-ylmethyl)-phenylamino]-5,8-dihydro-pyrido[2,3-d]pyrimidine-6-carboxylic acid methoxy-amide (Cpd 91)

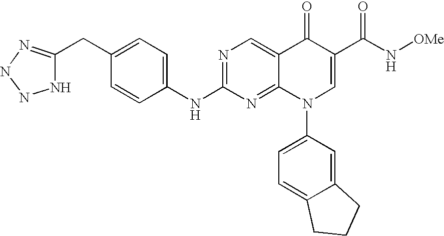

### A. 4-(1H-tetrazol-5-ylmethyl)-phenylamine

To a mixture of (4-amino-phenyl)-acetonitrile (1 g, 7.6 mmol) in DMF (20 mL) was added sodium azide (1 g, 15.2 mmol) and ammonium chloride (0.82 g, 15.2 mmol). The mixture was stirred at 110° C. for 5 h. After cooling, CH₂Cl₂ (100 mL) and water (100 mL) were added to the reaction. The organic layer was dried (Na₂SO₄) and filtered. The filtrate was concentrated, and the residue was purified by chromatography (silica, methanol/CH₂Cl₂ 1:9 v/v) to give the title compound as a brown oil (105 mg, 8%).

### B. 8-indan-5-yl-5-oxo-2-[4-(1H-tetrazol-5-ylmethyl)-phenylamino]-5,8-dihydro-pyrido[2,3-d]pyrimidine-6-carboxylic acid methoxy-amide

The title compound was prepared by reacting 4-(1H-tetrazol-5-ylmethyl)-phenylamine (15 mg, 0.086 mmol) with 8-indan-5-yl-2-methanesulfonyl-5-oxo-5,8-dihydro-pyrido[2,3-d]pyrimidine-6-carboxylic acid methoxy-amide (20 mg, 0.048 mmol). ¹H NMR (400 MHz, CDCl₃/CD₃OD 10:1 v/v) δ (ppm): 9.22 (s, 1H), 8.68 (s, 1H), 7.30 (d, 1H), 7.20 (br, 2H), 7.15 (s, 1H), 7.05 (d, 1H), 6.84 (br, 2H), 4.05 (s, 2H), 3.78 (s, 3H), 2.95 (m, 4H), 2.10 (m, 2H). Mass Spectrum (LCMS, ESI pos.) Calcd. For C₂₆H₂₃N₉O₃: 510.19 (M+H), Found 510.2.

### Example 19

### 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid cyclopentyloxy-amide (Cpd 92)

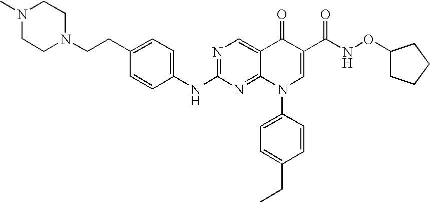

### A. O-cyclopentyl-hydroxylamine hydrochloride

A mixture of N-hydroxyphthalimide (3 g, 18.4 mmol), bromocyclopentane (2.13 mL, 18.4 mmol) and K₂CO₃ (2.5 g, 18.4 mmol) in DMSO (10 mL) was heated for 3 h at 80° C. After cooling, water (100 mL) was added. The mixture was extracted with EtOAc. The organic fractions were dried (Na₂SO₄) and filtered. The filtrate was concentrated and the residue was purified by chromatography (silica, 20% EtOAc in hexanes). The white solid product (4.2 g, 18.2 mmol) was dissolved in CH₂Cl₂ (100 mL) and methanol (10 mL). To the stirred solution was added hydrazine (1.76 mL, 36.4 mmol). After 24 h, a white precipitate was filtered and the filtrate was washed with 10% ammonium hydroxide and acidified with concentrated HCl. The organic fraction was concentrated and the title compound (white solid, 1.6 g, 64%) crystallized out upon standing.

### B. 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid cyclopentyloxy-amide

Using the procedure of Example 1, O-cyclopentyl-hydroxylamine hydrochloride was reacted with 8-indan-5-yl-2-methanesulfonyl-5-oxo-5,8-dihydro-pyrido[2,3-d]pyrimidine-6-carboxylic acid ethyl ester (Example 1, Step E). The product was reacted with 4-[2-(4-methyl-piperazin-1-yl)-ethyl]-phenylamine to provide the title compound. ¹H NMR (400 MHz, CDCl₃) 6 (PPM): 11.80 (s, 1H), 9.30 (s, 1H), 8.78 (s, 1H), 7.55 (br, 1H), 7.35 (d, 2H), 7.25 (d, 2H), 7.18 (br, 2H), 6.85 (br, 2H), 4.60 (m, 1H), 2.75 (q, 2H), 2.40-2.80 (m, 12H), 2.30 (s, 3H), 1.90 (m, 2H), 1.75 (m, 4H), 1.52 (m, 2H), 1.30 (t, 3H). Mass Spectrum (LCMS, ESI pos.) Calcd. For C₃₄H₄₁N₇O₃: 596.33 (M+H), Found 596.4.

### Example 20

### 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid (1-ethyl-propoxy)-amide (Cpd 93)

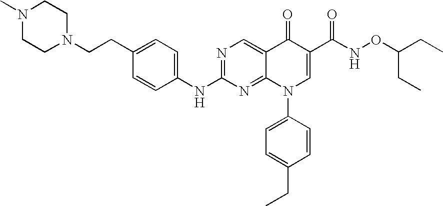

### A. O-(1-ethyl-propyl)-hydroxylamine hydrochloride

A mixture of N-hydroxyphthalimide (3 g, 18.4 mmol), O-(1-ethyl-propyl)-hydroxylamine (2.13 mL, 18.4 mmol) and K₂CO₃ (2.5 g, 18.4 mmol) in DMSO (10 mL) was heated for 3 h at 80° C. After cooling, water (100 mL) was added. The mixture was extracted with EtOAc. The organic fractions were dried (Na₂SO₄) and filtered. The filtrate was concentrated and the residue was purified by chromatography (silica, 20% EtOAc in hexanes). The white solid product (4.2 g, 18.2 mmol) was dissolved in CH₂Cl₂ (100 mL) and methanol (10 mL). To the stirred solution was added hydrazine (1.76 mL, 36.4 mmol). After 24 h, a white precipitate was filtered and the filtrate was washed with 10% ammonium hydroxide and acidified with concentrated HCl. The organic fraction was concentrated and the title compound (white solid, 1.6 g, 64%) crystallized out upon standing.

### B. 8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid (1-ethyl-propoxy)-amide

Using the procedure of Example 1, O-(1-ethyl-propyl)-hydroxylamine hydrochloride was reacted with 8-indan-5-yl-2-methanesulfonyl-5-oxo-5,8-dihydro-pyrido[2,3-d]pyrimidine-6-carboxylic acid ethyl ester (Example 1, Step E). The product was then reacted with 4-[2-(4-methyl-piperazin-1-yl)-ethyl]-phenylamine to provide the title compound. ¹H NMR (400 MHz, CDCl₃) δ (ppm): 11.80 (s, 1H), 9.40 (s, 1H), 8.80 (s, 1H), 7.60 (br, 1H), 7.40 (d, 2H), 7.32 (d, 2H), 7.22 (br, 2H), 6.95 (br, 2H), 3.83 (m, 1H), 2.80 (q, 2H), 2.40-2.75 (m, 12H), 2.35 (s, 3H), 1.70 (m, 4H), 1.35 (t, 3H), 1.00 (t, 6H). Mass Spectrum (LCMS, ESI pos.) Calcd. For C₃₄H₄₃N₇O₃: 598.34 (M+H), Found 598.3.

### Example 21

### 2-{4-[2-(2-hydroxy-ethylamino)-ethyl]-phenylamino}-8-indan-5-yl-5-oxo-5,8-dihydro-pyrido[2,3-d]pyrimidine-6-carboxylic acid methoxy-amide (Cpd 97)

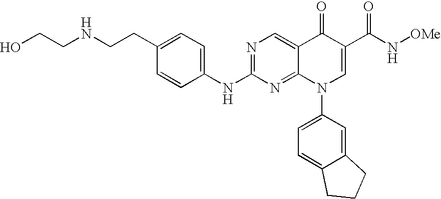

Using procedures described in Example 11, the title compound was prepared from 2-[4-(2-amino-ethyl)-phenylamino]-8-indan-5-yl-5-oxo-5,8-dihydro-pyrido[2,3-d]pyrimidine-6-carboxylic acid methoxy-amide and 2-bromoethanol. ¹H NMR (400 MHz, CDCl₃) δ (ppm): 11.98 (s, 1H), 9.40 (s, 1H), 8.82 (s, 1H), 7.60 (br, 1H), 7.42 (d, 1H), 7.25 (br, 3H), 7.18 (d, 1H), 6.94 (br, 2H), 3.90 (s, 3H), 3.63 (t, 2H), 3.06 (t, 2H), 3.00 (t, 2H), 2.62-2.95 (m, 6H), 2.22 (m, 2H). Mass Spectrum (LCMS, ESI pos.) Calcd. For C₂₈H₃₀N₆O₄: 515.23 (M+H), Found 515.3.

Using the procedure of Example 21 and reagents, starting materials and conditions known to those skilled in the art, the following compounds representative of the present invention were prepared:

| Cpd | Name and Data |
|---|---|
| 98 | 8-indan-5-yl-2-{4-[2-(2-methoxy-ethylamino)-ethyl]-phenylamino}-5-oxo-5,8-dihydro- |
| | pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.76 (s, 1H), |
| | 7.38 (d, 1H), 7.25 (br, 3H), 7.12 (d, 1H), 6.90 (br, 2H), 3.82 (s, 3H), 3.60 (t, 2H), 3.30 (s, |
| | 3H), 2.90-3.10 (m, 8H), 2.18 (m, 4H). Mass Spectrum (LCMS, ESI pos.) Calcd. For |
| | C₂₉H₃₂N₆O₄: 529.25 (M + H), Found 529.3. |
| 99 | 8-indan-5-yl-2-{4-[2-(2-methanesulfonyl-ethylamino)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃/CD₃OD 10:1 v/v) δ (ppm): 9.35 (s, 1H), 8.78 (s, 1H), |
| | 7.40 (d, 1H), 7.25 (br, 3H), 7.15 (d, 1H), 6.90 (br, 2H), 3.86 (s, 3H), 3.60 (t, 2H), 3.10 (m, |
| | 6H), 2.98 (t, 2H), 2.95 (s, 3H), 2.82 (m, 2H), 2.73 (m, 2H), 2.20 (m, 2H). Mass |
| | Spectrum (LCMS, ESI pos.) Calcd. For C₂₉H₃₂N₆O₅S: 577.22 (M + H), Found 577.3. |
| 100 | 2-(4-{2-[bis-(2-hydroxy-ethyl)-amino]-ethyl}-phenylamino)-8-indan-5-yl-5-oxo-5,8- |
| | dihydro-pyrido[2,3-d]pyrimidine-6-carboxylic acid methoxy-amide |
| | ¹H NMR (400 MHz, CDCl₃/CD₃OD 10:1 v/v) δ (ppm): 9.37 (s, 1H), 8.80 (s, 1H), |
| | 7.42 (d, 1H), 7.30 (br, 2H), 7.22 (s, 1H), 7.18 (d, 1H), 6.96 (br, 2H), 4.05 (m, 4H), 3.90 (s, |
| | 3H), 3.38 (m, 6H), 3.10 (m, 4H), 3.00 (t, 2H), 2.22 (m, 2H). Mass Spectrum (LCMS, |
| | ESI pos.) Calcd. For C₃₀H₃₄N₆O₅: 559.26 (M + H), Found 559.3. |

### Example 22

### (R)-2-{4-[2-(2-hydroxymethyl-pyrrolidin-1-yl)-ethyl]-phenylamino}-8-indan-5-yl-5-oxo-5,8-dihydro-pyrido[2,3-d]pyrimidine-6-carboxylic acid methoxy-amide (Cpd 101)

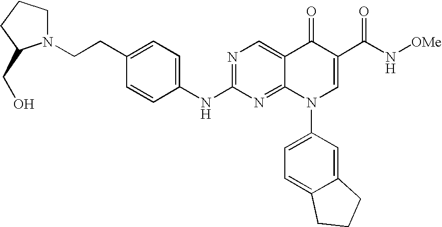

Using the procedure for preparation of 4-[2-(4-methyl-piperazin-1-yl)-ethyl]-phenylamine, (R)-4-[2-(2-hydroxymethyl-pyrrolidin-1-yl)-ethyl]-phenylamine was prepared from 1-(2-bromo-ethyl)-4-nitro-benzene and (R)-pyrrolidin-2-yl-methanol. The title compound was prepared by reacting the product with 8-indan-5-yl-2-methanesulfonyl-5-oxo-5,8-dihydro-pyrido[2,3-d]pyrimidine-6-carboxylic acid methoxy-amide. ¹H NMR (400 MHz, CDCl₃) δ (ppm): 11.98 (s, 1H), 9.38 (s, 1H), 8.80 (s, 1H), 7.57 (br, 1H), 7.42 (d, J=7.9 Hz, 1H), 7.21 (br, 3H), 7.18 (d, J=7.9 Hz, 1H), 6.90 (br, 2H), 3.90 (s, 3H), 3.50 (m, 2H), 3.05 (m, 2H), 3.00 (t, J=7.4 Hz, 2H), 2.50-2.90 (m, 7H), 2.25 (m, 2H), 1.80 (m, 4H). Mass Spectrum (LCMS, ESI pos.) Calcd. For C₃₁H₃₄N₆O₄: 555.26 (M+H), Found 555.4.

### Example 23

### 8-indan-5-yl-5-oxo-2-[4-(piperidin-1-ylcarbamoylmethyl)-phenylamino]-5,8-dihydro-pyrido[2,3-d]pyrimidine-6-carboxylic acid methoxy-amide (Cpd 102)

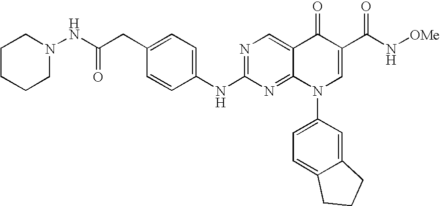

### A. [4-(8-indan-5-yl-6-methoxycarbamoyl-5-oxo-5,8-dihydro-pyrido[2,3-d]pyrimidin-2-ylamino)-phenyl]-acetic acid

Using procedures described in Example 8, Step B, (4-amino-phenyl)-acetic acid (25 mg, 0.16 mmol) and 8-indan-5-yl-2-methanesulfonyl-5-oxo-5,8-dihydro-pyrido[2,3-d]pyrimidine-6-carboxylic acid methoxy-amide (46 mg, 0.11 mmol) were reacted to provide the title compound as a yellow solid (50 mg, 94%).

### B. 8-indan-5-yl-5-oxo-2-[4-(piperidin-1-ylcarbamoylmethyl)-phenylamino]-5,8-dihydro-pyrido[2,3-d]pyrimidine-6-carboxylic acid methoxy-amide

To a solution of [4-(8-indan-5-yl-6-methoxycarbamoyl-5-oxo-5,8-dihydro-pyrido[2,3-d]pyrimidin-2-ylamino)-phenyl]-acetic acid (10 mg, 0.021 mmol) in DMF (0.5 mL) was added 1,1′-carbonyldiimidazole (4 mg, 0.031 mmol). The reaction mixture was stirred at rt for 1 h before 1-aminopiperidine (3.3 μL, 0.031 mmol) was added. After 1 h, water was added to quench the reaction. The mixture was extracted with CH₂Cl₂. The organic fractions were dried (Na₂SO₄) and filtered. The filtrate was concentrated, and the residue was purified (HPLC, 32 mL/min, 5-80% MeCN/H₂O (0.1% TFA v/v) gradient over 12 min) to give the title compound as a yellow solid (HCl salt, 4 mg, 32%). ¹H NMR (400 MHz, CDCl₃/CD₃OD 10:1 v/v) δ (ppm): 9.38 (s, 1H), 8.80 (s, 1H), 7.40 (d, 1H), 7.30 (br, 2H), 7.24 (s, 1H), 7.18 (d, J=7.9 Hz, 1H), 7.00 (br, 2H), 3.85 (s, 3H), 3.65 (s, 1H), 3.48 (s, 1H), 3.10 (m, 4H), 3.00 (t, J=7.4 Hz, 2H), 2.80 (m, 2H), 2.22 (m, 2H), 1.42-1.80 (m, 6H). Mass Spectrum (LCMS, ESI pos.) Calcd. For C₃₁H₃₃N₇O₄: 568.26 (M+H), Found 568.4.

Using the procedure of Example 23 and reagents, starting materials and conditions known to those skilled in the art, the following compounds representative of the present invention were prepared:

| Cpd | Name and Data |
|---|---|
| 103 | {4-[8-(4-ethyl-phenyl)-6-methoxycarbamoyl-5-oxo-5,8-dihydro-pyrido[2,3-d]pyrimidin- |
| | 2-ylamino]-phenyl}-acetic acid |
| | ¹H NMR (400 MHz, CDCl₃/CD₃OD 10:1 v/v) δ (ppm): 9.30 (s, 1H), 8.75 (s, 1H), |
| | 7.38 (d, 2H), 7.28 (d, 2H), 7.22 (br, 2H), 6.94 (br, 2H), 3.82 (s, 3H), 3.45 (s, 2H), 2.78 (q, |
| | 2H), 1.35 (t, 3H). Mass Spectrum (LCMS, ESI pos.) Calcd. For C₂₅H₂₃N₅O₅: 474.17 (M + H), |
| | Found 474.3. |
| 104 | 8-(4-ethyl-phenyl)-5-oxo-2-[4-(piperidin-1-ylcarbamoylmethyl)-phenylamino]-5,8- |
| | dihydro-pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.75 (s, 1H), |
| | 7.40 (d, 2H), 7.25 (d, 2H), 7.21 (br, 2H), 6.95 (br, 2H), 3.83 (s, 3H), 3.60 (s, 2H), 2.80 (m, |
| | 6H), 1.60 (m, 6H), 1.33 (t, 3H). Mass Spectrum (LCMS, ESI pos.) Calcd. For |
| | C₃₀H₃₃N₇O₄: 556.26 (M + H), Found 556.4. |
| 105 | 2-[4-(N′,N′-dimethyl-hydrazinocarbonylmethyl)-phenylamino]-8-(4-ethyl-phenyl)-5- |
| | oxo-5,8-dihydro-pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.75 (s, 1H), |
| | 7.40 (d, 2H), 7.25 (d, 2H), 7.21 (br, 2H), 6.95 (br, 2H), 3.83 (s, 3H), 3.68 (s, 1H), 3.40 (s, |
| | 1H), 2.80 (m, 2H), 2.40 (s, 6H), 1.35 (t, 3H). Mass Spectrum (LCMS, ESI pos.) Calcd. |
| | For C₂₇H₂₉N₇O₄: 516.23 (M + H), Found 516.4. |
| 106 | 8-(4-ethyl-phenyl)-2-[4-(N′-methyl-hydrazinocarbonylmethyl)-phenylamino]-5-oxo-5,8- |
| | dihydro-pyrido[2,3-d]pyrimidine-6-carboxylic acid methoxy-amide |
| | ¹H NMR (400 MHz, CDCl₃/CD₃OD 10:1 v/v) δ (ppm): 9.30 (s, 1H), 8.75 (s, 1H), |
| | 7.40 (d, 2H), 7.25 (d, 2H), 7.21 (br, 2H), 6.95 (br, 2H), 3.83 (s, 3H), 3.80 (s, 1H), 3.60 (s, |
| | 1H), 3.11 (s, 3H), 2.80 (m, 2H), 1.35 (t, 3H). Mass Spectrum (LCMS, ESI pos.) Calcd. |
| | For C₂₆H₂₇N₇O₄: 502.21 (M + H), Found 502.3. |

### Example 24

### 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide (Cpd 144)

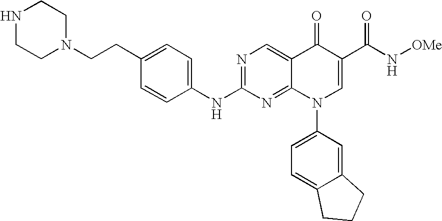

A solution of 4-[2-(4-nitro-phenyl)-ethyl]-piperazine-1-carboxylic acid tert-butyl ester (250 mg), ammonium formate (ca. 0.5 g) and Pd/C (10 mg) in MeOH (10 mL) was heated at 70° C. After 14 h, the reaction mixture was filtered and concentrated to provide the amine without further purification. A solution of 8-indan-5-yl-2-methanesulfonyl-5-oxo-5,8-dihydro-pyrido[2,3-d]pyrimidine-6-carboxylic acid methoxy-amide (20 mg, 0.048 mmol) and 4-[2-(4-amino-phenyl)-ethyl]-piperazine-1-carboxylic acid tert-butyl ester (37 mg, 0.12 mmol) in AcOH (1 mL) was heated at 110° C. After 10 min, the solution was concentrated. TFA (1 mL) and CH₂Cl₂ (1 mL) were added and progress of the reaction was monitored by LCMS. After 1 h, the reaction mixture was concentrated and the residue was purified (HPLC, C-18 YMC ODS-A 5μ 30×100 mm, 120 A column at 32 mL/min, 5-100% H₂O/MeCN (0.1% TFA v/v) gradient over 10 min.) to give the title compound (15.4 mg). ¹H NMR (TFA salt) (400 MHz, CDCl₃) δ (ppm): 9.27 (s, 1H), 8.72 (s, 1H), 7.37 (d, J=7.93 Hz, 2H), 7.15-7.07 (m, 2H), 7.19 (s, 1H), 7.31-7.24 (m, 2H), 3.81 (s, 3H), 3.50 (s, 4H), 3.34-3.23 (m, 3H), 3.17-3.08 (m, 3H), 3.06-2.99 (m, 2H), 2.93 (t, J=7.39 Hz, 4H), 2.16 (m, 2H); Mass Spectrum (LCMS, APCI pos.) Calcd. For: C₃₀H₃₃N₇O₃: 539.26; Found: 540.2 (M+H).

Using the procedure of Example 24 and reagents, starting materials and conditions known to those skilled in the art, the following compounds representative of the present invention were prepared:

| Cpd | Name and Data |
|---|---|
| 145 | 2-{4-[2-(4-acetyl-piperazin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (TFA salt) (400 MHz, CDCl3) δ (ppm): 9.31 (s, 1H), 8.77 (s, 1H), 7.41 (d, |
| | J = 7.96 Hz, 2H), 7.27 (dd, J = 1.23, 0.74 Hz, 2H), 7.20 (s, 1H), 7.17-7.10 (m, 2H), |
| | 3.84 (s, 3H), 3.57 (m 2H), 3.42-3.32 (m, 2H), 3.13 (s, 4H), 3.04 (br s, 9H), 2.97-2.82 (m, 2H), |
| | 2.21 (br s, 2H); Mass Spectrum (LCMS, APCI pos.) Calcd. For: C₃₂H₃₅N₇O₄: 581.28; |
| | Found: 582.3 (M + H). |

### Example 25

### 8-indan-5-yl-2-{4-[2-(4-methanesulfonyl-piperazin-1-yl)-ethyl]-phenylamino}-5-oxo-5,8-dihydro-pyrido[2,3-d]pyrimidine-6-carboxylic acid methoxy-amide (Cpd 146)

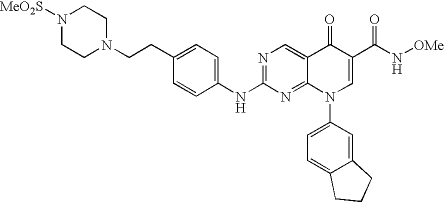

To a solution of 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide Cpd 144 (Example 24), 13.6 mg, 0.021 mmol) in CH₂Cl₂ (1 mL), was added methane sulfonylchloride (3.6 mg, 0.025 mmol) and triethylamine (5 mg, 0.050 mmol). After 10 min, the reaction mixture was concentrated and the residue was purified (HPLC, C-18 YMC ODS-A 5 30×100 mm, 120 A column at 32 mL/min, 5-100% H₂O/MeCN (0.1% TFA v/v) gradient over 10 min.) to afford the title compound (5.4 mg). ¹H NMR (TFA salt) (400 MHz, CDCl₃) δ (ppm): 9.29 (s, 1H), 8.76 (s, 1H), 7.37 (d, J=7.96 Hz, 2H), 7.33-7.23 (m, 3H), 7.10 (dd, J=7.86, 1.78 Hz, 2H), 3.83 (s, 3H), 3.67-3.53 (m, 4H), 3.50-3.27 (m, 2H), 3.18-3.08 (m, 2H), 3.08-2.90 (m, 8H), 2.84 (s, 3H), 2.17 (br s, 2H); Mass Spectrum (LCMS, APCI pos.) Calcd. For: C₃₁H₃₅N₇O₅S: 617.24; Found: 618.2 (M+H).

### Example 26

### (4-{2-[4-(8-indan-5-yl-6-methoxycarbamoyl-5-oxo-5,8-dihydro-pyrido[2,3-d]pyrimidin-2-ylamino)-phenyl]-ethyl}-piperazin-1-yl)-acetic acid (Cpd 147)

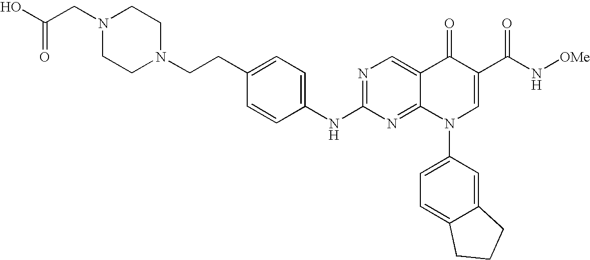

A solution of 2-bromoacetic acid (5.3 mg, 0.038 mmol), triethylamine (7.6 mg, 0.076 mmol) and 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide Cpd 144 (Example 24) (25 mg, 0.076 mmol) in CH₂Cl₂ (1 mL) was stirred at rt. After 14 h, the reaction mixture was concentrated and the residue was purified (HPLC, C-18 YMC ODS-A 5 30×100 mm, 120 A column at 32 mL/min, 5-100% H₂O/MeCN (0.1% TFA v/v) gradient over 10 min.) to afford the title compound (16.8 mg). ¹H NMR (TFA salt) (400 MHz, CDCl₃) δ (ppm): 9.37 (s, 1H), 8.82 (s, 1H), 7.35 (br s, 2H), 7.29-7.22 (m, 3H), 7.15-7.03 (m, 2H), 3.82 (s, 3H), 3.40 (s, 3H), 3.11 (br s, 6H), 3.01 (br s, 2H), 2.98-2.88 (m, 4H), 2.68-2.28 (m, 6H), 2.15 (br s, 2H); Mass Spectrum (LCMS, APCI pos.) Calcd. For: C₃₂H₃₅N₇O₅: 597.27; Found: 598.3 (M+H).

### Example 27

### 8-indan-5-yl-5-oxo-2-(4-{2-[4-(2,2,2-trifluoro-acetyl)-piperazin-1-yl]-ethyl}-phenylamino)-5,8-dihydro-pyrido[2,3-d]pyrimidine-6-carboxylic acid methoxy-amide (Cpd 148)

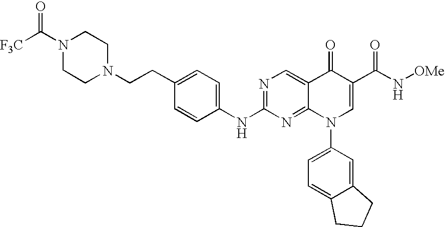

A solution of trifluoroacetic anhydride (8 mg, 0.038 mmol), triethylamine (7.6 mg, 0.076 mmol) and 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide Cpd 144 (Example 24) (25 mg, 0.076 mmol) in CH₂Cl₂ (1 mL) was stirred at rt for 10 min. The reaction mixture was concentrated and the residue was purified (HPLC, C-18 YMC ODS-A 5 30×100 mm, 120 A column at 32 mL/min, 5-100% H₂O/MeCN (0.1% TFA v/v) gradient over 10 min.) to afford the title compound (6.8 mg). ¹H NMR (TFA salt) (400 MHz, CDCl3) δ (ppm): 9.28 (s, 1H), 8.76 (s, 1H), 7.37 (d, J=7.86 Hz, 2H), 7.33-7.22 (m, 3H), 7.10 (dd, J=7.83, 1.80 Hz, 2H), 4.07-3.90 (m, 4H), 3.21-3.08 (m, 3H), 3.07-2.78 (m, 9H), 2.17 (br s, 2H); Mass Spectrum (LCMS, APCI pos.) Calcd. For: C₃₂H₃₂F₃N₇O₄: 635.25; Found: 636.3 (M+H).

### Example 28

### 8-indan-5-yl-5-oxo-2-(4-{2-[4-(2,2,2-trifluoro-ethyl)-piperazin-1-yl]-ethyl}-phenylamino)-5,8-dihydro-pyrido[2,3-d]pyrimidine-6-carboxylic acid methoxy-amide (Cpd 149)

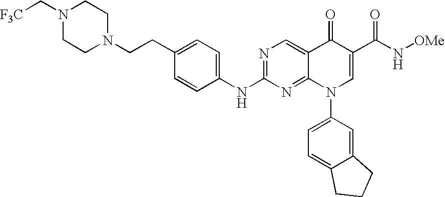

A solution of trifluoro-methanesulfonic acid 2,2,2-trifluoro-ethyl ester (9 mg, 0.038 mmol), triethylamine (7.6 mg, 0.076 mmol) and 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide Cpd 144 (from Example 24) (25 mg, 0.076 mmol) in CH₂Cl₂ (1 mL) was stirred for 10 min at rt. The reaction mixture was concentrated and the residue was purified (HPLC, C-18 YMC ODS-A 5μ30×100 mm, 120 A column at 32 mL/min, 5-100% H₂O/MeCN (0.1% TFA v/v) gradient over 10 min.) to afford the title compound (15.0 mg). ¹H NMR (TFA salt) (400 MHz, CDCl3) δ (ppm): 9.27 (s, 1H), 8.75 (s, 1H), 7.36 (d, J=7.76 Hz, 2H), 7.32-7.18 (m, 3H), 7.12-7.04 (m, 2H), 3.82 (s, 3H), 3.56 (d, J=11.02 Hz, 2H), 3.19-2.73 (m, 14H), 2.15 (m, 2H); Mass Spectrum (LCMS, APCI pos.) Calcd. For: C₃₂H₃₄F₃N₇O₃: 621.27; Found: 622.3 (M+H).

### Example 29

### 4-{2-[4-(8-indan-5-yl-6-methoxycarbamoyl-5-oxo-5,8-dihydro-pyrido[2,3-d]pyrimidin-2-ylamino)-phenyl]-ethyl}-piperazine-1-carboxylic acid methyl ester (Cpd 150)

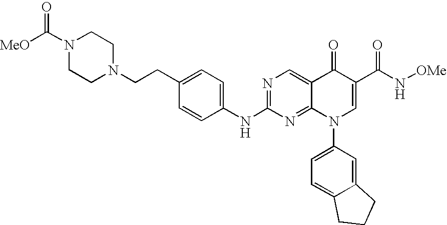

A solution of methylchloroformate (4 mg, 0.038 mmol), triethylamine (7.6 mg, 0.076 mmol) and 8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide Cpd 144 (Example 24) (25 mg, 0.076 mmol) in CH₂Cl₂ (1 mL) was stirred for 10 min at rt. The reaction mixture was concentrated and the residue was purified (HPLC, C-18 YMC ODS-A 5 30×100 mm, 120 A column at 32 mL/min, 5-100% H₂O/MeCN (0.1% TFA v/v) gradient over 10 min.) to afford the title compound (22.6 mg). ¹H NMR (TFA salt) (400 MHz, CDCl₃) δ (ppm): 9.34 (s, 1H), 8.83 (s, 1H), 7.44 (d, J=7.95 Hz, 2H), 7.39-7.28 (m, 3H), 7.21-7.13 (m, 2H), 3.89 (s, 3H), 3.74 (s, 3H), 3.70-3.61 (m, 2H), 3.59-3.29 (m, 2H), 3.24-2.91 (m, 10H), 2.85-2.57 (m, 2H), 2.23 (m, 2H); Mass Spectrum (LCMS, APCI pos.) Calcd. For: C₃₂H₃₅N₇O₅: 597.27; Found: 598.3 (M+H).

### Example 30

### 8-indan-5-yl-2-{4-[3-(4-methylpiperazin-1-yl)-propyl]-phenylamino}-5-oxo-5,8-dihydro-pyrido[2,3-d]pyrimidine-6-carboxylic acid methoxy-amide (Cpd 151)

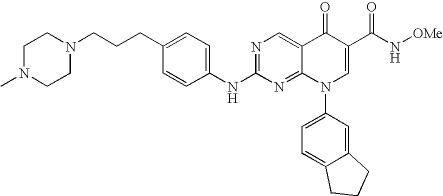

### Preparation of 4-[3-(4-methyl-piperazin-1-yl)-propyl]-phenylamine

To a solution of 1-methyl-piperazine (1.50 g, 15.0 mmol) in 10 mL of DMSO at 5-10° C. was added propargyl bromide (2.3 mL, 80% w/w in toluene, 15 mmol) followed by 2.0 g of K₂CO₃. The mixture was stirred at rt for 12 h and at 80° C. for 1 h. The reaction was quenched with brine and extracted with CH₂Cl₂ (3×). The combined organic extracts were washed with water and brine, dried over Na₂SO₄ and filtered. Concentration of the filtrate gave 1.7 g (82% yield) of 1-methyl-4-prop-2-ynyl-piperazine.

A mixture of 4-bromonitrobenzene (185 mg, 0.9 mmol), 1-methyl-4-prop-2-ynyl-piperazine (140 mg, 1.0 mmol), CuI (10 mg), PdCl₂(PPh₃)₂ (20 mg) and 5 mL of Et₃N in CH₂Cl₂ was heated at reflux for 12 h. The reaction mixture was concentrated in vacuo, and the residue was purified by chromatography (silica, 5% methanol in CH₂Cl₂) to give 160 mg (69% yield) of 1-methyl-4-[3-(4-nitrophenyl)-prop-2-ynyl]-piperazine.

A mixture of 1-methyl-4-[3-(4-nitrophenyl)-prop-2-ynyl]-piperazine (160 mg, 0.62 mmol) and 91 mg of Pd/C (10%) in 20 mL of methanol was shaken under H₂ (40 psi) for 2 h. The reaction mixture was filtered through a pad of Celite, and the filtrate was concentrated to give 110 mg (76% yield) of 4-[3-(4-methyl-piperazin-1-yl)-propyl]-phenylamine.

The title compound was prepared by reacting 4-[3-(4-methyl-piperazin-1-yl)-propyl]-phenylamine with 8-indan-5-yl-2-methanesulfonyl-5-oxo-5,8-dihydro-pyrido[2,3-d]pyrimidine-6-carboxylic acid methoxy-amide. ¹H NMR (400 MHz, CDCl₃) δ (ppm): ¹H NMR (400 MHz, CDCl₃) δ (ppm): 11.90 (s, 1H), 9.31 (s, 1H), 8.76 (s, 1H), 7.36 (d, 1H), 7.20 (s, 1H), 7.10 (d, 1H), 6.85 (br, 4H), 3.83 (s, 3H), 3.05-2.92 (m, 4H), 2.55-2.10 (m, 17H), 1.20 (m, 2H). Mass Spectrum (LCMS, APCI pos.) Calcd. For C₃₂H₃₇N₇O₃: 568.3 (M+H), Found: 568.4.

### Example 31

### 8-indan-5-yl-2-{4-[2-(4-methylpiperazin-1-yl)-2-oxo-ethyl]-phenylamino}-5-oxo-5,8-dihydro-pyrido[2,3-d]pyrimidine-6-carboxylic acid methoxy-amide (Cpd 152)

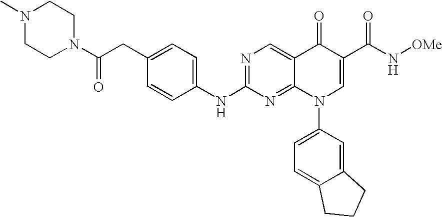

### Preparation of 2-(4-aminophenyl)-1-(4-methylpiperazin-1-yl)-ethanone

To a solution of (4-nitrophenyl)-acetic acid (555 mg, 3.0 mmol) and 10 μL of DMF in 10 mL of CH₂Cl₂ at 0° C., was slowly added oxalyl chloride (0.52 mL, 6 mmol), and the reaction was stirred at rt for 3 h. After removal of CH₂Cl₂ and excess oxalyl chloride under reduced pressure, the residue was dissolved in 10 mL of CH₂Cl₂, and to the solution at 0° C. was added a solution of 1-methylpiperazine (390 mg, 3.9 mmol) in 3 mL of CH₂Cl₂ followed by 0.5 mL of Et₃N. The mixture was stirred at rt for 2 h and quenched with water. The reaction mixture was extracted with CH₂Cl₂, and the organic fractions were washed with brine, dried over Na₂SO₄ and filtered. The filtrate was concentrated and the residue and 20 mg of Pd/C (10%) in 10 mL of MeOH was stirred under 1 atm of H₂ for 12 h. The reaction mixture was filtered through a pad of Celite, and the filtrate was concentrated to give 710 mg (100% yield in 2 steps) of 2-(4-aminophenyl)-1-(4-methylpiperazin-1-yl)-ethanone.

The title compound was prepared by reacting 2-(4-aminophenyl)-1-(4-methylpiperazin-1-yl)-ethanone with 8-indan-5-yl-2-methanesulfonyl-5-oxo-5,8-dihydro-pyrido[2,3-d]pyrimidine-6-carboxylic acid methoxy-amide. ¹H NMR (400 MHz, CDCl₃) δ (ppm): 11.90 (s, 1H), 9.30 (s, 1H), 8.76 (s, 1H), 7.36 (d, 1H), 7.24 (s, 1H), 7.10 (d, 2H), 6.88 (br, 4H), 3.83 (s, 3H), 3.58 (br, 4H), 3.37 (m, 2H), 3.05-2.92 (m, 4H), 2.19 (s, 3H), 2.16 (m, 6H). Mass Spectrum (LCMS, APCI pos.) Calcd. For C₃₁H₃₃N₇O₄: 568.26 (M+H), Found: 568.3.

### Example 32

### 8-indan-5-yl-2-[4-(3-methanesulfonylamino-3-oxo-propyl)-phenylamino]-5-oxo-5,8-dihydro-pyrido[2,3-d]pyrimidine-6-carboxylic acid methoxy-amide (Cpd 153)

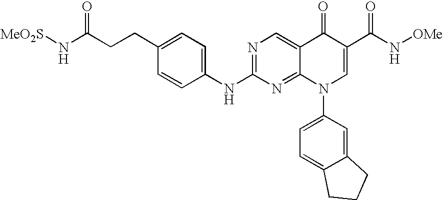

### Preparation of N-[3-(4-aminophenyl)-propionyl]-methanesulfonamide

To a solution of 3-(4-nitrophenyl)-propionic acid (205 mg, 1.0 mmol) in 3 mL of DMF was added carbonyl diimidazole (190 mg, 1.13 mmol). The mixture was stirred at rt for 1 h followed by addition of methanesulfonamide (120 mg, 1.2 mmol) and 0.17 mL of DBU. The reaction mixture was stirred overnight at rt, then quenched with water. The reaction mixture was extracted with CH₂Cl₂, and the organic fractions were washed with brine, dried over Na₂SO₄ and filtered. The filtrate was concentrated. The residue and 10 mg of Pd/C (10%) in 5 mL of MeOH was stirred under 1 atm of H₂ for 1 h. The reaction mixture was filtered through a pad of Celite and the filtrate was concentrated to give 90 mg (35% yield in 2 steps) of N-[3-(4-aminophenyl)-propionyl]-methanesulfonamide.

The title compound was prepared by reacting N-[3-(4-aminophenyl)-propionyl]-methanesulfonamide with 8-indan-5-yl-2-methanesulfonyl-5-oxo-5,8-dihydro-pyrido[2,3-d]pyrimidine-6-carboxylic acid methoxy-amide. ¹H NMR (400 MHz, CD₃OD) δ (ppm): 9.30 (s, 1H), 8.76 (s, 1H), 7.36 (d, 1H), 7.20 (br, 2H), 7.10 (d, 2H), 6.95 (d, 1H), 6.84 (br, 1H), 6.56 (br, 1H), 3.83 (s, 3H), 3.13 (s, 3H), 2.92-2.80 (m, 6H), 2.47 (m, 2H), 2.19 (m, 2H). Mass Spectrum (LCMS, APCI pos.) Calcd. For C₂₈H₂₈N₆O₆S: 577.18 (M+H), Found: 577.3.

### Example 33

### 8-(4-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide (Cpd 156)

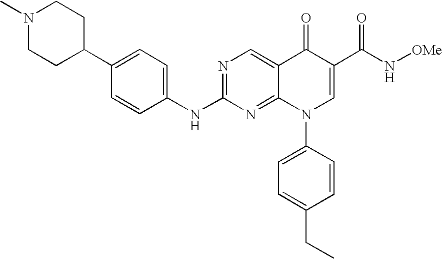

### A. 8-(4-ethyl-phenyl)-2-methylsulfanyl-5-oxo-5,8-dihydro-pyrido[2,3-d]pyrimidine-6-carboxylic acid ethyl ester

A solution of 3-(4-methoxy-2-methylsulfanyl-pyrimidin-5-yl)-3-oxo-propionic acid ethyl ester (Example 2, Step B) (10.8 g, 40 mmol,), 10 mL of acetic anhydride (109.5 mmol) and 10 mL of triethylorthoformate (66 mmol) was heated at 130° C. for 2 h. The reaction was cooled, concentrated, and the residue was dissolved in 60 mL of THF. To the solution was added 5.4 mL (42 mmol) of 4-ethynyl-phenylamine. After the mixture was stirred at rt for 2 h, 5.5 g of K₂CO₃ (40 mmol) was added. The mixture was stirred at rt for 14 h. Water was added, and the reaction mixture was extracted with CH₂Cl₂ (2×300 mL). The organic fractions were concentrated to give 14.9 g of the title compound as a white solid. ¹H NMR (400 MHz, CDCl₃) δ (ppm): 9.40 (s, 1H), 8.55 (s, 1H), 7.68 (d, 2H), 7.42 (d, 2H), 4.40 (q, 2H), 3.22 (s, 1H), 2.30 (s, 3H), 1.40 (t, 3H).

### B. 8-(4-ethyl-phenyl)-2-methylsulfanyl-5-oxo-5,8-dihydro-pyrido[2,3-d]pyrimidine-6-carboxylic acid

A solution of 8-(4-ethyl-phenyl)-2-methylsulfanyl-5-oxo-5,8-dihydro-pyrido[2,3-d]pyrimidine-6-carboxylic acid ethyl ester (14.9 g, 40.4 mmol) in 1,4-dioxane (100 mL) and 1 N HCl (100 mL) was heated at reflux. After 4 h, the reaction mixture was cooled to rt. The resulting precipitate was filtered and the solid was washed with water and dried under vacuum to afford 11.7 g of the title compound as an off-white solid. ¹H NMR (400 MHz, CDCl₃) δ (ppm): 9.79 (s, 1H), 8.70 (s, 1H), 7.75 (d, 2H), 7.45 (d, 2H), 4.40 (q, 2H), 3.22 (s, 1H), 3.20 (s, 3H), 1.40 (t, 3H).

### C. 8-(4-ethyl-phenyl)-2-methylsulfanyl-5-oxo-5,8-dihydro-pyrido[2,3-d]pyrimidine-6-carboxylic acid methoxy-amide

To a solution of 8-(4-ethyl-phenyl)-2-methylsulfanyl-5-oxo-5,8-dihydro-pyrido[2,3-d]pyrimidine-6-carboxylic acid (7.46 g, 21.9 mmol) in 100 mL of CH₂Cl₂ at 0° C. was added 2.6 mL of oxalyl chloride (29.8 mmol) and 20 μL of DMF. The solution was stirred at rt for 3 h. The solvent and excess oxalyl chloride was removed in vacuo. To the residue in 300 mL of CH₂Cl₂ at 0° C. was added MeONH₂.HCl (2.49 g, 30 mmol) followed by slow addition of Et₃N (8.5 mL, 60 mmol). The reaction mixture was stirred at 0° C. for 5 min and at rt for 20 min. Water was added and the reaction mixture was extracted with CH₂Cl₂ (400 mL×2). The combined organic fractions were washed with brine and concentrated in vacuo to give the title compound as an off-white solid (7.69 g). ¹H NMR (400 MHz, CDCl₃) δ (ppm): 9.45 (s, 1H), 8.88 (s, 1H), 7.35 (d, 2H), 7.31 (d, 2H), 3.90 (s, 3H), 2.75 (q, 2H), 2.36 (s, 3H), 1.27 (t, 3H).

### D. 8-(4-ethyl-phenyl)-2-methanesulfonyl-5-oxo-5,8-dihydro-pyrido[2,3-d]pyrimidine-6-carboxylic acid methoxy-amide

To a solution of 8-(4-ethyl-phenyl)-2-methylsulfanyl-5-oxo-5,8-dihydro-pyrido[2,3-d]pyrimidine-6-carboxylic acid methoxy-amide (7.69 g, 20.8 mmol) in 400 mL of CH₂Cl₂ at 0° C. was slowly added mCPBA (13.6 g, 55 mmol of a 70% w/w mixture). After stirring for 14 h, a solution of 10% NaS₂O₄ was added and the reaction mixture was extracted with CH₂Cl₂. The combined organic extracts were washed with sat. NaHCO₃, followed by water and then concentrated to afford 7.2 g of the title compound. ¹H NMR (400 MHz, CDCl₃) δ (ppm): 9.82 (s, 1H), 9.05 (s, 1H), 7.43 (d, 2H), 7.36 (d, 2H), 3.90 (s, 3H), 3.21 (s, 3H), 2.79 (q, 2H), 1.31 (t, 3H).

### E. 8-(4-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide

A mixture of 8-(4-ethyl-phenyl)-2-methanesulfonyl-5-oxo-5,8-dihydro-pyrido[2,3-d]pyrimidine-6-carboxylic acid methoxy-amide (1.5 g, 3.73 mmol) and 4-(1-methyl-piperidin-4-yl)-phenylamine (1.42 g, 7.46 mmol) in AcOH (10 mL) was heated at 110° C. for 15 min. The reaction mixture was concentrated and the remaining residue was partitioned between sat. NaHCO₃ and CH₂Cl₂. The organic layer was dried (MgSO₄), filtered, and the filtrate concentrated. MeOH (20 mL) was added and a fine precipitate formed that was collected by filtration to provide 800 mg of the title compound as an off-white solid. ¹H NMR (400 MHz, CDCl₃) δ (ppm): 9.31 (s, 1H), 8.76 (s, 1H), 7.38 (d, 2H), 7.25 (d, 2H), 3.85 (s, 3H), 2.77 (q, 2H), 2.41-2.58 (m, 5H), 1.40-1.62 (m, 4H), 1.35 (t, 3H). Mass Spectrum (LCMS, APCI pos.) Calcd. For: C₂₉H₃₂N₆O₃: 512.25; Found: 513.3 (M+H).

Using the procedure of Example 33 and reagents, starting materials and conditions known to those skilled in the art, the following compounds representative of the present invention were prepared:

| Cpd | Name and Data |
|---|---|
| 159 | 2-[4-(1-methylpiperidin-4-yl)-phenylamino]-5-oxo-8-(4-trifluoromethoxyphenyl)-5,8- |
| | dihydropyrido[2,3-d]pyrimidine-6-carboxylic acid methoxy-amide |
| | ¹H NMR (400 MHz, CDCl₃) δ (ppm): 11.92 (s, 1H), 9.38 (s, 1H), 8.78 (s, 1H), 7.68 (br, |
| | 1H), 7.47 (m, 4H), 7.20 (br, 2H), 6.98 (br, 2H), 3.88 (s, 3H), 3.00 (m, 2H), 2.42 (m, 1H), |
| | 2.33 (s, 3H), 2.06 (m, 2H), 1.77 (m, 4H). Mass Spectrum (LCMS, ESI pos.) Calcd. For |
| | C₂₈H₂₇F₃N₆O₄: 569.2 (M + H), Found: 569.5. |
| 160 | 2-[4-(1-ethyl-piperidin-4-yl)-phenylamino]-5-oxo-8-(4-trifluoromethoxy-phenyl)-5,8- |
| | dihydro-pyrido[2,3-d]pyrimidine-6-carboxylic acid methoxy-amide |
| | Cpd 160 may be prepared using the procedure of Example 33. |
| 161 | 2-[4-(1-isopropyl-piperidin-4-yl)-phenylamino]-5-oxo-8-(4-trifluoromethoxy-phenyl)-5,8- |
| | dihydro-pyrido[2,3-d]pyrimidine-6-carboxylic acid methoxy-amide |
| | Cpd 161 may be prepared using the procedure of Example 33. |
| 163 | 2-[4-(1-methyl-piperidin-4-yl)-phenylamino]-5-oxo-8-(4-trifluoromethyl-phenyl)-5,8- |
| | dihydro-pyrido[2,3-d]pyrimidine-6-carboxylic acid methoxy-amide |
| | Cpd 163 may be prepared using the procedure of Example 33. |
| 164 | 2-[4-(1-ethyl-piperidin-4-yl)-phenylamino]-5-oxo-8-(4-trifluoromethyl-phenyl)-5,8- |
| | dihydro-pyrido[2,3-d]pyrimidine-6-carboxylic acid methoxy-amide |
| | Cpd 164 may be prepared using the procedure of Example 33. |
| 165 | 2-[4-(1-isopropyl-piperidin-4-yl)-phenylamino]-5-oxo-8-(4-trifluoromethyl-phenyl)-5,8- |
| | dihydro-pyrido[2,3-d]pyrimidine-6-carboxylic acid methoxy-amide |
| | Cpd 165 may be prepared using the procedure of Example 33. |
| 166 | 2-{4-[1-(2-hydroxy-ethyl)-piperidin-4-yl]-phenylamino}-5-oxo-8-(4-trifluoromethyl- |
| | phenyl)-5,8-dihydro-pyrido[2,3-d]pyrimidine-6-carboxylic acid methoxy-amide |
| | Cpd 166 may be prepared using the procedure of Example 33. |
| 167 | 2-[4-(1-methyl-piperidin-4-yl)-phenylamino]-5-oxo-8-(4-propyl-phenyl)-5,8-dihydro- |
| | pyrido[2,3-d]pyrimidine-6-carboxylic acid methoxy-amide |
| | Cpd 167 may be prepared using the procedure of Example 33. |
| 168 | 2-[4-(1-ethyl-piperidin-4-yl)-phenylamino]-5-oxo-8-(4-propyl-phenyl)-5,8-dihydro- |
| | pyrido[2,3-d]pyrimidine-6-carboxylic acid methoxy-amide |
| | Cpd 168 may be prepared using the procedure of Example 33. |
| 169 | 2-[4-(1-isopropyl-piperidin-4-yl)-phenylamino]-5-oxo-8-(4-propyl-phenyl)-5,8-dihydro- |
| | pyrido[2,3-d]pyrimidine-6-carboxylic acid methoxy-amide |
| | Cpd 169 may be prepared using the procedure of Example 33. |
| 170 | 2-{4-[1-(2-hydroxy-ethyl)-piperidin-4-yl]-phenylamino}-5-oxo-8-(4-propyl-phenyl)-5,8- |
| | dihydro-pyrido[2,3-d]pyrimidine-6-carboxylic acid methoxy-amide |
| | Cpd 170 may be prepared using the procedure of Example 33. |
| 175 | 2-{4-[2-(4-methylpiperazin-1-yl)-ethyl]-phenylamino}-5-oxo-8-(4-trifluoromethoxy- |
| | phenyl)-5,8-dihydropyrido[2,3-d]pyrimidine-6-carboxylic acid methoxy-amide |
| | ¹H NMR (TFA salt) (400 MHz, CDCl₃) δ (ppm): 9.32 (s, 1H), 8.73 (s, 1H), 7.47 (m, 4H), |
| | 7.22 (br, 2H), 6.90 (br, 2H), 3.83 (s, 3H), 3.46 (m, 10H), 3.10 (m, 2H), 2.85 (s, 3H). Mass |
| | Spectrum (LCMS, ESI pos.) Calcd. For C₂₉H₃₀F₃N₇O₄: 598.2 (M + H), Found: 598.6. |
| 207 | 8-(4-ethyl-phenyl)-2-{4-[1-(2-methoxy-ethyl)-piperidin-4-yl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 11.98 (s, 1H), 9.39 (s, 1H), 8.80 (s, 1H), 7.62 (br, |
| | 1H), 7.41 (d, 2H), 7.35 (d, 2H), 7.23 (br, 2H), 6.96 (br, 2H), 3.90 (s, 3H), 3.55 (t, 2H), |
| | 3.39 (s, 3H), 3.10 (d, 2H), 2.82 (q, 2H), 2.62 (t, 2H), 2.42 (br, 1H), 2.10 (m, 2H), 1.70 (m, |
| | 4H), 1.40 (t, 3H). Mass Spectrum (LCMS, ESI pos.) Calcd. For C₃₁H₃₆N₆O₄: 557.67 (M + H), |
| | Found: 557.6. |
| 208 | 8-(4,4-dimethyl-cyclohexyl)-2-[4-(1-ethyl-piperidin-4-yl)-phenylamino]-5-oxo-5,8- |
| | dihydro-pyrido[2,3-d]pyrimidine-6-carboxylic acid methoxy-amide |
| | ¹H NMR (400 MHz, CDCl₃) δ (ppm): 12.00 (s, 1H), 9.38 (s, 1H), 8.80 (s, 1H), 7.95 (br, |
| | 1H), 7.57 (d, 2H), 7.25 (d, 2H), 5.05 (m, 1H), 3.88 (s, 3H), 3.10 (d, 2H), 2.52 (m, 1H), |
| | 2.45 (q, 2H), 2.00 (dt, 2H), 1.90 (m, 8H), 1.65 (d, 2H), 1.43 (m, 2H), 1.13 (t, 3H), 1.04 (s, |
| | 3H), 1.02 (s, 3H). Mass Spectrum (LCMS, ESI pos.) Calcd. For C₃₀H₄₀N₆O₃: 533.69 (M + H), |
| | Found: 533.8. |
| 209 | 8-(4,4-dimethyl-cyclohexyl)-2-[4-(1-methyl-piperidin-4-yl)-phenylamino]-5-oxo-5,8- |
| | dihydro-pyrido[2,3-d]pyrimidine-6-carboxylic acid methoxy-amide |
| | ¹H NMR (400 MHz, CDCl₃) δ (ppm): 12.00 (s, 1H), 9.40 (s, 1H), 8.80 (s, 1H), 7.65 (br, |
| | 1H), 7.57 (d, 2H), 7.23 (d, 2H), 5.10 (m, 1H), 3.88 (s, 3H), 3.05 (d, 2H), 2.52 (m, 1H), |
| | 2.36 (s, 3H), 2.10 (m, 2H), 1.90 (m, 8H), 1.65 (d, 2H), 1.50 (m, 2H), 1.04 (s, 3H), 1.02 (s, 3H). |
| | Mass Spectrum (LCMS, ESI pos.) Calcd. For C₂₉H₃₈N₆O₃: 519.66 (M + H), Found: 519.6. |
| 210 | 8-(4,4-dimethyl-cyclohexyl)-2-{4-[1-(2-hydroxy-ethyl)-piperidin-4-yl]-phenylamino}-5- |
| | oxo-5,8-dihydro-pyrido[2,3-d]pyrimidine-6-carboxylic acid methoxy-amide |
| | ¹H NMR (400 MHz, CDCl₃) δ (ppm): 12.00 (s, 1H), 9.38 (s, 1H), 8.80 (s, 1H), 7.65 (br, |
| | 1H), 7.57 (d, 2H), 7.23 (d, 2H), 5.05 (m, 1H), 3.89 (s, 3H), 3.64 (t, 2H), 3.05 (d, 2H), |
| | 2.58 (m, 3H), 2.20 (dt, 2H), 1.63-1.98 (m, 10H), 1.50 (m, 2H), 1.05 (s, 3H), 1.03 (s, 3H). Mass |
| | Spectrum (LCMS, ESI pos.) Calcd. For C₃₀H₄₀N₆O₄: 549.68 (M + H), Found: 549.8. |
| 211 | 8-(4-ethyl-phenyl)-2-{4-[1-(2-methoxy-ethyl)-piperidin-4-yl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 11.90 (s, 1H), 9.38 (s, 1H), 8.78 (s, 1H), 7.90 (d, |
| | 1H), 7.78 (m, 2H), 7.71 (br, 1H), 7.63 (d, 1H), 7.15 (br, 2H), 6.95 (br, 2H), 3.92 (s, 3H), |
| | 3.00 (d, 2H), 2.41 (br, 1H), 2.35 (s, 3H), 2.05 (m, 2H), 1.78 (br, 4H). Mass Spectrum |
| | (LCMS, ESI pos.) Calcd. For C₂₈H₂₇F₃N₆O₃: 553.56 (M + H), Found: 553.6. |
| 212 | 8-(3-ethyl-phenyl)-2-[4-(1-methyl-piperidin-4-yl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.40 (s, 1H), 8.80 (s, 1H), 7.70 (br, |
| | 1H), 7.50 (m, 2H), 7.23 (m, 4H), 6.95 (br, 2H), 3.93 (s, 3H), 3.10 (d, 2H), 2.77 (q, 2H), |
| | 2.43 (br, 4H), 2.20 (br, 2H), 1.85 (br, 4H), 1.30 (t, 3H). Mass Spectrum (LCMS, ESI pos.) |
| | Calcd. For C₂₉H₃₂N₆O₃: 513.62 (M + H), Found: 513.6. |
| 213 | 8-(4-ethyl-phenyl)-2-[4-(3-methoxy-propylamino)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.30 (s, 1H), 8.80 (s, 1H), 7.52 (s, |
| | 1H), 7.41 (d, 2H), 7.34 (d, 2H), 7.10 (d, 2H), 6.34 (d, 2H), 3.90 (s, 3H), 3.53 (t, 2H), |
| | 3.38 (s, 3H), 3.20 (br, 2H), 2.80 (q, 2H), 1.86 (m, 2H), 1.38 (t, 3H). Mass Spectrum (LCMS, |
| | ESI pos.) Calcd. For C₂₇H₃₀N₆O₄: 503.57 (M + H), Found: 503.4. |
| 214 | 8-indan-5-yl-2-(4-methanesulfonylaminocarbonyl-phenylamino)-5-oxo-5,8-dihydro- |
| | pyrido[2,3-d]pyrimidine-6-carboxylic acid methoxy-amide |
| | ¹H NMR (400 MHz, CDCl₃ + CD₃OD) δ (ppm): 9.41 (s, 1H), 8.82 (s, 1H), 7.65 (br, 2H), |
| | 7.50 (br, 2H), 7.45 (d, 1H), 7.26 (s, 1H), 7.19 (d, 1H), 3.91 (s, 3H), 3.40 (s, 3H), 3.12 (t, |
| | 2H), 3.01 (t, 2H), 2.23 (t, 2H). Mass Spectrum (LCMS, ESI pos.) Calcd. For |
| | C28H28F2N6O3:S: 549.58 (M + H), Found: 549.3. |
| 215 | 8-(4-ethyl-phenyl)-2-[3-(2-methoxy-ethoxy)-phenylamino]-5-oxo-5,8-dihydro-pyrido[2,3- |
| | d]pyrimidine-6-carboxylic acid methoxy-amide |
| | ¹H NMR (400 MHz, CDCl₃) δ (ppm): 11.96 (s, 1H), 9.40 (s, 1H), 8.80 (s, 1H), 7.58 (br, |
| | 1H), 7.42 (d, 2H), 7.35 (d, 2H), 7.05 (d, 1H), 6.99 (br, 1H), 6.82 (br, 1H), 6.61 (d, 1H), |
| | 4.00 (br, 2H), 3.90 (s, 3H), 3.45 (s, 3H), 2.82 (q, 2H), 1.37 (t, 3H). Mass Spectrum |
| | (LCMS, ESI pos.) Calcd. For C₂₆H₂₇N₅O₅: 490.53 (M + H), Found: 490.4. |
| 216 | 8-(4-ethyl-phenyl)-2-[3-(3-methoxy-propoxy)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.40 (s, 1H), 8.80 (s, 1H), 7.66 (br, |
| | 1H), 7.42 (d, 2H), 7.35 (d, 2H), 7.03 (d, 1H), 6.99 (br, 1H), 6.82 (br, 1H), 6.60 (d, 1H), |
| | 3.95 (br, 2H), 3.90 (s, 3H), 3.55 (t, 2H), 3.36 (s, 3H), 2.82 (q, 2H), 2.02 (m, 2H), 1.37 (t, |
| | 3H). Mass Spectrum (LCMS, ESI pos.) Calcd. For C₂₇H₂₉N₅O₅: 504.56 (M + H), Found: |
| | 504.5. |
| 217 | 8-(4-ethyl-phenyl)-2-[4-(2-methanesulfonyl-ethylamino)-phenylamino]-5-oxo-5,8- |
| | dihydro-pyrido[2,3-d]pyrimidine-6-carboxylic acid methoxy-amide |
| | ¹H NMR (400 MHz, CDCl₃) δ (ppm): 12.00 (s, 1H), 9.35 (s, 1H), 8.80 (s, 1H), 7.52 (s, |
| | 1H), 7.40 (d, 2H), 7.35 (d, 2H), 7.19 (d, 2H), 6.39 (d, 2H), 4.14 (br, 1H), 3.90 (s, 3H), |
| | 3.70 (br, 2H), 3.26 (m, 2H), 2.99 (s, 3H), 2.80 (q, 2H), 1.39 (t, 3H). Mass Spectrum (LCMS, |
| | ESI pos.) Calcd. For C₂₆H₂₈N₆O₅S: 537.61 (M + H), Found: 537.4. |
| 218 | {4-[4-(8-indan-5-yl-6-methoxycarbamoyl-5-oxo-5,8-dihydro-pyrido[2,3-d]pyrimidin-2- |
| | ylamino)-phenyl]-piperidin-1-yl}-acetic acid ethyl ester |
| | ¹H NMR (400 MHz, CDCl₃) δ (ppm): 11.99 (s, 1H), 9.38 (s, 1H), 8.82 (s, 1H), 7.78 (br, |
| | 1H), 7.41 (d, 1H), 7.26 (br, 3H), 7.19 (d, 1H), 6.97 (br, 2H), 4.23 (q, 2H), 3.91 (s, 3H), |
| | 3.25 (s, 2H), 3.07 (m, 4H), 3.00 (t, 2H), 2.44 (m, 1H), 2.00-2.36 (m, 4H), 1.80 (m, 4H), |
| | 1.35 (t, 3H). Mass Spectrum (LCMS, ESI pos.) Calcd. For C₃₃H₃₆N₆O₅: 597.69 (M + H), |
| | Found: 597.6. |
| 219 | {4-[4-(8-indan-5-yl-6-methoxycarbamoyl-5-oxo-5,8-dihydro-pyrido[2,3-d]pyrimidin-2- |
| | ylamino)-phenyl]-piperidin-1-yl}-acetic acid |
| | ¹H NMR (400 MHz, CD₃OD) δ (ppm): 9.24 (s, 1H), 8.66 (s, 1H), 7.45 (d, 1H), 7.36 (br, |
| | 3H), 7.24 (d, 1H), 6.97 (br, 2H), 4.15 (s, 2H), 3.82 (s, 3H), 3.79 (d, 2H), 3.25 (m, 2H), |
| | 3.10 (br, 2H), 3.00 (t, 2H), 2.85 (m, 1H), 2.25 (m, 2H), 2.04 (m, 4H). Mass Spectrum |
| | (LCMS, ESI pos.) Calcd. For C₃₁H₃₂N₆O₅: 569.63 (M + H), Found: 569.5. |
| 220 | 8-indan-5-yl-2-{4-[2-(4-methyl-3-oxo-piperazin-1-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ (ppm): 11.99 (s, 1H), 9.39 (s, 1H), 8.82 (s, 1H), 7.78 (br, |
| | 1H), 7.42 (d, 1H), 7.25 (br, 3H), 7.17 (d, 1H), 6.92 (br, 2H), 3.91 (s, 3H), 3.35 (t, 2H), |
| | 3.20 (s, 2H), 3.05 (t, 2H), 3.00 (t, 2H), 2.98 (s, 3H), 2.75 (m, 4H), 2.62 (m, 2H), 2.23 (m, |
| | 2H). Mass Spectrum (LCMS, ESI pos.) Calcd. For C₃₁H₃₃N₇O₄: 568.65 (M + H), Found: |
| | 568.6. |
| 221 | 8-(4-ethyl-phenyl)-2-{4-[2-(4-methyl-3-oxo-piperazin-1-yl)-ethyl]-phenylamino}-5-oxo- |
| | 5,8-dihydro-pyrido[2,3-d]pyrimidine-6-carboxylic acid methoxy-amide |
| | ¹H NMR (400 MHz, CDCl₃) δ (ppm): 11.99 (s, 1H), 9.37 (s, 1H), 8.80 (s, 1H), 8.05 (br, |
| | 1H), 7.42 (d, 2H), 7.35 (d, 2H), 7.17 (br, 2H), 6.92 (br, 2H), 3.90 (s, 3H), 3.36 (t, 2H), |
| | 3.20 (s, 2H), 2.98 (s, 3H), 2.83 (q, 2H), 2.72 (m, 4H), 2.60 (m, 2H), 1.40 (t, 3H). Mass |
| | Spectrum (LCMS, ESI pos.) Calcd. For C₃₀H₃₃N₇O₄: 556.64 (M + H), Found: 556.6. |
| 222 | 8-indan-5-yl-2-[4-(1-methyl-piperidin-4-yl)-phenylamino]-5-oxo-5,8-dihydro-pyrido[2,3- |
| | d]pyrimidine-6-carboxylic acid |
| | ¹H NMR (400 MHz, CDCl₃ + CD₃OD) δ (ppm): 9.42 (s, 1H), 8.82 (s, 1H), 7.45 (d, 1H), |
| | 7.30 (br, 2H), 7.24 (s, 1H), 7.17 (d, 1H), 6.97 (br, 2H), 3.62 (d, 2H), 3.10 (br, 2H), 3.00 (t, |
| | 2H), 2.85 (m, 5H), 2.62 (m, 1H), 2.40 (m, 2H), 2.25 (m, 2H), 1.97 (d, 2H). Mass Spectrum |
| | (LCMS, ESI pos.) Calcd. For C₂₉H₂₉N₅O₃: 496.58 (M + H), Found: 496.4. |
| 223 | 8-indan-5-yl-5-oxo-2-{4-[2-(3-oxo-piperazin-1-yl)-ethyl]-phenylamino}-5,8-dihydro- |
| | pyrido[2,3-d]pyrimidine-6-carboxylic acid methoxy-amide |
| | ¹H NMR (400 MHz, CDCl₃ + CD₃OD) δ (ppm): 9.38 (s, 1H), 8.80 (s, 1H), 7.45 (d, 1H), |
| | 7.25 (br, 3H), 7.20 (d, 1H), 6.94 (br, 2H), 3.90 (s, 3H), 3.40 (m, 2H), 3.21 (s, 2H), 3.10 (t, |
| | 2H), 3.01 (t, 2H), 2.70 (m, 6H), 2.25 (m, 2H). Mass Spectrum (LCMS, ESI pos.) Calcd. |
| | For C₃₀H₃₁N₇O₄: 554.62 (M + H), Found: 554.5. |
| 224 | 8-(4-ethyl-phenyl)-5-oxo-2-{4-[2-(3-oxo-piperazin-1-yl)-ethyl]-phenylamino}-5,8- |
| | dihydro-pyrido[2,3-d]pyrimidine-6-carboxylic acid methoxy-amide |
| | ¹H NMR (400 MHz, CDCl₃) δ (ppm): 11.98 (s, 1H), 9.37 (s, 1H), 8.81 (s, 1H), 8.05 (br, |
| | 1H), 7.42 (d, 2H), 7.35 (d, 2H), 7.25 (br, 2H), 6.94 (br, 2H), 6.30 (br, 1H), 3.90 (s, 3H), |
| | 3.40 (m, 2H), 3.21 (s, 2H), 2.82 (q, 2H), 2.73 (m, 4H), 2.65 (m, 2H), 1.40 (t, 3H). Mass |
| | Spectrum (LCMS, ESI pos.) Calcd. For C₂₉H₃₁N₇O₄: 542.61 (M + H), Found: 542.4. |
| 225 | 8-(6,7-dihydro-5H-[1]pyrindin-3-yl)-2-[4-(1-methyl-piperidin-4-yl)-phenylamino]-5-oxo- |
| | 5,8-dihydro-pyrido[2,3-d]pyrimidine-6-carboxylic acid methoxy-amide |
| | ¹H NMR (400 MHz, CDCl₃) δ (ppm): 11.90 (s, 1H), 9.38 (s, 1H), 8.78 (s, 1H), 8.44 (d, |
| | 1H), 7.58 (s, 1H), 7.52 (br, 1H), 7.23 (br, 2H), 7.02 (br, 2H), 3.91 (s, 3H), 3.20 (t, 2H), |
| | 3.05 (t, 2H), 2.98 (d, 2H), 2.42 (m, 1H), 2.30 (m, 5H), 2.05 (dt, 2H), 1.77 (m, 4H). Mass |
| | Spectrum (LCMS, ESI pos.) Calcd. For C₂₉H₃₁N₇O₃: 526.61 (M + H), Found: 526.3. |
| 226 | 8-(6,7-dihydro-5H-[1]pyrindin-3-yl)-2-[4-(2-methanesulfonyl-ethyl)-phenylamino]-5-oxo- |
| | 5,8-dihydro-pyrido[2,3-d]pyrimidine-6-carboxylic acid methoxy-amide |
| | ¹H NMR (400 MHz, CDCl₃ + CD₃OD) δ (ppm): 9.38 (s, 1H), 8.78 (s, 1H), 8.44 (s, 1H), |
| | 7.60 (s, 1H), 7.27 (br, 2H), 7.02 (br, 2H), 3.91 (s, 3H), 3.25 (t, 2H), 3.20 (t, 2H), 3.10 (m, |
| | 4H), 2.83 (s, 3H), 2.34 (m, 2H). Mass Spectrum (LCMS, ESI pos.) Calcd. For |
| | C₂₆H₂₆N₆O₅S: 535.6 (M + H), Found: 535.3. |
| 227 | 8-(6,7-dihydro-5H-[1]pyrindin-3-yl)-2-[4-(2-hydroxy-ethyl)-phenylamino]-5-oxo-5,8- |
| | dihydro-pyrido[2,3-d]pyrimidine-6-carboxylic acid methoxy-amide |
| | ¹H NMR (400 MHz, CDCl₃ + CD₃OD) δ (ppm): 9.38 (s, 1H), 8.75 (s, 1H), 8.38 (s, 1H), |
| | 7.60 (s, 1H), 7.22 (br, 2H), 7.01 (br, 2H), 3.91 (s, 3H), 3.80 (t, 2H), 3.18 (t, 2H), 3.05 (t, |
| | 2H), 2.80 (t, 2H), 2.34 (m, 2H). Mass Spectrum (LCMS, ESI pos.) Calcd. For C₂₅H₂₄N₆O₄: |
| | 473.5 (M + H), Found: 473.3. |
| 228 | 2-[3-(2-dimethylamino-acetylamino)-phenylamino]-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.38 (s, 1H), 9.10 (s, 1H), 8.80 (s, |
| | 1H), 8.14 (br, 1H), 7.72 (br, 1H), 7.42 (d, 1H), 7.35 (d, 2H), 7.35 (d, 2H), 6.99 (br, 2H), |
| | 3.91 (s, 3H), 3.10 (s, 2H), 2.81 (q, 2H), 2.38 (s, 6H), 1.38 (t, 3H). Mass Spectrum (LCMS, |
| | ESI pos.) Calcd. For C₂₇H₂₉N₇O₄: 516.57 (M + H), Found: 516.4. |
| 229 | 2-[3-(2-dimethylamino-acetylamino)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.38 (s, 1H), 9.10 (s, 1H), 8.82 (s, |
| | 1H), 8.14 (br, 1H), 7.75 (br, 1H), 7.42 (m, 2H), 7.24 (s, 1H), 7.17 (d, 1H), 6.99 (br, 2H), |
| | 3.91 (s, 3H), 3.10 (s, 2H), 3.05 (t, 2H), 3.00 (t, 2H), 2.40 (s, 6H), 1.23 (m, 2H). Mass |
| | Spectrum (LCMS, ESI pos.) Calcd. For C₂₈H₂₉N₇O₄: 528.59 (M + H), Found: 528.5. |
| 230 | 2-[3-(2-dimethylamino-ethanesulfonyl)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ (ppm): 11.95 (s, 1H), 9.41 (s, 1H), 8.84 (s, 1H), 7.94 (d, |
| | 1H), 7.78 (br, 1H), 7.56 (d, 1H), 7.42 (d, 1H), 7.24 (s, 1H), 7.19 (d, 1H), 3.91 (s, 3H), |
| | 3.24 (t, 2H), 3.09 (t, 2H), 3.00 (t, 2H), 2.66 (t, 2H), 2.23 (m, 2H), 1.99 (S, 6H). Mass Spectrum |
| | (LCMS, ESI pos.) Calcd. For C₂₈H₃₀N₆O₅S: 563.65 (M + H), Found: 563.4. |
| 231 | 8-indan-5-yl-2-[3-(2-methylamino-ethanesulfonyl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 11.93 (s, 1H), 9.40 (s, 1H), 8.82 (s, 1H), 7.94 (d, |
| | 1H), 7.78 (br, 1H), 7.54 (d, 1H), 7.42 (d, 1H), 7.26 (s, 1H), 7.19 (d, 1H), 5.58 (br, 1H), |
| | 3.91 (s, 3H), 3.35 (t, 2H), 3.09 (t, 2H), 3.00 (m, 4H), 2.41 (s, 3H), 2.23 (m, 2H). Mass |
| | Spectrum (LCMS, ESI pos.) Calcd. For C₂₇H₂₈N₆O₅S: 549.62 (M + H), Found: 549.4. |
| 232 | 2-{4-[2-(3,3-difluoro-piperidin-1-yl)-ethyl]-phenylamino}-8-(4-ethyl-phenyl)-5-oxo-5,8- |
| | dihydro-pyrido[2,3-d]pyrimidine-6-carboxylic acid methoxy-amide |
| | ¹H NMR (400 MHz, CDCl₃) δ (ppm): 11.97 (s, 1H), 9.33 (s, 1H), 8.82 (s, 1H), 8.17 (m, |
| | 1H), 7.42 (d, 2H, J = 8.2), 7.33 (d, 2H, J = 8.4), 7.28 (s, 2H), 6.92 (s, 2H), 3.90 (s, 3H), |
| | 2.68 (m, 10H), 1.90 (dd, 2H, J = 5.6, 12.3), 1.80 (m, 2H), 1.39 (t, 3H, J = 7.6). Mass |
| | Spectrum (LCMS, ESI pos.) Calcd. For C₃₀H₃₂F₂N₆O₃: 563.62 (M + H), Found: 563.3. |
| 233 | 2-{4-[2-(3,3-difluoro-piperidin-1-yl)-ethyl]-phenylamino}-8-(4-ethyl-phenyl)-5-oxo-5,8- |
| | dihydro-pyrido[2,3-d]pyrimidine-6-carboxylic acid methoxy-amide |
| | ¹H NMR (400 MHz, CDCl₃) δ (ppm): 11.98 (s, 1H), 9.33 (s, 1H), 8.79 (s, 1H), 8.00 (br, |
| | 1H), 7.42 (d, 1H, J = 7.7), 7.27 (s, 3H), 7.17 (d, 1H, J = 7.7), 6.92 (br, 2H), 3.88 (s, 3H), |
| | 3.02 (m, 4H), 2.68 (m, 6H), 2.51 (m, 2H), 2.23 (m, 2H), 1.89 (m, 4H). Mass Spectrum |
| | (LCMS, ESI pos.) Calcd. For C₃₁H₃₂F₂N₆O₃: 575.64 (M + H), Found: 575.3. |
| 234 | 2-{4-[2-(4,4-difluoro-piperidin-1-yl)-ethyl]-phenylamino}-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ (ppm): 11.96 (s, 1H), 9.37 (s, 1H), 8.83 (s, 1H), 7.59 (br, |
| | 1H), 7.42 (d, 1H, J = 8.1), 7.26 (s, 3H), 7.18 (d, 1H, J = 7.9), 6.91 (br, 2H), 3.90 (s, 3H), |
| | 3.07 (m, 2H), 3.00 (m, 2H), 2.73 (m, 2H), 2.62 (m, 6H), 2.23 (m, 2H), 2.02 (m, 4H). Mass |
| | Spectrum (LCMS, ESI pos.) Calcd. For C₃₁H₃₂F₂N₆O₃: 575.64 (M + H), Found: 575.3. |
| 235 | 8-indan-5-yl-2-[4-(1-methyl-1,2,3,6-tetrahydro-pyridin-4-yl)-phenylamino]-5-oxo-5,8- |
| | dihydro-pyrido[2,3-d]pyrimidine-6-carboxylic acid methoxy-amide |
| | ¹H NMR (400 MHz, CDCl₃) δ (ppm): 11.97 (s, 1H), 9.37 (s, 1H), 8.84 (s, 1H), 7.72 (br, |
| | 1H), 7.43 (d, 1H, J = 8.0), 7.26 (s, 3H), 7.17 (m, 3H), 5.98 (s, 1H), 3.90 (s, 3H), 3.15 (s, |
| | 2H), 3.08 (t, 2H), 3.00 (t, 2H, J = 7.4), 2.70 (m, 2H), 2.55 (s, 2H), 2.44 (s, 3H), 2.24 (m, |
| | 2H). Mass Spectrum (LCMS, ESI pos.) Calcd. For C₃₀H₃₀N₆O₃: 523.61 (M + H), Found: |
| | 523.3. |
| 236 | 2-{4-[(3S,4S)-(3,4-dihydroxy-1-methyl-piperidin-4-yl)]-phenylamino}-8-indan-5-yl-5- |
| | oxo-5,8-dihydro-pyrido[2,3-d]pyrimidine-6-carboxylic acid methoxy-amide |
| | ¹H NMR (400 MHz, CDCl₃ + CD₃OD) δ (ppm): 9.34 (s, 1H), 8.76 (s, 1H), 7.44 (d, 1H, J = 8.0), |
| | 7.34 (br, 3H), 7.27 (s, 1H), 7.19 (m, 3H), 4.04 (m, 1H), 3.89 (s, 3H), 3.10 (m, 2H), |
| | 3.01 (t, 2H, J = 7.4), 2.84 (m, 1H), 2.65 (m, 1H), 2.46 (m, 1H), 2.37 (s, 3H), 2.35 (m, 1H), |
| | 2.25 (m, 2H), 1.92 (m, 1H), 1.79 (m, 1H). Mass Spectrum (LCMS, ESI pos.) Calcd. For |
| | C₃₀H₃₂N₆O₅: 557.63 (M + H), Found: 557.4. |
| 237 | 2-[4-(3,3-difluoro-1-methyl-piperidin-4-yl)-phenylamino]-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ (ppm): δ 12.01 (s, 1H), 9.35 (s, 1H), 8.83 (s, 1H), 7.42 (d, |
| | 1H, J = 7.9), 7.29 (s, 2H), 7.26 (s, 1H), 7.17 (d, 1H, J = 7.9), 7.04 (s, 2H), 3.90 (s, 3H), |
| | 3.19 (m, 1H), 3.03 (m, 5H), 2.80 (m, 1H), 2.41 (s, 3H), 2.26 (m, 5H), 1.84 (m, 1H). Mass |
| | Spectrum (LCMS, ESI pos.) Calcd. For C₃₀H₃₀F₂N₆O₃: 561.61 (M + H), Found: 561.4. |
| 238 | 2-{4-[2-(4-hydroxy-piperidin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.99 (s, 1H), 9.33 (s, 1H), 8.81 (s, 1H), 8.46 (br, 1H), |
| | 7.43 (d, 1H, J = 7.9), 7.28 (m, 3H), 7.18 (d, 1H, J = 7.9), 6.92 (m, 2H), 3.90 (s, 3H), 3.72 (m, |
| | 1H), 3.08 (t, 2H), 2.99 (t, 2H, J = 7.6), 2.85 (m, 2H), 2.72 (m, 2H), 2.49 (m, 2H), 2.21 (m, |
| | 4H), 1.99 (m, 2H), 1.59 (m, 2H). Mass Spectrum (LCMS, ESI pos.) Calcd. For |
| | C₃₁H₃₄N₆O₄: 555.65 (M + H), Found: 555.4. |
| 239 | 2-{4-{2-[(3R)-(3-hydroxy-piperidin-1-yl)]-ethyl}-phenylamino}-8-indan-5-yl-5-oxo-5,8- |
| | dihydro-pyrido[2,3-d]pyrimidine-6-carboxylic acid methoxy-amide ¹H NMR (400 MHz, |
| | CDCl₃) δ 11.99 (s, 1H), 9.34 (s, 1H), 8.83 (s, 1H), 8.10 (br, 1H), 7.42 (d, 1H, J = 7.9), |
| | 7.27 (m, 3H), 7.17 (d, 1H, J = 7.9), 6.90 (m, 2H), 3.90 (s, 3H), 3.81 (m, 1H), 3.05 (m, 2H), |
| | 3.02 (t, 2H), 2.72 (m, 2H), 2.55 (m, 4H), 2.37 (m, 1H), 2.23 (m, 2H), 1.80 (m, 1H), 1.57 (m, |
| | 4H). Mass Spectrum (LCMS, ESI pos.) Calcd. For C₃₁H₃₄N₆O₄: 555.65 (M + H), Found: |
| | 555.4. |
| 240 | 8-(4-ethyl-phenyl)-2-(4-{2-[(3R)-(3-hydroxy-piperidin-1-yl)]-ethyl}-phenylamino)-5-oxo- |
| | 5,8-dihydro-pyrido[2,3-d]pyrimidine-6-carboxylic acid methoxy-amide |
| | ¹H NMR (400 MHz, CDCl₃) δ 11.97 (s, 1H), 9.37 (s, 1H), 8.82 (s, 1H), 7.60 (m, 1H), |
| | 7.43 (d, 2H, J = 8.1), 7.34 (d, 2H, J = 8.3), 7.25 (m, 2H), 6.90 (m, 2H), 3.90 (s, 3H), 3.84 (m, |
| | 1H), 2.83 (m, 2H), 2.70 (m, 2H), 2.53 (m, 4H), 2.32 (m, 1H), 1.80 (m, 1H), 1.57 (m, 4H), |
| | 1.39 (t, 3H, J = 7.6). Mass Spectrum (LCMS, ESI pos.) Calcd. For C₃₀H₃₄N₆O₄: |
| | 543.64 (M + H), Found: 543.3. |
| 241 | 2-{4-[2-(3,3-difluoro-pyrrolidin-1-yl)-ethyl]-phenylamino}-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.35 (s, 1H), 8.84 (s, 1H), 7.80 (m, 1H), |
| | 7.42 (d, 1H, J = 7.8), 7.27 (m, 3H), 7.17 (d, 1H, J = 7.9), 6.92 (br, 2H), 3.90 (s, 3H), 3.08 (m, |
| | 2H), 3.00 (t, 2H, J = 7.4), 2.94 (t, 2H, J = 13.2), 2.78 (t, 2H, J = 7.0), 2.69 (m, 4H), |
| | 2.28 (m, 4H). Mass Spectrum (LCMS, ESI pos.) Calcd. For C₃₀H₃₀F₂N₆O₃: 561.61 (M + H), |
| | Found: 561.3. |
| 242 | 8-(4-ethyl-phenyl)-5-oxo-2-(4-piperidin-2-yl-phenylamino)-5,8-dihydro-pyrido[2,3- |
| | d]pyrimidine-6-carboxylic acid methoxy-amide |
| | ¹H NMR (400 MHz, CDCl₃) δ 11.98 (s, 1H), 9.35 (s, 1H), 8.82 (s, 1H), 7.93 (s, 1H), |
| | 7.42 (d, 2H, J = 8.2), 7.31 (d, 2H, J = 8.2), 7.27 (m, 2H), 7.10 (m, 2H), 3.89 (s, 3H), 3.49 (m, |
| | 1H), 3.17 (d, 1H, J = 11.5), 2.80 (m, 3H), 1.87 (m, 1H), 1.67 (m, 3H), 1.44 (m, 5H). Mass |
| | Spectrum (LCMS, ESI pos.) Calcd. For C₂₈H₃₀N₆O₃: 499.59 (M + H), Found: 499.4. |
| 243 | 8-indan-5-yl-2-[4-(1-methyl-piperidin-2-yl)-phenylamino]-5-oxo-5,8-dihydro-pyrido[2,3- |
| | d]pyrimidine-6-carboxylic acid methoxy-amide |
| | ¹H NMR (400 MHz, CDCl₃) δ 11.99 (s, 1H), 9.35 (s, 1H), 8.83 (s, 1H), 7.95 (m, 1H), |
| | 7.43 (d, 1H, J = 7.9), 7.26 (m, 4H, J = 5.9), 7.18 (d, 1H, J = 7.9), 7.01 (m, 2H), 3.90 (s, 3H), |
| | 3.01 (m, 5H), 2.69 (m, 1H), 2.24 (m, 2H), 2.10 (m, 1H), 2.03 (s, 3H), 1.83 (m, 1H), |
| | 1.71 (m, 3H), 1.53 (m, 1H), 1.35 (m, 1H). Mass Spectrum (LCMS, ESI pos.) Calcd. For |
| | C₃₀H₃₂N₆O₃: 525.63 (M + H), Found: 525.4. |
| 244 | 2-[4-(1-ethyl-piperidin-2-yl)-phenylamino]-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.99 (s, 1H), 9.35 (s, 1H), 8.83 (s, 1H), 8.16 (m, 1H), |
| | 7.42 (d, 1H), 7.27 (m, 3H), 7.18 (d, 1H, J = 7.9), 7.00 (m, 2H), 3.90 (s, 3H), 3.09 (m, 3H), |
| | 3.00 (m, 3H), 2.47 (m, 2H), 2.25 (m, 2H), 2.03 (m, 2H), 1.70 (m, 3H), 1.49 (m, 1H), 1.32 (m, |
| | 1H), 0.89 (m, 3H). Mass Spectrum (LCMS, ESI pos.) Calcd. For C₃₁H₃₄N₆O₃: 539.66 (M + H), |
| | Found: 539.4. |
| 245 | 8-(4-ethyl-phenyl)-2-[4-(1-ethyl-piperidin-2-yl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 11.99 (s, 1H), 9.35 (s, 1H), 8.83 (s, 1H), 7.94 (m, 1H), |
| | 7.42 (d, 2H, J = 8.3), 7.33 (d, 2H, J = 8.4), 7.27 (br, 2H), 7.07 (br, 2H), 3.90 (s, 3H), 3.13 (m, |
| | 1H), 2.91 (m, 1H), 2.83 (q, 2H, J = 7.6), 2.46 (m, 1H), 2.03 (m, 1H), 1.91 (m, 1H), |
| | 1.66 (m, 4H), 1.49 (m, 1H), 1.39 (t, 4H, J = 7.6), 0.88 (t, 3H). Mass Spectrum (LCMS, ESI |
| | pos.) Calcd. For C₃₀H₃₄N₆O₃: 527.64 (M + H), Found: 527.4. |
| 246 | 8-indan-5-yl-5-oxo-2-(4-piperidin-2-yl-phenylamino)-5,8-dihydro-pyrido[2,3- |
| | d]pyrimidine-6-carboxylic acid methoxy-amide |
| | ¹H NMR (400 MHz, CDCl₃) δ 11.97 (br, 1H), 9.31 (s, 1H), 8.83 (s, 1H), 8.33 (br, 1H), |
| | 7.42 (d, 1H, J = 7.7), 7.26 (s, 1H), 7.26 (br, 2H), 7.16 (d, 1H, J = 7.8), 7.09 (br, 2H), |
| | 5.69 (m, 1H), 3.90 (s, 3H), 3.50 (m, 1H), 3.18 (d, 1H, J = 11.5), 3.08 (m, 2H), 2.99 (t, 2H, J = 7.4), |
| | 2.78 (t, 1H, J = 11.5), 2.24 (m, 2H), 1.88 (m, 2H), 1.69 (m, 2H), 1.48 (m, 2H). Mass |
| | Spectrum (LCMS, ESI pos.) Calcd. For C₂₉H₃₀N₆O₃: 511.6 (M + H), Found: 511.4. |
| 247 | 2-[3-(1-aza-bicyclo[2.2.2]oct-3-yloxy)-phenylamino]-8-(4-ethyl-phenyl)-5-oxo-5,8- |
| | dihydro-pyrido[2,3-d]pyrimidine-6-carboxylic acid methoxy-amide |
| | ¹H NMR (400 MHz, CDCl₃) δ 11.97 (s, 1H), 9.37 (s, 1H), 8.81 (s, 1H), 7.41 (d, 2H, J = 8.5), |
| | 7.34 (d, 2H, J = 8.4), 7.11 (m, 1H), 6.90 (m, 1H), 6.67 (m, 1H), 6.53 (m, 1H), |
| | 4.28 (br, 1H), 3.90 (s, 3H), 3.23 (m, 1H), 2.95 (m, 1H), 2.81 (m, 6H), 2.12 (br, 1H), 1.95 (m, |
| | 1H), 1.73 (m, 1H), 1.54 (m, 1H), 1.35 (t, 4H, J = 7.6). Mass Spectrum (LCMS, ESI pos.) |
| | Calcd. For C₃₀H₃₂N₆O₄: 541.63 (M + H), Found: 541.3. |
| 248 | 8-(4-ethyl-phenyl)-5-oxo-2-[3-(piperidin-3-yloxy)-phenylamino]-5,8-dihydro-pyrido[2,3- |
| | d]pyrimidine-6-carboxylic acid methoxy-amide |
| | ¹H NMR (400 MHz, CDCl₃) δ 11.97 (s, 1H), 9.37 (s, 1H), 8.81 (s, 1H), 7.41 (d, 2H, J = 8.5), |
| | 7.34 (d, 2H, J = 8.4), 7.11 (m, 1H), 6.90 (m, 1H), 6.67 (m, 1H), 6.53 (m, 1H), |
| | 4.15 (m, 1H), 3.90 (s, 3H), 3.13 (d, 1H), 2.81 (m, 5H), 2.02 (m, 1H), 1.70 (m, 2H), 1.49 (m, |
| | 1H), 1.35 (t, 3H, J = 7.6). Mass Spectrum (LCMS, ESI pos.) Calcd. For C₂₈H₃₀N₆O₄: |
| | 515.59 (M + H), Found: 515.3. |
| 249 | 2-{4-[(3R,4R)-(3,4-dihydroxy-1-methyl-piperidin-4-yl)]-phenylamino}-8-(4-ethyl- |
| | phenyl)-5-oxo-5,8-dihydro-pyrido[2,3-d]pyrimidine-6-carboxylic acid methoxy-amide |
| | ¹H NMR (400 MHz, CDCl₃ + CD₃OD) δ 9.31 (s, 1H), 8.73 (s, 1H), 7.43 (d, 2H, J = 8.5), |
| | 7.31 (m, 4H), 7.20 (m, 2H), 4.07 (m, 1H), 3.89 (s, 3H), 2.84 (m, 3H), 2.63 (m, 1H), |
| | 2.46 (m, 1H), 2.35 (m, 4H), 1.95 (m, 1H), 1.80 (m, 1H), 1.38 (t, 3H, J = 7.6). Mass Spectrum |
| | (LCMS, ESI pos.) Calcd. For C₂₉H₃₂N₆O₅: 545.62 (M + H), Found: 545.3. |
| 250 | 2-{4-[(3R,4R)-(3,4-dihydroxy-1-methyl-piperidin-4-yl)]-phenylamino}-8-indan-5-yl-5- |
| | oxo-5,8-dihydro-pyrido[2,3-d]pyrimidine-6-carboxylic acid methoxy-amide |
| | ¹H NMR (400 MHz, CDCl₃ + CD₃OD) δ 9.32 (s, 1H), 8.78 (s, 1H), 7.43 (d, 1H, J = 7.9), |
| | 7.30 (m, 2H), 7.26 (s, 1H), 7.21 (m, 2H), 7.17 (d, 1H, J = 7.9), 4.07 (m, 1H), 3.89 (s, 3H), |
| | 3.09 (m, 2H), 3.00 (t, 2H, J = 7.4), 2.84 (m, 1H), 2.64 (m, 1H), 2.44 (m, 1H), 2.38 (s, 3H), |
| | 2.33 (t, 1H, J = 10.5), 2.24 (m, 2H), 1.93 (m, 1H), 1.82 (m, 1H). Mass Spectrum (LCMS, |
| | ESI pos.) Calcd. For C₃₀H₃₂N₆O₅: 557.63 (M + H), Found: 557.3. |
| 251 | 2-[3-(1-ethyl-piperidin-3-yloxy)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.97 (s, 1H), 9.38 (s, 1H), 8.82 (s, 1H), 7.60 (m, 1H), |
| | 7.42 (d, 1H, J = 7.9), 7.25 (s, 1H), 7.19 (d, 1H, J = 7.9), 7.08 (d, 1H, J = 7.3), 6.95 (m, 1H), |
| | 6.77 (m, 1H), 6.63 (d, 1H, J = 7.3), 4.31 (m, 1H), 3.90 (s, 3H), 3.06 (t, 2H, J = 7.4), 3.00 (t, |
| | 2H, J = 7.4), 2.77 (m, 1H), 2.47 (q, 2H, J = 7.2), 2.21 (p, 2H, J = 7.6), 2.05 (m, 4H), |
| | 1.80 (m, 1H), 1.64 (m, 1H), 1.42 (m, 1H), 1.08 (t, 3H, J = 7.2). Mass Spectrum (LCMS, ESI |
| | pos.) Calcd. For C₃₁H₃₄N₆O₄: 555.65 (M + H), Found: 555.3. |
| 252 | 2-(4-{2-[(3R)-(3-fluoro-pyrrolidin-1-yl)]-ethyl}-phenylamino)-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.35 (s, 1H), 8.84 (s, 1H), 7.83 (s, 1H), |
| | 7.42 (d, 1H, J = 7.9), 7.26 (m, 3H), 7.16 (d, 1H, J = 7.9), 6.93 (m, 2H), 5.19 (dt, 1H, J = 55.4), |
| | 3.90 (s, 3H), 3.07 (t, 2H, J = 7.2), 3.00 (t, 2H, J = 7.4), 2.91 (m, 2H), 2.73 (m, 5H), |
| | 2.49 (m, 1H), 2.15 (m, 4H). Mass Spectrum (LCMS, ESI pos.) Calcd. For C₃₀H₃₁FN₆O₃: |
| | 543.62 (M + H), Found: 543.3. |
| 253 | 2-(4-{2-[(3S)-(3-fluoro-pyrrolidin-1-yl)]-ethyl}-phenylamino)-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.35 (s, 1H), 8.84 (s, 1H), 7.83 (s, 1H), |
| | 7.42 (d, 1H, J = 7.9), 7.26 (m, 3H), 7.16 (d, 1H, J = 7.9), 6.93 (m, 2H), 5.19 (dt, 1H, J = 55.4), |
| | 3.90 (s, 3H), 3.07 (t, 2H, J = 7.2), 3.00 (t, 2H, J = 7.4), 2.91 (m, 2H), 2.73 (m, 5H), |
| | 2.49 (m, 1H), 2.15 (m, 4H). Mass Spectrum (LCMS, ESI pos.) Calcd. For C₃₀H₃₁FN₆O₃: |
| | 543.62 (M + H), Found: 543.3. |
| 254 | 2-{4-[2-(3-hydroxy-azetidin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃ + CD₃OD) δ 9.36 (s, 1H), 8.82 (s, 1H), 7.43 (d, 1H, J = 8.0), |
| | 7.27 (s, 1H, J = 4.2), 7.22 (m, 2H), 7.17 (d, 1H, J = 8.1), 6.88 (m, 2H), 4.41 (m, 1H), |
| | 3.90 (s, 3H), 3.63 (m, 2H), 3.08 (m, 2H), 3.02 (m, 2H), 2.93 (m, 2H), 2.66 (m, 2H), 2.59 (m, |
| | 2H), 2.24 (m, 2H). Mass Spectrum (LCMS, ESI pos.) Calcd. For C₂₉H₃₀N₆O₄: 527.6 (M + H), |
| | Found: 527.4. |
| 255 | 2-(4-{2-[(3S)-(3-hydroxy-pyrrolidin-1-yl)]-ethyl}-phenylamino)-8-indan-5-yl-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.35 (s, 1H), 8.83 (s, 1H), 7.79 (s, 1H), |
| | 7.42 (d, 1H, J = 7.9), 7.27 (m, 3H), 7.17 (d, 1H, J = 7.9), 6.92 (br, 2H), 4.37 (m, 1H), 3.90 (s, |
| | 3H), 3.08 (t, 2H, J = 7.4), 3.00 (t, 2H, J = 7.4), 2.93 (m, 1H), 2.74 (m, 3H), 2.65 (m, 2H), |
| | 2.57 (dd, 1H, J = 5.2, 10.0), 2.35 (dd, 1H, J = 8.8, 15.1), 2.22 (m, 3H), 1.77 (m, 1H). |
| | Mass Spectrum (LCMS, ESI pos.) Calcd. For C₃₀H₃₂N₆O₄: 541.63 (M + H), Found: 541.4. |
| 256 | 2-{4-[2-(3-fluoro-piperidin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.98 (s, 1H), 9.35 (s, 1H), 8.84 (s, 1H), 7.84 (m, 1H), |
| | 7.42 (d, 1H, J = 7.9), 7.27 (m, 3H), 7.17 (d, 1H, J = 7.8), 6.92 (m, 2H), 4.68 (d, 1H, J = 48.1), |
| | 3.90 (s, 3H), 3.08 (m, 2H), 3.00 (t, 2H, J = 7.4), 2.73 (m, 3H), 2.57 (m, 4H), 2.40 (m, 1H), |
| | 2.23 (m, 2H), 1.86 (m, 2H), 1.68 (m, 1H), 1.59 (m, 1H). Mass Spectrum (LCMS, ESI |
| | pos.) Calcd. For C₃₁H₃₃FN₆O₃: 557.65 (M + H), Found: 557.4. |
| 257 | 8-(4-ethyl-phenyl)-2-[4-(2-methoxy-ethoxy)-phenylamino]-5-oxo-5,8-dihydro-pyrido[2,3- |
| | d]pyrimidine-6-carboxylic acid methoxy-amide |
| | ¹H NMR (400 MHz, CDCl₃) δ (ppm): 11.99 (s, 1H), 9.31 (s, 1H), 8.81 (s, 1H), 7.97 (s, |
| | 1H), 7.40 (d, 2H, J = 8.3), 7.31 (d, 2H, J = 8.4), 7.25 (d, 2H, J = 14.1), 6.66 (d, 2H, J = 14.1), |
| | 4.05 (m, 2H), 3.90 (s, 3H), 3.74 (dd, 2H, J = 3.9, 5.5), 3.45 (s, 3H), 2.82 (q, 2H, J = 7.6), |
| | 1.37 (t, 3H, J = 7.5). Mass Spectrum (LCMS, ESI pos.) Calcd. For C₂₆H₂₇N₅O₅: |
| | 490.54 (M + H), Found: 490.3. |
| 258 | 8-indan-5-yl-2-[4-(2-methoxy-ethoxy)-phenylamino]-5-oxo-5,8-dihydro-pyrido[2,3- |
| | d]pyrimidine-6-carboxylic acid methoxy-amide |
| | ¹H NMR (400 MHz, CDCl₃) δ (ppm): 11.99 (s, 1H), 9.32 (s, 1H), 8.82 (s, 1H), 7.78 (s, |
| | 1H), 7.40 (d, 1H, J = 7.8), 7.26 (m, 3H), 7.16 (d, 1H, J = 7.9), 6.66 (m, 2H), 4.07 (m, 2H), |
| | 3.90 (s, 3H), 3.75 (m, 2H), 3.46 (s, 3H), 3.07 (m, 2H), 2.99 (t, 2H, J = 7.4), 2.23 (m, 2H). |
| | Mass Spectrum (LCMS, ESI pos.) Calcd. For C₂₇H₂₇N₅O₅: 502.55 (M + H), Found: 502.3. |
| 259 | 8-(1H-inden-5-yl)-2-[4-(1-methyl-piperidin-4-yl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ (ppm): 12.00 (s, 1H), 9.34 (s, 1H), 8.89 (s, 1H), 7.90 (s, |
| | 1H), 7.61 (dd, 1H, J = 7.9, 28.5), 7.47 (d, 1H, J = 15.5), 7.32 (d, 1H, J = 7.9), 7.21 (dd, 2H, |
| | J = 2.0, 7.8), 7.05 (s, 1H), 6.92 (d, 1H, J = 5.6), 6.80 (s, 2H), 3.91 (s, 3H), 3.59 (s, 1H), |
| | 3.49 (s, 1H), 2.96 (d, 2H, J = 11.2), 2.32 (s, 3H), 2.02 (t, 2H, J = 10.3), 1.72 (m, 5H). Mass |
| | Spectrum (LCMS, ESI pos.) Calcd. For C₃₀H₃₀N₆O₃: 523.61 (M + H), Found: 523.3. |
| 260 | 2-[4-(2-azetidin-1-yl-ethyl)-phenylamino]-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.98 (s, 1H), 9.34 (s, 1H), 8.82 (s, 1H), 7.92 (s, 1H), |
| | 7.42 (d, 2H, J = 8.3), 7.33 (d, 2H, J = 8.3), 7.26 (m, 2H), 6.91 (m, 2H), 3.90 (s, 3H), 3.21 (t, 4H, |
| | J = 7.0), 2.83 (q, 2H, J = 7.6), 2.58 (m, 4H), 2.08 (p, 2H, J = 7.1), 1.38 (t, 3H, J = 7.6). |
| | Mass Spectrum (LCMS, ESI pos.) Calcd. For C₂₈H₃₀N₆O₃: 499.59 (M + H), Found: 499.5. |
| 261 | 8-indan-5-yl-2-[3-(2-methoxy-ethoxy)-phenylamino]-5-oxo-5,8-dihydro-pyrido[2,3- |
| | d]pyrimidine-6-carboxylic acid methoxy-amide |
| | ¹H NMR (400 MHz, CDCl₃) δ 11.97 (s, 1H), 9.37 (s, 1H), 8.82 (s, 1H), 7.69 (br, 1H), |
| | 7.41 (d, 1H, J = 8.0), 7.26 (s, 1H), 7.18 (d, 1H, J = 7.9), 7.05 (d, 1H, J = 6.2), 6.87 (m, 2H), |
| | 6.62 (d, 1H, J = 7.6), 3.96 (br, 2H), 3.91 (s, 3H), 3.70 (m, 2H), 3.44 (s, 3H), 3.05 (t, 2H, J = 7.4), |
| | 2.98 (t, 2H, J = 7.4), 2.21 (p, 2H, J = 7.5). Mass Spectrum (LCMS, ESI pos.) |
| | Calcd. For C₂₇H₂₇N₅O₅: 502.55 (M + H), Found: 502.4. |
| 262 | 2-[4-(2-azetidin-1-yl-ethyl)-phenylamino]-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.99 (s, 1H), 9.34 (s, 1H), 8.84 (s, 1H), 7.95 (s, 1H), |
| | 7.42 (d, 1H, J = 7.9), 7.27 (m, 3H), 7.16 (d, 1H, J = 7.8), 6.91 (br, 2H), 3.90 (s, 3H), 3.18 (t, 4H, |
| | J = 7.0), 3.08 (t, 2H, J = 7.2), 3.00 (t, 2H, J = 7.4), 2.57 (m, 2H), 2.24 (m, 2H), 2.06 (m, |
| | 4H). Mass Spectrum (LCMS, ESI pos.) Calcd. For C₂₉H₃₀N₆O₃: 511.6 (M + H), Found: |
| | 511.5. |
| 263 | 2-{3-[(3S)-(1-ethyl-pyrrolidin-3-yloxy)]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.97 (s, 1H), 9.37 (s, 1H), 8.82 (s, 1H), 7.65 (br, 1H), |
| | 7.42 (d, 1H, J = 7.9), 7.25 (s, 1H), 7.19 (d, 1H, J = 7.9), 7.07 (m, 1H), 6.93 (m, 1H), 6.67 (m, |
| | 1H), 6.54 (m, 1H), 4.70 (br, 1H), 3.90 (s, 3H), 3.05 (t, 2H, J = 7.5), 2.99 (t, 2H, J = 7.4), |
| | 2.81 (m, 3H), 2.53 (m, 3H), 2.22 (m, 3H), 1.94 (s, 1H), 1.13 (t, 3H, J = 7.2). Mass |
| | Spectrum (LCMS, ESI pos.) Calcd. For C₃₀H₃₂N₆O₄: 541.63 (M + H), Found: 541.4. |
| 264 | 2-[4-(1-aza-bicyclo[2.2.2]oct-3-yloxy)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 12.00 (s, 1H), 9.32 (s, 1H), 8.83 (s, 1H), 7.83 (s, 1H), |
| | 7.41 (d, 1H, J = 7.8), 7.26 (m, 3H), 7.16 (d, 1H, J = 7.9), 6.58 (br, 2H), 4.28 (br, 1H), 3.90 (s, |
| | 3H), 3.23 (m, 1H), 3.04 (m, 2H), 3.00 (t, 3H, J = 7.4), 2.83 (m, 4H), 2.22 (m, 2H), 2.12 (s, |
| | 1H), 1.96 (m, 1H), 1.75 (m, 1H), 1.55 (m, 1H), 1.39 (m, 1H). Mass Spectrum (LCMS, |
| | ESI pos.) Calcd. For C₃₁H₃₂N₆O₄: 553.64 (M + H), Found: 553.4. |
| 265 | 8-(4-ethyl-phenyl)-2-{4-[2-(3-fluoro-azetidin-1-yl)-ethyl]-phenylamino}-5-oxo-5,8- |
| | dihydro-pyrido[2,3-d]pyrimidine-6-carboxylic acid methoxy-amide |
| | ¹H NMR (400 MHz, CDCl₃) δ 11.97 (s, 1H), 9.35 (s, 1H), 8.82 (s, 1H), 7.80 (s, 1H), |
| | 7.42 (d, 2H, J = 8.3), 7.33 (d, 2H, J = 8.4), 7.25 (br, 2H), 6.91 (br, 2H), 5.10 (dp, 1H, J = 5.3, |
| | 57.3), 3.89 (s, 3H), 3.65 (m, 2H), 3.10 (m, 2H), 2.83 (q, 2H, J = 7.6), 2.69 (t, 2H, J = 7.4), |
| | 2.58 (t, 2H, J = 7.4), 1.39 (t, 3H, J = 7.6), 1.13 (t, 3H, J = 7.2). Mass Spectrum (LCMS, |
| | ESI pos.) Calcd. For C₂₈H₂₉FN₆O₃: 517.58 (M + H), Found: 517.4. |
| 266 | 2-{4-[2-(3-fluoro-azetidin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.98 (s, 1H), 9.35 (s, 1H), 8.84 (s, 1H), 7.84 (s, 1H), |
| | 7.42 (d, 1H, J = 8.0), 7.27 (m, 3H), 7.17 (d, 1H, J = 7.9), 6.90 (br, 2H), 5.10 (dp, 1H, J = 5.4, |
| | 57.4), 3.90 (s, 3H), 3.66 (m, 2H), 3.14 (m, 1H), 3.10 (m, 3H), 3.00 (t, 2H, J = 7.4), 2.71 (t, |
| | 2H, J = 7.4), 2.60 (t, 2H, J = 7.4), 2.24 (m, 2H). Mass Spectrum (LCMS, ESI pos.) Calcd. |
| | For C₂₉H₂₉FN₆O₃: 529.59 (M + H), Found: 529.4. |
| 267 | 2-[3-(1-aza-bicyclo[2.2.2]oct-3-yloxy)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.98 (s, 1H), 9.36 (s, 1H), 8.82 (s, 1H), 7.80 (br, 1H), |
| | 7.40 (d, 1H, J = 7.9), 7.25 (s, 1H), 7.18 (d, 1H, J = 7.8), 7.10 (m, 1H), 6.91 (m, 1H), 6.70 (br, |
| | 1H), 6.54 (m, 1H), 4.26 (m, 1H), 3.90 (s, 3H), 3.22 (m, 1H), 3.04 (t, 2H, J = 7.4), 2.94 (m, |
| | 3H), 2.88 (m, 4H), 2.21 (p, 2H, J = 7.6), 2.11 (s, 1H), 1.93 (m, 1H), 1.72 (m, 1H), 1.53 (m, |
| | 1H), 1.37 (m, 1H). Mass Spectrum (LCMS, ESI pos.) Calcd. For C₃₁H₃₂N₆O₄: 553.64 (M + H), |
| | Found: 553.5. |
| 268 | 2-[4-(1-aza-bicyclo[2.2.2]oct-3-yloxy)-phenylamino]-8-(4-ethyl-phenyl)-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.34 (s, 1H), 8.81 (s, 1H), 7.70 (s, 1H), |
| | 7.42 (d, 2H, J = 8.3), 7.33 (d, 2H, J = 8.3), 7.21 (m, 2H), 6.58 (m, 2H), 4.26 (br, 1H), 3.90 (s, |
| | 3H), 3.22 (m, 1H), 2.96 (m, 1H), 2.85 (m, 6H), 2.10 (s, 1H), 1.94 (m, 1H), 1.74 (m, 1H), |
| | 1.53 (m, 1H), 1.38 (m, 4H). Mass Spectrum (LCMS, ESI pos.) Calcd. For C₃₀H₃₂N₆O₄: |
| | 541.63 (M + H), Found: 541.5. |
| 269 | 2-{4-[2-(3,3-difluoro-azetidin-1-yl)-ethyl]-phenylamino}-8-(4-ethyl-phenyl)-5-oxo-5,8- |
| | dihydro-pyrido[2,3-d]pyrimidine-6-carboxylic acid methoxy-amide |
| | ¹H NMR (400 MHz, CDCl₃) δ 11.98 (s, 1H), 9.35 (s, 1H), 8.83 (s, 1H), 7.89 (s, 1H), |
| | 7.43 (d, 2H, J = 8.3), 7.33 (d, 2H, J = 8.4), 7.26 (m, 2H), 6.92 (br, 2H), 3.90 (s, 3H), 3.53 (t, 4H, |
| | J = 12.0), 2.83 (q, 2H, J = 7.6), 2.75 (t, 2H, J = 7.2), 2.61 (t, 2H, J = 7.4), 1.39 (t, 3H, J = 7.6). |
| | Mass Spectrum (LCMS, ESI pos.) Calcd. For C₂₈H₂₈F₂N₆O₃: 535.57 (M + H), |
| | Found: 535.4. |
| 270 | 2-{4-[2-(3,3-difluoro-azetidin-1-yl)-ethyl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.99 (s, 1H), 9.34 (s, 1H), 8.84 (s, 1H), 7.88 (br, 1H), |
| | 7.42 (d, 1H, J = 7.9), 7.27 (m, 3H), 7.17 (d, 1H, J = 7.8), 6.91 (br, 2H), 3.90 (s, 3H), 3.54 (t, 4H, |
| | J = 12.0), 3.08 (m, 2H), 3.00 (t, 2H, J = 7.4), 2.75 (t, 2H, J = 7.4), 2.62 (t, 2H, J = 7.3), |
| | 2.24 (m, 2H). Mass Spectrum (LCMS, ESI pos.) Calcd. For C₂₉H₂₈F₂N₆O₃: 547.58 (M + H), |
| | Found: 547.4. |
| 271 | 8-indan-5-yl-2-[3-(1-methyl-piperidin-3-yl)-phenylamino]-5-oxo-5,8-dihydro-pyrido[2,3- |
| | d]pyrimidine-6-carboxylic acid methoxy-amide |
| | ¹H NMR (300 MHz, CDCl₃) δ (ppm): 11.89 (s, 1H), 9.32 (s, 1H), 8.74 (s, 1H), 7.39 (d, 1H, j = 10 Hz), |
| | 7.35 (d, 1H, j = 20 Hz), 7.15 (d, 1H, j = 10 Hz), 7.028 (t, 1H, j = 20 Hz), 6.85 (m, 1H), |
| | 6.48 (d, 1H, j = 20 Hz), 6.43 (m, 1H), 3.83 (s, 3H), 3.12 (bs, 2H), 2.997 (t, 2H), 2.993 (t, 2H), |
| | 2.43 (m, 2H), 2.33 (s, 3H), 2.15 (m, 2H), 2.095 (m, 1H), 1.78 (m, 4H). Mass Spectrum |
| | (LCMS, ESI pos.) Calcd. For C30H32N6O3: 524.61 (M + H), Found: 525. |
| 272 | 2-[3-(4-hydroxy-1-methyl-piperidin-4-yl)-phenylamino]-8-indan-5-yl-5-oxo-5,8-dihydro- |
| | pyrido[2,3-d]pyrimidine-6-carboxylic acid methoxy-amide |
| | ¹H NMR (300 MHz, CDCl₃) δ (ppm): 11.89 (s, 1H), 9.32 (s, 1H), 8.70 (s, 1H), 7.46 (d, 2H, j = 20 Hz), |
| | 7.32 (d, 2H, j = 20 Hz), 7.17 (m, 2H, j = 10 Hz), 7.06 (d, 2H, j = 20 Hz), 3.83 (s, 3H), |
| | 2.987 (t, 2H), 2.929 (t, 2H), 2.77 (s, 3H), 2.59 (m, 2H), 2.54 (m, 6H), 2.16 (m, 2H). Mass |
| | Spectrum (LCMS, ESI pos.) Calcd. For C30H32N6O4: 540.61 (M + H), Found: 541. |
| 273 | 8-indan-5-yl-2-[3-(1-methyl-piperidin-4-yl)-phenylamino]-5-oxo-5,8-dihydro-pyrido[2,3- |
| | d]pyrimidine-6-carboxylic acid methoxy-amide |
| | ¹H NMR (300 MHz, CDCl₃) δ (ppm): 11.89 (s, 1H), 9.32 (s, 1H), 8.74 (s, 1H), 7.47 (bs, 1H), |
| | 7.35 (d, 1H, j = 20 Hz), 7.11 (d, 1H, j = 10 Hz), 7.028 (t, 1H, j = 20 Hz), 6.85 (m, 1H), 6.48 (d, |
| | 1H, j = 20 Hz), 6.43 (m, 1H), 3.83 (s, 3H), 3.12 (bs, 2H), 2.997 (m, 8H), 2.33 (s, 3H), 2.15 (m, |
| | 2H), 2.095 (m, 1H), 1.78 (m, 4H). Mass Spectrum (LCMS, ESI pos.) Calcd. For |
| | C30H32N6O3: 524.61 (M + H), Found: 525. |
| 274 | 2-[3-(3-hydroxy-pyrrolidin-3-yl)-phenylamino]-8-indan-5-yl-5-oxo-5,8-dihydro- |
| | pyrido[2,3-d]pyrimidine-6-carboxylic acid methoxy-amide |
| | ¹H NMR (300 MHz, CDCl₃) δ (ppm): 11.89 (s, 1H), 9.32 (s, 1H), 8.74 (s, 1H), 7.595 (d, 1H, |
| | j = 10 Hz), 7.38 (d, 1H, j = 20 Hz ), 7.216 (m, 1H), 7.12 (d, 1H, j = 20 Hz), 7.061 (m, 1H), |
| | 3.83 (s, 3H), 3.61 (m, 2H), 3.39 (m, 2H), 3.03 (t, 2H), 2.96 (t, 2H), 2.28 (m, 2H), 2.19 (m, 2H). |
| | Mass Spectrum (LCMS, ESI pos.) Calcd. For C28H28N6O4: 512.56 (M + H), Found: |
| | 513. |
| 275 | 2-[3-(3-hydroxy-piperidin-3-yl)-phenylamino]-8-indan-5-yl-5-oxo-5,8-dihydro- |
| | pyrido[2,3-d]pyrimidine-6-carboxylic acid methoxy-amide |
| | ¹H NMR (300 MHz, CDCl₃) δ (ppm): 11.89 (s, 1H), 9.32 (s, 1H), 8.74 (s, 1H), 7.595 (d, 1H, |
| | j = 10 Hz), 7.38 (d, 1H, j = 20 Hz), 7.216 (m, 1H), 7.12 (d, 1H, j = 20 Hz), 7.061 (m, 1H), |
| | 3.83 (s, 3H), 3.61 (m, 2H), 3.39 (m, 2H), 3.03 (t, 2H), 2.96 (t, 2H), 2.28 (m, 2H), 2.19 (m, 4H). |
| | Mass Spectrum (LCMS, ESI pos.) Calcd. For C29H30N6O4: 526.59 (M + H), Found: |
| | 527. |
| 276 | 2-[3-(1-ethyl-3-hydroxy-piperidin-3-yl)-phenylamino]-8-indan-5-yl-5-oxo-5,8-dihydro- |
| | pyrido[2,3-d]pyrimidine-6-carboxylic acid methoxy-amide |
| | ¹H NMR (300 MHz, CDCl₃) δ (ppm): 11.89 (s, 1H), 9.29 (s, 1H), 8.72 (s, 1H), 7.52 (d, 1H, j = 20 Hz), |
| | 7.32 (d, 1H, j = 20 Hz), 7.186 (m, 1H), 7.057 (m, 1H), 6.864 (m, 1H), 6.76 (d, 1H, j = 20 Hz), |
| | 6.53 (d, 1H, j = 20 Hz), 3.83 (s, 3H), 2.98 (m, 2H), 2.98 (t, 2H), 2.92 (t, 2H), 2.52 (m, |
| | 4H), 2.15 (m, 2H), 2.02 (m, 4H), 1.06 (t, 3H). Mass Spectrum (LCMS, ESI pos.) Calcd. |
| | For C31H34N6O4: 554.64 (M + H), Found: 555. |
| 277 | 2-{3-[4-hydroxy-1-(2,2,2-trifluoro-ethyl)-piperidin-4-yl]-phenylamino}-8-indan-5-yl-5- |
| | oxo-5,8-dihydro-pyrido[2,3-d]pyrimidine-6-carboxylic acid methoxy-amide |
| | ¹H NMR (300 MHz, CDCl₃) δ (ppm): 11.89 (s, 1H), 9.29 (s, 1H), 8.72 (s, 1H), 7.52 (d, 1H, j = 20 Hz), |
| | 7.32 (d, 1H, j = 20 Hz), 7.186 (m, 1H), 7.057 (m, 1H), 6.864 (m, 1H), 6.76 (d, 1H, j = 20 Hz), |
| | 6.53 (d, 1H, j = 20 Hz), 3.83 (s, 3H), 3.29 (m, 2H), 3.08 (m, 2H), 3.07 (t, 2H), 3.00 (t, |
| | 2H), 2.46 (m, 2H), 2.22 (m, 2H), 1.70 (m, 4H). Mass Spectrum (LCMS, ESI pos.) Calcd. |
| | For C31H31F3N6O4: 608.61 (M + H), Found: 609. |
| 278 | 2-[3-(1-ethyl-3-hydroxy-piperidin-3-yl)-phenylamino]-8-(4-ethyl-phenyl)-5-oxo-5,8- |
| | dihydro-pyrido[2,3-d]pyrimidine-6-carboxylic acid methoxy-amide |
| | ¹H NMR (300 MHz, CDCl₃) δ (ppm): 9.35 (s, 1H), 8.77 (s, 1H), 7.39 (d, 2H, j = 20 Hz), |
| | 7.31 (d, 2H, j = 20 Hz), 7.10 (t, 1H), 6.93 (m, 1H), 6.80 (d, 1H, j = 20 Hz), 6.57 (d, 1H, j = 20 Hz), |
| | 3.87 (s, 3H), 3.05 (m, 2H), 2.79 (q, 2H), 2.57 (m, 4H), 2.11 (m, 4H), 1.34 (t, 3H), |
| | 1.12 (t, 3H). Mass Spectrum (LCMS, ESI pos.) Calcd. For C30H34N6O4: 542.63 (M + H), |
| | Found: 543. |
| 279 | 2-(1-acetyl-2,3-dihydro-1H-indol-5-ylamino)-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.94 (s, 1H), 9.33 (s, 1H), 8.79 (s, 1H), 7.43 (d, 2H, J = 8.3), |
| | 7.34 (d, 2H, J = 8.3), 7.08 (d, 1H, J = 8.7), 4.02 (t, 2H, J = 8.5), 3.90 (s, 3H), 2.97 (s, |
| | 1H), 2.82 (m, 2H), 2.21 (s, 2H), 2.11 (s, 3H), 1.38 (t, 3H, J = 7.6). Mass Spectrum |
| | (LCMS, ESI pos.) Calcd. For: C₂₇H₂₆N₆O₄: 498.5; Found: 499.5 (M + 1). |
| 280 | 8-(4-ethyl-phenyl)-2-(1-methanesulfonyl-2,3-dihydro-1H-indol-5-ylamino)-5-oxo-5,8- |
| | dihydro-pyrido[2,3-d]pyrimidine-6-carboxylic acid methoxy-amide |
| | ¹H NMR (400 MHz, CDCl₃) δ 11.92 (s, 1H), 9.35 (s, 1H), 8.79 (s, 1H), 7.43 (d, 2H, J = 8.3), |
| | 7.34 (d, 2H, J = 8.4), 3.94 (t, 2H, J = 8.4), 3.90 (s, 3H), 2.94 (m, 1H), 2.82 (m, 5H), |
| | 2.11 (m, 3H), 1.37 (t, 3H, J = 7.6). Mass Spectrum (LCMS, ESI pos.) Calcd. For: |
| | C₂₆H₂₆N₆O₅S: 534.6; Found: 535.8 (M + 1). |
| 281 | 2-(1-acetyl-2,3-dihydro-1H-indol-6-ylamino)-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.96 (s, 1H), 9.37 (s, 1H), 8.79 (s, 1H), 7.99 (m, 1H), |
| | 7.37 (dd, 4H, J = 8.3, 25.8), 4.06 (t, 2H, J = 8.5), 3.90 (s, 3H), 3.13 (t, 2H, J = 8.4), 2.81 (q, 2H, |
| | J = 7.6), 2.22 (s, 3H), 2.10 (s, 3H), 1.36 (t, 3H, J = 7.6). Mass Spectrum (LCMS, ESI pos.) |
| | Calcd. For: C₂₇H₂₆N₆O₄: 498.5; Found: 499.6 (M + 1). |
| 282 | 8-(4-ethyl-phenyl)-5-oxo-2-(1,2,3,4-tetrahydro-isoquinolin-7-ylamino)-5,8-dihydro- |
| | pyrido[2,3-d]pyrimidine-6-carboxylic acid methoxy-amide |
| | ¹H NMR (400 MHz, CDCl₃) δ 9.39 (m, 1H), 8.78 (m, 1H), 7.44 (m, 4H), 7.37 (m, 4H), |
| | 4.22 (m, 2H), 3.90 (m, 2H), 3.42 (m, 3H), 2.77 (m, 2H), 2.08 (m, 22H), 1.36 (m, 3H). |
| | Mass Spectrum (LCMS, ESI pos.) Calcd. For: C₂₆H₂₆N₆O₃: 470.5; Found: 471.3 (M + 1). |
| 283 | 8-indan-5-yl-5-oxo-2-(4-[1,2,4]triazol-1-ylmethyl-phenylamino)-5,8-dihydro-pyrido[2,3- |
| | d]pyrimidine-6-carboxylic acid methoxy-amide |
| | ¹H NMR (400 MHz, CDCl₃) δ 11.93 (s, 1H), 9.38 (s, 1H), 8.84 (s, 1H), 8.01 (d, 3H, J = 18.6), |
| | 7.40 (d, 3H, J = 8.0), 7.07 (d, 3H, J = 58.2), 5.28 (s, 2H), 3.89 (d, 3H, J = 9.1), |
| | 3.01 (m, 4H), 2.20 (m, 2H). Mass Spectrum (LCMS, ESI pos.) Calcd. For: C₂₇H₂₄N₈O₃: 508.5; |
| | Found: 509.1 (M + 1). |
| 284 | 8-indan-5-yl-5-oxo-2-(4-pyrazol-1-yl-phenylamino)-5,8-dihydro-pyrido[2,3-d]pyrimidine- |
| | 6-carboxylic acid methoxy-amide |
| | ¹H NMR (400 MHz, CDCl₃) δ 11.95 (s, 1H), 9.39 (s, 1H), 8.84 (s, 1H), 7.75 (m, 3H), |
| | 7.44 (d, 3H, J = 7.7), 7.17 (s, 1H), 3.89 (d, 3H, J = 11.0), 3.05 (dt, 4H, J = 7.3, 32.8), 2.24 (m, |
| | 2H). Mass Spectrum (LCMS, ESI pos.) Calcd. For: C₂₇H₂₃N₇O₃: 493.5; Found: |
| | 494.4 (M + 1). |
| 285 | 8-(4-ethyl-phenyl)-2-(1H-indol-5-ylamino)-5-oxo-5,8-dihydro-pyrido[2,3-d]pyrimidine-6- |
| | carboxylic acid methoxy-amide |
| | ¹H NMR (400 MHz, CDCl₃) δ 12.00 (s, 1H), 9.37 (s, 1H), 8.79 (s, 1H), 7.77 (s, 1H), |
| | 7.41 (d, 5H, J = 14.4), 3.90 (s, 3H), 2.85 (s, 2H), 1.38 (t, 3H, J = 7.6). Mass Spectrum (LCMS, |
| | ESI pos.) Calcd. For: C₂₅H₂₂N₆O₃: 454.5; Found: 455.3 (M + 1). |
| 286 | 8-(4-ethyl-phenyl)-2-(1H-indol-6-ylamino)-5-oxo-5,8-dihydro-pyrido[2,3-d]pyrimidine-6- |
| | carboxylic acid methoxy-amide |
| | ¹H NMR (400 MHz, CDCl₃) δ 11.98 (s, 1H), 9.37 (m, 1H), 8.80 (s, 1H), 7.45 (m, 6H, J = 17.1), |
| | 6.88 (m, 2H), 3.91 (s, 3H), 2.91 (m, 2H), 1.43 (m, 3H). Mass Spectrum (LCMS, |
| | ESI pos.) Calcd. For: C₂₅H₂₂N₆O₃: 454.5; Found: 455.2 (M + 1). |
| 287 | 8-indan-5-yl-2-(1H-indol-5-ylamino)-5-oxo-5,8-dihydro-pyrido[2,3-d]pyrimidine-6- |
| | carboxylic acid methoxy-amide |
| | ¹H NMR (400 MHz, CDCl₃) δ 12.00 (s, 1H), 9.35 (s, 1H), 8.80 (s, 1H), 7.79 (m, 3H), |
| | 7.42 (m, 3H), 7.05 (m, 1H), 3.90 (s, 3H), 3.05 (m, 4H), 2.22 (m, 3H). Mass Spectrum (LCMS, |
| | ESI pos.) Calcd. For: C₂₆H₂₂N₆O₃: 466.5; Found: 467.5 (M + 1). |
| 288 | 8-(4-ethyl-phenyl)-2-(1H-indazol-6-ylamino)-5-oxo-5,8-dihydro-pyrido[2,3-d]pyrimidine- |
| | 6-carboxylic acid methoxy-amide |
| | ¹H NMR (400 MHz, CDCl₃) δ 11.92 (s, 1H), 9.38 (s, 1H), 8.84 (s, 1H), 7.96 (s, 1H), |
| | 7.53 (m, 4H), 7.42 (m, 3H, J = 8.3), 6.98 (s, 1H, J = 8.7), 3.92 (s, 3H, J = 4.4), 2.92 (m, 2H), |
| | 1.42 (t, 3H, J = 7.7). Mass Spectrum (LCMS, ESI pos.) Calcd. For: C₂₄H₂₁N₇O₃: 455.5; |
| | Found: 456.5 (M + 1). |
| 289 | 8-indan-5-yl-2-(1H-indol-6-ylamino)-5-oxo-5,8-dihydro-pyrido[2,3-d]pyrimidine-6- |
| | carboxylic acid methoxy-amide |
| | ¹H NMR (400 MHz, CDCl₃) δ 11.99 (s, 1H), 9.40 (s, 1H), 8.81 (s, 1H), 7.52 (m, 6H), |
| | 6.98 (m, 2H), 3.91 (s, 3H), 2.99 (m, 4H), 2.25 (m, 2H). Mass Spectrum (LCMS, ESI pos.) |
| | Calcd. For: C₂₆H₂₂N₆O₃: 466.5; Found: 467.7 (M + 1). |
| 290 | 8-(4-ethyl-phenyl)-2-(1-methyl-1H-indol-5-ylamino)-5-oxo-5,8-dihydro-pyrido[2,3- |
| | d]pyrimidine-6-carboxylic acid methoxy-amide |
| | ¹H NMR (400 MHz, CDCl₃) δ 12.00 (s, 1H), 9.36 (s, 1H), 8.79 (s, 1H), 7.50 (m, 6H), |
| | 7.03 (m, 2H), 3.90 (s, 3H), 2.85 (m, 2H), 1.54 (s, 3H), 1.38 (t, 3H, J = 7.6). Mass Spectrum |
| | (LCMS, ESI pos.) Calcd. For: C₂₆H₂₄N₆O₃: 468.5; Found: 469.7 (M + 1). |
| 291 | 8-indan-5-yl-2-(1-methyl-1H-indol-5-ylamino)-5-oxo-5,8-dihydro-pyrido[2,3- |
| | d]pyrimidine-6-carboxylic acid methoxy-amide |
| | ¹H NMR (400 MHz, CDCl₃) δ 12.03 (s, 1H), 9.35 (s, 1H), 8.81 (s, 1H), 7.78 (m, 4H), |
| | 7.50 (m, 1H), 7.03 (s, 4H), 3.75 (s, 3H, J = 8.2), 3.05 (m, 4H), 2.20 (m, 5H). Mass Spectrum |
| | (LCMS, ESI pos.) Calcd. For: C₂₇H₂₄N₆O₃: 480.5; Found: 481.4 (M + 1). |
| 292 | 8-indan-5-yl-5-oxo-2-{4-[1-(2,2,2-trifluoro-ethyl)-piperidin-4-yl]-phenylamino}-5,8- |
| | dihydro-pyrido[2,3-d]pyrimidine-6-carboxylic acid methoxy-amide |
| | ¹H NMR (400 MHz, CDCl₃) δ 11.83 (s, 1H), 9.36 (s, 1H), 8.83 (s, 1H), 7.53 (m, 3H), |
| | 6.99 (m, 4H), 3.90 (s, 3H), 3.75 (m, 4H), 3.08 (dd, 5H, J = 18.2, 25.6), 2.68 (m, 2H), 2.23 (m, |
| | 2H), 1.80 (m, 4H). Mass Spectrum (LCMS, ESI pos.) Calcd. For: C₃₁H₃₁F₃N₆O₃: 592.6; |
| | Found: 593.2 (M + 1). |
| 293 | 8-indan-5-yl-5-oxo-2-{4-[1-(2,2,3,3,3-pentafluoro-propyl)-piperidin-4-yl]-phenylamino}- |
| | 5,8-dihydro-pyrido[2,3-d]pyrimidine-6-carboxylic acid methoxy-amide |
| | ¹H NMR (400 MHz, CDCl₃) δ 11.88 (s, 1H), 9.39 (s, 1H), 8.84 (s, 1H), 7.47 (m, 5H), |
| | 7.00 (m, 2H), 3.90 (s, 3H), 3.75 (m, 4H), 3.08 (dd, 4H, J = 19.5, 24.3), 2.68 (m, 3H), 2.28 (m, |
| | 1H), 1.86 (m, 4H), 1.42 (t, 10H, J = 7.3). Mass Spectrum (LCMS, ESI pos.) Calcd. For: |
| | C₃₂H₃₁F₅N₆O₃: 642.6; Found: 643.9 (M + 1). |
| 294 | 2-(6,7,9,10,12,13,15,16,18,19-decahydro-5,8,11,14,17,20-hexaoxa-benzocyclooctadecen- |
| | 2-ylamino)-8-(4-ethyl-phenyl)-5-oxo-5,8-dihydro-pyrido[2,3-d]pyrimidine-6-carboxylic |
| | acid methoxy-amide |
| | ¹H NMR (400 MHz, CDCl₃) δ 11.95 (s, 1H), 9.35 (s, 1H), 8.77 (s, 1H), 7.36 (dd, 4H, J = 8.3, |
| | 27.7), 3.89 (s, 3H), 3.73 (m, 20H), 2.80 (q, 2H, J = 7.5), 1.35 (t, 3H, J = 7.6). Mass |
| | Spectrum (LCMS, ESI pos.) Calcd. For: C₃₃H₃₉N₅O₉: 649.7; Found: 650.6 (M + 1). |
| 295 | 8-(4-ethyl-phenyl)-5-oxo-2-[4-(1-phenethyl-piperidin-4-yl)-phenylamino]-5,8-dihydro- |
| | pyrido[2,3-d]pyrimidine-6-carboxylic acid methoxy-amide |
| | ¹H NMR (400 MHz, CDCl₃) δ 11.95 (s, 1H), 9.37 (s, 1H), 8.83 (s, 1H), 7.32 (m, 4H), |
| | 7.22 (m, 4H, J = 6.9), 3.90 (s, 3H), 3.05 (m, 10H), 2.74 (m, 3H), 2.24 (s, 3H), 2.03 (s, 2H). |
| | Mass Spectrum (LCMS, ESI pos.) Calcd. For: C₃₆H₃₈N₆O₃: 602.7; Found: 603.6 (M + 1). |
| 296 | 8-(4-ethyl-phenyl)-5-oxo-2-{4-[(2S,3S,4S,5S,6R)(3,4,5-trihydroxy-6-hydroxymethyl- |
| | tetrahydro-pyran-2-yloxy)]-phenylamino}-5,8-dihydro-pyrido[2,3-d]pyrimidine-6- |
| | carboxylic acid methoxy-amide ¹H NMR (400 MHz, DMSO) δ 11.90 (s, 1H), 10.43 (s, |
| | 1H), 9.22 (s, 1H), 8.50 (s, 1H), 7.46 (m, 4H), 7.33 (m, 4H), 6.75 (s, 1H), 5.18 (s, 1H), |
| | 3.72 (s, 3H), 3.48 (m, 7H), 2.76 (m, 2H), 2.48 (m, 4H), 1.28 (t, 3H, J = 7.6). Mass Spectrum |
| | (LCMS, ESI pos.) Calcd. For: C₂₉H₃₁N₅O₉: 593.6; Found: 594.4 (M + 1). |
| 297 | 8-(4-ethyl-phenyl)-2-(6,7,9,10,12,13,15,16-octahydro-5,8,11,14,17-pentaoxa- |
| | benzocyclopentadecen-2-ylamino)-5-oxo-5,8-dihydro-pyrido[2,3-d]pyrimidine-6- |
| | carboxylic acid methoxy-amide |
| | ¹H NMR (400 MHz, CDCl₃) δ 11.95 (s, 1H), 9.34 (s, 1H), 8.76 (s, 1H), 7.36 (m, 7H, J = 8.4, |
| | 25.8), 4.07 (s, 2H), 3.87 (m, 7H), 3.74 (s, 10H), 2.80 (q, 2H, J = 7.6), 1.34 (t, 3H, J = 7.6). |
| | Mass Spectrum (LCMS, ESI pos.) Calcd. For: C₃₁H₃₅N₅O₈: 605.7; Found: |
| | 606.8 (M + 1). |
| 298 | 8-indan-5-yl-5-oxo-2-[4-(1-propyl-piperidin-4-yl)-phenylamino]-5,8-dihydro-pyrido[2,3- |
| | d]pyrimidine-6-carboxylic acid methoxy-amide |
| | ¹H NMR (400 MHz, CDCl₃) δ 11.94 (s, 1H), 9.37 (s, 1H), 8.83 (s, 1H), 7.44 (m, 4H, J = 7.5), |
| | 7.18 (m, 3H), 3.90 (s, 3H), 3.68 (d, 2H, J = 11.8), 3.13 (m, 2H), 2.99 (dd, 5H, J = 10.1, |
| | 17.7), 2.65 (s, 4H), 2.26 (m, 2H), 2.02 (m, 4H), 1.04 (t, 3H, J = 7.4). Mass Spectrum |
| | (LCMS, ESI pos.) Calcd. For: C₃₂H₃₆N₆O₃: 552.7; Found: 553.7 (M + 1). |
| 299 | 8-(4-ethyl-phenyl)-2-(3′-methoxy-biphenyl-4-ylamino)-5-oxo-5,8-dihydro-pyrido[2,3- |
| | d]pyrimidine-6-carboxylic acid methoxy-amide |
| | ¹H NMR (400 MHz, CDCl₃) δ 11.95 (s, 1H), 9.37 (s, 1H), 8.84 (s, 1H), 7.39 (dt, 10H, J = 8.2, |
| | 17.6), 7.09 (d, 1H, J = 7.7), 6.89 (dd, 1H, J = 2.2, 7.8), 3.91 (s, 3H), 3.84 (s, 3H), |
| | 2.84 (q, 2H, J = 7.6), 2.13 (s, 1H, J = 1.6), 1.39 (t, 3H, J = 7.6). Mass Spectrum (LCMS, ESI |
| | pos.) Calcd. For: C₃₀H₂₇N₅O₄: 521.6; Found: 522.8 (M + 1). |
| 300 | 8-indan-5-yl-5-oxo-2-phenylamino-5,8-dihydro-pyrido[2,3-d]pyrimidine-6-carboxylic |
| | acid methoxy-amide |
| | ¹H NMR (400 MHz, CDCl₃) δ 11.96 (s, 1H), 9.36 (s, 1H), 8.84 (s, 1H), 7.43 (d, 2H, J = 7.9), |
| | 7.34 (m, 2H), 7.17 (m, 2H), 7.10 (s, 1H), 7.02 (s, 1H), 3.91 (s, 3H), 3.08 (t, 2H, J = 7.4), |
| | 3.01 (t, 2H, J = 7.4), 2.23 (m, 2H), 2.12 (s, 1H). Mass Spectrum (LCMS, ESI pos.) |
| | Calcd. For: C₂₄H₂₁N₅O₃: 427.5; Found: 428.1 (M + 1). |
| 301 | 2-(1-ethyl-1H-benzoimidazol-5-ylamino)-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.99 (s, 1H), 9.39 (s, 1H), 8.82 (s, 1H), 7.88 (s, 1H), |
| | 7.43 (m, 3H), 7.18 (m, 3H), 7.00 (s, 1H), 4.20 (m, 2H), 3.90 (s, 3H), 3.49 (s, 2H), 3.02 (m, 4H), |
| | 2.22 (dd, 2H, J = 7.5, 14.9), 1.55 (m, 3H). Mass Spectrum (LCMS, ESI pos.) Calcd. For: |
| | C₂₇H₂₅N₇O₃: 495.5; Found: 496.1 (M + 1). |
| 302 | 2-(1H-benzoimidazol-5-ylamino)-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.81 (s, 1H), 9.66 (s, 1H), 8.98 (s, 1H), 8.94 (s, 1H), |
| | 7.50 (m, 2H), 7.32 (m, 2H), 7.21 (m, 2H), 7.05 (s, 1H, J = 2.2), 3.93 (s, 3H), 3.12 (t, 2H, J = 7.6), |
| | 3.05 (t, 2H, J = 7.5), 2.28 (m, 2H). Mass Spectrum (LCMS, ESI pos.) Calcd. For: |
| | C₂₅H₂₁N₇O₃: 467.5; Found: 468.2 (M + 1). |
| 303 | 2-[4-(1-methyl-piperidin-4-yl)-phenylamino]-8-naphthalen-2-yl-5-oxo-5,8-dihydro- |
| | pyrido[2,3-d]pyrimidine-6-carboxylic acid methoxy-amide |
| | ¹H NMR (400 MHz, CDCl₃) δ 11.96 (s, 1H), 9.40 (s, 1H), 8.96 (s, 1H), 8.08 (d, 4H, J = 8.8), |
| | 7.92 (d, 4H, J = 10.7), 7.66 (m, 2H), 7.51 (m, 1H), 3.91 (s, 3H, J = 10.8), 2.83 (s, 3H, |
| | J = 4.0), 2.70 (m, 1H), 2.12 (m, 4H), 1.75 (m, 4H). Mass Spectrum (LCMS, ESI pos.) |
| | Calcd. For: C₃₁H₃₀N₆O₃: 534.6; Found: 535.3 (M + 1). |
| 304 | 8-(4-ethyl-phenyl)-5-oxo-2-(2-oxo-2,3-dihydro-1H-indol-5-ylamino)-5,8-dihydro- |
| | pyrido[2,3-d]pyrimidine-6-carboxylic acid methoxy-amide |
| | ¹H NMR (400 MHz, CDCl₃) δ 11.92 (s, 1H), 9.32 (s, 1H), 8.78 (s, 1H), 8.01 (s, 1H), |
| | 7.43 (d, 2H, J = 8.1), 7.34 (d, 2H, J = 8.3), 7.26 (m, 3H), 3.90 (s, 3H), 3.30 (s, 2H), 2.85 (m, |
| | 2H), 1.37 (t, 3H, J = 7.6). Mass Spectrum (LCMS, ESI pos.) Calcd. For: C₂₅H₂₂N₆O₄: |
| | 470.5; Found: 471.8 (M + 1). |
| 305 | 8-(4-ethyl-phenyl)-5-oxo-2-(3,4,5-trimethoxy-phenylamino)-5,8-dihydro-pyrido[2,3- |
| | d]pyrimidine-6-carboxylic acid methoxy-amide |
| | ¹H NMR (400 MHz, CDCl₃) δ 11.95 (s, 1H), 9.38 (s, 1H), 8.72 (s, 1H), 7.34 (m, 4H), |
| | 6.65 (m, 2H), 3.90 (s, 6H), 3.79 (s, 3H), 2.77 (q, 2H, J = 7.6), 1.33 (t, 3H, J = 7.6). Mass |
| | Spectrum (LCMS, ESI pos.) Calcd. For: C₂₆H₂₇N₅O₆: 505.5; Found: 506.9 (M + 1). |
| 306 | 2-(4′-dimethylamino-biphenyl-4-ylamino)-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.97 (s, 1H), 9.37 (s, 1H), 8.83 (s, 1H), 7.41 (m, 10H), |
| | 6.81 (s, 2H), 3.91 (s, 3H), 3.01 (s, 6H), 2.84 (m, 2H), 1.40 (t, 3H, J = 7.6). Mass Spectrum |
| | (LCMS, ESI pos.) Calcd. For: C₃₁H₃₀N₆O₃: 534.6; Found: 535.2 (M + 1). |
| 307 | 8-(4-ethyl-phenyl)-5-oxo-2-[4-(piperidin-4-yloxy)-phenylamino]-5,8-dihydro-pyrido[2,3- |
| | d]pyrimidine-6-carboxylic acid methoxy-amide |
| | ¹H NMR (400 MHz, CDCl₃) δ 11.94 (s, 1H), 9.32 (s, 1H), 8.81 (s, 1H), 7.41 (m, 8H), |
| | 3.90 (s, 3H), 3.28 (m, 5H), 2.84 (m, 4H), 2.11 (m, 4H), 1.38 (t, 3H, J = 7.6). Mass Spectrum |
| | (LCMS, ESI pos.) Calcd. For: C₂₈H₃₀N₆O₄: 514.6; Found: 515.5 (M + 1). |
| 308 | 2-[3,4-bis-(2-methoxy-ethoxy)-phenylamino]-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.96 (s, 1H), 9.37 (s, 1H), 8.77 (s, 1H), 7.36 (dd, 6H, J = 8.3, |
| | 27.8), 7.00 (s, 1H), 4.05 (m, 4H), 3.90 (s, 3H), 3.66 (m, 4H), 3.44 (d, 6H, J = 5.8), |
| | 2.79 (m, 2H), 1.34 (t, 3H, J = 7.6). Mass Spectrum (LCMS, ESI pos.) Calcd. For: |
| | C₂₉H₃₃N₅O₇: 563.6; Found: 564.4 (M + 1). |
| 309 | 8-(4-ethyl-phenyl)-2-[4-(1-methyl-piperidin-4-yloxy)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 11.96 (s, 1H), 9.74 (s, 1H), 9.37 (s, 1H), 8.81 (s, 1H), |
| | 7.42 (m, 4H), 7.24 (m, 4H), 4.62 (m, 1H), 3.90 (s, 3H), 3.30 (m, 4H), 2.81 (m, 3H, J = 4.8), |
| | 2.30 (m, 2H), 2.11 (m, 2H), 1.39 (s, 3H). Mass Spectrum (LCMS, ESI pos.) Calcd. For: |
| | C₂₉H₃₂N₆O₄: 528.6; Found: 529.7 (M + 1). |
| 310 | 8-(4-ethyl-phenyl)-2-[4-(1-ethyl-piperidin-4-yloxy)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 11.95 (s, 1H), 9.37 (s, 1H), 8.81 (s, 1H), 7.36 (m, 8H), |
| | 6.61 (s, 1H), 4.59 (m, 1H), 3.90 (s, 3H), 3.39 (m, 2H), 3.10 (m, 4H), 2.82 (d, 2H, J = 7.6), |
| | 2.68 (m, 1H), 2.14 (d, 2H, J = 14.7), 1.53 (dd, 6H, J = 13.2, 20.6), 1.37 (br s, 2H). Mass |
| | Spectrum (LCMS, ESI pos.) Calcd. For: C₃₀H₃₄N₆O₄: 542.6; Found: 543.6 (M + 1). |
| 311 | 8-indan-5-yl-5-oxo-2-[4-(piperidin-4-yloxy)-phenylamino]-5,8-dihydro-pyrido[2,3- |
| | d]pyrimidine-6-carboxylic acid methoxy-amide |
| | ¹H NMR (400 MHz, CDCl₃) δ 9.34 (s, 1H), 8.81 (s, 1H), 7.44 (d, 2H, J = 7.8), 7.29 (m, |
| | 4H), 7.18 (d, 1H, J = 7.6), 6.63 (s, 1H), 4.55 (br s, 1H), 3.89 (s, 3H), 3.41 (dt, 1H, J = 1.7, |
| | 3.3), 3.33 (m, 2H), 3.22 (d, 2H, J = 12.9), 3.03 (dd, 4H, J = 14.0, 21.5), 2.23 (m, 3H), |
| | 2.13 (m, 2H). Mass Spectrum (LCMS, ESI pos.) Calcd. For: C₂₉H₃₀N₆O₄: 526.6; Found: |
| | 527.6 (M + 1). |
| 312 | 2-[4-(1-ethyl-piperidin-4-yloxy)-phenylamino]-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ 11.95 (s, 1H), 9.37 (s, 1H), 8.82 (s, 1H), 7.52 (s, 1H), |
| | 7.42 (d, 2H, J = 8.0), 7.17 (d, 4H, J = 7.0), 4.59 (m, 1H), 3.90 (s, 3H), 3.39 (m, 2H), 3.07 (m, |
| | 8H), 2.70 (s, 2H), 2.21 (dd, 4H, J = 11.2, 18.5), 1.52 (t, 3H, J = 7.3). Mass Spectrum |
| | (LCMS, ESI pos.) Calcd. For: C₃₁H₃₄N₆O₄: 554.7; Found: 555.7 (M + 1). |
| 313 | 8-indan-5-yl-2-[4-(1-methyl-piperidin-4-yloxy)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 11.95 (s, 1H), 9.72 (br s, 1H), 9.36 (s, 1H), 8.82 (s, 1H), |
| | 7.43 (s, 1H), 7.19 (m, 6H), 4.59 (m, 1H), 3.90 (s, 3H), 3.35 (s, 2H), 3.04 (m, 4H), 2.24 (d, |
| | 4H, J = 7.5), 2.11 (s, 3H), 1.58 (br s, 4H). Mass Spectrum (LCMS, ESI pos.) Calcd. For: |
| | C₃₀H₃₂N₆O₄: 540.6; Found: 541.3 (M + 1). |
| 314 | 2-[3,4-bis-(2-methoxy-ethoxy)-phenylamino]-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.97 (s, 1H), 9.36 (s, 1H), 8.78 (s, 1H), 7.88 (s, 1H), |
| | 7.39 (d, 4H, J = 8.0), 7.17 (d, 1H, J = 8.0), 6.98 (m, 2H), 4.23 (d, 2H, J = 9.9), 4.10 (s, 2H), |
| | 3.90 (s, 3H), 3.72 (dd, 4H, J = 10.7, 15.5), 3.46 (m, 6H), 3.04 (t, 2H, J = 7.4), 2.97 (m, |
| | 2H), 2.20 (m, 2H). Mass Spectrum (LCMS, ESI pos.) Calcd. For: C₃₀H₃₃N₅O₇: 575.6; |
| | Found: 576.6 (M + 1). |
| 315 | 8-indan-5-yl-5-oxo-2-[4-(2aR,1R,7S,6aS)-(1,4,4,7-tetramethyl-3,5,11-trioxa- |
| | tricyclo[5.3.1.0^{2,6}]undec-9-yl)-phenylamino]-5,8-dihydro-pyrido[2,3-d]pyrimidine-6- |
| | carboxylic acid methoxy-amide |
| | ¹H NMR (400 MHz, CDCl₃) δ 11.96 (s, 1H), 9.32 (s, 1H), 8.84 (s, 1H), 7.44 (m, 2H), |
| | 7.17 (m, 2H), 6.92 (m, 3H), 4.50 (s, 1H), 4.28 (s, 2H), 3.90 (s, 3H), 3.06 (d, 6H, J = 33.1), |
| | 2.26 (br s, 2H), 1.93 (d, 2H, J = 6.8), 1.53 (m, 6H), 1.33 (m, 10H). Mass Spectrum (LCMS, |
| | ESI pos.) Calcd. For: C₃₆H₃₉N₅O₆: 637.7; Found: 638.9 (M + 1). |
| 316 | 8-(4-ethyl-phenyl)-5-oxo-2-phenylamino-5,8-dihydro-pyrido[2,3-d]pyrimidine-6- |
| | carboxylic acid methoxy-amide |
| | ¹H NMR (400 MHz, CDCl₃) δ 9.39 (s, 1H), 8.83 (s, 1H), 7.45 (d, 3H, J = 8.2), 7.36 (d, 4H, |
| | J = 8.2), 7.07 (m, 2H), 3.91 (s, 3H), 2.84 (m, 2H), 1.39 (t, 3H, J = 7.6). Mass Spectrum |
| | (LCMS, ESI pos.) Calcd. For: C₂₃H₂₁N₅O₃: 415.5; Found: 416.5 (M + 1). |
| 317 | 8-(4-ethyl-phenyl)-5-oxo-2-(4-pyrrol-1-yl-phenylamino)-5,8-dihydro-pyrido[2,3- |
| | d]pyrimidine-6-carboxylic acid methoxy-amide |
| | ¹H NMR (400 MHz, CDCl₃) δ 11.95 (s, 1H), 9.40 (s, 1H), 8.83 (s, 1H), 7.45 (d, 4H, J = 8.4), |
| | 7.36 (d, 4H, J = 5.9), 6.99 (d, 2H, J = 3.1), 6.35 (m, 2H), 3.91 (s, 3H), 2.83 (q, 2H, J = 7.5), |
| | 1.39 (t, 3H, J = 7.6). Mass Spectrum (LCMS, ESI pos.) Calcd. For: C₂₇H₂₄N₆O₃: |
| | 480.5; Found: 481.1 (M + 1). |
| 318 | 8-(4-ethyl-phenyl)-5-oxo-2-{4-[1-(2,2,2-trifluoro-ethyl)-piperidin-4-yloxy]-phenylamino}- |
| | 5,8-dihydro-pyrido[2,3-d]pyrimidine-6-carboxylic acid methoxy-amide |
| | ¹H NMR (400 MHz, CDCl₃) δ 11.45 (s, 1H), 10.79 (s, 1H), 9.20 (s, 1H), 8.84 (s, 1H), |
| | 7.46 (m, 5H), 7.32 (m, 3H), 3.90 (s, 3H), 3.73 (d, 1H, J = 9.1), 3.55 (m, 2H), 3.40 (m, 2H), |
| | 2.83 (m, 4H), 2.18 (m, 4H), 1.40 (m, 3H). Mass Spectrum (LCMS, ESI pos.) Calcd. For: |
| | C₃₀H₃₁F₃N₆O₄: 596.6; Found: 597.2 (M + 1). |
| 319 | 2-(3-dimethylamino-phenylamino)-8-(4-ethyl-phenyl)-5-oxo-5,8-dihydro-pyrido[2,3- |
| | d]pyrimidine-6-carboxylic acid methoxy-amide |
| | ¹H NMR (400 MHz, CDCl₃) δ 11.94 (s, 1H), 9.40 (s, 1H), 8.79 (m, 1H), 7.42 (d, 2H, J = 8.6), |
| | 7.34 (d, 2H, J = 8.4), 7.25 (m, 3H), 3.90 (s, 3H), 2.93 (br s, 6H), 2.80 (q, 2H, J = 7.6), |
| | 1.36 (t, 3H, J = 7.6). Mass Spectrum (LCMS, ESI pos.) Calcd. For: C₂₅H₂₆N₆O₃: 458.5; |
| | Found: 459.9 (M + 1). |
| 320 | 8-(4-ethyl-phenyl)-2-(3-morpholin-4-yl-phenylamino)-5-oxo-5,8-dihydro-pyrido[2,3- |
| | d]pyrimidine-6-carboxylic acid methoxy-amide ¹H NMR (400 MHz, CDCl₃) δ 11.95 (s, |
| | 1H), 9.36 (s, 1H), 8.79 (s, 1H), 7.40 (d, 2H, J = 8.6), 7.33 (m, 4H), 7.02 (t, 3H, J = 9.2), |
| | 3.91 (s, 3H), 3.81 (m, 4H), 3.02 (br s, 4H), 2.81 (q, 2H, J = 7.6), 1.35 (t, 3H, J = 7.6). |
| | Mass Spectrum (LCMS, ESI pos.) Calcd. For: C₂₇H₂₈N₆O₄: 500.6; Found: 501.3 (M + 1). |
| 321 | 8-(4-ethyl-phenyl)-2-[3-(4-methyl-piperazin-1-yl)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 11.94 (s, 1H), 9.40 (s, 1H), 8.80 (s, 1H), 7.32 (m, 8H), |
| | 3.90 (s, 3H), 3.60 (m, 2H), 3.31 (m, 2H), 2.95 (s, 2H), 2.85 (s, 3H), 2.81 (q, 2H, J = 7.6), |
| | 1.66 (m, 4H), 1.35 (t, 3H, J = 7.6). Mass Spectrum (LCMS, ESI pos.) Calcd. For: C₂₈H₃₁N₇O₃: |
| | 513.6; Found: 514.4 (M + 1). |
| 322 | 8-(4-ethyl-phenyl)-2-[3-(1-ethyl-piperidin-4-yloxy)-phenylamino]-5-oxo-5,8-dihydro- |
| | pyrido[2,3-d]pyrimidine-6-carboxylic acid methoxy-amide |
| | ¹H NMR (400 MHz, CDCl₃) δ 9.39 (s, 1H), 9.31 (s, 1H), 8.83 (s, 1H), 7.44 (m, 4H), |
| | 7.33 (m, 3H), 6.61 (s, 1H), 4.59 (m, 1H), 3.89 (s, 3H), 3.37 (m, 2H), 3.11 (m, 2H), 2.82 (m, |
| | 4H), 1.52 (m, 4H), 1.50 (t, 3H, J = 7.4), 1.36 (m, 3H). Mass Spectrum (LCMS, ESI pos.) |
| | Calcd. For: C₃₀H₃₄N₆O₄: 542.6; Found: 543.4 (M + 1). |
| 323 | 2-[3,4-bis-(3-methoxy-propoxy)-phenylamino]-8-(4-ethyl-phenyl)-5-oxo-5,8-dihydro- |
| | pyrido[2,3-d]pyrimidine-6-carboxylic acid methoxy-amide |
| | ¹H NMR (400 MHz, CDCl₃) δ 11.97 (s, 1H), 9.36 (s, 1H), 8.78 (s, 1H), 7.31 (m, 6H), |
| | 6.98 (m, 1H), 3.90 (s, 3H), 3.56 (dd, 4H, J = 5.3, 11.5), 3.35 (d, 6H, J = 7.0), 2.80 (q, 2H, J = 7.5), |
| | 2.05 (m, 4H), 1.65 (br s, 4H), 1.33 (m, 3H). Mass Spectrum (LCMS, ESI pos.) |
| | Calcd. For: C₃₁H₃₇N₅O₇: 591.7; Found: 592.5 (M + 1). |
| 324 | 8-indan-5-yl-2-[3-(4-methyl-piperazin-1-yl)-phenylamino]-5-oxo-5,8-dihydro-pyrido[2,3- |
| | d]pyrimidine-6-carboxylic acid methoxy-amide |
| | ¹H NMR (400 MHz, CDCl₃) δ 11.95 (s, 1H), 9.38 (s, 1H), 8.81 (s, 1H), 7.41 (m, 4H), |
| | 7.16 (m, 2H, J = 7.8), 6.61 (s, 1H), 3.90 (s, 3H), 3.62 (m, 4H), 3.30 (m, 2H), 3.05 (t, 2H, J = 7.3), |
| | 2.98 (t, 2H, J = 7.4), 2.87 (s, 3H), 2.22 (dd, 2H, J = 7.4, 14.9), 2.01 (br s, 2H). Mass |
| | Spectrum (LCMS, ESI pos.) Calcd. For: C₂₉H₃₁N₇O₃: 525.6; Found: 526.6 (M + 1). |
| 325 | 8-indan-5-yl-2-(3-morpholin-4-yl-phenylamino)-5-oxo-5,8-dihydro-pyrido[2,3- |
| | d]pyrimidine-6-carboxylic acid methoxy-amide |
| | ¹H NMR (400 MHz, CDCl₃) δ 11.97 (s, 1H), 9.37 (s, 1H), 8.81 (s, 1H), 7.40 (m, 4H), |
| | 7.16 (m, 3H), 3.90 (m, 3H), 3.88 (br s, 2H), 3.07 (m, 4H), 2.99 (t, 2H, J = 7.5), 2.41 (m, 4H), |
| | 2.22 (m, 2H). Mass Spectrum (LCMS, ESI pos.) Calcd. For: C₂₈H₂₈N₆O₄: 512.6; Found: |
| | 513.5 (M + 1). |
| 326 | 2-(3-dimethylamino-phenylamino)-8-indan-5-yl-5-oxo-5,8-dihydro-pyrido[2,3- |
| | d]pyrimidine-6-carboxylic acid methoxy-amide |
| | ¹H NMR (400 MHz, CDCl₃) δ 11.95 (s, 1H), 9.41 (s, 1H), 8.79 (s, 1H, J = 0.6), 7.41 (m, |
| | 4H), 7.18 (m, 3H), 3.90 (s, 3H), 3.05 (t, 2H, J = 7.5), 2.99 (t, 2H, J = 7.5), 2.91 (s, 6H), |
| | 2.22 (m, 2H). Mass Spectrum (LCMS, ESI pos.) Calcd. For: C₂₆H₂₆N₆O₃: 470.5; Found: |
| | 471.1 (M + 1). |

### Example 34

### 8-indan-5-yl-2-[4-(1-methyl-piperidin-4-yl)-phenylamino]-5-oxo-5,8-dihydro-pyrido[2,3-d]pyrimidine-6-carboxylic acid methoxy-amide (Cpd 138)

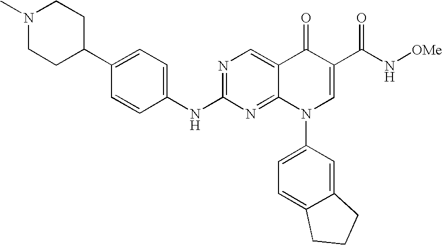

### A. 8-indan-5-yl-2-[4-(1-methyl-piperidin-4-yl)-phenylamino]-5-oxo-5,8-dihydro-pyrido[2,3-d]pyrimidine-6-carboxylic acid methyl ester

A solution of 8-indan-5-yl-2-methanesulfonyl-5-oxo-5,8-dihydro-pyrido[2,3-d]pyrimidine-6-carboxylic acid methyl ester (75 mg, 0.19 mmol) and 4-(1-methyl-piperidin-4-yl)-phenylamine (90 mg, 0.46 mmol) in AcOH (1 mL) was heated at 110° C. After 10 min, the reaction mixture was concentrated and the residue was purified (HPLC, C-18 YMC ODS-A 5μ 30×100 mm, 120 A column at 32 mL/min, 5-100% H₂O/MeCN (0.1% TFA v/v) gradient over 10 min.) to give the title compound (30 mg). ¹H NMR (TFA salt) (400 MHz, CDCl₃) δ (ppm): 9.29 (s, 1H), 8.58 (s, 1H), 7.42 (d, J=7.92 Hz, 2H), 7.33-7.27 (m, 3H), 7.16 (dd, J=7.87, 2.08 Hz, 2H), 3.88 (s, 3H), 3.71 (d, J=11.30 Hz, 2H), 3.06 (t, J=7.36 Hz, 2H), 2.98 (d, J=7.40 Hz, 2H), 2.87 (d, J=15.45 Hz, 2H), 2.66 (t, 1H), 2.24-2.08 (m, 2H), 1.97 (d, J=13.92 Hz, 2H); Mass Spectrum (LCMS, APCI pos.) Calcd. For: C₃₀H₃₁N₅O₃: 509.24; Found: 510.2 (M+H).

### B. 8-indan-5-yl-2-[4-(1-methyl-piperidin-4-yl)-phenylamino]-5-oxo-5,8-dihydro-pyrido[2,3-d]pyrimidine-6-carboxylic acid methoxy-amide

A solution of 8-indan-5-yl-2-[4-(1-methyl-piperidin-4-yl)-phenylamino]-5-oxo-5,8-dihydro-pyrido[2,3-d]pyrimidine-6-carboxylic acid methyl ester (15 mg) and O-methyl-hydroxylamine (0.5 g) in 1:1 TEA and MeOH (2 mL) in a sealed tube was heated at 110° C. for 2 days. After cooling, water was added and the solution was directly purified (HPLC, C-18 YMC ODS-A 5μ 30×100 mm, 120 A column at 32 mL/min, 5-100% H₂O/MeCN (0.1% TFA v/v) gradient over 10 min.) to provide the title compound (4.5 mg). ¹H NMR (TFA salt) (400 MHz, CDCl₃) δ (ppm): 9.39 (s, 1H), 8.87 (s, 1H), 7.48-7.39 (m, 2H), 7.34-7.29 (m, 3H), 7.21-7.16 (m, 2H), 3.91 (s, 3H), 3.78-3.67 (m, 2H), 3.14-3.05 (m, 2H), 2.99 (t, J=7.37 Hz, 2H), 2.85 (d, J=3.92 Hz, 2H), 2.71-2.61 (m, 1H), 2.31-2.16 (m, 2H), 2.06-1.91 (m, 2H); Mass Spectrum (LCMS, APCI pos.) Calcd. For: C₃₀H₃₂N₆O₃: 524.25; Found: 526.2 (M+H).

### Example 35

### 8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide (Cpd 95)

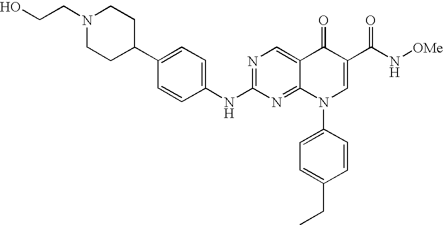

### A. 8-(4-ethyl-phenyl)-5-oxo-2-(4-piperidin-4-yl-phenylamino)-5,8-dihydro-pyrido[2,3-d]pyrimidine-6-carboxylic acid methoxy-amide

A mixture of 8-(4-ethyl-phenyl)-2-methanesulfonyl-5-oxo-5,8-dihydro-pyrido[2,3-d]pyrimidine-6-carboxylic acid methoxy-amide (Example 33, Step D, 1.79 g, 4.45 mmol) and 4-(4-amino-phenyl)-piperidine-1-carboxylic acid tert-butyl ester (Example 4, Step B) (1.32 g, 4.78 mmol) in AcOH (10 mL) was heated at 110° C. for 15 min. The reaction mixture was concentrated, and methanol (10 mL) was added to the remaining residue. The resulting precipitates were collected by filtration and washed with methanol. The yellow solid was treated with TFA/CH₂Cl₂ (10 mL, 1:1 v/v) at rt for 2 h and the solvent was evaporated under vacuo. The residue was redissolved in CH₂Cl₂ (200 mL), and washed with sat. NaHCO₃ solution and brine. The organic layer was dried (Na₂SO₄) filtered, and the filtrate was concentrated to provide the title compound as a yellow solid (0.9 g), which was used in the next step without further purification.

### B. 8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide

A solution of 8-(4-ethyl-phenyl)-5-oxo-2-(4-piperidin-4-yl-phenylamino)-5,8-dihydro-pyrido[2,3-d]pyrimidine-6-carboxylic acid methoxy-amide (0.9 g, 1.8 mmol), 2-bromoethanol (200 μL, 2.16 mmol) and triethylamine (500 μL, 3.6 mmol) in DMSO (10 mL) was heated for 3 h at 60° C. After cooling, water (100 mL) was added, and the aqueous layer was extracted with CH₂Cl₂ (2×100 mL). The organic layer was washed with sat. NaHCO₃ solution and brine, and was dried (Na₂SO₄) filtered, and the filtrate was concentrated in vacuo. The residue was purified by chromatography (silica gel, 0.7 N NH₃ in CH₃OH/CH₂Cl₂ 0.1:10-1:10 v/v) to provide the title compound as a yellow solid, which was converted to a HCl salt (0.5 g, 48%). ¹H NMR (400 MHz, CDCl₃) δ (ppm): 11.96 (s, 1H), 9.35 (s, 1H), 8.80 (s, 1H), 7.79 (br, 1H), 7.40 (d, 2H), 7.30 (d, 2H), 7.25 (br, 2H), 6.93 (br, 2H), 3.90 (s, 3H), 3.62 (t, 2H), 3.00 (d, 2H), 2.92 (br, 1H), 2.81 (q, 2H), 2.59 (t, 2H), 2.42 (m, 1H), 2.17 (t, 2H), 1.60-1.80 (m, 4H), 1.38 (t, 3H). Mass Spectrum (LCMS, ESI pos.) Calcd. For C₃₀H₃₄N₆O₄: 543.26 (M+H), Found 543.3.

Using the procedure of Example 35 and reagents, starting materials and conditions known to those skilled in the art, the following compounds representative of the present invention were prepared:

| Cpd | Name and Data |
|---|---|
| 88 | 2-{4-[1-(2-hydroxy-ethyl)-piperidin-4-yl]-phenylamino}-8-indan-5-yl-5-oxo-5,8-dihydro- |
| | pyrido[2,3-d]pyrimidine-6-carboxylic acid methoxy-amide |
| | ¹H NMR (400 MHz, CDCl₃) δ (ppm): 11.98 (s, 1H), 9.38 (s, 1H), 8.82 (s, 1H), 7.64 (br, |
| | 1H), 7.42 (d, 1H), 7.25 (br, 3H), 7.18 (d, 1H), 6.95 (br, 2H), 3.90 (s, 3H), 3.66 (t, 2H), |
| | 3.10 (m, 4H), 3.00 (t, 2H), 2.62 (m, 3H), 2.46 (m, 1H), 2.22 (m, 4H), 1.88 (m, 4H). Mass |
| | Spectrum (LCMS, ESI pos.) Calcd. For C₃₁H₃₄N₆O₄: 555.24 (M + H), Found 555.5. |
| 162 | 2-{4-[1-(2-hydroxyethyl)-piperidin-4-yl]-phenylamino}-5-oxo-8-(4-trifluoromethoxy- |
| | phenyl)-5,8-dihydropyrido[2,3-d]pyrimidine-6-carboxylic acid methoxy-amide |
| | ¹H NMR (400 MHz, CDCl₃) δ (ppm): 9.26 (s, 1H), 8.67 (s, 1H), 7.42 (m, 4H), 7.18 (br, |
| | 2H), 6.90 (br, 2H), 3.82 (s, 3H), 3.62 (t, 2H), 3.02 (m, 2H), 2.56 (t, 2H), 2.40 (m, 1H), |
| | 2.13 (m, 2H), 1.80-1.60 (m, 4H). Mass Spectrum (LCMS, ESI pos.) Calcd. For: |
| | C₂₉H₂₉F₃N₆O₅: 599.22; Found: 599.5 (M + H). |
| 176 | 5-oxo-2-(4-piperidin-4-yl-phenylamino)-8-(4-trifluoromethoxyphenyl)-5,8-dihydro- |
| | pyrido[2,3-d]pyrimidine-6-carboxylic acid methoxy-amide |
| | ¹H NMR (400 MHz, CDCl₃) δ (ppm): 9.24 (s, 1H), 8.66 (s, 1H), 7.42 (m, 4H), 7.18 (d, |
| | 2H), 6.88 (d, 2H), 3.82 (s, 3H), 3.13 (m, 2H), 2.68 (m, 2H), 2.51 (m, 1H), 1.75 (m, 2H), |
| | 1.53 (m, 2H). Mass Spectrum (LCMS, ESI pos.) Calcd. For C₂₇H₂₅F₃N₆O₄: 555.2 (M + H), |
| | Found: 555.5. |

### Example 36

### 5-oxo-2-[4-(1-sulfamoyl-piperidin-4-yl)-phenylamino]-8-(4-trifluoromethoxyphenyl)-5,8-dihydropyrido[2,3-d]pyrimidine-6-carboxylic acid methoxy-amide (Cpd 177)

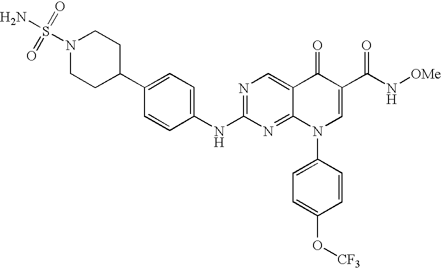

A mixture of sulfamide (10 mg, 0.1 mmol) and 5-oxo-2-(4-piperidin-4-yl-phenylamino)-8-(4-trifluoromethoxyphenyl)-5,8-dihydropyrido[2,3-d]pyrimidine-6-carboxylic acid methoxy-amide (10 mg, 0.02 mmol) in 1,4-dioxane (1 mL) was heated at reflux for 0.5 h. The solution was concentrated and purified (reverse phase HPLC using a C-18 YMC ODS-A 5μ 30×100 mm, 120 A column at 32 mL/min, 5-100% H₂O/MeCN (0.1% TFA v/v) gradient over 10 min.) to afford the title compound. ¹H NMR (400 MHz, CDCl₃) δ (ppm): 9.28 (s, 1H), 8.70 (s, 1H), 7.44 (m, 4H), 7.18 (br, 2H), 6.90 (br, 2H), 3.82 (s, 3H), 3.77 (m, 2H), 2.66 (m, 3H), 1.80-1.60 (m, 4H). Mass Spectrum (LCMS, ESI pos.) Calcd. For: C₂₇H₂₆F₃N₇O₆S: 634.16; Found: 634.5 (M+H).

Using the procedure of Example 36 and reagents, starting materials and conditions known to those skilled in the art, the following compounds representative of the present invention were prepared:

| Cpd | Name and Data |
|---|---|
| 178 | 8-indan-5-yl-5-oxo-2-[4-(1-sulfamoyl-piperidin-4-yl)-phenylamino]-5,8-dihydro- |
| | pyrido[2,3-d]pyrimidine-6-carboxylic acid methoxy-amide |
| | ¹H NMR (400 MHz, CD₃OD) δ (ppm): 9.25 (s, 1H), 8.70 (s, 1H), 7.20 (d, 1H), 7.24 (br, |
| | 3H), 7.13 (d, 1H), 6.83 (br, 2H), 3.80 (s, 3H), 3.72 (m, 2H), 3.04 (m, 2H), 2.83 (m, 2H), |
| | 2.66 (m, 2H), 2.44 (m, 1H), 2.18 (m, 2H), 1.85-1.60 (m, 4H). Mass Spectrum (LCMS, |
| | ESI pos.) Calcd. For: C₂₉H₃₁N₇O₅S: 590.21; Found: 590.5 (M + H). |
| 179 | 8-(4-ethyl-phenyl)-5-oxo-2-[4-(1-sulfamoyl-piperidin-4-yl)-phenylamino]-5,8-dihydro- |
| | pyrido[2,3-d]pyrimidine-6-carboxylic acid methoxy-amide |
| | ¹H NMR (400 MHz, CDCl₃) δ (ppm): 9.27 (s, 1H), 8.72 (s, 1H), 7.38 (d, 2H), 7.30 (d, |
| | 2H), 7.22 (br, 2H), 6.86 (br, 2H), 3.82 (s, 3H), 3.77 (m, 2H), 2.78 (m, 2H), 2.66 (m, 2H), |
| | 2.44 (m, 1H), 1.85-1.60 (m, 4H), 1.34 (t, 3H). Mass Spectrum (LCMS, ESI pos.) Calcd. |
| | For: C₂₈H₃₁N₇O₅S: 578.21; Found: 578.5 (M + H). |

### Example 37

### 8-indan-5-yl-2-[4-(1-isopropyl-piperidin-4-yl)-phenylamino]-5-oxo-5,8-dihydro-pyrido[2,3-d]pyrimidine-6-carboxylic acid methoxy-amide (Cpd 111)

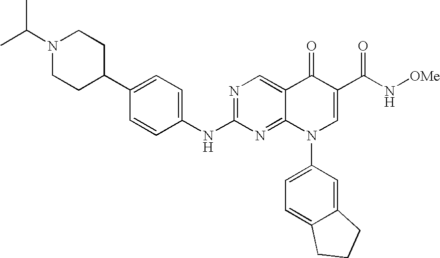

To a solution of 8-indan-5-yl-5-oxo-2-(4-piperidin-4-yl-phenylamino)-5,8-dihydro-pyrido[2,3-d]pyrimidine-6-carboxylic acid methoxy-amide Cpd 75 (10 mg, 0.02 mmol) in dichloroethane (0.5 mL) was added acetone (10 μL), sodium triacetoxyborohydride (6.4 mg, 0.03 mmol) and a catalytic amount of acetic acid. The sealed tube was heated for 12 h at 80° C. After aqueous work-up, the reaction mixture was extracted with EtOAc. The organic fractions were dried (Na₂SO₄) and filtered. The filtrate was concentrated in vacuo, and the residue was purified (HPLC, 32 mL/min, 5-80% MeCN/H₂O (0.1% TFA v/v) gradient over 12 min), to give the title compound as a yellow solid (1.3 mg, 11%). ¹H NMR (400 MHz, CDCl₃) δ (ppm): 11.90 (s, 1H), 9.30 (s, 1H), 8.78 (s, 1H), 7.50 (br, 1H), 7.36 (d, 1H), 7.20 (br, 3H), 7.12 (d, 1H), 6.90 (br, 2H), 3.82 (s, 3H), 3.03 (m, 3H), 2.95 (t, 2H), 2.40 (br, 1H), 2.20 (m, 2H), 2.00 (br, 4H), 1.80 (m, 4H), 1.20 (s, 6H). Mass Spectrum (LCMS, ESI pos.) Calcd. For C₃₂H₃₆N₆O₃: 553.28 (M+H), Found 553.5.

### Example 38

### 8-cyclohexyl-2-[4-(1-methyl-piperidin-4-yl)-phenylamino]-5-oxo-5,8-dihydro-pyrido[2,3-d]pyrimidine-6-carboxylic acid methoxy-amide (Cpd 140)

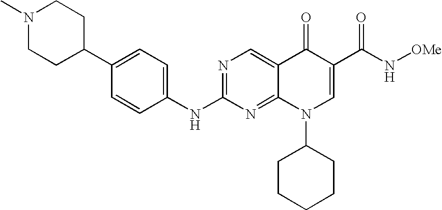

### A. 8-cyclohexyl-2-[4-(1-methyl-piperidin-4-yl)-phenylamino]-5-oxo-5,8-dihydro-pyrido[2,3-d]pyrimidine-6-carboxylic acid ethyl ester

A solution of 8-cyclohexyl-2-methanesulfonyl-5-oxo-5,8-dihydro-pyrido[2,3-d]pyrimidine-6-carboxylic acid ethyl ester (from Example 3, Step E, 75 mg, 0.17 mmol) and 4-(1-methyl-piperidin-4-yl)-phenylamine (81 mg, 0.42 mmol) in AcOH (1 mL) was heated at 110° C. After 10 min, the solution was concentrated and purified (HPLC, C-18 YMC ODS-A 5μ 30×100 mm, 120 A column at 32 mL/min, 5-100% H₂O/MeCN (0.1% TFA v/v) gradient over 10 min.) to give the title compound (18 mg). ¹H NMR (TFA salt) (400 MHz, CDCl₃) δ (ppm): 9.29 (s, 1H), 8.56 (s, 1H), 7.64 (d, J=8.49 Hz, 2H), 7.26-7.19 (m, 2H), 4.38 (q, J=7.11 Hz, 2H), 3.74 (d, J=11.61 Hz, 2H), 2.88 (d, J=2.92 Hz, 2H), 2.77 (m, 1H), 2.26 (d, J=11.24 Hz, 2H), 2.05 (m, 5H), 1.85 (s, 1H), 1.68 (dd, J=11.89, 2.39 Hz, 2H), 1.51 (d, J=12.88 Hz, 2H), 1.39 (t, J=7.12 Hz, 3H), 1.31 (m, 1H); Mass Spectrum (LCMS, APCI pos.) Calcd. For: C₂₈H₃₅N₅O₃: 489.27; Found: 490.3 (M+H).

### B. 8-cyclohexyl-2-[4-(1-methyl-piperidin-4-yl)-phenylamino]-5-oxo-5,8-dihydro-pyrido[2,3-d]pyrimidine-6-carboxylic acid methoxy-amide

A solution of 8-cyclohexyl-2-[4-(1-methyl-piperidin-4-yl)-phenylamino]-5-oxo-5,8-dihydro-pyrido[2,3-d]pyrimidine-6-carboxylic acid ethyl ester (9 mg) and O-methyl-hydroxylamine (0.5 g) in 1:1 TEA and MeOH (2 mL) in a sealed tube was heated at 110° C. for 2 days. After cooling, water was added and the solution was directly purified (HPLC, C-18 YMC ODS-A 5μ 30×100 mm, 120 A column at 32 mL/min, 5-100% H₂O/MeCN (0.1% TFA v/v) gradient over 10 min.) to provide the title compound (2.2 mg). ¹H NMR (TFA salt) (400 MHz, CDCl₃) δ (ppm): 9.39 (s, 1H), 8.82 (s, 1H), 7.68-7.60 (m, 2H), 7.29 (br s, 2H), 3.89 (s, 3H), 3.77-3.65 (m, 2H), 2.86 (d, J=4.29 Hz, 6H), 2.66-2.56 (m, 4H), 2.17-2.01 (m, 1H), 1.93-1.86 (m, 2H), 1.82-1.73 (m, 2H), 1.60-1.48 (m, 2H), 2.49-2.35 (m, 1H); Mass Spectrum (LCMS, APCI pos.) Calcd. For: C₂₇H₃₄N₆O₃: 490.27; Found: 491.3 (M+H).

### Example 39

### 8-indan-5-yl-2-[4-(1-methyl-piperidin-4-yl)-phenylamino]-5-oxo-5,8-dihydro-pyrido[2,3-d]pyrimidine-6-carboxylic acid ethoxy-amide (Cpd 139)

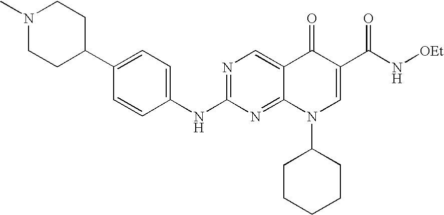

A solution of 8-indan-5-yl-2-[4-(1-methyl-piperidin-4-yl)-phenylamino]-5-oxo-5,8-dihydro-pyrido[2,3-d]pyrimidine-6-carboxylic acid methyl ester (from Example 34, Step A) (15 mg) and O-ethyl-hydroxylamine (0.5 g) in 1:1 TEA and MeOH (2 mL) in a sealed tube was heated at 110° C. for 2 days. After cooling, water was added and the solution was directly purified (HPLC, C-18 YMC ODS-A 5μ 30×100 mm, 120 A column at 32 mL/min, 5-100% H₂O/MeCN (0.1% TFA v/v) gradient over 10 min.) to provide the title compound (7.5 mg). ¹H NMR (TFA salt) (400 MHz, CDCl₃) δ (ppm): 9.36 (s, 1H), 8.83 (s, 1H), 7.43 (d, J=7.91 Hz, 2H), 7.37-7.28 (m, 3H), 7.16 (dd, J=7.91, 1.92 Hz, 2H), 4.10 (q, J=7.03 Hz, 2H), 3.81-3.67 (m, 2H), 3.07 (d, J=7.25 Hz, 2H), 2.99 (t, J=7.42 Hz, 2H), 2.88-2.80 (m, 2H), 2.74-2.58 (m, 1H), 2.22 (dd, J=14.31, 6.76 Hz, 2H), 2.00 (d, J=0.68 Hz, 2H), 1.36 (t, J=7.04 Hz, 3H); Mass Spectrum (LCMS, APCI pos.) Calcd. For: C₃₁H₃₄N₆O₃: 538.27; Found: 539.3 (M+H).

### Example 40

### 8-indan-5-yl-2-[4-(4-methyl-piperazine-1-sulfonyl)-phenylamino]-5-oxo-5,8-dihydro-pyrido[2,3-d]pyrimidine-6-carboxylic acid methoxy-amide (Cpd 154)

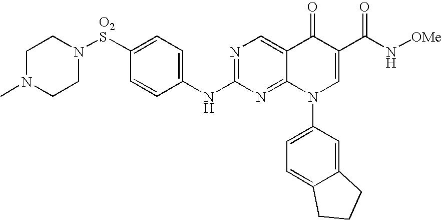

Using procedures described in Example 3, Step F, the title compound was prepared from 4-(4-methyl-piperazine-1-sulfonyl)-phenylamine (41 mg, 0.18 mmol) and 8-indan-5-yl-2-methanesulfonyl-5-oxo-5,8-dihydro-pyrido[2,3-d]pyrimidine-6-carboxylic acid methoxy-amide (30 mg, 0.072 mmol). The title compound was obtained as a yellow solid (6.3 mg). ¹H NMR (400 MHz, CDCl₃/CD₃OD 10:1 v/v) δ (ppm): 9.43 (s, 1H), 8.88 (s, 1H), 7.74 (m, 1H), 7.42-7.58 (m, 6H), 3.91 (s, 3H), 3.52 (d, 4H), 3.20 (m, 2H), 2.28-3.11 (m, 8H), 2.83 (s, 3H). Mass Spectrum (LCMS, ESI pos.) Calcd. For C₂₉H₃₁N₇O₅S: 589.21; Found 590.2 (M+H).

### Example 41

### 8-(4-ethyl-phenyl)-2-[4-(1-ethyl-piperidin-4-yl)-phenylamino]-5-oxo-5,8-dihydro-pyrido[2,3-d]pyrimidine-6-carboxylic acid methoxy-amide (Cpd 157)

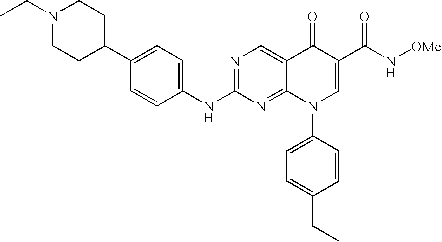

### A. 8-(4-ethyl-phenyl)-2-methanesulfonyl-5-oxo-5,8-dihydro-pyrido[2,3-d]pyrimidine-6-carboxylic acid ethyl ester

To a solution of 8-(4-ethyl-phenyl)-2-methylsulfanyl-5-oxo-5,8-dihydro-pyrido[2,3-d]pyrimidine-6-carboxylic acid ethyl ester (Example 33) (Step A, 2.5 g, 6.8 mmol) in 150 mL of CH₂Cl₂, was added 3-chloroperoxybenzoic acid (m-CPBA, 77%, 3.8 g, 17 mmol) portionwise. The reaction mixture was stirred at rt for 5 h. An aqueous solution of 10% sodium thiosulfate (20 mL) was added to quench the reaction. After 30 min, a saturated sodium bicarbonate solution was added. The reaction mixture was extracted with CH₂Cl₂. The combined CH₂Cl₂ fractions were washed with NaHCO₃ solution twice, dried over Na₂SO₄ and filtered. The filtrate was concentrated and the product was recrystallized from EtOAc/Hex. A creamy solid was obtained (1.8 g, 66%).

### B. 2-[4-(1-tert-butoxycarbonyl-piperidin-4-yl)-phenylamino]-8-(4-ethyl-phenyl)-5-oxo-5,8-dihydro-pyrido[2,3-d]pyrimidine-6-carboxylic acid ethyl ester

8-(4-ethyl-phenyl)-2-methanesulfonyl-5-oxo-5,8-dihydro-pyrido[2,3-d]pyrimidine-6-carboxylic acid ethyl ester (1 g, 2.4 mmol) and 4-(4-amino-phenyl)-piperidine-1-carboxylic acid tert-butyl ester (Example 4 Step B) (0.7 g, 2.5 mmol) were combined in AcOH (10 mL) and heated at 110° C. for 15 min. The reaction mixture was concentrated and methanol (10 mL) was added to the remaining residue. The title compound was obtained as a precipitate and collected by filtration, then washed with methanol and used in the next step without further purification.

### C. 2-[4-(1-tert-butoxycarbonyl-piperidin-4-yl)-phenylamino]-8-(4-ethyl-phenyl)-5-oxo-5,8-dihydro-pyrido[2,3-d]pyrimidine-6-carboxylic acid

To a solution of 2-[4-(1-tert-butoxycarbonyl-piperidin-4-yl)-phenylamino]-8-(4-ethyl-phenyl)-5-oxo-5,8-dihydro-pyrido[2,3-d]pyrimidine-6-carboxylic acid ethyl ester (1.1 g, 1.8 mmol) in THF (10 mL) and methanol (10 mL) was added 10 mL of a 1N NaOH solution. The mixture was stirred at 50° C. for 2 h. The organic solvents were evaporated in vacuo. Water (20 mL) was added and pH was adjusted to 4 by 1N HCl solution. The aqueous mixture was extracted with CH₂Cl₂, and the organic layer was dried over Na₂SO₄, and filtered. The filtrate was concentrated to dryness in vacuo to provide the title compound as a yellow solid (0.8 g).

### D. 4-{4-[8-(4-ethyl-phenyl)-6-methoxycarbamoyl-5-oxo-5,8-dihydro-pyrido[2,3-d]pyrimidin-2-ylamino]-phenyl}-piperidine-1-carboxylic acid tert-butyl ester

To a solution of 2-[4-(1-tert-butoxycarbonyl-piperidin-4-yl)-phenylamino]-8-(4-ethyl-phenyl)-5-oxo-5,8-dihydro-pyrido[2,3-d]pyrimidine-6-carboxylic acid (0.8 g, 1.4 mmol) in 100 mL of CH₂Cl₂ at 0° C. was added 0.37 mL of oxalyl chloride (29.8 mmol) and 20 μL of DMF. The solution was stirred at room temperature for 3 h. The solvent and excess oxalyl chloride was removed in vacuo. The residue was dissolved in 80 mL of CH₂Cl₂. To this solution was added MeONH₂.HCl (70 mg, 0.84 mmol) followed by slow addition of Et₃N (200 μL, 0.84 mmol). The reaction mixture was stirred at 0° C. for 5 min and at rt for 20 min. Water was added and the reaction mixture was extracted with CH₂Cl₂ (50 mL×2). The combined organic fractions were washed with brine and concentrated in vacuo to give the title compound as an off-white solid (0.3 g).

### E. 8-(4-ethyl-phenyl)-2-[4-(1-ethyl-piperidin-4-yl)-phenylamino]-5-oxo-5,8-dihydro-pyrido[2,3-d]pyrimidine-6-carboxylic acid methoxy-amide

4-{4-[8-(4-ethyl-phenyl)-6-methoxycarbamoyl-5-oxo-5,8-dihydro-pyrido[2,3-d]pyrimidin-2-ylamino]-phenyl}-piperidine-1-carboxylic acid tert-butyl ester (20 mg, 0.03 mmol) was treated with TFA/CH₂Cl₂ (2 mL, 1:1 v/v) at rt for 2 h. The reaction mixture was concentrated in vacuo. The residue was dissolved in DMSO (1 mL), and to the stirred solution was added 2-iodoethane (3.2 μL, 0.04 mmol) and triethylamine (14 μL, 0.10 mmol). After 16 h, water was added and the reaction mixture was extracted with CH₂Cl₂. The combined organic extracts were dried (Na₂SO₄), filtered, and the filtrate concentrated and purified (HPLC, C-18 YMC ODS-A 5 30×100 mm, 120 A column at 32 mL/min, 10-70% H₂O/MeCN (0.1% TFA v/v) gradient over 12 min) to afford the title compound (3.4 mg). ¹H NMR (400 MHz, CDCl₃) δ (ppm): 11.97 (s, 1H), 9.38 (s, 1H), 8.80 (s, 1H), 7.60 (br, 1H), 7.40 (d, 2H), 7.32 (d, 2H), 7.22 (br, 2H), 6.96 (br, 2H), 3.90 (s, 3H), 3.08 (d, 2H), 2.83 (q, 2H), 2.75 (q, 2H), 2.01 (m, 1H), 1.62-1.80 (m, 6H), 1.39 (t, 3H), 1.15 (t, 3H). Mass Spectrum (LCMS, ESI pos.) Calcd. For: C₃₀H₃₄N₆O₃: 526.27; Found: 527.6 (M+H).

Using the procedure of Example 41 (Steps D and E) and 2-[4-(1-tert-butoxycarbonyl-piperidin-4-yl)-phenylamino]-8-(4-ethyl-phenyl)-5-oxo-5,8-dihydro-pyrido[2,3-d]pyrimidine-6-carboxylic acid (from Example 41, Step C), the following compounds representative of the present invention were prepared:

| Cpd | Name and Data |
|---|---|
| 158 | 8-(4-ethyl-phenyl)-2-[4-(1-isopropyl-piperidin-4-yl)-phenylamino]-5-oxo-5,8-dihydro- |
| | pyrido[2,3-d]pyrimidine-6-carboxylic acid methoxy-amide |
| | Cpd 158 may be prepared using the procedure of Example 41. |
| 181 | 8-(4-ethyl-phenyl)-2-[4-(1-ethyl-piperidin-4-yl)-phenylamino]-5-oxo-5,8-dihydro- |
| | pyrido[2,3-d]pyrimidine-6-carboxylic acid ethoxy-amide |
| | ¹H NMR (400 MHz, CDCl₃) δ (ppm): 11.92 (s, 1H), 9.38 (s, 1H), 8.80 (s, 1H), 7.62 (br, |
| | 1H), 7.40 (d, 2H), 7.32 (d, 2H), 7.22 (br, 2H), 6.96 (br, 2H), 4.10 (q, 2H), 3.08 (d, 2H), |
| | 2.81 (q, 2H), 2.44 (q, 2H), 2.00 (t, 2H), 1.62-1.80 (m, 5H), 1.38 (m, 6H), 1.15 (t, 3H). |
| | Mass Spectrum (LCMS, ESI pos.) Calcd. For: C₃₁H₃₆N₆O₃: 540.28; Found: 541.6 (M + H). |
| 182 | 8-(4-ethyl-phenyl)-2-[4-(1-ethyl-piperidin-4-yl)-phenylamino]-5-oxo-5,8-dihydro- |
| | pyrido[2,3-d]pyrimidine-6-carboxylic acid isopropoxy-amide |
| | ¹H NMR (400 MHz, CDCl₃) δ (ppm): 11.80 (s, 1H), 9.38 (s, 1H), 8.80 (s, 1H), 7.58 (br, |
| | 1H), 7.41 (d, 2H), 7.35 (d, 2H), 7.22 (br, 2H), 6.97 (br, 2H), 4.26 (m, 1H), 3.08 (d, 2H), |
| | 2.82 (q, 2H), 2.44 (q, 2H), 2.00 (t, 2H), 1.62-1.80 (m, 5H), 1.35 (m, 9H), 1.12 (t, 3H). |
| | Mass Spectrum (LCMS, ESI pos.) Calcd. For: C₃₂H₃₈N₆O₃: 554.30; Found: 555.6 (M + H). |
| 183 | 8-(4-ethyl-phenyl)-2-{4-[1-(2-hydroxy-ethyl)-piperidin-4-yl]-phenylamino}-5-oxo-5,8- |
| | dihydro-pyrido[2,3-d]pyrimidine-6-carboxylic acid ethoxy-amide |
| | ¹H NMR (400 MHz, CDCl₃) δ (ppm): 11.90 (s, 1H), 9.38 (s, 1H), 8.80 (s, 1H), 7.60 (br, |
| | 1H), 7.41 (d, 2H), 7.35 (d, 2H), 7.22 (br, 2H), 6.95 (br, 2H), 4.10 (q, 2H), 3.62 (t, 2H), |
| | 3.03 (d, 2H), 2.82 (q, 2H), 2.59 (t, 2H), 2.42 (m, 1H), 2.18 (t, 2H), 1.65-1.82 (m, 4H), |
| | 1.39 (m, 6H). Mass Spectrum (LCMS, ESI pos.) Calcd. For: C₃₁H₃₆N₆O₄: 556.28; |
| | Found: 557.6 (M + H). |
| 184 | 8-(4-ethyl-phenyl)-2-{4-[1-(2-hydroxy-ethyl)-piperidin-4-yl]-phenylamino}-5-oxo-5,8- |
| | dihydro-pyrido[2,3-d]pyrimidine-6-carboxylic acid isopropoxy-amide |
| | ¹H NMR (400 MHz, CDCl₃) δ (ppm): 11.90 (s, 1H), 9.38 (s, 1H), 8.80 (s, 1H), 7.60 (br, |
| | 1H), 7.41 (d, 2H), 7.35 (d, 2H), 7.22 (br, 2H), 6.95 (br, 2H), 4.25 (m, 1H), 3.62 (t, 2H), |
| | 3.03 (d, 2H), 2.82 (q, 2H), 2.59 (t, 2H), 2.43 (m, 1H), 2.20 (t, 2H), 1.65-1.82 (m, 4H), |
| | 1.39 (t, 3H), 1.34 (s, 3H), 1.36 (s, 3H). Mass Spectrum (LCMS, ESI pos.) Calcd. For: |
| | C₃₂H₃₈N₆O₄: 570.30; Found: 571.6 (M + H). |
| 185 | 8-(4-ethyl-phenyl)-2-{4-[1-(3-hydroxy-propyl)-piperidin-4-yl]-phenylamino}-5-oxo- |
| | 5,8-dihydro-pyrido[2,3-d]pyrimidine-6-carboxylic acid methoxy-amide |
| | ¹H NMR (400 MHz, CDCl₃) δ (ppm): 11.90 (s, 1H), 9.32 (s, 1H), 8.78 (s, 1H), 7.57 (br, |
| | 1H), 7.39 (d, 2H), 7.25 (d, 2H), 7.20 (br, 2H), 6.90 (br, 2H), 3.83 (s, 3H), 3.79 (t, 2H), |
| | 3.12 (d, 2H), 2.78 (q, 2H), 2.60 (t, 2H), 2.40 (m, 1H), 2.00 (t, 2H), 1.50-1.80 (m, 6H), |
| | 1.32 (t, 3H). Mass Spectrum (LCMS, ESI pos.) Calcd. For: C₃₁H₃₆N₆O₄: 556.28; Found: |
| | 557.6 (M + H). |
| 186 | 8-(4-ethyl-phenyl)-2-{4-[1-(3-hydroxy-propyl)-piperidin-4-yl]-phenylamino}-5-oxo- |
| | 5,8-dihydro-pyrido[2,3-d]pyrimidine-6-carboxylic acid ethoxy-amide |
| | ¹H NMR (400 MHz, CDCl₃) δ (ppm): 11.90 (s, 1H), 9.38 (s, 1H), 8.80 (s, 1H), 7.57 (br, |
| | 1H), 7.42 (d, 2H), 7.35 (d, 2H), 7.22 (br, 2H), 6.94 (br, 2H), 4.10 (q, 2H), 3.82 (t, 2H), |
| | 3.19 (d, 2H), 2.82 (q, 2H), 2.65 (t, 2H), 2.45 (m, 1H), 2.00 (t, 2H), 1.60-1.82 (m, 6H), |
| | 1.40 (m, 6H). Mass Spectrum (LCMS, ESI pos.) Calcd. For: C₃₂H₃₈N₆O₄: 570.30; |
| | Found: 571.6 (M + H). |
| 187 | 8-(4-ethyl-phenyl)-2-{4-[1-(3-hydroxy-propyl)-piperidin-4-yl]-phenylamino}-5-oxo- |
| | 5,8-dihydro-pyrido[2,3-d]pyrimidine-6-carboxylic acid isopropoxy-amide |
| | ¹H NMR (400 MHz, CDCl₃) δ (ppm): 11.91 (s, 1H), 9.38 (s, 1H), 8.80 (s, 1H), 7.62 (br, |
| | 1H), 7.42 (d, 2H), 7.35 (d, 2H), 7.24 (br, 2H), 6.95 (br, 2H), 4.26 (m, 1H), 3.82 (t, 2H), |
| | 3.19 (d, 2H), 2.82 (q, 2H), 2.65 (t, 2H), 2.45 (m, 1H), 2.05 (t, 2H), 1.60-1.90 (m, 6H), |
| | 1.39 (t, 3H), 1.34 (s, 3H), 1.36 (s, 3H). Mass Spectrum (LCMS, ESI pos.) Calcd. For: |
| | C₃₃H₄₀N₆O₄: 584.31; Found: 585.7 (M + H). |
| 188 | 8-(4-ethyl-phenyl)-5-oxo-2-{4-[1-(2,2,2-trifluoro-ethyl)-piperidin-4-yl]-phenylamino}- |
| | 5,8-dihydro-pyrido[2,3-d]pyrimidine-6-carboxylic acid methoxy-amide |
| | ¹H NMR (400 MHz, CDCl₃) δ (ppm): 11.90 (s, 1H), 9.32 (s, 1H), 8.75 (s, 1H), 7.50 (br, |
| | 1H), 7.38 (d, 2H), 7.30 (d, 2H), 7.20 (br, 2H), 6.90 (br, 2H), 3.83 (s, 3H), 3.01 (d, 2H), |
| | 2.98 (q, 2H), 2.80 (q, 2H), 2.40 (m, 3H), 1.70 (m, 4H), 1.35 (t, 3H). Mass Spectrum |
| | (LCMS, ESI pos.) Calcd. For: C₃₀H₃₁F₃N₆O₃: 580.24; Found: 581.6 (M + H). |
| 189 | 8-(4-ethyl-phenyl)-5-oxo-2-{4-[1-(2,2,2-trifluoro-ethyl)-piperidin-4-yl]-phenylamino}- |
| | 5,8-dihydro-pyrido[2,3-d]pyrimidine-6-carboxylic acid ethoxy-amide |
| | ¹H NMR (400 MHz, CDCl₃) δ (ppm): 11.90 (s, 1H), 9.38 (s, 1H), 8.80 (s, 1H), 7.55 (br, |
| | 1H), 7.42 (d, 2H), 7.35 (d, 2H), 7.22 (br, 2H), 6.95 (br, 2H), 4.10 (q, 2H), 3.08 (d, 2H), |
| | 3.01 (q, 2H), 2.83 (q, 2H), 2.44 (m, 3H), 1.78 (m, 4H), 1.38 (m, 6H). Mass Spectrum |
| | (LCMS, ESI pos.) Calcd. For: C₃₁H₃₃F₃N₆O₃: 594.26; Found: 595.6 (M + H). |

### Example 42

### 8-indan-5-yl-5-oxo-2-{4-[2-(1H-[1,2,4]triazol-3-yl)-ethyl]-phenylamino}-5,8-dihydro-pyrido[2,3-d]pyrimidine-6-carboxylic acid methoxy-amide (Cpd 190)

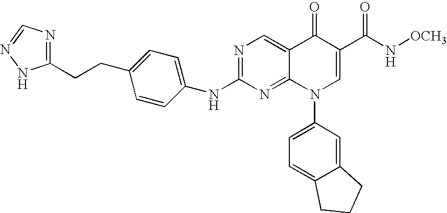

### A. 3-[2-(4-nitrophenyl)-ethyl]-1H-[1,2,4]triazole

To a solution of 3-(4-nitrophenyl)-propionic acid (250 mg, 1.3 mmol) and 10 μL of DMF in 5 mL of CH₂Cl₂ at 0° C. was slowly added oxalyl chloride (0.3 mL, 3 mmol). The reaction was stirred at rt for 1 h. The reaction mixture was concentrated, and the residue was dissolved in 5 mL of CH₂Cl₂, and 1 mL of 28% NH₃ (aq.) was added at 0° C. The mixture was stirred at rt for 0.5 h, then quenched with water. The mixture was extracted with CH₂Cl₂. The organic fractions were washed with brine, dried over Na₂SO₄, and filtered. The filtrate was concentrated under reduced pressure. The residue was dissolved in 2 mL of N,N-dimethylformamide dimethyl acetal and the solution was stirred at 120° C. for 2 h. The reaction mixture was concentrated to give a yellow solid. A mixture of this residue with hydrazine hydrate (100 mg, 2.0 mmol) in 1 mL of acetic acid was stirred at 90° C. for 2 h. The reaction mixture was concentrated in vacuo. Water was added and the aqueous solution was neutralized with sat. NaHCO₃ (aq) and extracted with EtOAc. The organic fraction was concentrated under reduced pressure to provide the title compound (150 mg).

### B. 3-[2-(4-aminophenyl)-ethyl]-[1,2,4]triazole-1-carboxylic acid tert-butyl ester

A mixture of 3-[2-(4-nitrophenyl)-ethyl]-1H-[1,2,4]triazole (50 mg, 0.23 mmol), (Boc)₂O (70 mg, 0.3 mmol), 0.11 mL of Et₃N, and 5 mg of DMAP in 2 mL of CH₂Cl₂ was stirred at rt for 2 h. The solution was loaded to a silica gel column (EtOAc/hexanes, 1:10 v/v). The product in 5 mL of MeOH and 10 mg of Pd/C (10%) was stirred under 1 atm of H₂ for 1 h. The reaction mixture was filtered through celite and the filtrate was concentrated to provide the title compound (50 mg).

### C. 8-indan-5-yl-5-oxo-2-{4-[2-(1H-[1,2,4]triazol-3-yl)-ethyl]-phenylamino}-5,8-dihydro-pyrido[2,3-d]pyrimidine-6-carboxylic acid methoxy-amide

The title compound was prepared by reacting 3-[2-(4-aminophenyl)-ethyl]-[1,2,4]triazole-1-carboxylic acid tert-butyl ester with 8-indan-5-yl-2-methanesulfonyl-5-oxo-5,8-dihydro-pyrido[2,3-d]pyrimidine-6-carboxylic acid methoxy-amide. The Boc group was removed by treatment with 1:1 TFA/CH₂C₁₂. ¹H NMR (400 MHz, DMSO-d₆) δ (ppm): 11.9 (s, 1H), 10.4 (br, 1H), 9.21 (s, 1H), 8.80 (s, 1H), 7.45 (br, 2H), 7.30 (br, 4H), 6.84 (br, 2H), 4.00-2.00 (m, 13H). Mass Spectrum (LCMS, ESI pos.) Calcd. For C₂₈H₂₆N₈O₃: 523.2 (M+H), Found: 523.3.

### Example 43

### 2-{4-[2-(5-methyl-1H-[1,2,4]triazol-3-yl)-ethyl]-phenylamino}-5-oxo-8-(4-trifluoromethoxyphenyl)-5,8-dihydropyrido[2,3-d]pyrimidine-6-carboxylic acid methoxy-amide (Cpd 191)

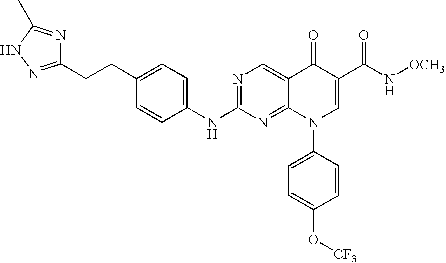

### A. 3-(4-nitrophenyl)-propionic acid hydrazide

To a stirred solution of 3-(4-nitrophenyl)-propionic acid (490 mg, 2.5 mmol) in 10 mL of THF and 1 mL of Et₃N at 0° C., was slowly added ethyl chloroformate (0.26 mL, 2.5 mmol). After 30 min, was added 0.3 g of H₂NNH₂.H₂O. The reaction mixture was allowed to warm up to rt and stirred at rt for 30 min. The reaction mixture was concentrated under reduced pressure. The residue was dissolved in EtOAc and washed with water. The organic fractions were concentration under reduced pressure to provide the title compound (480 mg).

### B. 5-methyl-3-[2-(4-nitrophenyl)-ethyl]-[1,2,4]triazole-1-carboxylic acid tert-butyl ester

To 1 mL of methanol was slowly added NaH (140 mg, 60% in wax, 3.5 mmol). To this solution was added acetamidine hydrochloride (303 mg, 3.04 mmol) in 4 mL of ethanol. The solution was stirred at rt for 20 min, whereupon it was filtered. To the filtrate was added 430 mg of 3-(4-nitrophenyl)-propionic acid hydrazide and the mixture was stirred for 5 h. The reaction mixture was concentrated under reduced pressure, and the residue was purified by chromatography (silica, 10-30% methanol in CH₂Cl₂) to provide a white solid. The white solid was heated (neat) to its melting temperature (around 220° C.) for 30 min then used in the next step without further purification.

To a stirred solution of 5-methyl-3-[2-(4-nitrophenyl)-ethyl]-1H-[1,2,4]triazole (180 mg, 0.8 mmol) and 370 mg of Boc₂O in 8 mL of CH₂Cl₂ was added 0.3 mL of Et₃N followed by 5 mg of DMAP. The reaction mixture was stirred for 2 h. The reaction mixture was concentrated under reduced pressure, and the residue was purified by chromatography (silica, methanol/CH₂Cl₂ 1:100-3:100 v/v) to give the title compound (250 mg).

### C. 3-[2-(4-aminophenyl)-ethyl]-5-methyl-[1,2,4]triazole-1-carboxylic acid tert-butyl ester

A solution of 5-methyl-3-[2-(4-nitrophenyl)-ethyl]-[1,2,4]triazole-1-carboxylic acid tert-butyl ester (250 mg, 0.75 mmol) in 10 mL of methanol and 30 mg of Pd/C (10%) was stirred under 1 atm of H₂ overnight. The reaction mixture was filtered through a pad of celite, and the filtrate was concentrated to give the title compound (216 mg).

### D. 2-{4-[2-(5-methyl-1H-[1,2,4]triazol-3-yl)-ethyl]-phenylamino}-5-oxo-8-(4-trifluoromethoxyphenyl)-5,8-dihydropyrido[2,3-d]pyrimidine-6-carboxylic acid methoxy-amide

The title compound was prepared by reacting 3-[2-(4-aminophenyl)-ethyl]-5-methyl-[1,2,4]triazole-1-carboxylic acid tert-butyl ester with 8-(4-trifluoromethoxyphenyl)-5,8-dihydro-pyrido[2,3-d]pyrimidine-6-carboxylic acid methoxy-amide (Example 3, Step F). The Boc group was removed by treatment with 1:1 TFA/CH₂C₁₂. ¹H NMR (400 MHz, CDCl₃) δ (ppm): 9.28 (s, 1H), 8.70 (s, 1H), 7.44 (m, 4H), 7.15 (br, 2H), 6.87 (br, 2H), 3.83 (s, 3H), 2.90 (br, 4H), 2.35 (s, 3H). Mass Spectrum (LCMS, ESI pos.) Calcd. For C₂₇H₂₃F₃N₈O₄: 581.18 (M+H), Found: 581.6.

### Example 44

### 8-indan-5-yl-5-oxo-2-[3-(2-pyrrolidin-1-yl-ethylcarbamoyl)-phenylamino]-5,8-dihydro-pyrido[2,3-d]pyrimidine-6-carboxylic acid methoxy-amide (Cpd 192)

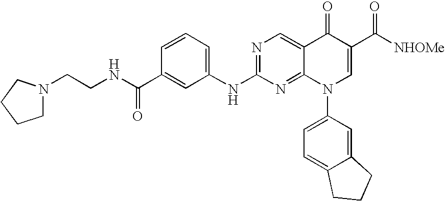

### A. 3-amino-N-(2-pyrrolidin-1-yl-ethyl)-benzamide

To a solution of 1 g of 3-nitro-benzoyl chloride in 25 mL of dichloromethane at 0° C. was added 1.029 mL of diisopropylamine followed by 737 mg of 2-pyrrolidin-1-yl-ethylamine and the reaction was stirred for 4 h with gradual warming to rt. The reaction mixture was washed twice with water, dried over sodium sulfate, filtered, and the filtrate was concentrated in vacuo. The crude residue was dissolved in 25 mL of EtOH, the vessel was purged with argon, then approximately 100 mg of 10% Pd on carbon was added. The vessel was evacuated and purged with hydrogen via balloon and stirred for 2 h at rt. The reaction mixture was filtered and the filtrate concentrated in vacuo. Trituration of the crude material in 5% ethyl acetate/hexane afforded the title compound (1.1 g).

### B. 8-indan-5-yl-5-oxo-2-[3-(2-pyrrolidin-1-yl-ethylcarbamoyl)phenylamino]-5,8-dihydro-pyrido[2,3-d]pyrimidine-6-carboxylic acid methoxy-amide

A solution of 110 mg of 8-indan-5-yl-2-methanesulfonyl-5-oxo-5,8-dihydro-pyrido[2,3-d]pyrimidine-6-carboxylic acid methoxy-amide, 68 mg of 3-amino-N-(2-pyrrolidin-1-yl-ethyl)-benzamide, and 92 mg of silver triflate in 2 mL of 1,4 dioxane was heated at 107° C. for 3 h. The reaction mixture was diluted with water and ethyl acetate, then filtered. The filtrate was extracted three times with ethyl acetate and the combined organics were dried over sodium sulfate, filtered and the filtrate was concentrated in vacuo. Purification of the crude material on a 2000 micron Prep. TLC plate using 10% methanol saturated with ammonia/dichloromethane as the eluent afforded 17 mg of the title compound. Mass Spectrum (LCMS, ESI pos.) Calcd. For C₃₁H₃₃N₇O₄: 567.64 (M+H), Found: 568.2.

### Example 45

### 2-{3-[(1-ethyl-pyrrolidin-2-ylmethyl)-carbamoyl]-phenylamino}-8-indan-5-yl-5-oxo-5,8-dihydro-pyrido[2,3-d]pyrimidine-6-carboxylic acid methoxy-amide (Cpd 193)

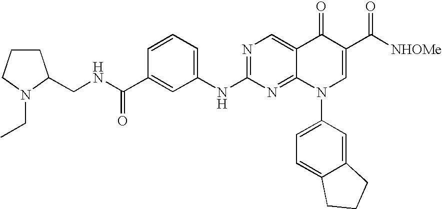

### A. 3-amino-N-(1-ethyl-pyrrolidin-2-ylmethyl)-benzamide

To a solution of 1 g of 3-nitro-benzoyl chloride in 25 mL of dichloromethane at 0° C. was added 1.029 mL of diisopropylamine followed by 827 mg of C-(1-ethyl-pyrrolidin-2-yl)-methylamine, and the reaction was stirred 4 h with gradual warming to rt. The reaction was washed twice with water, dried over sodium sulfate, filtered and the filtrate was concentrated in vacuo. The residue was dissolved in 25 mL of EtOH, and the vessel was purged with Argon. Approximately 100 mg of 10% Pd on carbon was added, the vessel was evacuated, purged with hydrogen via balloon and stirred for 2 h at rt. The reaction mixture was filtered, and concentrated in vacuo. Trituration of the crude material in 5% ethyl acetate/hexane afforded 1.2 g of the title compound. MS (LCMS, ESI pos.) Calcd. For C₃₂H₃₅N₇O₄: 581.67 (M+H), Found: 582.3/583.3.

### B. 2-{3-[(1-ethyl-pyrrolidin-2-ylmethyl)-carbamoyl]-phenylamino}-8-indan-5-yl-5-oxo-5,8-dihydro-pyrido[2,3-d]pyrimidine-6-carboxylic acid methoxy-amide

110 mg of 8-indan-5-yl-2-methanesulfonyl-5-oxo-5,8-dihydro-pyrido[2,3-d]pyrimidine-6-carboxylic acid methoxy-amide, 68 mg of 3-amino-N-(1-ethyl-pyrrolidin-2-ylmethyl)-benzamide, and 92 mg of silver triflate were taken up in 2 mL of 1,4 dioxane and the reaction was heated to 107° C. for 3 hours. The reaction was then diluted with water and ethyl acetate then filtered. The filtrate was then extracted with EtOAc (3×) and the combined organics are dried over sodium sulfate and concentrated in vacuo. Purification of the crude material on a 2000 micron Prep. TLC plate using 10% methanol saturated with ammonia/dichloromethane as the eluent afforded 18 mg (11%) of the title compound. Mass Spectrum (LCMS, ESI pos.) Calcd. For C₃₂H₃₅N₇O₄: 581.67 (M+H), Found: 582.3/583.3.

### Example 46

### 8-indan-5-yl-5-oxo-2-[3-(2-pyrrolidin-1-yl-ethyl)-phenylamino]-5,8-dihydro-pyrido[2,3-d]pyrimidine-6-carboxylic acid methoxy-amide (Cpd 194)

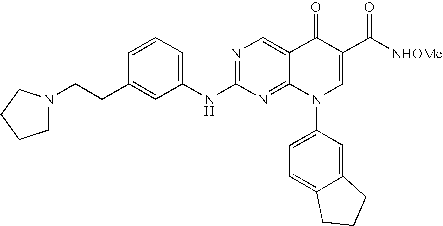

### A. methanesulfonic acid 2-(3-nitro-phenyl)-ethyl ester

To a solution of 5 g of 2-(3-nitro-phenyl)-ethanol and 4.5 mL of triethylamine in 50 mL of dichloromethane was added dropwise 3.7 g of methanesulfonyl chloride, and the reaction was stirred for 2 h. The reaction mixture was washed twice with water, and the organic layer was dried over sodium sulfate and filtered. The filtrate was concentrated in vacuo to afford 6.6 g of the title compound.

### B. 3-(2-pyrrolidin-1-yl-ethyl)-phenylamine

To a solution of 2 g of methanesulfonic acid 2-(3-nitro-phenyl)-ethyl ester in 40 mL of anhydrous DMF was added 3 g of cesium carbonate. To the reaction mixture was added 600 mg of pyrrolidine, and the reaction mixture was heated at 100° C. for 2 days. The reaction was cooled, diluted with water, and extracted three times with ether. The combined organic fractions were washed twice with water, dried over sodium sulfate, filtered, and the filtrate was concentrated in vacuo. The residue was purified by chromatography (silica, methanol:dichloromethane, 1:20) to give 800 mg of the title compound as a crude product. The product was hydrogenated according to the procedure of Example 44 to give 750 mg of the title compound.

### C. 8-indan-5-yl-5-oxo-2-[3-(2-pyrrolidin-1-yl-ethyl)-phenylamino]-5,8-dihydro-pyrido[2,3-d]pyrimidine-6-carboxylic acid methoxy-amide

125 mg of 8-indan-5-yl-2-methanesulfonyl-5-oxo-5,8-dihydro-pyrido[2,3-d]pyrimidine-6-carboxylic acid methoxy-amide, 75 mg of 3-(2-pyrrolidin-1-yl-ethyl)phenylamine, and 104 mg of silver triflate were taken up in 3 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 17 mg (10%) of the title compound. Calcd. For C₃₀H₃₂N₆O₃: 524.61 (M+H), Found: 525.2/526.3.

### Example 47

### 2-[3-(3,5-dimethyl-piperazin-1-yl)-phenylamino]-8-indan-5-yl-5-oxo-5,8-dihydro-pyrido[2,3-d]pyrimidine-6-carboxylic acid methoxy-amide (Cpd 195)

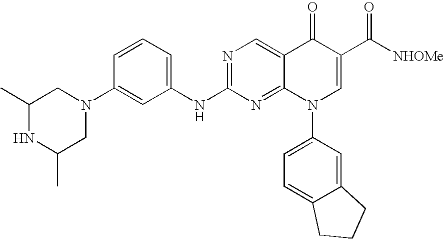

### A. 3-(3,5-dimethyl-piperazin-1-yl)-phenylamine

A solution of 1 g of 1-fluoro-3-nitro-benzene and 1.6 g of 2,6 dimethylpiperazine was stirred neat at 100° C. for 3 days. The reaction was approximately 30% complete (assessed via TLC) after 3 days. The reaction was diluted with ethyl acetate and washed three times with water. The combined organic fractions were dried over sodium sulfate, filtered, and the filtrate was concentrated in vacuo. The residue was triturated in hexane and decanted to remove the remaining 1-fluoro-3-nitro-benzene starting material. The residue was purified on three 2000 micron prep. TLC plates using 10% methanol saturated with ammonia/dichloromethane to give 300 mg of the title compound.

### B. 2-[3-(3,5-dimethyl-piperazin-1-yl)-phenylamino]-8-indan-5-yl-5-oxo-5,8-dihydro-pyrido[2,3-d]pyrimidine-6-carboxylic acid methoxy-amide

150 mg of 8-indan-5-yl-2-methanesulfonyl-5-oxo-5,8-dihydro-pyrido[2,3-d]pyrimidine-6-carboxylic acid methoxy-amide, 85 mg of 3-(3,5-dimethyl-piperazin-1-yl)-phenylamine, and 125 mg of silver triflate were taken up in 3 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded (8 mg, 4%) of the title compound. Mass Spectrum (LCMS, ESI pos.) Calcd. For C₃₀H₃₃N₇O₃: 539.63 (M+H), Found: 540.1/541.2/542.2.

### Example 48

### 2-[3-(4-acetyl-piperazin-1-yl)-phenylamino]-8-indan-5-yl-5-oxo-5,8-dihydro pyrido[2,3-d]pyrimidine-6-carboxylic acid methoxy-amide (Cpd 196)

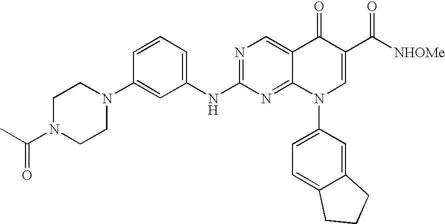

### A. 1-(3-nitro-phenyl)-piperazine

A solution of 5 g of 1-fluoro-3-nitro-benzene and 20 g of piperazine-1-carboxylic acid tert-butyl ester in 30 mL of DMSO were heated at 100° C. for 4 days. The reaction mixture was cooled, diluted with ethyl acetate, washed once with water and twice with 2N HCl. The combined organic fractions were dried over sodium sulfate, filtered and the filtrate was concentrated in vacuo. The residue was triturated in 10% ethyl acetate/hexane and decanted to remove starting 1-fluoro-3-nitro-benzene to give 2.9 g of 4-(3-nitro-phenyl)-piperazine-1-carboxylic acid tert-butyl ester. This material was stirred in 25 mL of 1:1 trifluoroacetic acid/dichloromethane for 2 h at rt. The reaction was concentrated in vacuo to give 2.9 g of the title compound as a TFA salt.

### B. 1-[4-(3-amino-phenyl)-piperazin-1-yl]-ethanone

To a solution of 500 mg of 1-(3-nitro-phenyl)-piperazine in 20 mL of dichloromethane and 500 μL of triethyl amine was added 135 mg of acetyl chloride, and the reaction mixture was stirred for 2 h at rt. The reaction was washed twice with water, dried over sodium sulfate, filtered and the filtrate was concentrated in vacuo to provide the title compound.

### C. 2-[3-(4-acetyl-piperazin-1-yl)-phenylamino]-8-indan-5-yl-5-oxo-5,8-dihydro pyrido[2,3-d]pyrimidine-6-carboxylic acid methoxy-amide

100 mg of 8-indan-5-yl-2-methanesulfonyl-5-oxo-5,8-dihydro-pyrido[2,3-d]pyrimidine-6-carboxylic acid methoxy-amide, 63 mg of 1-[4-(3-amino-phenyl)-piperazin-1-yl]-ethanone, and 87 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded the title compound (11 mg, 8%). ¹H NMR (300 MHz, CDCl₃) δ (ppm): 11.87 (s, 1H), 9.31 (s, 1H), 8.71 (s, 1H), 7.74 (bs, 1H), 7.34 (d, 1H), 7.18 (m, 3H), 7.10 (d, 1H), 6.96 (bs, 1H), 6.79 (d, 1H), 3.83 (s, 3H), 3.68 (m, 4H), 3.53 (m, 4H), 2.99 (t, 2H), 2.91 (t, 2H), 2.15 (m, 2H), 2.07 (s, 3H). Mass Spectrum (LCMS, ESI pos.) Calcd. For C₃₀H₃₁N₇O₄: 553.61 (M+H), Found: 554.1/555.1.

### Example 49

### 2-[3-(1-acetyl-piperidin-4-yl)-phenylamino]-8-indan-5-yl-5-oxo-5,8-dihydro-pyrido[2,3-d]pyrimidine-6-carboxylic acid methoxy-amide (Cpd 197)

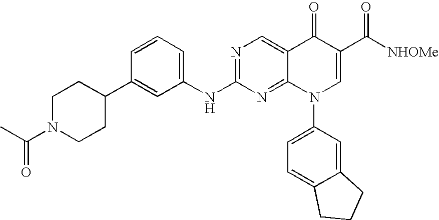

### A. 4-(3-nitro-phenyl)-3,6-dihydro-2H-pyridine-1-carboxylic acid tert-butyl ester

To a solution of 703 μL of diisopropylamine in 10 mL of anhydrous THF at −78° C. under argon, was added dropwise 2.1 mL of n-BuLi (2.5M in hexanes). After stirring for 20 min at −78° C., was added dropwise a solution of 1 g of 4-oxo-piperidine-1-carboxylic acid tert-butyl ester in 5 mL of anhydrous THF. After stirring for 30 min, was added dropwise a solution of 1.88 g of bis-C,C,C-trifluoro-N-phenyl-methanesulfonamide in 10 mL of anhydrous THF. The reaction was stirred overnight with gradual warming to room temperature. The reaction mixture was diluted with ethyl acetate and washed twice with water. The organic layer was dried over sodium sulfate, filtered, and the filtrate was concentrated in vacuo to give 1.66 g of product. A mixture of this material, 1.2 g of 3-nitrophenylboronic acid, 1.4 g of lithium chloride, 260 mg of tetrakistriphenylphosphine palladium (0), and 12.6 mL of 2 M sodium carbonate in 15 mL of DMF was heated in a sealed tube at reflux for 3 h. The reaction mixture was cooled, diluted with water and extracted with EtOAc (3×). The combined organic fractions were dried over sodium sulfate, the filtered and concentrated. The residue was purified by chromatography (silica, ethyl acetate:hexanes, 1:4) to give the title compound (148 mg).

### B. 1-[4-(3-amino-phenyl)-piperidin-1-yl]-ethanone

A solution of 148 mg of 4-(3-nitro-phenyl)-3,6-dihydro-2H-pyridine-1-carboxylic acid tert-butyl ester in 2 mL of 1:1 trifluoroacetic acid/dichloromethane was stirred for 2 h at rt. The reaction was concentrated in vacuo, dissolved in 2 mL of dichloromethane. To this solution was added 250 μL of triethylamine followed by 50 μL of acetyl chloride, and the reaction was for stirred 1 h at rt. The reaction was concentrated in vacuo. The reaction was then concentrated in vacuo. The residue was taken up in 5 mL of ethanol and the vessel was purged with Argon then approximately 25 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo to afford the title compound after filtration (95 mg, 97%).

### C. 2-[3-(1-acetyl-piperidin-4-yl)-phenylamino]-8-indan-5-yl-5-oxo-5,8-dihydro-pyrido[2,3-d]pyrimidine-6-carboxylic acid methoxy-amide

100 mg of 8-indan-5-yl-2-methanesulfonyl-5-oxo-5,8-dihydro-pyrido[2,3-d]pyrimidine-6-carboxylic acid methoxy-amide, 63 mg of 1-[4-(3-amino-phenyl)-piperidin-1-yl]-ethanone, and 87 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics were dried over sodium sulfate and concentrated in vacuo. Purification of the crude material on a 2000 micron Prep. TLC plate using 10% methanol saturated with ammonia/dichloromethane as the eluent afforded (12 mg. 8%) the title compound. ¹H NMR (300 MHz, CDCl₃) δ (ppm): 11.82 (s, 1H), 9.30 (s, 1H), 8.47 (s, 1H), 8.27 (bs, 1H), 7.34 (d, 1H), 7.18 (m, 3H), 7.10 (d, 1H), 6.96 (bs, 1H), 6.79 (d, 1H), 4.70 (m, 1H), 3.83 (s, 3H), 2.99 (t, 2H), 2.91 (t, 2H),), 2.54 (m, 2H), 2.15 (m, 2H), 2.07 (s, 3H), 1.74 (m, 2H), 1.17 (m, 2H). Mass Spectrum (LCMS, ESI pos.) Calcd. For C₃₁H₃₂N₆O₄: 552.62 (M+H), Found: 553.2/554.2.

### Example 50

### 8-indan-5-yl-2-[3-(2-morpholin-4-yl-ethylcarbamoyl)-phenylamino]-5-oxo-5,8-dihydro-pyrido[2,3-d]pyrimidine-6-carboxylic acid methoxy-amide (Cpd 198)

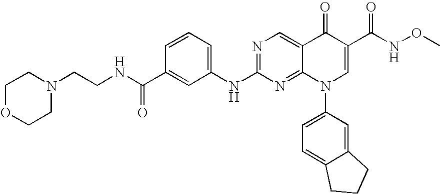

### A. 3-amino-N-(2-morpholin-4-yl-ethyl)-benzamide

1 g of 3-nitro-benzoyl chloride was taken up in 25 mL of dichloromethane and the reaction was cooled to 0° C. 1.029 mL of diisopropylamine was then added, followed by 832 mg of 2-morpholin-4-yl-ethylamine. The mixture was stirred for 4 hours with gradual warming to room temperature. The reaction mixture was then washed twice with water, dried over sodium sulfate and concentrated in vacuo to provide a crude material that was taken up in 25 mL of ethanol. The solution was put in a reaction vessel that was purged with Argon and approximately 100 mg of 10% Pd on carbon was added. The vessel was evacuated and purged with hydrogen via balloon. The mixture was stirred for 2 hours at room temperature. The vessel was again evacuated and purged with Argon then the reaction mixture was filtered and concentrated in vacuo. Trituration of the crude material in 5% ethyl acetate/hexane afforded the title compound (1.2 g, 90%).

### B. 8-indan-5-yl-2-[3-(2-morpholin-4-yl-ethylcarbamoyl)-phenylamino]-5-oxo-5,8-dihydro-pyrido[2,3-d]pyrimidine-6-carboxylic acid methoxy-amide

110 mg of 8-indan-5-yl-2-methanesulfonyl-5-oxo-5,8-dihydro-pyrido[2,3-d]pyrimidine-6-carboxylic acid methoxy-amide, 68 mg of 3-Amino-N-(2-morpholin-4-yl-ethyl)-benzamide, and 92 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 24 mg (15%) of the title compound. Mass Spectrum (LCMS, ESI pos.) Calcd. For C₃₁H₃₃N₇O₅: 583.64 (M+H), Found: 584.2/585.2.

### Example 51

### 2-[3-(2-dimethylamino-ethylcarbamoyl)-phenylamino]-8-indan-5-yl-5-oxo-5,8-dihydro-pyrido[2,3-d]pyrimidine-6-carboxylic acid methoxy-amide (Cpd 199)

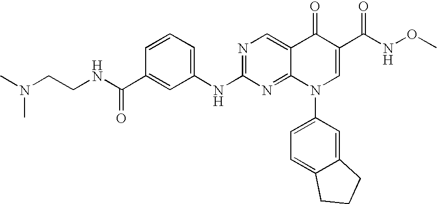

### A. 3-amino-N-(2-dimethylamino-ethyl)-benzamide

1 g of 3-nitro-benzoyl chloride was taken in 25 mL of dichloromethane and the reaction was cooled to 0° C. 1.029 mL of diisopropylamine was added followed by 569 mg of N,N-dimethyl-ethane-1,2-diamine. The reaction was stirred for 4 hours with gradual warming to room temperature. The reaction mixture was then washed twice with water, dried over sodium sulfate, and concentrated in vacuo. The crude material was then taken in 25 mL of ethanol and the vessel was purged with Argon then approximately 100 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo. Trituration of the crude material in 5% ethyl acetate/hexane afforded 1 g (90%) of the title compound.

### B. 2-[3-(2-dimethylamino-ethylcarbamoyl)-phenylamino]-8-indan-5-yl-5-oxo-5,8-dihydro-pyrido[2,3-d]pyrimidine-6-carboxylic acid methoxy-amide

110 mg of 8-indan-5-yl-2-methanesulfonyl-5-oxo-5,8-dihydro-pyrido[2,3-d]pyrimidine-6-carboxylic acid methoxy-amide, 68 mg of 3-amino-N-(2-dimethylamino-ethyl)-benzamide, and 92 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 17 mg (11%) of the title compound. Mass Spectrum (LCMS, ESI pos.) Calcd. For C₂₉H₃₁N₇O₄: 541.60 (M+H), Found: 542.2/543.2.

### Example 52

### 8-indan-5-yl-2-{3-[2-(4-methyl-piperazin-1-yl)-ethyl]-phenylamino}-5-oxo-5,8-dihydro-pyrido[2,3-d]pyrimidine-6-carboxylic acid methoxy-amide (Cpd 200)

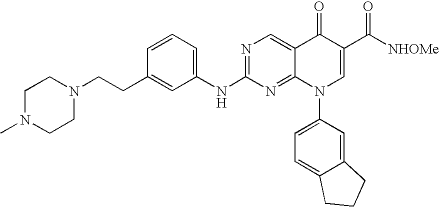

### A. 3-[2-(4-methyl-piperazin-1-yl)-ethyl]-phenylamine

2 g of methanesulfonic acid 2-(3-nitro-phenyl)-ethyl ester was taken in 40 mL of anhydrous DMF and 3 g of cesium carbonate was added. 817 mg of N-methyl piperazine was added and the reaction mixture was heated to 100° C. for 2 days. The reaction was then cooled and diluted with water then extracted three times with ether. The combined organics are washed twice with water, dried over sodium sulfate, and concentrated in vacuo. Purification via column chromatography using 5% methanol/dichloromethane afforded 850 mg (43%) of the desired compound. This material was then taken in 25 mL of ethanol and the vessel was purged with Argon then approximately 100 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo to afford the title compound (800 mg, 98%).

### B. 8-indan-5-yl-2-{3-[2-(4-methyl-piperazin-1-yl)-ethyl]-phenylamino}-5-oxo-5,8-dihydro-pyrido[2,3-d]pyrimidine-6-carboxylic acid methoxy-amide

125 mg of 8-indan-5-yl-2-methanesulfonyl-5-oxo-5,8-dihydro-pyrido[2,3-d]pyrimidine-6-carboxylic acid methoxy-amide, 3-[2-(4-methyl-piperazin-1-yl)-ethyl]-phenylamine, and 104 mg of silver triflate were taken up in 3 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 10 mg (6%) of the title compound. Mass Spectrum (LCMS, ESI pos.) Calcd. For C₃₁H₃₅N₇O₃: 553.65 (M+H), Found: 554.3/555.3.

### Example 53

### 8-indan-5-yl-2-[3-(4-methanesulfonyl-piperazin-1-yl)-phenylamino]-5-oxo-5,8-dihydro-pyrido[2,3-d]pyrimidine-6-carboxylic acid methoxy-amide (Cpd 201)

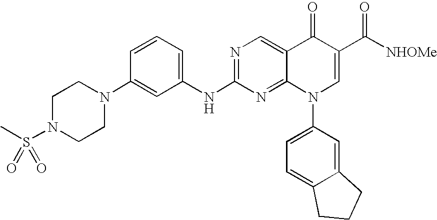

### A. 3-(4-methanesulfonyl-piperazin-1-yl)-phenylamine

400 mg of 1-(3-nitro-phenyl)-piperazine was taken in 20 mL of dichloromethane and 600 μL of triethyl amine was added. 243 mg of methanesulfonyl chloride was added and the mixture was stirred at room temperature for 2 hours. The reaction mixture was then washed twice with water, dried over sodium sulfate, and concentrated in vacuo. The resulting material was then taken up in 25 mL of ethanol and the vessel was purged with Argon then approximately 50 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo to afford 100 mg (20%) of the title compound.

### B. 8-indan-5-yl-2-[3-(4-methanesulfonyl-piperazin-1-yl)-phenylamino]-5-oxo-5,8-dihydro-pyrido[2,3-d]pyrimidine-6-carboxylic acid methoxy-amide

100 mg of 8-indan-5-yl-2-methanesulfonyl-5-oxo-5,8-dihydro-pyrido[2,3-d]pyrimidine-6-carboxylic acid methoxy-amide, 73 mg of 3-(4-methanesulfonyl-piperazin-1-yl)-phenylamine, and 87 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded (6 mg, 4%) of the title compound. ¹H NMR (300 MHz, CDCl₃) δ (ppm): 11.95 (s, 1H), 9.32 (s, 1H), 8.72 (s, 1H), 7.74 (bs, 1H), 7.34 (d, 1H), 7.18 (m, 3H), 7.10 (d, 1H), 6.96 (bs, 1H), 6.79 (d, 1H), 3.83 (s, 3H), 3.37 (m, 4H), 3.15 (m, 4H), 2.99 (t, 2H), 2.91 (t, 2H), 2.77 (s, 3H), 2.15 (m, 2H). Mass Spectrum (LCMS, ESI pos.) Calcd. For C₂₉H₃₁N₇O₅S: 589.67 (M+H), Found: 590.1/591.2.

### Example 54

### 8-indan-5-yl-5-oxo-2-[3-(4-trifluoromethanesulfonyl-piperazin-1-yl)-phenylamino]-5,8-dihydro-pyrido[2,3-d]pyrimidine-6-carboxylic acid methoxy-amide (Cpd 202)

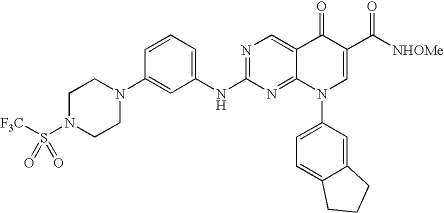

### A. 3-(4-trifluoromethanesulfonyl-piperazin-1-yl)-phenylamine

400 mg of 1-(3-nitro-phenyl)-piperazine was taken in 20 mL of dichloromethane and 600 μL of triethyl amine was added. 360 mg of trifluoromethanesulfonyl chloride was added and the reaction was stirred at room temperature for 2 hours. The reaction was then washed twice with water, dried over sodium sulfate, and concentrated in vacuo. This material was then taken in 25 mL of ethanol and the vessel was purged with Argon then approximately 50 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo to afford 103 mg (17%) of the title compound.

### B. 8-indan-5-yl-5-oxo-2-[3-(4-trifluoromethanesulfonyl-piperazin-1-yl)-phenylamino]-5,8-dihydro-pyrido[2,3-d]pyrimidine-6-carboxylic acid methoxy-amide

100 mg of 8-indan-5-yl-2-methanesulfonyl-5-oxo-5,8-dihydro-pyrido[2,3-d]pyrimidine-6-carboxylic acid methoxy-amide, 82 mg of 3-(4-trifluoromethanesulfonyl-piperazin-1-yl)-phenylamine, and 87 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 6 mg (5%) of the title compound. ¹H NMR (300 MHz, CDCl₃) δ (ppm): 11.85 (s, 1H), 9.30 (s, 1H), 8.72 (s, 1H), 7.74 (bs, 1H), 7.34 (d, 1H), 7.18 (m, 3H), 7.10 (d, 1H), 6.96 (bs, 1H), 6.79 (d, 1H), 3.83 (s, 3H), 3.47 (m, 4H), 3.07 (m, 4H), 2.97 (t, 2H), 2.93 (t, 2H), 2.14 (m, 2H). Mass Spectrum (LCMS, ESI pos.) Calcd. For C₂₉H₂₈F₃N₇O₅S: 643.64 (M+H), Found: 644.1/645.1.

### Example 55

### 8-indan-5-yl-2-{3-[2-(4-methanesulfonyl-piperazin-1-yl)-ethyl]-phenylamino}-5-oxo-5,8-dihydro-pyrido[2,3-d]pyrimidine-6-carboxylic acid methoxy-amide (Cpd 203)

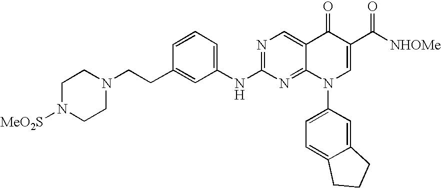

### A. 1-[2-(3-nitro-phenyl)-ethyl]-piperazine

2.4 g of methanesulfonic acid 2-(3-nitro-phenyl)-ethyl ester was taken in 100 mL of anhydrous DMF and 3.5 g of cesium carbonate was added. 1.9 g of piperazine-1-carboxylic acid tert-butyl ester was added and the reaction mixture was heated to 100° C. for 4 hours. The reaction was then cooled and diluted with water then extracted three times with ether. The combined organics are washed twice with water, dried over sodium sulfate, and concentrated in vacuo. Purification via column chromatography using 5% methanol/dichloromethane afforded 3.2 g (100%) of the desired compound. This material was then taken in 50 mL of 1:1 trifluoroacetic acid/dichloromethane and stirred for 2 hours at room temperature. The reaction was then concentrated in vacuo to afford 2.3 g (98%) of the title compound.

### B. 3-[2-(4-methanesulfonyl-piperazin-1-yl)-ethyl]-phenylamine

500 mg of 1-[2-(3-nitro-phenyl)-ethyl]-piperazine was taken in 20 mL of dichloromethane and 700 μL of triethyl amine was added. 268 mg of methanesulfonyl chloride was added and the reaction was stirred at room temperature for 2 hours. The reaction was then washed twice with water, dried over sodium sulfate, and concentrated in vacuo. The product was taken up in 25 mL of ethanol and the vessel was purged with Argon then approximately 50 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo. Purification of the crude material on a 2000 micron Prep. TLC plate using 5% methanol saturated with ammonia/dichloromethane as the eluent afforded 100 mg (17%) of the title compound.

### C. 8-indan-5-yl-2-{3-[2-(4-methanesulfonyl-piperazin-1-yl)-ethyl]-phenylamino}-5-oxo-5,8-dihydro-pyrido[2,3-d]pyrimidine-6-carboxylic acid methoxy-amide

100 mg of 8-indan-5-yl-2-methanesulfonyl-5-oxo-5,8-dihydro-pyrido[2,3-d]pyrimidine-6-carboxylic acid methoxy-amide, 82 mg of 3-[2-(4-methanesulfonyl-piperazin-1-yl)-ethyl]-phenylamine, and 87 mg of silver triflate were taken up in 2 mL of 1,4 dioxane and the reaction was heated to 107° C. for 3 hours. The reaction was then diluted with water and ethyl acetate then filtered. The filtrate was then extracted three times with ethyl acetate and the combined organics are dried over sodium sulfate and concentrated in vacuo. Purification of the crude material on a 2000 micron Prep. TLC plate using 10% methanol saturated with ammonia/dichloromethane as the eluent afforded 6 mg (8%) of the title compound. ¹H NMR (300 MHz, CDCl₃) δ (ppm): 11.85 (s, 1H), 9.30 (s, 1H), 8.72 (s, 1H), 7.74 (bs, 1H), 7.34 (d, 1H), 7.18 (m, 3H), 7.10 (d, 1H), 6.96 (bs, 1H), 6.79 (d, 1H), 4.11 (t, 2H), 3.83 (s, 3H), 3.47 (m, 4H), 3.07 (m, 4H), 2.97 (t, 2H), 2.93 (t, 2H), 2.69 (s, 3H), 2.66 (t, 2H), 2.14 (m, 2H). Mass Spectrum (LCMS, ESI pos.) Calcd. For C₃₁H₃₅N₇O₅S: 617.72 (M+H), Found: 662/663.

### Example 56

### 2-{3-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide (Cpd 204)

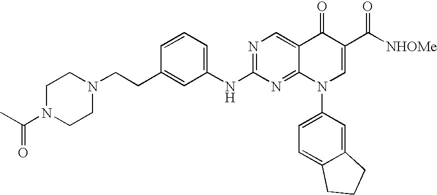

### A. 1-{4-[2-(3-amino-phenyl)-ethyl]-piperazin-1-yl}-ethanone

500 mg of 1-[2-(3-nitro-phenyl)-ethyl]-piperazine was taken in 20 mL of dichloromethane and 700 μL of triethyl amine was added. 200 mg of acetyl chloride was added and the reaction was stirred at room temperature for 3 hours. The reaction was then washed twice with water, dried over sodium sulfate, and concentrated in vacuo. This material was then taken in 25 mL of ethanol and the vessel was purged with Argon then approximately 50 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo. Purification of the crude material on a 2000 micron Prep. TLC plate using 5% methanol saturated with ammonia/dichloromethane as the eluent afforded 80 mg (17%) of the title compound.

### B. 2-{3-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide

70 mg of 8-indan-5-yl-2-methanesulfonyl-5-oxo-5,8-dihydro-pyrido[2,3-d]pyrimidine-6-carboxylic acid methoxy-amide, 50 mg of 1-{4-[2-(3-amino-phenyl)-ethyl]-piperazin-1-yl}-ethanone, and 60 mg of silver triflate were taken up in 2 mL of 1,4 dioxane and the reaction was heated to 107° C. for 3 hours. The reaction was then diluted with water and ethyl acetate then filtered. The filtrate was then concentrated in vacuo. Purification of the crude material on a 2000 micron Prep. TLC plate using 10% methanol saturated with ammonia/dichloromethane as the eluent afforded 16 mg (17%) of the title compound. ¹H NMR (300 MHz, CDCl₃) δ (ppm): 11.88 (s, 1H), 9.31 (s, 1H), 8.74 (s, 1H), 7.46 (s, 1H), 7.34 (d, 1H), 7.27 (bs, 1H), 7.18 (m, 3H), 7.10 (d, 1H), 6.99 (bs, 1H), 6.81 (d, 1H), 3.83 (s, 3H), 3.58 (m, 4H), 3.38 (m, 4H), 2.96 (t, 2H), 2.92 (t, 2H), 2.15 (m, 2H), 2.03 (s, 3H). Mass Spectrum (LCMS, ESI pos.) Calcd. For C₃₂H₃₅N₇O₄: 581.67 (M+H), Found: 582.2/583.2.

### Example 57

### 2-{3-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide (Cpd 205)

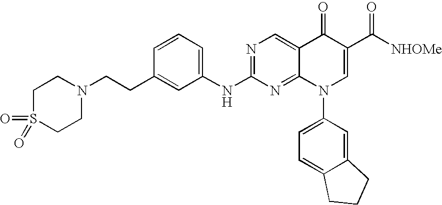

### A. 3-[2-(1,1-dioxo-1λ⁶-thiomorpholin-4-yl)-ethyl]-phenylamine

2.1 g of methanesulfonic acid 2-(3-nitro-phenyl)-ethyl ester was taken in 100 mL of anhydrous DMF and 3.1 g of cesium carbonate was added. 1.15 g of thiomorpholine 1,1-dioxide was added and the reaction mixture was heated to 75° C. for 16 hours. The reaction was then cooled and diluted with water then extracted three times with ether. The combined organics are washed twice with water, dried over sodium sulfate, and concentrated in vacuo. This material was then taken in 25 mL of ethanol and the vessel was purged with Argon then approximately 50 mg of 10% Pd on carbon was added. The vessel was then evacuated and purged with hydrogen via balloon and stirred for 2 hours at room temperature. The vessel was again evacuated and purged with Argon then the reaction mixture was filtered and concentrated in vacuo. Purification via column chromatography using 5% methanol/dichloromethane afforded 480 mg (23%) of the title compound.

### B. 2-{3-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide

70 mg of 8-indan-5-yl-2-methanesulfonyl-5-oxo-5,8-dihydro-pyrido[2,3-d]pyrimidine-6-carboxylic acid methoxy-amide, 50 mg of 3-[2-(1,1-dioxo-1λ⁶-thiomorpholin-4-yl)-ethyl]-phenylamine, and 60 mg of silver triflate were taken up in 2 mL of 1,4-dioxane and the reaction was heated to 107° C. for 3 hours. The reaction was then diluted with water and ethyl acetate then filtered. The filtrate was then concentrated in vacuo. Purification of the crude material on a 2000 micron Prep. TLC plate using 10% methanol saturated with ammonia/dichloromethane as the eluent afforded 10 mg (10%) of the title compound. ¹H NMR (300 MHz, CDCl₃) δ (ppm): 11.96 (s, 1H), 9.32 (s, 1H), 8.70 (s, 1H), 7.35 (d, 1H), 7.2 (m, 2H), 6.96 (bs, 1H), 6.8 (d, 1H), 4.15 (m, 2H), 3.82 (s, 3H), 3.76 (m, 2H), 2.97 (t, 2H), 2.93 (t, 2H), 2.69 (m, 4H), 2.14 (m, 2H). Mass Spectrum (LCMS, ESI pos.) Calcd. For C₃₀H₃₂N₆O₅S: 588.68 (M+H), Found: 633.1/634.1.

Using the procedure of Example 57 and reagents, starting materials and conditions known to those skilled in the art, the following compounds representative of the present invention were prepared:

| Cpd | Name and Data |
|---|---|
| 206 | 2-{4-[2-(1,1-dioxo-1λ⁶-thiomorpholin-4-yl)-ethyl]-phenylamino}- |
| | 8-indan-5-yl-5-oxo-5,8-dihydro-pyrido[2,3-d]pyrimidine-6- |
| | carboxylic acid methoxy-amide |
| | ¹H NMR (400 MHz, CDCl₃) δ (ppm): 11.95 (s, 1H), 9.40 |
| | (s, 1H), 8.82 (s, 1H), 7.65 (s, 1H), 7.40 (d, 1H), 7.24 (s, 1H), |
| | 7.18 (d, 1H), 6.92 (br, 4H), 3.83 (s, 3H), 3.15-3.00 (m, 16H), |
| | 2.25 (m, 2H). Mass Spectrum (LCMS, APCI pos.) Calcd. |
| | For C₃₀H₃₂N₆O₅S: 589.2 (M + H), Found: 589.3. |

### Example 58

### 8-indan-5-yl-5-oxo-2-[4-(2-pyrrolidin-1-yl-ethyl)-phenylamino]-5,8-dihydro-pyrido[2,3-d]pyrimidine-6-carboxylic acid thiazol-2-ylamide (Cpd 131)

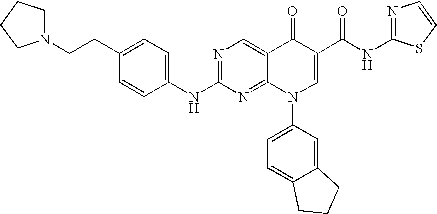

A solution of 8-indan-5-yl-5-oxo-2-[4-(2-pyrrolidin-1-yl-ethyl)-phenylamino]-5,8-dihydro-pyrido[2,3-d]pyrimidine-6-carboxylic acid methyl ester (10 mg) and thiazol-2-ylamine hydrochloride (20 mg) in 1:1 TEA and MeOH (2 mL) was heated in a sealed tube at 110° C. for 2 days. After cooling, water was added and the solution was directly purified (HPLC, C-18 YMC ODS-A 5μ 30×100 mm, 120 A column at 32 mL/min, 5-100% H₂O/MeCN (0.1% TFA v/v) gradient over 10 min.) to provide the title compound (2.5 mg). ¹H NMR (TFA salt) (400 MHz, CDCl₃) δ (ppm): 9.46 (s, 1H), 8.92 (s, 1H), 7.44-7.35 (m, 3H), 7.30-7.23 (m, 4H), 7.13-7.06 (m, 2H), 3.83 (m, 5H), 3.22-3.13 (m, 2H), 3.01-3.07 (m, 4H), 2.78-2.53 (m, 6H), 2.16-2.20 (m, 4H); Mass Spectrum (LCMS, APCI pos.) Calcd. For: C₃₂H₃₁N₇O₂S: 577.23; Found: 578.2 (M+H).

### BIOLOGICAL EXAMPLES

### Example 1

### Autophosphorylation, Fluorescence Polarization Competition Immunoassay

An autophosphorylation, fluorescence polarization competition immunoassay was used to determine the potency for c-fms inhibition exhibited by selected compounds of Formula I. The assay was performed in black 96-well microplates (LJL BioSystems). The assay buffer used was 100 mM 4-(2-hydroxyethyl)piperazine 1-ethanesulfonic acid (HEPES), pH 7.5, 1 mM 1,4-dithio-DL-threitol (DTT), 0.01% (v/v) Tween-20. Compounds were diluted in assay buffer containing 4% dimethylsulfoxide (DMSO) just prior to the assay. To each well, 5 μL of compound were added followed by the addition of 3 μL of a mix containing 33 nM c-fms and 16.7 mM MgCl₂ (Sigma) in assay buffer. The kinase reaction was initiated by adding 2 μL of 5 mM ATP (Sigma) in assay buffer. The final concentrations in the assay were 10 nM c-fms, 1 mM ATP, 5 mM MgCl₂, 2% DMSO. Control reactions were ran in each plate: in positive and negative control wells, assay buffer (made 4% in DMSO) was substituted for the compound; in addition, positive control wells received 1.2 μL of 50 mM ethylenediaminetetraacetic acid (EDTA).

The plates were incubated at room temperature for 45 min. At the end of the incubation, the reaction was quenched with 1.2 μL of 50 mM EDTA (EDTA was not added to the positive control wells at this point; see above). Following a 5-min incubation, each well received 10 μL of a 1:1:3 mixture of anti-phosphotyrosine antibody, 10×, PTK green tracer, 10× (vortexed), FP dilution buffer, respectively (all from PanVera, cat. # P2837). The plate was covered, incubated for 30 min at room temperature and the fluorescence polarization was read on the Analyst. The instrument settings were: 485 nm excitation filter; 530 nm emission filter; Z height: middle of well; G factor: 0.93. Under these conditions, the fluorescence polarization values for positive and negative controls were approximately 300 and 150, respectively, and were used to define the 100% and 0% inhibition of the c-fms reaction.

The IC₅₀ values shown in Table 1 are averages of three independent measurements.

TABLE 1
c-fms Autophosphorylation IC₅₀ Values

| Cpd | IC₅₀ (μM) |
|---|---|
| 112 | 0.004 |
| 113 | >1 |
| 119 | 0.0026 |

### Example 2

### Peptide (Non-Phosphorylated) Assay

A fluorescence polarization competition immunoassay was used to measure compound inhibition of CSF-1R phosphorylation of tyrosine on a synthetic CSF-1R₅₅₅₋₅₆₈ peptide (SYEGNSYTFIDPTQ). The assay was performed in black 96-well microplates (Cat # 42-000-0117, Molecular Devices, Sunnyvale, Calif.). To each well, 5 μL of compound (in 4% DMSO) were mixed with 2 μL of 3.5 nM CSF-1R, 25 mM MgCl₂ in assay buffer (100 mM HEPES, pH 7.5, 1 mM DTT, 0.01% Tween-20), and 2 μL of 1540 μM peptide in assay buffer. The kinase reaction was initiated by adding 1 μL of 10 mM ATP in assay buffer. The final concentrations in the 10 μL reaction mixture were 100 mM HEPES, pH 7.5, 1 mM DTT, 0.01% Tween-20, 2% DMSO, 308 μM SYEGNSYTFIDPTQ, 1 mM ATP, 5 mM MgCl₂, and 0.7 nM CSF-1R. Positive and negative control wells were included on each plate, where 4% DMSO in assay buffer was substituted for the compound; in addition, positive control wells received 1.2 μL of 50 mM EDTA before the start of the reaction.

The plates were covered and incubated at room temperature for 80 min. Reactions were stopped by addition of 1.2 μL of 50 mM EDTA. Each well then received 10 μL of a 1:1:3 mixture of 10× anti-phosphotyrosine antibody, 10×PTK green tracer, and FP dilution buffer, respectively (Cat. # P2837, Invitrogen, Carlsbad, Calif.). The plates were covered, incubated for 30 min at room temperature, and the fluorescence polarization was read on an Analyst plate reader (Molecular Devices). Instrument settings were: 485 nm excitation, 530 nm emission, with a 505 nm cut-off filter; Z height: middle of well; G factor: 0.93. Under these conditions, the fluorescence polarization values for positive and negative controls were approximately 290 and 160, respectively, and were used to define 100% and 0% inhibition of the CSF-1R reaction.

The IC₅₀ values reported in Table 2 are the mean of at least three determinations.

TABLE 2
c-fms Peptide IC₅₀ Values

| Cpd | IC₅₀ (μM) |
|---|---|
| 1 | 0.0045 |
| 2 | 0.00056 |
| 3 | 0.028 |
| 4 | 0.0022 |
| 5 | 0.0015 |
| 6 | 0.0031 |
| 7 | 0.0017 |
| 8 | 0.0016 |
| 9 | 0.00024 |
| 10 | 0.0021 |
| 11 | >0.3 |
| 12 | 0.0013 |
| 13 | 0.0016 |
| 14 | 0.0041 |
| 15 | 0.00019 |
| 16 | 0.00064 |
| 17 | 0.00094 |
| 18 | 0.0003 |
| 19 | 0.001 |
| 20 | 0.003 |
| 21 | 0.001 |
| 22 | 0.0015 |
| 23 | 0.003 |
| 24 | 0.0018 |
| 25 | 0.0021 |
| 26 | 0.0014 |
| 27 | 0.0016 |
| 28 | 0.0022 |
| 29 | 0.001 |
| 30 | 0.014 |
| 31 | 0.0026 |
| 32 | 0.0037 |
| 33 | 0.02 |
| 34 | 0.0038 |
| 35 | 0.0022 |
| 36 | 0.0021 |
| 37 | 0.0015 |
| 38 | 0.002 |
| 39 | 0.0011 |
| 40 | 0.0068 |
| 41 | 0.0041 |
| 42 | 0.057 |
| 43 | 0.031 |
| 44 | 0.11 |
| 45 | 0.0019 |
| 46 | 0.0026 |
| 47 | 0.0019 |
| 48 | 0.0015 |
| 49 | 0.0017 |
| 50 | 0.0012 |
| 51 | 0.00063 |
| 52 | 0.0008 |
| 53 | 0.0005 |
| 54 | 0.0018 |
| 55 | 0.00051 |
| 56 | 0.0011 |
| 57 | 0.0006 |
| 58 | 0.0016 |
| 59 | 0.0045 |
| 60 | 0.007 |
| 61 | 0.00068 |
| 62 | 0.00054 |
| 63 | 0.00052 |
| 64 | 0.18 |
| 65 | 0.00068 |
| 66 | 0.0025 |
| 67 | 0.001 |
| 68 | 0.0007 |
| 69 | 0.0012 |
| 70 | 0.0017 |
| 71 | 0.00064 |
| 72 | 0.00084 |
| 73 | 0.00084 |
| 74 | 0.00062 |
| 75 | 0.00053 |
| 76 | 0.00083 |
| 77 | 0.00095 |
| 78 | 0.00072 |
| 79 | 0.00087 |
| 80 | 0.0013 |
| 81 | 0.003 |
| 82 | 0.0036 |
| 83 | 0.001 |
| 84 | 0.002 |
| 85 | 0.0022 |
| 86 | 0.0018 |
| 87 | 0.0024 |
| 88 | 0.0013 |
| 89 | 0.0014 |
| 90 | 0.0024 |
| 91 | 0.002 |
| 92 | 0.0049 |
| 93 | 0.0049 |
| 94 | 0.0009 |
| 95 | 0.0018 |
| 96 | 0.0009 |
| 97 | 0.00063 |
| 98 | 0.0014 |
| 99 | 0.0009 |
| 100 | 0.00064 |
| 101 | 0.00055 |
| 102 | 0.0023 |
| 103 | 0.0072 |
| 104 | 0.003 |
| 105 | 0.0014 |
| 106 | 0.0016 |
| 107 | 0.0015 |
| 108 | 0.00088 |
| 109 | 0.0011 |
| 110 | 0.00046 |
| 111 | 0.00058 |
| 112 | 0.0003 |
| 114 | 0.0096 |
| 115 | 0.00029 |
| 116 | 0.0053 |
| 117 | 0.0065 |
| 118 | 0.0045 |
| 120 | 0.017 |
| 121 | 0.0026 |
| 122 | 0.0066 |
| 123 | 0.0045 |
| 124 | 0.0045 |
| 125 | 0.0057 |
| 126 | 0.0018 |
| 128 | 0.0011 |
| 129 | 0.0023 |
| 130 | 0.0016 |
| 132 | 0.012 |
| 133 | 0.0012 |
| 134 | 0.0068 |
| 135 | 0.066 |
| 136 | 0.0042 |
| 137 | 0.016 |
| 138 | 0.00078 |
| 139 | 0.001 |
| 140 | 0.0048 |
| 141 | 0.0013 |
| 142 | 0.066 |
| 143 | 0.13 |
| 144 | 0.0004 |
| 145 | 0.0012 |
| 146 | 0.0012 |
| 147 | 0.00094 |
| 148 | 0.0012 |
| 149 | 0.0026 |
| 150 | 0.00075 |
| 151 | 0.0015 |
| 152 | 0.0015 |
| 153 | 0.0032 |
| 154 | 0.0073 |
| 155 | 0.00012 |
| 156 | 0.0003 |
| 157 | 0.0004 |
| 159 | 0.00066 |
| 162 | 0.0021 |
| 171 | 0.00076 |
| 172 | 0.0023 |
| 173 | 0.00077 |
| 174 | 0.0021 |
| 175 | 0.001 |
| 176 | 0.00059 |
| 177 | 0.0066 |
| 178 | 0.0026 |
| 179 | 0.0018 |
| 181 | 0.0002 |
| 182 | 0.0007 |
| 183 | 0.0004 |
| 184 | 0.0009 |
| 185 | 0.0006 |
| 186 | 0.0006 |
| 187 | 0.0007 |
| 188 | 0.0041 |
| 189 | 0.0097 |
| 190 | 0.0042 |
| 191 | 0.0056 |
| 192 | 0.0063 |
| 193 | 0.0025 |
| 194 | 0.00065 |
| 195 | 0.00096 |
| 196 | 0.0017 |
| 197 | 0.0014 |
| 198 | 0.0042 |
| 199 | 0.0014 |
| 200 | 0.0013 |
| 201 | 0.0018 |
| 202 | 0.068 |
| 203 | 0.0014 |
| 204 | 0.0012 |
| 205 | 0.001 |
| 206 | 0.0012 |
| 207 | 0.0003 |
| 208 | 0.0004 |
| 209 | 0.0004 |
| 210 | 0.00025 |
| 211 | 0.0008 |
| 212 | 0.00036 |
| 213 | 0.0011 |
| 214 | 0.0016 |
| 215 | 0.001 |
| 216 | 0.0016 |
| 217 | 0.00062 |
| 218 | 0.0012 |
| 219 | 0.0022 |
| 220 | 0.00075 |
| 221 | 0.00087 |
| 222 | 0.029 |
| 223 | 0.00063 |
| 224 | 0.00058 |
| 225 | 0.007 |
| 226 | 0.0077 |
| 227 | 0.01 |
| 228 | 0.00068 |
| 229 | 0.00042 |
| 230 | 0.0011 |
| 231 | 0.00071 |
| 232 | 0.00072 |
| 233 | 0.0017 |
| 234 | 0.0017 |
| 235 | 0.00054 |
| 236 | 0.00047 |
| 237 | 0.00071 |
| 238 | 0.00064 |
| 239 | 0.00032 |
| 240 | 0.00028 |
| 241 | 0.0017 |
| 242 | 0.00059 |
| 243 | 0.00052 |
| 244 | 0.00064 |
| 245 | 0.00029 |
| 246 | 0.00038 |
| 247 | 0.00059 |
| 248 | 0.00043 |
| 249 | 0.00055 |
| 250 | 0.00039 |
| 251 | 0.00093 |
| 252 | 0.00057 |
| 253 | 0.00066 |
| 254 | 0.00052 |
| 255 | 0.00067 |
| 256 | 0.00088 |
| 257 | 0.0012 |
| 258 | 0.00094 |
| 259 | 0.0007 |
| 260 | 0.00061 |
| 261 | 0.00095 |
| 262 | 0.00059 |
| 263 | 0.00056 |
| 264 | 0.00038 |
| 265 | 0.00057 |
| 266 | 0.00062 |
| 267 | 0.00042 |
| 268 | 0.0004 |
| 269 | 0.0015 |
| 270 | 0.0016 |
| 271 | 0.0032 |
| 272 | 0.00088 |
| 273 | 0.001 |
| 274 | 0.00044 |
| 275 | 0.00066 |
| 276 | 0.00077 |
| 277 | 0.0027 |
| 278 | 0.0049 |
| 279 | 0.0003 |
| 280 | 0.0004 |
| 281 | 0.0015 |
| 282 | 0.0002 |
| 283 | 0.0009 |
| 284 | 0.0022 |
| 285 | 0.0008 |
| 286 | 0.0013 |
| 287 | 0.0009 |
| 288 | 0.0015 |
| 289 | 0.002 |
| 290 | 0.0009 |
| 291 | 0.0013 |
| 292 | 0.006 |
| 293 | 0.0029 |
| 294 | 0.0004 |
| 295 | 0.0012 |
| 296 | 0.0008 |
| 297 | 0.0004 |
| 298 | 0.0004 |
| 299 | 0.032 |
| 300 | 0.0015 |
| 301 | 0.0009 |
| 302 | 0.319 |
| 303 | 0.001 |
| 304 | 0.0006 |
| 305 | 0.0007 |
| 306 | 0.014 |
| 307 | 0.0004 |
| 308 | 0.0009 |
| 309 | 0.0003 |
| 310 | 0.0005 |
| 311 | 0.0003 |
| 312 | 0.0002 |
| 313 | 0.0003 |
| 314 | 0.0005 |
| 315 | 0.0005 |
| 316 | 0.0018 |
| 317 | 0.0055 |
| 318 | 0.0053 |
| 319 | 0.0017 |
| 320 | 0.0005 |
| 321 | 0.0005 |
| 322 | 0.0008 |
| 323 | 0.001 |
| 324 | 0.0005 |
| 325 | 0.0005 |
| 326 | 0.0011 |

While the foregoing specification teaches the principles of the present invention, with examples vided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. A compound of Formula I: 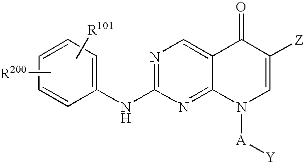
or a salt, stereoisomer, tautomer, crystalline, polymorph, amorphous, solvate, hydrate, ester, prodrug or metabolite form thereof, wherein:
A is absent or C₁₋₈alkyl;
Y is C₃₋₁₄cycloalkyl, aryl, heterocyclyl or heteroaryl each optionally substituted with one, two or three substituents selected from C₁₋₈alkyl, C₂₋₈alkenyl, C₂₋₈alkynyl, C₁₋₈alkoxy, halo-C₁₋₈alkyl, halo-C₁₋₈alkoxy, halogen, hydroxy or amino, wherein heteroaryl is not thiazole;
Z is R¹-amino-carbonyl or heterocyclyl-carbonyl;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₁₋₈alkyl-amino-C₁₋₈alkyl, C₃₋₁₄cycloalkyl-oxy or heteroaryl;
R¹⁰¹ is one or two substituents each selected from hydrogen, halogen or hydroxy;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵—C₃₋₁₄cycloalkyl, R⁵—C₃₋₁₄cycloalkyl-C₁₋₈alkyl, R⁵-aryl, R⁵-aryl-C₁₋₈alkyl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl;
alternatively, R₂₀₀ is a ring selected from heterocyclyl or heteroaryl fused on two adjacent carbon atoms of the phenyl ring of Formula (I) to form an R⁵ substituted bicyclic heterocyclyl or heteroaryl ring;
R² is one, two or three optional substituents each selected from halogen, hydroxy, C₁₋₈alkoxy, carboxy, amino-carbonyl, C₁₋₈alkyl-amino-carbonyl, amino-amino-carbonyl, C₁₋₈alkyl-amino-amino-carbonyl, C₁₋₈alkyl-sulfonyl, amino-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl, R⁵-heterocyclyl-amino-carbonyl or R⁵-heterocyclyl-sulfonyl;
R³ is one, two or three optional substituents each selected from halogen, hydroxy, C₁₋₈alkoxy, amino, C₁₋₈alkyl-amino or R⁵-heterocyclyl;
R⁴ is two substituents each selected from hydrogen, C₁₋₈alkyl, C₁₋₈alkoxy-C₁₋₈alkyl, hydroxy-C₁₋₈alkyl, amino-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or R⁵-heterocyclyl-C₁₋₈alkyl;
R⁵ is one, two, three or four substituents each selected from hydrogen, halogen, hydroxy, oxo, carboxy, R⁶—C₁₋₈alkyl, R⁶—C₁₋₈alkoxy, amino, C₁₋₈alkyl-amino, C₁₋₈alkyl-sulfonyl, amino-sulfonyl, C₁₋₈alkyl-amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, amino, C₁₋₈alkyl-amino, C₁₋₈alkoxy, carboxy, C₁₋₈alkoxy-carbonyl, C₁₋₈alkyl-sulfonyl, C₁₋₈alkyl-sulfonyl-amino-carbonyl, aryl or heterocyclyl, wherein heterocyclyl is optionally substituted with one, two or three substituents each selected from oxo or C₁₋₈alkyl.

2. The compound of claim 1, wherein
Y is C₃₋₁₄cycloalkyl, aryl or heterocyclyl each optionally substituted with one, two or three substituents each selected from C₁₋₈alkyl, C₂₋₈alkynyl, halo-C₁₋₈alkyl, halo-C₁₋₈alkoxy or halogen;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₃₋₁₄cycloalkyl-oxy or heteroaryl;
R^{10l} is hydrogen;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-sulfonyl, R⁴-amino-C₁₋₈alkyl-sulfonyl, R⁵-aryl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl;
R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl or R⁵-heterocyclyl-amino-carbonyl;
R³ is one optional substituent selected from C₁₋₈alkoxy or R⁵-heterocyclyl;
R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or
R⁵-heterocyclyl-C₁₋₈alkyl;
R⁵ is one, two, three or four substituents each selected from hydrogen, halogen, hydroxy, oxo, carboxy, R⁶—C₁₋₈alkyl, R⁶—C₁₋₈alkoxy, C₁₋₈alkyl-amino, C₁₋₈alkyl-sulfonyl, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or aryl.

3. The compound of claim 1, wherein
Y is cyclopropyl, cyclopentyl, cyclohexyl, 1H-indenyl, indanyl, phenyl, naphthalenyl or 6,7-dihydro-5H-cyclopenta[b]pyridinyl each optionally substituted with one, two or three substituents each selected from C₁₋₈alkyl, C₂₋₈alkynyl, halo-C₁₋₈alkyl, halo-C₁₋₈alkoxy or halogen;
Z is R¹-amino-carbonyl, morpholinyl-carbonyl or piperidinyl-carbonyl;
R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, cyclopentyl-oxy or thiazolyl;
R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵-phenyl, R⁵-pyrrolidinyl, R⁵-piperazinyl, R⁵-piperidinyl, R⁵-morpholinyl, R⁵-(1,2,3,6-tetrahydropyridinyl), R⁵-(3,5,11-trioxa-tricyclo[5.3.1.0^{2,6}]undecanyl), R⁵-1H-pyrrolyl, R⁵-1H-pyrazolyl, R⁵-1H-[1,2,4]triazolyl, R⁵-pyrrolidinyl-oxy, R⁵-piperidinyl-oxy, R⁵-pyranyl-oxy, R⁵-(1-azabicyclo[2.2.2]octyl)-oxy, R⁵-pyrrolidinyl-carbonyl, R⁵-piperidinyl-carbonyl, R⁵-morpholinyl-carbonyl, R⁵-piperazinyl-sulfonyl, R⁵-azetidinyl-C₁₋₈alkyl, R⁵-pyrrolidinyl-C₁₋₈alkyl, R⁵-piperazinyl-C₁₋₈alkyl, R⁵-piperidinyl-C₁₋₈alkyl, R⁵-morpholinyl-C₁₋₈alkyl, R⁵-thiomorpholinyl-C₁₋₈alkyl, R⁵-oxazolidinyl-C₁₋₈alkyl, R⁵-(7-aza-bicyclo[2.2.1]heptyl)-C₁₋₈alkyl, 1,5-dioxa-9-aza-spiro[5.5]undec-9-yl-C₁₋₈alkyl, R⁵-(1H-[1,2,4]triazolyl)-C₁₋₈alkyl, R⁵-1H-tetrazol-5-yl-C₁₋₈alkyl, R⁵-imidazolyl-C₁₋₈alkyl or R⁵-pyridinyl-C₁₋₈alkyl;
alternatively, R₂₀₀ is a ring selected from pyrrolidinyl, piperidinyl, 1,4,7,10,13-pentaoxa-cyclopentadecane, 1,4,7,10,13,16-hexaoxa-cyclooctadecane, pyrrolyl, imidazolyl or pyrazolyl fused on two adjacent carbon atoms of the phenyl ring of Formula (I) to form an R⁵ substituted 2,3-dihydro-1H-indolyl, 1,2,3,4-tetrahydroisoquinolinyl, 6,7,9,10,12,13,15,16-octahydro-5,8,11,14,17-pentaoxa-benzocyclopentadecenyl, 6,7,9,10,12,13,15,16,18,19-decahydro-5,8,11,14,17,20-hexaoxa-benzocyclooctadecenyl, 1H-indolyl, 1H-benzimidazolyl or 1H-indazolyl ring;
R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-piperazinyl-carbonyl or R⁵-piperidinyl-amino-carbonyl;
R³ is one optional substituent selected from C₁₋₈alkoxy or R⁵-morpholinyl;
R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, R⁵-adamantanyl, R⁵-bicyclo[2.2.1]heptyl, R⁵-piperidinyl, R⁵-tetrahydro-pyranyl, R⁵-pyrrolidinyl-C₁₋₈alkyl or R⁵-morpholinyl-C₁₋₈alkyl;
R⁵ is one, two, three or four substituents each selected from hydrogen, halogen, hydroxy, oxo, carboxy, R⁶—C₁₋₈alkyl, C₁₋₈alkyl-sulfonyl, R⁶—C₁₋₈alkoxy, C₁₋₈alkyl-amino, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl; and
R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or phenyl.

4. The compound of claim 1, wherein A is absent.

5. The compound of claim 1, wherein A is C₁₋₈alkyl.

6. The compound of claim 1, wherein Y is C₃₋₁₄cycloalkyl, aryl or heterocyclyl each optionally substituted with one, two or three substituents each selected from C₁₋₈alkyl, C₂₋₈alkynyl, halo-C₁₋₈alkyl, halo-C₁₋₈alkoxy or halogen;

7. The compound of claim 1, wherein Y is cyclopropyl, cyclopentyl, cyclohexyl, 1H-indenyl, indanyl, phenyl, naphthalenyl or 6,7-dihydro-5H-cyclopenta[b]pyridinyl each optionally substituted with one, two or three substituents each selected from C₁₋₈alkyl, C₂₋₈alkynyl, halo-C₁₋₈alkyl, halo-C₁₋₈alkoxy or halogen;

8. The compound of claim 1, wherein Z is R¹-amino-carbonyl.

9. The compound of claim 1, wherein Z is heterocyclyl-carbonyl.

10. The compound of claim 1, wherein Z is morpholinyl-carbonyl or piperidinyl-carbonyl.

11. The compound of claim 1, wherein R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, C₃₋₁₄cycloalkyl-oxy or heteroaryl.

12. The compound of claim 1, wherein R¹ is one substituent selected from hydrogen, C₁₋₈alkyl, C₁₋₈alkoxy, C₁₋₈alkoxy-C₁₋₈alkyl, hydroxy, hydroxy-C₁₋₈alkyl, amino-C₁₋₈alkyl, cyclopentyl-oxy or thiazolyl.

13. The compound of claim 1, wherein R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-sulfonyl, R⁴-amino-C₁₋₈alkyl-sulfonyl, R⁵-aryl, R⁵-heterocyclyl, R⁵-heterocyclyl-oxy, R⁵-heterocyclyl-carbonyl, R⁵-heterocyclyl-sulfonyl, R⁵-heterocyclyl-C₁₋₈alkyl, R⁵-heteroaryl or R⁵-heteroaryl-C₁₋₈alkyl.

14. The compound of claim 1, wherein R²⁰⁰ is one substituent selected from hydrogen, R²—C₁₋₈alkyl, R³—C₁₋₈alkoxy, R⁴-amino, R⁴-amino-C₁₋₈alkyl, R⁴-amino-carbonyl, R⁴-amino-C₁₋₈alkyl-carbonyl, R⁴-amino-sulfonyl, R⁴-amino-C₁₋₈alkyl-sulfonyl, R⁵-phenyl, R⁵-pyrrolidinyl, R⁵-piperazinyl, R⁵-piperidinyl, R⁵-morpholinyl, R⁵-(1,2,3,6-tetrahydropyridinyl), R⁵-(3,5,11-trioxa-tricyclo[5.3.1.0^{2,6}]undecanyl), R⁵-1H-pyrrolyl, R⁵-1H-pyrazolyl, R⁵-1H-[1,2,4]triazolyl, R⁵-pyrrolidinyl-oxy, R⁵-piperidinyl-oxy, R⁵-pyranyl-oxy, R⁵-(1-azabicyclo[2.2.2]octyl)-oxy, R⁵-pyrrolidinyl-carbonyl, R⁵-piperidinyl-carbonyl, R⁵-morpholinyl-carbonyl, R⁵-piperazinyl-sulfonyl, R⁵-azetidinyl-C₁₋₈alkyl, R⁵-pyrrolidinyl-C₁₋₈alkyl, R⁵-piperazinyl-C₁₋₈alkyl, R⁵-piperidinyl-C₁₋₈alkyl, R⁵-morpholinyl-C₁₋₈alkyl, R⁵-thiomorpholinyl-C₁₋₈alkyl, R⁵-oxazolidinyl-C₁₋₈alkyl, R⁵-(7-aza-bicyclo[2.2.1]heptyl)-C₁₋₈alkyl, 1,5-dioxa-9-aza-spiro[5.5]undec-9-yl-C₁₋₈alkyl, R⁵-(1H-[1,2,4]triazolyl)-C₁₋₈alkyl, R⁵-1H-tetrazol-5-yl-C₁₋₈alkyl, R⁵-imidazolyl-C₁₋₈alkyl or R⁵-pyridinyl-C₁₋₈alkyl.

15. The compound of claim 1, wherein R²⁰⁰ is a ring selected from pyrrolidinyl, piperidinyl, 1,4,7,10,13-pentaoxa-cyclopentadecane, 1,4,7,10,13,16-hexaoxa-cyclooctadecane, pyrrolyl, imidazolyl or pyrazolyl fused on two adjacent carbon atoms of the phenyl ring of Formula (I) to form an R⁵ substituted 2,3-dihydro-1H-indolyl, 1,2,3,4-tetrahydroisoquinolinyl, 6,7,9,10,12,13,15,16-octahydro-5,8,11,14,17-pentaoxa-benzocyclopentadecenyl, 6,7,9,10,12,13,15,16,18,19-decahydro-5,8,11,14,17,20-hexaoxa-benzocyclooctadecenyl, 1H-indolyl, 1H-benzimidazolyl or 1H-indazolyl ring.

16. The compound of claim 1, wherein R² is one, two or three optional substituents each selected from hydroxy, C₁₋₈alkoxy, carboxy, C₁₋₈alkyl-amino-carbonyl, C₁₋₈alkyl-amino-amino-carbonyl, C₁₋₈alkyl-sulfonyl, C₁₋₈alkyl-amino-sulfonyl, C₁₋₈alkyl-carbonyl-amino-sulfonyl, C₁₋₈alkyl-sulfonyl-amino-carbonyl, R⁵-heterocyclyl-carbonyl or R⁵-heterocyclyl-amino-carbonyl.

17. The compound of claim 1, wherein R³ is one optional substituent selected from C₁₋₈alkoxy or R⁵-heterocyclyl.

18. The compound of claim 1, wherein R⁴ is two substituents each selected from hydrogen, C₁₋₈alkyl, C₁₋₈alkoxy-C₁₋₈alkyl, hydroxy-C₁₋₈alkyl, C₁₋₈alkyl-amino-C₁₋₈alkyl, amino-C₁₋₈alkyl-carbonyl, C₁₋₈alkyl-amino-C₁₋₈alkyl-carbonyl, C₁₋₈alkyl-sulfonyl, C₁₋₈alkyl-sulfonyl-C₁₋₈alkyl, C₃₋₁₄cycloalkyl, R⁵-heterocyclyl or R⁵-heterocyclyl-C₁₋₈alkyl.

19. The compound of claim 1, wherein R⁵ is one, two, three or four substituents each selected from hydrogen, halogen, hydroxy, oxo, carboxy, R⁶—C₁₋₈alkyl, R⁶—C₁₋₈alkoxy, amino, C₁₋₈alkyl-amino, C₁₋₈alkyl-sulfonyl, amino-sulfonyl, R⁶—C₁₋₈alkyl-carbonyl, C₁₋₈alkoxy-carbonyl, halo-C₁₋₈alkyl, halo-C₁₋₈alkyl-carbonyl or halo-C₁₋₈alkyl-sulfonyl.

20. The compound of claim 1, wherein R⁶ is one, two, three, four or five substituents each selected from hydrogen, halogen, hydroxy, C₁₋₈alkoxy, carboxy, C₁₋₈alkoxy-carbonyl, C₁₋₈alkyl-sulfonyl, C₁₋₈alkyl-sulfonyl-amino-carbonyl or aryl.

21. A compound selected from the group consisting of:
8-indan-5-yl-5-oxo-2-(3-piperazin-1-yl-phenylamino)-5,8-dihydro-pyrido[2,3-d]pyrimidine-6-carboxylic acid isopropylamide,
8-indan-5-yl-5-oxo-2-(3-piperazin-1-yl-phenylamino)-5,8-dihydro-pyrido[2,3-d]pyrimidine-6-carboxylic acid methoxy-amide,
8-(3-chloro-4-fluoro-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
8-(3-chloro-4-fluoro-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy amide,
8-indan-5-yl-5-oxo-2-[4-(2-pyrrolidin-1-yl-ethyl)-phenylamino]-5,8-dihydro-pyrido[2,3-d]pyrimidine-6-carboxylic acid amide,
2-{4-[(2S)-2-hydroxymethyl-pyrrolidin-1-yl]-phenylamino}-8-indan-5-yl-5-oxo-5,8-dihydro-pyrido[2,3-d]pyrimidine-6-carboxylic acid amide,
(S)-2-[4-(2-hydroxymethyl-pyrrolidin-1-yl)-phenylamino]-8-indan-5-yl-5-oxo-5,8-dihydro-pyrido[2,3-d]pyrimidine-6-carboxylic acid methoxy-amide,
8-indan-5-yl-5-oxo-2-[4-(2-oxo-pyrrolidin-1-ylmethyl)-phenylamino]-5,8-dihydro-pyrido[2,3-d]pyrimidine-6-carboxylic acid amide,
8-indan-5-yl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
8-cyclohexyl-5-oxo-2-(4-pyrrolidin-1-yl-phenylamino)-5,8-dihydro-pyrido[2,3-d]pyrimidine-6-carboxylic acid methoxy-amide,
8-cyclohexyl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
8-cyclohexyl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
8-cyclohexyl-5-oxo-2-[4-(2-pyrrolidin-1-yl-ethyl)-phenylamino]-5,8-dihydro-pyrido[2,3-d]pyrimidine-6-carboxylic acid methoxy-amide,
8-cyclohexyl-5-oxo-2-[4-(2-pyrrolidin-1-yl-ethyl)-phenylamino]-5,8-dihydro-pyrido[2,3-d]pyrimidine-6-carboxylic acid ethoxy-amide,
8-cyclohexyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
8-cyclohexyl-5-oxo-2-[4-(2-pyrrolidin-1-yl-ethyl)-phenylamino]-5,8-dihydro-pyrido[2,3-d]pyrimidine-6-carboxylic acid hydroxyamide,
8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-cyclohexyl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
8-cyclohexyl-2-[4-(2-morpholin-4-yl-ethyl)-phenylamino]-5-oxo-5,8-dihydro-pyrido[2,3-d]pyrimidine-6-carboxylic acid methoxy-amide,
8-cyclohexyl-2-[4-(2-morpholin-4-yl-ethyl)-phenylamino]-5-oxo-5,8-dihydro-pyrido[2,3-d]pyrimidine-6-carboxylic acid ethoxy-amide,
(S)-8-cyclohexyl-5-oxo-2-[4-(2-oxo-oxazolidin-4-ylmethyl)-phenylamino]-5,8-dihydro-pyrido[2,3-d]pyrimidine-6-carboxylic acid methoxy-amide,
(S)-8-cyclohexyl-5-oxo-2-[4-(2-oxo-oxazolidin-4-ylmethyl)-phenylamino]-5,8-dihydro-pyrido[2,3-d]pyrimidine-6-carboxylic acid ethoxy-amide,
8-cyclohexyl-2-[4-(3,5-dimethyl-piperazin-1-yl)-phenylamino]-5-oxo-5,8-dihydro-pyrido[2,3-d]pyrimidine-6-carboxylic acid methoxy-amide,
8-cyclohexyl-2-[4-(3,5-dimethyl-piperazin-1-yl)-phenylamino]-5-oxo-5,8-dihydro-pyrido[2,3-d]pyrimidine-6-carboxylic acid ethoxy-amide,
8-cyclohexyl-5-oxo-2-(4-piperidin-4-yl-phenylamino)-5,8-dihydro-pyrido[2,3-d]pyrimidine-6-carboxylic acid methoxy-amide,
8-cyclohexyl-5-oxo-2-(4-piperidin-4-yl-phenylamino)-5,8-dihydro-pyrido[2,3-d]pyrimidine-6-carboxylic acid ethoxy-amide,
2-[4-(1-acetyl-piperidin-4-yl)-phenylamino]-8-cyclohexyl-5-oxo-5,8-dihydro-pyrido[2,3-d]pyrimidine-6-carboxylic acid methoxy-amide,
2-[4-(1-acetyl-piperidin-4-yl)-phenylamino]-8-cyclohexyl-5-oxo-5,8-dihydro-pyrido[2,3-d]pyrimidine-6-carboxylic acid ethoxy-amide,
8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
8-(4-ethynyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-(4-ethynyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
8-(4-chloro-phenyl)-2-{4-[2-(4-methyl-piperazin-1-yl)-ethyl]-phenylamino}-5-oxo-5,8-dihydro-pyrido[2,3-d]pyrimidine-6-carboxylic acid amide,
8-(4-chloro-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-cyclopentyl-5-oxo-2-(4-pyrrolidin-1-yl-phenylamino)-5,8-dihydro-pyrido[2,3-d]pyrimidine-6-carboxylic acid methoxy-amide,
8-cyclopentyl-5-oxo-2-[4-(pyrrolidine-1-carbonyl)-phenylamino]-5,8-dihydro-pyrido[2,3-d]pyrimidine-6-carboxylic acid methoxy-amide,
8-cyclopentyl-5-oxo-2-[4-(2-oxo-pyrrolidin-1-ylmethyl)-phenylamino]-5,8-dihydro-pyrido[2,3-d]pyrimidine-6-carboxylic acid methoxy-amide,
8-cyclopentyl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
8-cyclopentyl-5-oxo-2-[4-(2-pyrrolidin-1-yl-ethyl)-phenylamino]-5,8-dihydro-pyrido[2,3-d]pyrimidine-6-carboxylic acid methoxy-amide,
8-cyclopentyl-5-oxo-2-[4-(2-pyrrolidin-1-yl-ethyl)-phenylamino]-5,8-dihydro-pyrido[2,3-d]pyrimidine-6-carboxylic acid ethoxy-amide,
8-cyclopentyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
8-cyclopentyl-6-(morpholine-4-carbonyl)-2-[4-(2-pyrrolidin-1-yl-ethyl)-phenylamino]-8H-pyrido[2,3-d]pyrimidin-5-one,
8-cyclopentyl-5-oxo-2-[4-(2-pyrrolidin-1-yl-ethyl)-phenylamino]-5,8-dihydro-pyrido[2,3-d]pyrimidine-6-carboxylic acid (2-hydroxy-ethyl)-amide,
8-cyclopentyl-5-oxo-2-[4-(2-pyrrolidin-1-yl-ethyl)-phenylamino]-5,8-dihydro-pyrido[2,3-d]pyrimidine-6-carboxylic acid (2-amino-ethyl)-amide,
8-cyclopentyl-5-oxo-2-(4-piperidin-4-yl-phenylamino)-5,8-dihydro-pyrido[2,3-d]pyrimidine-6-carboxylic acid methoxy-amide,
2-[4-(1-acetyl-piperidin-4-yl)-phenylamino]-8-cyclopentyl-5-oxo-5,8-dihydro-pyrido[2,3-d]pyrimidine-6-carboxylic acid methoxy-amide,
2-[4-(1-acetyl-piperidin-4-yl)-phenylamino]-8-cyclopentyl-5-oxo-5,8-dihydro-pyrido[2,3-d]pyrimidine-6-carboxylic acid ethoxy-amide,
8-cyclopentyl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-cyclopentyl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
8-(3,4-dimethyl-phenyl)-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
8-(3,4-dimethyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
8-(3,4-dimethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-(3,4-dimethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
8-(4-ethyl-phenyl)-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
8-(4-ethyl-phenyl)-5-oxo-2-[4-(2-pyrrolidin-1-yl-ethyl)-phenylamino]-5,8-dihydro-pyrido[2,3-d]pyrimidine-6-carboxylic acid methoxy-amide,
8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
8-(4-ethyl-phenyl)-5-oxo-2-(4-piperidin-4-yl-phenylamino)-5,8-dihydro-pyrido[2,3-d]pyrimidine-6-carboxylic acid isopropoxy-amide,
2-[4-(1-acetyl-piperidin-4-yl)-phenylamino]-8-(4-ethyl-phenyl)-5-oxo-5,8-dihydro-pyrido[2,3-d]pyrimidine-6-carboxylic acid isopropoxy-amide,
8-(4-ethyl-phenyl)-2-[4-(1-methanesulfonyl-piperidin-4-yl)-phenylamino]-5-oxo-5,8-dihydro-pyrido[2,3-d]pyrimidine-6-carboxylic acid isopropoxy-amide,
8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid methoxy-amide,
8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid ethoxy-amide,
8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid isopropoxy-amide,
8-indan-5-yl-2-{4-[2-(4-methyl-piperazin-1-yl)-ethyl]-phenylamino}-6-(piperidine-1-carbonyl)-8H-pyrido[2,3-d]pyrimidin-5-one,
8-indan-5-yl-2-{4-[2-(4-methyl-piperazin-1-yl)-ethyl]-phenylamino}-5-oxo-5,8-dihydro-pyrido[2,3-d]pyrimidine-6-carboxylic acid (2-hydroxy-ethyl)-amide,
8-indan-5-yl-5-oxo-2-(3-piperazin-1-yl-phenylamino)-5,8-dihydro-pyrido[2,3-d]pyrimidine-6-carboxylic acid (2-hydroxy-ethyl)-amide,
(S)-8-indan-5-yl-5-oxo-2-[4-(2-oxo-oxazolidin-4-ylmethyl)-phenylamino]-5,8-dihydro-pyrido[2,3-d]pyrimidine-6-carboxylic acid methoxy-amide,
(S)-8-indan-5-yl-5-oxo-2-[4-(2-oxo-oxazolidin-4-ylmethyl)-phenylamino]-5,8-dihydro-pyrido[2,3-d]pyrimidine-6-carboxylic acid ethoxy-amide,
2-[4-(1-acetyl-piperidin-4-yl)-phenylamino]-8-indan-5-yl-5-oxo-5,8-dihydro-pyrido[2,3-d]pyrimidine-6-carboxylic acid methoxy-amide,
2-[4-(1-acetyl-piperidin-4-yl)-phenylamino]-8-indan-5-yl-5-oxo-5,8-dihydro-pyrido[2,3-d]pyrimidine-6-carboxylic acid ethoxy-amide,
8-indan-5-yl-2-[4-(2-methanesulfonyl-ethyl)-phenylamino]-5-oxo-5,8-dihydro-pyrido[2,3-d]pyrimidine-6-carboxylic acid methoxy-amide,
8-indan-5-yl-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
2-{4-[2-(1,5-dioxa-9-aza-spiro[5.5]undec-9-yl)-ethyl]-phenylamino}-8-indan-5-yl-5-oxo-5,8-dihydro-pyrido[2,3-d]pyrimidine-6-carboxylic acid methoxy-amide,
8-indan-5-yl-2-(4-{2-[methyl-(tetrahydro-pyran-4-yl)-amino]-ethyl}-phenylamino)-5-oxo-5,8-dihydro-pyrido[2,3-d]pyrimidine-6-carboxylic acid methoxy-amide,
8-indan-5-yl-5-oxo-2-(4-piperidin-4-yl-phenylamino)-5,8-dihydro-pyrido[2,3-d]pyrimidine-6-carboxylic acid methoxy-amide,
8-indan-5-yl-2-[4-(1-methanesulfonyl-piperidin-4-yl)-phenylamino]-5-oxo-5,8-dihydro-pyrido[2,3-d]pyrimidine-6-carboxylic acid methoxy-amide,
8-indan-5-yl-2-[4-(2-isopropylsulfamoyl-ethyl)-phenylamino]-5-oxo-5,8-dihydro-pyrido[2,3-d]pyrimidine-6-carboxylic acid methoxy-amide,
8-indan-5-yl-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide,
2-[4-(2-hydroxy-ethyl)-phenylamino]-8-indan-5-yl-5-oxo-5,8-dihydro-pyrido[2,3-d]pyrimidine-6-carboxylic acid methoxy-amide,
8-indan-5-yl-2-[4-(2-morpholin-4-yl-ethyl)-phenylamino]-5-oxo-5,8-dihydro-pyrido[2,3-d]pyrimidine-6-carboxylic acid methoxy-amide,
8-indan-5-yl-2-[4-(2-methoxy-ethyl)-phenylamino]-5-oxo-5,8-dihydro-pyrido[2,3-d]pyrimidine-6-carboxylic acid methoxy-amide,
8-indan-5-yl-2-[4-(2-methanesulfonylamino-ethyl)-phenylamino]-5-oxo-5,8-dihydro-pyrido[2,3-d]pyrimidine-6-carboxylic acid methoxy-amide,
8-indan-5-yl-5-oxo-2-{4-[2-(4-oxo-piperidin-1-yl)-ethyl]-phenylamino}-5,8-dihydro-pyrido[2,3-d]pyrimidine-6-carboxylic acid methoxy-amide,
3-{4-[4-(8-indan-5-yl-6-methoxycarbamoyl-5-oxo-5,8-dihydro-pyrido[2,3-d]pyrimidin-2-ylamino)-phenyl]-piperidin-1-yl}-propionic acid,
{4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid,
{4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester,
8-indan-5-yl-2-{4-[1-(2-methanesulfonylamino-2-oxo-ethyl)-piperidin-4-yl]-phenylamino}-5-oxo-5,8-dihydro-pyrido[2,3-d]pyrimidine-6-carboxylic acid methoxy-amide,
2-{4-[1-(2-hydroxy-ethyl)-piperidin-4-yl]-phenylamino}-8-indan-5-yl-5-oxo-5,8-dihydro-pyrido[2,3-d]pyrimidine-6-carboxylic acid methoxy-amide,
8-indan-5-yl-2-[4-(2-methylsulfamoyl-ethyl)-phenylamino]-5-oxo-5,8-dihydro-pyrido[2,3-d]pyrimidine-6-carboxylic acid methoxy-amide,
2-[4-(2-acetylsulfamoyl-ethyl)-phenylamino]-8-indan-5-yl-5-oxo-5,8-dihydro-pyrido[2,3-d]pyrimidine-6-carboxylic acid methoxy-amide,
8-indan-5-yl-5-oxo-2-[4-(1H-tetrazol-5-ylmethyl)-phenylamino]-5,8-dihydro-pyrido[2,3-d]pyrimidine-6-carboxylic acid methoxy-amide,
8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid cyclopentyloxy-amide,
8-(4-ethyl-phenyl)-2-{4-[2-(4-methyl-piperazin-1-yl)-ethyl]-phenylamino}-5-oxo-5,8-dihydro-pyrido[2,3-d]pyrimidine-6-carboxylic acid (1-ethyl-propoxy)-amide,
(4-{4-[8-(4-ethyl-phenyl)-6-methoxycarbamoyl-5-oxo-5,8-dihydro-pyrido[2,3-d]pyrimidin-2-ylamino]-phenyl}-piperidin-1-yl)-acetic acid,
8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide,
8-(4-ethyl-phenyl)-2-{4-[1-(2-methanesulfonyl-ethyl)-piperidin-4-yl]-phenylamino}-5-oxo-5,8-dihydro-pyrido[2,3-d]pyrimidine-6-carboxylic acid methoxy-amide,
2-{4-[2-(2-hydroxy-ethylamino)-ethyl]-phenylamino}-8-indan-5-yl-5-oxo-5,8-dihydro-pyrido[2,3-d]pyrimidine-6-carboxylic acid methoxy-amide,
8-indan-5-yl-2-{4-[2-(2-methoxy-ethylamino)-ethyl]-phenylamino}-5-oxo-5,8-dihydro-pyrido[2,3-d]pyrimidine-6-carboxylic acid methoxy-amide,
8-indan-5-yl-2-{4-[2-(2-methanesulfonyl-ethylamino)-ethyl]-phenylamino}-5-oxo-5,8-dihydro-pyrido[2,3-d]pyrimidine-6-carboxylic acid methoxy-amide,
2-(4-{2-[bis-(2-hydroxy-ethyl)-amino]-ethyl}-phenylamino)-8-indan-5-yl-5-oxo-5,8-dihydro-pyrido[2,3-d]pyrimidine-6-carboxylic acid methoxy-amide,
(R)-2-{4-[2-(2-hydroxymethyl-pyrrolidin-1-yl)-ethyl]-phenylamino}-8-indan-5-yl-5-oxo-5,8-dihydro-pyrido[2,3-d]pyrimidine-6-carboxylic acid methoxy-amide,
8-indan-5-yl-5-oxo-2-[4-(piperidin-1-ylcarbamoylmethyl)-phenylamino]-5,8-dihydro-pyrido[2,3-d]pyrimidine-6-carboxylic acid methoxy-amide,
{4-[8-(4-ethyl-phenyl)-6-methoxycarbamoyl-5-oxo-5,8-dihydro-pyrido[2,3-d]pyrimidin-2-ylamino]-phenyl}-acetic acid,
8-(4-ethyl-phenyl)-5-oxo-2-[4-(piperidin-1-ylcarbamoylmethyl)-phenylamino]-5,8-dihydro-pyrido[2,3-d]pyrimidine-6-carboxylic acid methoxy-amide,
2-[4-(N′,N′-dimethyl-hydrazinocarbonylmethyl)-phenylamino]-8-(4-ethyl-phenyl)-5-oxo-5,8-dihydro-pyrido[2,3-d]pyrimidine-6-carboxylic acid methoxy-amide,
8-(4-ethyl-phenyl)-2-[4-(N′-methyl-hydrazinocarbonylmethyl)-phenylamino]-5-oxo-5,8-dihydro-pyrido[2,3-d]pyrimidine-6-carboxylic acid methoxy-amide,
2-{4-[2-(adamantan-2-ylamino)-ethyl]-phenylamino}-8-indan-5-yl-5-oxo-5,8-dihydro-pyrido[2,3-d]pyrimidine-6-carboxylic acid methoxy-amide,
2-{4-[2-(bicyclo[2.2.1]hept-2-ylamino)-ethyl]-phenylamino}-8-indan-5-yl-5-oxo-5,8-dihydro-pyrido[2,3-d]pyrimidine-6-carboxylic acid methoxy-amide,
(R)-1-{2-[4-(8-indan-5-yl-6-methoxycarbamoyl-5-oxo-5,8-dihydro-pyrido[2,3-d]pyrimidin-2-ylamino)-phenyl]-ethyl}-pyrrolidine-2-carboxylic acid,
2-{4-[2-(7-aza-bicyclo[2.2.1]hept-7-yl)-ethyl]-phenylamino}-8-indan-5-yl-5-oxo-5,8-dihydro-pyrido[2,3-d]pyrimidine-6-carboxylic acid methoxy-amide,
8-indan-5-yl-2-[4-(1-isopropyl-piperidin-4-yl)-phenylamino]-5-oxo-5,8-dihydro-pyrido[2,3-d]pyrimidine-6-carboxylic acid methoxy-amide,
8-indan-5-yl-5-oxo-2-[3-(piperidine-1-carbonyl)-phenylamino]-5,8-dihydro-pyrido[2,3-d]pyrimidine-6-carboxylic acid amide,
8-benzyl-2-[4-(4-methyl-piperazin-1-yl)-phenylamino]-5-oxo-5,8-dihydro-pyrido[2,3-d]pyrimidine-6-carboxylic acid amide,
2-[3-(3-dimethylamino-propyl)-phenylamino]-8-indan-5-yl-5-oxo-5,8-dihydro-pyrido[2,3-d]pyrimidine-6-carboxylic acid amide,
2-[3-(3-dimethylamino-propyl)-phenylamino]-8-indan-5-yl-5-oxo-5,8-dihydro-pyrido[2,3-d]pyrimidine-6-carboxylic acid methylamide,
2-[3-(3-dimethylamino-propyl)-phenylamino]-8-indan-5-yl-5-oxo-5,8-dihydro-pyrido[2,3-d]pyrimidine-6-carboxylic acid ethylamide,
2-(4-dimethylcarbamoylmethyl-phenylamino)-8-indan-5-yl-5-oxo-5,8-dihydro-pyrido[2,3-d]pyrimidine-6-carboxylic acid amide,
8-indan-5-yl-2-[3-(morpholine-4-carbonyl)-phenylamino]-5-oxo-5,8-dihydro-pyrido[2,3-d]pyrimidine-6-carboxylic acid amide,
2-[4-(2-dimethylamino-ethyl)-phenylamino]-8-indan-5-yl-5-oxo-5,8-dihydro-pyrido[2,3d]pyrimidine-6-carboxylic acid amide,
8-indan-5-yl-5-oxo-2-[4-(pyrrolidine-1-carbonyl)-phenylamino]-5,8-dihydro-pyrido[2,3-d]pyrimidine-6-carboxylic acid amide,
2-(4-dimethylcarbamoyl-phenylamino)-8-indan-5-yl-5-oxo-5,8-dihydro-pyrido[2,3-d]pyrimidine-6-carboxylic acid amide,
8-indan-5-yl-5-oxo-2-(4-pyrrolidin-1-ylmethyl-phenylamino)-5,8-dihydro-pyrido[2,3-d]pyrimidine-6-carboxylic acid amide,
8-indan-5-yl-5-oxo-2-(3-pyrrolidin-1-yl-phenylamino)-5,8-dihydro-pyrido[2,3-d]pyrimidine-6-carboxylic acid amide,
2-[4-(1-acetyl-piperidin-4-yl)-phenylamino]-8-indan-5-yl-5-oxo-5,8-dihydro-pyrido[2,3-d]pyrimidine-6-carboxylic acid amide,
8-indan-5-yl-2-[4-(4-methyl-piperazin-1-ylmethyl)-phenylamino]-5-oxo-5,8-dihydro-pyrido[2,3-d]pyrimidine-6-carboxylic acid methoxy-amide,
2-(3-dimethylsulfamoyl-phenylamino)-8-indan-5-yl-5-oxo-5,8-dihydro-pyrido[2,3-d]pyrimidine-6-carboxylic acid methoxy-amide,
8-indan-5-yl-5-oxo-2-[4-(2-pyrrolidin-1-yl-ethyl)-phenylamino]-5,8-dihydro-pyrido[2,3-d]pyrimidine-6-carboxylic acid methoxy-amide,
8-indan-5-yl-5-oxo-2-[4-(2-pyrrolidin-1-yl-ethyl)-phenylamino]-5,8-dihydro-pyrido[2,3-d]pyrimidine-6-carboxylic acid (2-methoxy-ethyl)-amide,
8-indan-5-yl-5-oxo-2-[4-(2-pyrrolidin-1-yl-ethyl)-phenylamino]-5,8-dihydro-pyrido[2,3-d]pyrimidine-6-carboxylic acid isopropoxy-amide,
8-indan-5-yl-5-oxo-2-{4-[2-(2-oxo-pyrrolidin-1-yl)-ethyl]-phenylamino}-5,8-dihydro-pyrido[2,3-d]pyrimidine-6-carboxylic acid methoxy-amide,
8-indan-5-yl-5-oxo-2-{4-[2-(2-oxo-pyrrolidin-1-yl)-ethyl]-phenylamino}-5,8-dihydro-pyrido[2,3-d]pyrimidine-6-carboxylic acid ethoxy-amide,
2-(4-dimethylsulfamoyl-phenylamino)-8-indan-5-yl-5-oxo-5,8-dihydro-pyrido[2,3-d]pyrimidine-6-carboxylic acid ethoxy-amide,
2-(4-dimethylsulfamoyl-phenylamino)-8-indan-5-yl-5-oxo-5,8-dihydro-pyrido[2,3-d]pyrimidine-6-carboxylic acid isopropoxy-amide,
8-indan-5-yl-2-[4-(1-methyl-piperidin-4-yl)-phenylamino]-5-oxo-5,8-dihydro-pyrido[2,3-d]pyrimidine-6-carboxylic acid methoxy-amide,
8-indan-5-yl-2-[4-(1-methyl-piperidin-4-yl)-phenylamino]-5-oxo-5,8-dihydro-pyrido[2,3-d]pyrimidine-6-carboxylic acid ethoxy-amide,
8-cyclohexyl-2-[4-(1-methyl-piperidin-4-yl)-phenylamino]-5-oxo-5,8-dihydro-pyrido[2,3-d]pyrimidine-6-carboxylic acid methoxy-amide,
8-cyclohexyl-2-[4-(1-methyl-piperidin-4-yl)-phenylamino]-5-oxo-5,8-dihydro-pyrido[2,3-d]pyrimidine-6-carboxylic acid ethoxy-amide,
8-cyclopropyl-5-oxo-2-[4-(2-pyrrolidin-1-yl-ethyl)-phenylamino]-5,8-dihydro-pyrido[2,3-d]pyrimidine-6-carboxylic acid ethoxy-amide,
8-cyclopropyl-5-oxo-2-[4-(2-pyrrolidin-1-yl-ethyl)-phenylamino]-5,8-dihydro-pyrido[2,3-d]pyrimidine-6-carboxylic acid isopropoxy-amide,
8-indan-5-yl-5-oxo-2-[4-(2-piperazin-1-yl-ethyl)-phenylamino]-5,8-dihydro-pyrido[2,3-d]pyrimidine-6-carboxylic acid methoxy-amide,
2-{4-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide,
8-indan-5-yl-2-{4-[2-(4-methanesulfonyl-piperazin-1-yl)-ethyl]-phenylamino}-5-oxo-5,8-dihydro-pyrido[2,3-d]pyrimidine-6-carboxylic acid methoxy-amide,
(4-{2-[4-(8-indan-5-yl-6-methoxycarbamoyl-5-oxo-5,8-dihydro-pyrido[2,3-d]pyrimidin-2-ylamino)-phenyl]-ethyl}-piperazin-1-yl)-acetic acid,
8-indan-5-yl-5-oxo-2-(4-{2-[4-(2,2,2-trifluoro-acetyl)-piperazin-1-yl]-ethyl}-phenylamino)-5,8-dihydro-pyrido[2,3-d]pyrimidine-6-carboxylic acid methoxy-amide,
8-indan-5-yl-5-oxo-2-(4-{2-[4-(2,2,2-trifluoro-ethyl)-piperazin-1-yl]-ethyl}-phenylamino)-5,8-dihydro-pyrido[2,3-d]pyrimidine-6-carboxylic acid methoxy-amide,
4-{2-[4-(8-indan-5-yl-6-methoxycarbamoyl-5-oxo-5,8-dihydro-pyrido[2,3-d]pyrimidin-2-ylamino)-phenyl]-ethyl}-piperazine-1-carboxylic acid methyl ester,
8-indan-5-yl-2-{4-[3-(4-methylpiperazin-1-yl)-propyl]-phenylamino}-5-oxo-5,8-dihydro-pyrido[2,3-d]pyrimidine-6-carboxylic acid methoxy-amide,
8-indan-5-yl-2-{4-[2-(4-methylpiperazin-1-yl)-2-oxo-ethyl]-phenylamino}-5-oxo-5,8-dihydro-pyrido[2,3-d]pyrimidine-6-carboxylic acid methoxy-amide,
8-indan-5-yl-2-[4-(3-methanesulfonylamino-3-oxo-propyl)-phenylamino]-5-oxo-5,8-dihydro-pyrido[2,3-d]pyrimidine-6-carboxylic acid methoxy-amide,
8-indan-5-yl-2-[4-(4-methyl-piperazine-1-sulfonyl)-phenylamino]-5-oxo-5,8-dihydro-pyrido[2,3-d]pyrimidine-6-carboxylic acid methoxy-amide,
2-[4-(1-ethyl-piperidin-4-yl)-phenylamino]-8-indan-5-yl-5-oxo-5,8-dihydro-pyrido[2,3-d]pyrimidine-6-carboxylic acid methoxy-amide,
8-(4-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide,
8-(4-ethyl-phenyl)-2-[4-(1-ethyl-piperidin-4-yl)-phenylamino]-5-oxo-5,8-dihydro-pyrido[2,3-d]pyrimidine-6-carboxylic acid methoxy-amide,
2-[4-(1-methylpiperidin-4-yl)-phenylamino]-5-oxo-8-(4-trifluoromethoxyphenyl)-5,8-dihydropyrido[2,3-d]pyrimidine-6-carboxylic acid methoxy-amide,
2-{4-[1-(2-hydroxyethyl)-piperidin-4-yl]-phenylamino}-5-oxo-8-(4-trifluoromethoxy-phenyl)-5,8-dihydropyrido[2,3-d]pyrimidine-6-carboxylic acid methoxy-amide,
2-[4-(2-imidazol-1-yl-ethyl)-phenylamino]-8-indan-5-yl-5-oxo-5,8-dihydro-pyrido[2,3-d]pyrimidine-6-carboxylic acid methoxy-amide,
8-indan-5-yl-2-{4-[2-(5-methyl-1H-[1,2,4]triazol-3-yl)-ethyl]-phenylamino}-5-oxo-5,8-dihydro-pyrido[2,3-d]pyrimidine-6-carboxylic acid methoxy-amide,
8-indan-5-yl-5-oxo-2-(4-pyridin-4-ylmethyl-phenylamino)-5,8-dihydro-pyrido[2,3-d]pyrimidine-6-carboxylic acid methoxy-amide,
8-indan-5-yl-5-oxo-2-(3-[1,2,4]triazol-1-yl-phenylamino)-5,8-dihydro-pyrido[2,3-d]pyrimidine-6-carboxylic acid methoxy-amide,
2-{4-[2-(4-methylpiperazin-1-yl)-ethyl]-phenylamino}-5-oxo-8-(4-trifluoromethoxy-phenyl)-5,8-dihydropyrido[2,3-d]pyrimidine-6-carboxylic acid methoxy-amide,
5-oxo-2-(4-piperidin-4-yl-phenylamino)-8-(4-trifluoromethoxyphenyl)-5,8-dihydro-pyrido[2,3-d]pyrimidine-6-carboxylic acid methoxy-amide,
5-oxo-2-[4-(1-sulfamoyl-piperidin-4-yl)-phenylamino]-8-(4-trifluoromethoxyphenyl)-5,8-dihydropyrido[2,3-d]pyrimidine-6-carboxylic acid methoxy-amide,
8-indan-5-yl-5-oxo-2-[4-(1-sulfamoyl-piperidin-4-yl)-phenylamino]-5,8-dihydro-pyrido[2,3-d]pyrimidine-6-carboxylic acid methoxy-amide,
8-(4-ethyl-phenyl)-5-oxo-2-[4-(1-sulfamoyl-piperidin-4-yl)-phenylamino]-5,8-dihydro-pyrido[2,3-d]pyrimidine-6-carboxylic acid methoxy-amide,
8-(4-ethyl-phenyl)-2-[4-(1-ethyl-piperidin-4-yl)-phenylamino]-5-oxo-5,8-dihydro-pyrido[2,3-d]pyrimidine-6-carboxylic acid ethoxy-amide,
8-(4-ethyl-phenyl)-2-[4-(1-ethyl-piperidin-4-yl)-phenylamino]-5-oxo-5,8-dihydro-pyrido[2,3-d]pyrimidine-6-carboxylic acid isopropoxy-amide,
8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid ethoxy-amide,
8-(4-ethyl-phenyl)-2-{4-[1-(2-hydroxy-ethyl)-piperidin-4-yl]-phenylamino}-5-oxo-5,8-dihydro-pyrido[2,3-d]pyrimidine-6-carboxylic acid isopropoxy-amide,
8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid methoxy-amide,
8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid ethoxy-amide,
8-(4-ethyl-phenyl)-2-{4-[1-(3-hydroxy-propyl)-piperidin-4-yl]-phenylamino}-5-oxo-5,8-dihydro-pyrido[2,3-d]pyrimidine-6-carboxylic acid isopropoxy-amide,
8-(4-ethyl-phenyl)-5-oxo-2-{4-[1-(2,2,2-trifluoro-ethyl)-piperidin-4-yl]-phenylamino}-5,8-dihydro-pyrido[2,3-d]pyrimidine-6-carboxylic acid methoxy-amide,
8-(4-ethyl-phenyl)-5-oxo-2-{4-[1-(2,2,2-trifluoro-ethyl)-piperidin-4-yl]-phenylamino}-5,8-dihydro-pyrido[2,3-d]pyrimidine-6-carboxylic acid ethoxy-amide,
8-indan-5-yl-5-oxo-2-{4-[2-(1H-[1,2,4]triazol-3-yl)-ethyl]-phenylamino}-5,8-dihydro-pyrido[2,3-d]pyrimidine-6-carboxylic acid methoxy-amide,
2-{4-[2-(5-methyl-1H-[1,2,4]triazol-3-yl)-ethyl]-phenylamino}-5-oxo-8-(4-trifluoromethoxyphenyl)-5,8-dihydropyrido[2,3-d]pyrimidine-6-carboxylic acid methoxy-amide,
8-indan-5-yl-5-oxo-2-[3-(2-pyrrolidin-1-yl-ethylcarbamoyl)-phenylamino]-5,8-dihydro-pyrido[2,3-d]pyrimidine-6-carboxylic acid methoxy-amide,
2-{3-[(1-ethyl-pyrrolidin-2-ylmethyl)-carbamoyl]-phenylamino}-8-indan-5-yl-5-oxo-5,8-dihydro-pyrido[2,3-d]pyrimidine-6-carboxylic acid methoxy-amide,
8-indan-5-yl-5-oxo-2-[3-(2-pyrrolidin-1-yl-ethyl)-phenylamino]-5,8-dihydro-pyrido[2,3-d]pyrimidine-6-carboxylic acid methoxy-amide,
2-[3-(3,5-dimethyl-piperazin-1-yl)-phenylamino]-8-indan-5-yl-5-oxo-5,8-dihydro-pyrido[2,3-d]pyrimidine-6-carboxylic acid methoxy-amide,
2-[3-(4-acetyl-piperazin-1-yl)-phenylamino]-8-indan-5-yl-5-oxo-5,8-dihydro pyrido[2,3-d]pyrimidine-6-carboxylic acid methoxy-amide,
2-[3-(1-acetyl-piperidin-4-yl)-phenylamino]-8-indan-5-yl-5-oxo-5,8-dihydro-pyrido[2,3-d]pyrimidine-6-carboxylic acid methoxy-amide,
8-indan-5-yl-2-[3-(2-morpholin-4-yl-ethylcarbamoyl)-phenylamino]-5-oxo-5,8-dihydro-pyrido[2,3-d]pyrimidine-6-carboxylic acid methoxy-amide,
2-[3-(2-dimethylamino-ethylcarbamoyl)-phenylamino]-8-indan-5-yl-5-oxo-5,8-dihydro-pyrido[2,3-d]pyrimidine-6-carboxylic acid methoxy-amide,
8-indan-5-yl-2-{3-[2-(4-methyl-piperazin-1-yl)-ethyl]-phenylamino}-5-oxo-5,8-dihydro-pyrido[2,3-d]pyrimidine-6-carboxylic acid methoxy-amide,
8-indan-5-yl-2-[3-(4-methanesulfonyl-piperazin-1-yl)-phenylamino]-5-oxo-5,8-dihydro-pyrido[2,3-d]pyrimidine-6-carboxylic acid methoxy-amide,
8-indan-5-yl-5-oxo-2-[3-(4-trifluoromethanesulfonyl-piperazin-1-yl)-phenylamino]-5,8-dihydro-pyrido[2,3-d]pyrimidine-6-carboxylic acid methoxy-amide,
8-indan-5-yl-2-{3-[2-(4-methanesulfonyl-piperazin-1-yl)-ethyl]-phenylamino}-5-oxo-5,8-dihydro-pyrido[2,3-d]pyrimidine-6-carboxylic acid methoxy-amide,
2-{3-[2-(4-acetyl-piperazin-1-yl)-ethyl]-phenylamino}-8-indan-5-yl-5-oxo-5,8-dihydro-pyrido[2,3-d]pyrimidine-6-carboxylic acid methoxy-amide,
2-{3-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide,
2-{4-[2-(1,1-dioxo-1λ⁶-thiomorpholin-4-yl)-ethyl]-phenylamino}-8-indan-5-yl-5-oxo-5,8-dihydro-pyrido[2,3-d]pyrimidine-6-carboxylic acid methoxy-amide,
8-(4-ethyl-phenyl)-2-{4-[1-(2-methoxy-ethyl)-piperidin-4-yl]-phenylamino}-5-oxo-5,8-dihydro-pyrido[2,3-d]pyrimidine-6-carboxylic acid methoxy-amide,
8-(4,4-dimethyl-cyclohexyl)-2-[4-(1-ethyl-piperidin-4-yl)-phenylamino]-5-oxo-5,8-dihydro-pyrido[2,3-d]pyrimidine-6-carboxylic acid methoxy-amide,
8-(4,4-dimethyl-cyclohexyl)-2-[4-(1-methyl-piperidin-4-yl)-phenylamino]-5-oxo-5,8-dihydro-pyrido[2,3-d]pyrimidine-6-carboxylic acid methoxy-amide,
8-(4,4-dimethyl-cyclohexyl)-2-{4-[1-(2-hydroxy-ethyl)-piperidin-4-yl]-phenylamino}-5-oxo-5,8-dihydro-pyrido[2,3-d]pyrimidine-6-carboxylic acid methoxy-amide,
8-(4-ethyl-phenyl)-2-{4-[1-(2-methoxy-ethyl)-piperidin-4-yl]-phenylamino}-5-oxo-5,8-dihydro-pyrido[2,3-d]pyrimidine-6-carboxylic acid methoxy-amide,
8-(3-ethyl-phenyl)-2-[4-(1-methyl-piperidin-4-yl)-phenylamino]-5-oxo-5,8-dihydro-pyrido[2,3-d]pyrimidine-6-carboxylic acid methoxy-amide,
8-(4-ethyl-phenyl)-2-[4-(3-methoxy-propylamino)-phenylamino]-5-oxo-5,8-dihydro-pyrido[2,3-d]pyrimidine-6-carboxylic acid methoxy-amide,
8-indan-5-yl-2-(4-methanesulfonylaminocarbonyl-phenylamino)-5-oxo-5,8-dihydro-pyrido[2,3-d]pyrimidine-6-carboxylic acid methoxy-amide,
8-(4-ethyl-phenyl)-2-[3-(2-methoxy-ethoxy)-phenylamino]-5-oxo-5,8-dihydro-pyrido[2,3-d]pyrimidine-6-carboxylic acid methoxy-amide,
8-(4-ethyl-phenyl)-2-[3-(3-methoxy-propoxy)-phenylamino]-5-oxo-5,8-dihydro-pyrido[2,3-d]pyrimidine-6-carboxylic acid methoxy-amide,
8-(4-ethyl-phenyl)-2-[4-(2-methanesulfonyl-ethylamino)-phenylamino]-5-oxo-5,8-dihydro-pyrido[2,3-d]pyrimidine-6-carboxylic acid methoxy-amide,
{4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid ethyl ester,
{4-[4-(8-indan-5-yl-6-methoxycarbamoyl-5-oxo-5,8-dihydro-pyrido[2,3-d]pyrimidin-2-ylamino)-phenyl]-piperidin-1-yl}-acetic acid,
8-indan-5-yl-2-{4-[2-(4-methyl-3-oxo-piperazin-1-yl)-ethyl]-phenylamino}-5-oxo-5,8-dihydro-pyrido[2,3-d]pyrimidine-6-carboxylic acid methoxy-amide,
8-(4-ethyl-phenyl)-2-{4-[2-(4-methyl-3-oxo-piperazin-1-yl)-ethyl]-phenylamino}-5-oxo-5,8-dihydro-pyrido[2,3-d]pyrimidine-6-carboxylic acid methoxy-amide,
8-indan-5-yl-2-[4-(1-methyl-piperidin-4-yl)-phenylamino]-5-oxo-5,8-dihydro-pyrido[2,3-d]pyrimidine-6-carboxylic acid,
8-indan-5-yl-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
8-(4-ethyl-phenyl)-5-oxo-2-{4-[2-(3-oxo-piperazin-1-yl)-ethyl]-phenylamino}-5,8-dihydro-pyrido[2,3-d]pyrimidine-6-carboxylic acid methoxy-amide,
8-(6,7-dihydro-5H-[1]pyrindin-3-yl)-2-[4-(1-methyl-piperidin-4-yl)-phenylamino]-5-oxo-5,8-dihydro-pyrido[2,3-d]pyrimidine-6-carboxylic acid methoxy-amide,
8-(6,7-dihydro-5H-[1]pyrindin-3-yl)-2-[4-(2-methanesulfonyl-ethyl)-phenylamino]-5-oxo-5,8-dihydro-pyrido[2,3-d]pyrimidine-6-carboxylic acid methoxy-amide,
8-(6,7-dihydro-5H-[1]pyrindin-3-yl)-2-[4-(2-hydroxy-ethyl)-phenylamino]-5-oxo-5,8-dihydro-pyrido[2,3-d]pyrimidine-6-carboxylic acid methoxy-amide,
2-[3-(2-dimethylamino-acetylamino)-phenylamino]-8-(4-ethyl-phenyl)-5-oxo-5,8-dihydro-pyrido[2,3-d]pyrimidine-6-carboxylic acid methoxy-amide,
2-[3-(2-dimethylamino-acetylamino)-phenylamino]-8-indan-5-yl-5-oxo-5,8-dihydro-pyrido[2,3-d]pyrimidine-6-carboxylic acid methoxy-amide,
2-[3-(2-dimethylamino-ethanesulfonyl)-phenylamino]-8-indan-5-yl-5-oxo-5,8-dihydro-pyrido[2,3-d]pyrimidine-6-carboxylic acid methoxy-amide,
8-indan-5-yl-2-[3-(2-methylamino-ethanesulfonyl)-phenylamino]-5-oxo-5,8-dihydro-pyrido[2,3-d]pyrimidine-6-carboxylic acid methoxy-amide,
2-{4-[2-(3,3-difluoro-piperidin-1-yl)-ethyl]-phenylamino}-8-(4-ethyl-phenyl)-5-oxo-5,8-dihydro-pyrido[2,3-d]pyrimidine-6-carboxylic acid methoxy-amide,
2-{4-[2-(3,3-difluoro-piperidin-1-yl)-ethyl]-phenylamino}-8-(4-ethyl-phenyl)-5-oxo-5,8-dihydro-pyrido[2,3-d]pyrimidine-6-carboxylic acid methoxy-amide,
2-{4-[2-(4,4-difluoro-piperidin-1-yl)-ethyl]-phenylamino}-8-indan-5-yl-5-oxo-5,8-dihydro-pyrido[2,3-d]pyrimidine-6-carboxylic acid methoxy-amide,
8-indan-5-yl-2-[4-(1-methyl-1,2,3,6-tetrahydro-pyridin-4-yl)-phenylamino]-5-oxo-5,8-dihydro-pyrido[2,3-d]pyrimidine-6-carboxylic acid methoxy-amide,
2-{4-[(3S,4S)-(3,4-dihydroxy-1-methyl-piperidin-4-yl)]-phenylamino}-8-indan-5-yl-5-oxo-5,8-dihydro-pyrido[2,3-d]pyrimidine-6-carboxylic acid methoxy-amide,
2-[4-(3,3-difluoro-1-methyl-piperidin-4-yl)-phenylamino]-8-indan-5-yl-5-oxo-5,8-dihydro-pyrido[2,3-d]pyrimidine-6-carboxylic acid methoxy-amide,
2-{4-[2-(4-hydroxy-piperidin-1-yl)-ethyl]-phenylamino}-8-indan-5-yl-5-oxo-5,8-dihydro-pyrido[2,3-d]pyrimidine-6-carboxylic acid methoxy-amide,
2-{4-{2-[(3R)-(3-hydroxy-piperidin-1-yl)]-ethyl}-phenylamino}-8-indan-5-yl-5-oxo-5,8-dihydro-pyrido[2,3-d]pyrimidine-6-carboxylic acid methoxy-amide,
8-(4-ethyl-phenyl)-2-(4-{2-[(3R)-(3-hydroxy-piperidin-1-yl)]-ethyl}-phenylamino)-5-oxo-5,8-dihydro-pyrido[2,3-d]pyrimidine-6-carboxylic acid methoxy-amide,
2-{4-[2-(3,3-difluoro-pyrrolidin-1-yl)-ethyl]-phenylamino}-8-indan-5-yl-5-oxo-5,8-dihydro-pyrido[2,3-d]pyrimidine-6-carboxylic acid methoxy-amide,
8-(4-ethyl-phenyl)-5-oxo-2-(4-piperidin-2-yl-phenylamino)-5,8-dihydro-pyrido[2,3-d]pyrimidine-6-carboxylic acid methoxy-amide,
8-indan-5-yl-2-[4-(1-methyl-piperidin-2-yl)-phenylamino]-5-oxo-5,8-dihydro-pyrido[2,3-d]pyrimidine-6-carboxylic acid methoxy-amide,
2-[4-(1-ethyl-piperidin-2-yl)-phenylamino]-8-indan-5-yl-5-oxo-5,8-dihydro-pyrido[2,3-d]pyrimidine-6-carboxylic acid methoxy-amide,
8-(4-ethyl-phenyl)-2-[4-(1-ethyl-piperidin-2-yl)-phenylamino]-5-oxo-5,8-dihydro-pyrido[2,3-d]pyrimidine-6-carboxylic acid methoxy-amide,
8-indan-5-yl-5-oxo-2-(4-piperidin-2-yl-phenylamino)-5,8-dihydro-pyrido[2,3-d]pyrimidine-6-carboxylic acid methoxy-amide,
2-[3-(1-aza-bicyclo[2.2.2]oct-3-yloxy)-phenylamino]-8-(4-ethyl-phenyl)-5-oxo-5,8-dihydro-pyrido[2,3-d]pyrimidine-6-carboxylic acid methoxy-amide,
8-(4-ethyl-phenyl)-5-oxo-2-[3-(piperidin-3-yloxy)-phenylamino]-5,8-dihydro-pyrido[2,3-d]pyrimidine-6-carboxylic acid methoxy-amide,
2-{4-[(3R,4R)-(3,4-dihydroxy-1-methyl-piperidin-4-yl)]-phenylamino}-8-(4-ethyl-phenyl)-5-oxo-5,8-dihydro-pyrido[2,3-d]pyrimidine-6-carboxylic acid methoxy-amide,
2-{4-[(3R,4R)-(3,4-dihydroxy-1-methyl-piperidin-4-yl)]-phenylamino}-8-indan-5-yl-5-oxo-5,8-dihydro-pyrido[2,3-d]pyrimidine-6-carboxylic acid methoxy-amide,
2-[3-(1-ethyl-piperidin-3-yloxy)-phenylamino]-8-indan-5-yl-5-oxo-5,8-dihydro-pyrido[2,3-d]pyrimidine-6-carboxylic acid methoxy-amide,
2-(4-{2-[(3R)-(3-fluoro-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
2-(4-{2-[(3S)-(3-fluoro-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
2-{4-[2-(3-hydroxy-azetidin-1-yl)-ethyl]-phenylamino}-8-indan-5-yl-5-oxo-5,8-dihydro-pyrido[2,3-d]pyrimidine-6-carboxylic acid methoxy-amide,
2-(4-{2-[(3S)-(3-hydroxy-pyrrolidin-1-yl)]-ethyl}-phenylamino)-8-indan-5-yl-5-oxo-5,8-dihydro-pyrido[2,3-d]pyrimidine-6-carboxylic acid methoxy-amide,
2-{4-[2-(3-fluoro-piperidin-1-yl)-ethyl]-phenylamino}-8-indan-5-yl-5-oxo-5,8-dihydro-pyrido[2,3-d]pyrimidine-6-carboxylic acid methoxy-amide,
8-(4-ethyl-phenyl)-2-[4-(2-methoxy-ethoxy)-phenylamino]-5-oxo-5,8-dihydro-pyrido[2,3-d]pyrimidine-6-carboxylic acid methoxy-amide,
8-indan-5-yl-2-[4-(2-methoxy-ethoxy)-phenylamino]-5-oxo-5,8-dihydro-pyrido[2,3-d]pyrimidine-6-carboxylic acid methoxy-amide,
8-(1H-inden-5-yl)-2-[4-(1-methyl-piperidin-4-yl)-phenylamino]-5-oxo-5,8-dihydro-pyrido[2,3-d]pyrimidine-6-carboxylic acid methoxy-amide,
2-[4-(2-azetidin-1-yl-ethyl)-phenylamino]-8-(4-ethyl-phenyl)-5-oxo-5,8-dihydro-pyrido[2,3-d]pyrimidine-6-carboxylic acid methoxy-amide,
8-indan-5-yl-2-[3-(2-methoxy-ethoxy)-phenylamino]-5-oxo-5,8-dihydro-pyrido[2,3-d]pyrimidine-6-carboxylic acid methoxy-amide,
2-[4-(2-azetidin-1-yl-ethyl)-phenylamino]-8-indan-5-yl-5-oxo-5,8-dihydro-pyrido[2,3-d]pyrimidine-6-carboxylic acid methoxy-amide,
2-{3-[(3S)-(1-ethyl-pyrrolidin-3-yloxy)]-phenylamino}-8-indan-5-yl-5-oxo-5,8-dihydro-pyrido[2,3-d]pyrimidine-6-carboxylic acid methoxy-amide,
2-[4-(1-aza-bicyclo[2.2.2]oct-3-yloxy)-phenylamino]-8-indan-5-yl-5-oxo-5,8-dihydro-pyrido[2,3-d]pyrimidine-6-carboxylic acid methoxy-amide,
8-(4-ethyl-phenyl)-2-{4-[2-(3-fluoro-azetidin-1-yl)-ethyl]-phenylamino}-5-oxo-5,8-dihydro-pyrido[2,3-d]pyrimidine-6-carboxylic acid methoxy-amide,
2-{4-[2-(3-fluoro-azetidin-1-yl)-ethyl]-phenylamino}-8-indan-5-yl-5-oxo-5,8-dihydro-pyrido[2,3-d]pyrimidine-6-carboxylic acid methoxy-amide,
2-[3-(1-aza-bicyclo[2.2.2]oct-3-yloxy)-phenylamino]-8-indan-5-yl-5-oxo-5,8-dihydro-pyrido[2,3-d]pyrimidine-6-carboxylic acid methoxy-amide,
2-[4-(1-aza-bicyclo[2.2.2]oct-3-yloxy)-phenylamino]-8-(4-ethyl-phenyl)-5-oxo-5,8-dihydro-pyrido[2,3-d]pyrimidine-6-carboxylic acid methoxy-amide,
2-{4-[2-(3,3-difluoro-azetidin-1-yl)-ethyl]-phenylamino}-8-(4-ethyl-phenyl)-5-oxo-5,8-dihydro-pyrido[2,3-d]pyrimidine-6-carboxylic acid methoxy-amide,
2-{4-[2-(3,3-difluoro-azetidin-1-yl)-ethyl]-phenylamino}-8-indan-5-yl-5-oxo-5,8-dihydro-pyrido[2,3-d]pyrimidine-6-carboxylic acid methoxy-amide,
8-indan-5-yl-2-[3-(1-methyl-piperidin-3-yl)-phenylamino]-5-oxo-5,8-dihydro-pyrido[2,3-d]pyrimidine-6-carboxylic acid methoxy-amide,
2-[3-(4-hydroxy-1-methyl-piperidin-4-yl)-phenylamino]-8-indan-5-yl-5-oxo-5,8-dihydro-pyrido[2,3-d]pyrimidine-6-carboxylic acid methoxy-amide,
8-indan-5-yl-2-[3-(1-methyl-piperidin-4-yl)-phenylamino]-5-oxo-5,8-dihydro-pyrido[2,3-d]pyrimidine-6-carboxylic acid methoxy-amide,
2-[3-(3-hydroxy-pyrrolidin-3-yl)-phenylamino]-8-indan-5-yl-5-oxo-5,8-dihydro-pyrido[2,3-d]pyrimidine-6-carboxylic acid methoxy-amide,
2-[3-(3-hydroxy-piperidin-3-yl)-phenylamino]-8-indan-5-yl-5-oxo-5,8-dihydro-pyrido[2,3-d]pyrimidine-6-carboxylic acid methoxy-amide,
2-[3-(1-ethyl-3-hydroxy-piperidin-3-yl)-phenylamino]-8-indan-5-yl-5-oxo-5,8-dihydro-pyrido[2,3-d]pyrimidine-6-carboxylic acid methoxy-amide,
2-{3-[4-hydroxy-1-(2,2,2-trifluoro-ethyl)-piperidin-4-yl]-phenylamino}-8-indan-5-yl-5-oxo-5,8-dihydro-pyrido[2,3-d]pyrimidine-6-carboxylic acid methoxy-amide,
2-[3-(1-ethyl-3-hydroxy-piperidin-3-yl)-phenylamino]-8-(4-ethyl-phenyl)-5-oxo-5,8-dihydro-pyrido[2,3-d]pyrimidine-6-carboxylic acid methoxy-amide,
2-(1-acetyl-2,3-dihydro-1H-indol-5-ylamino)-8-(4-ethyl-phenyl)-5-oxo-5,8-dihydro-pyrido[2,3-d]pyrimidine-6-carboxylic acid methoxy-amide,
8-(4-ethyl-phenyl)-2-(1-methanesulfonyl-2,3-dihydro-1H-indol-5-ylamino)-5-oxo-5,8-dihydro-pyrido[2,3-d]pyrimidine-6-carboxylic acid methoxy-amide,
2-(1-acetyl-2,3-dihydro-1H-indol-6-ylamino)-8-(4-ethyl-phenyl)-5-oxo-5,8-dihydro-pyrido[2,3-d]pyrimidine-6-carboxylic acid methoxy-amide,
8-(4-ethyl-phenyl)-5-oxo-2-(1,2,3,4-tetrahydro-isoquinolin-7-ylamino)-5,8-dihydro-pyrido[2,3-d]pyrimidine-6-carboxylic acid methoxy-amide,
8-indan-5-yl-5-oxo-2-(4-[1,2,4]triazol-1-ylmethyl-phenylamino)-5,8-dihydro-pyrido[2,3-d]pyrimidine-6-carboxylic acid methoxy-amide,
8-indan-5-yl-5-oxo-2-(4-pyrazol-1-yl-phenylamino)-5,8-dihydro-pyrido[2,3-d]pyrimidine-6-carboxylic acid methoxy-amide,
8-(4-ethyl-phenyl)-2-(1H-indol-5-ylamino)-5-oxo-5,8-dihydro-pyrido[2,3-d]pyrimidine-6-carboxylic acid methoxy-amide,
8-(4-ethyl-phenyl)-2-(1H-indol-6-ylamino)-5-oxo-5,8-dihydro-pyrido[2,3-d]pyrimidine-6-carboxylic acid methoxy-amide,
8-indan-5-yl-2-(1H-indol-5-ylamino)-5-oxo-5,8-dihydro-pyrido[2,3-d]pyrimidine-6-carboxylic acid methoxy-amide,
8-(4-ethyl-phenyl)-2-(1H-indazol-6-ylamino)-5-oxo-5,8-dihydro-pyrido[2,3-d]pyrimidine-6-carboxylic acid methoxy-amide,
8-indan-5-yl-2-(1H-indol-6-ylamino)-5-oxo-5,8-dihydro-pyrido[2,3-d]pyrimidine-6-carboxylic acid methoxy-amide,
8-(4-ethyl-phenyl)-2-(1-methyl-1H-indol-5-ylamino)-5-oxo-5,8-dihydro-pyrido[2,3-d]pyrimidine-6-carboxylic acid methoxy-amide,
8-indan-5-yl-2-(1-methyl-1H-indol-5-ylamino)-5-oxo-5,8-dihydro-pyrido[2,3-d]pyrimidine-6-carboxylic acid methoxy-amide,
8-indan-5-yl-5-oxo-2-{4-[1-(2,2,2-trifluoro-ethyl)-piperidin-4-yl]-phenylamino}-5,8-dihydro-pyrido[2,3-d]pyrimidine-6-carboxylic acid methoxy-amide,
8-indan-5-yl-5-oxo-2-{4-[1-(2,2,3,3,3-pentafluoro-propyl)-piperidin-4-yl]-phenylamino}-5,8-dihydro-pyrido[2,3-d]pyrimidine-6-carboxylic acid methoxy-amide,
2-(6,7,9,10,12,13,15,16,18,19-decahydro-5,8,11,14,17,20-hexaoxa-benzocyclooctadecen-2-ylamino)-8-(4-ethyl-phenyl)-5-oxo-5,8-dihydro-pyrido[2,3-d]pyrimidine-6-carboxylic acid methoxy-amide,
8-(4-ethyl-phenyl)-5-oxo-2-[4-(1-phenethyl-piperidin-4-yl)-phenylamino]-5,8-dihydro-pyrido[2,3-d]pyrimidine-6-carboxylic acid methoxy-amide,
8-(4-ethyl-phenyl)-5-oxo-2-{4-[(2S,3S,4S,5S,6R)(3,4,5-trihydroxy-6-hydroxymethyl-tetrahydro-pyran-2-yloxy)]-phenylamino}-5,8-dihydro-pyrido[2,3-d]pyrimidine-6-carboxylic acid methoxy-amide,
8-(4-ethyl-phenyl)-2-(6,7,9,10,12,13,15,16-octahydro-5,8,11,14,17-pentaoxa-benzocyclopentadecen-2-ylamino)-5-oxo-5,8-dihydro-pyrido[2,3-d]pyrimidine-6-carboxylic acid methoxy-amide,
8-indan-5-yl-5-oxo-2-[4-(1-propyl-piperidin-4-yl)-phenylamino]-5,8-dihydro-pyrido[2,3-d]pyrimidine-6-carboxylic acid methoxy-amide,
8-(4-ethyl-phenyl)-2-(3′-methoxy-biphenyl-4-ylamino)-5-oxo-5,8-dihydro-pyrido[2,3-d]pyrimidine-6-carboxylic acid methoxy-amide,
8-indan-5-yl-5-oxo-2-phenylamino-5,8-dihydro-pyrido[2,3-d]pyrimidine-6-carboxylic acid methoxy-amide,
2-(1-ethyl-1H-benzoimidazol-5-ylamino)-8-indan-5-yl-5-oxo-5,8-dihydro-pyrido[2,3-d]pyrimidine-6-carboxylic acid methoxy-amide,
2-(1H-benzoimidazol-5-ylamino)-8-indan-5-yl-5-oxo-5,8-dihydro-pyrido[2,3-d]pyrimidine-6-carboxylic acid methoxy-amide,
2-[4-(1-methyl-piperidin-4-yl)-phenylamino]-8-naphthalen-2-yl-5-oxo-5,8-dihydro-pyrido[2,3-d]pyrimidine-6-carboxylic acid methoxy-amide,
8-(4-ethyl-phenyl)-5-oxo-2-(2-oxo-2,3-dihydro-1H-indol-5-ylamino)-5,8-dihydro-pyrido[2,3-d]pyrimidine-6-carboxylic acid methoxy-amide,
8-(4-ethyl-phenyl)-5-oxo-2-(3,4,5-trimethoxy-phenylamino)-5,8-dihydro-pyrido[2,3-d]pyrimidine-6-carboxylic acid methoxy-amide,
2-(4′-dimethylamino-biphenyl-4-ylamino)-8-(4-ethyl-phenyl)-5-oxo-5,8-dihydro-pyrido[2,3-d]pyrimidine-6-carboxylic acid methoxy-amide,
8-(4-ethyl-phenyl)-5-oxo-2-[4-(piperidin-4-yloxy)-phenylamino]-5,8-dihydro-pyrido[2,3-d]pyrimidine-6-carboxylic acid methoxy-amide,
2-[3,4-bis-(2-methoxy-ethoxy)-phenylamino]-8-(4-ethyl-phenyl)-5-oxo-5,8-dihydro-pyrido[2,3-d]pyrimidine-6-carboxylic acid methoxy-amide,
8-(4-ethyl-phenyl)-2-[4-(1-methyl-piperidin-4-yloxy)-phenylamino]-5-oxo-5,8-dihydro-pyrido[2,3-d]pyrimidine-6-carboxylic acid methoxy-amide,
8-(4-ethyl-phenyl)-2-[4-(1-ethyl-piperidin-4-yloxy)-phenylamino]-5-oxo-5,8-dihydro-pyrido[2,3-d]pyrimidine-6-carboxylic acid methoxy-amide,
8-indan-5-yl-5-oxo-2-[4-(piperidin-4-yloxy)-phenylamino]-5,8-dihydro-pyrido[2,3-d]pyrimidine-6-carboxylic acid methoxy-amide,
2-[4-(1-ethyl-piperidin-4-yloxy)-phenylamino]-8-indan-5-yl-5-oxo-5,8-dihydro-pyrido[2,3-d]pyrimidine-6-carboxylic acid methoxy-amide,
8-indan-5-yl-2-[4-(1-methyl-piperidin-4-yloxy)-phenylamino]-5-oxo-5,8-dihydro-pyrido[2,3-d]pyrimidine-6-carboxylic acid methoxy-amide,
2-[3,4-bis-(2-methoxy-ethoxy)-phenylamino]-8-indan-5-yl-5-oxo-5,8-dihydro-pyrido[2,3-d]pyrimidine-6-carboxylic acid methoxy-amide,
8-indan-5-yl-5-oxo-2-[4-(2aR,1R,7S,6aS)-(1,4,4,7-tetramethyl-3,5,11-trioxa-tricyclo[5.3.1.0^{2,6}]undec-9-yl)-phenylamino]-5,8-dihydro-pyrido[2,3-d]pyrimidine-6-carboxylic acid methoxy-amide,
8-(4-ethyl-phenyl)-5-oxo-2-phenylamino-5,8-dihydro-pyrido[2,3-d]pyrimidine-6-carboxylic acid methoxy-amide,
8-(4-ethyl-phenyl)-5-oxo-2-(4-pyrrol-1-yl-phenylamino)-5,8-dihydro-pyrido[2,3-d]pyrimidine-6-carboxylic acid methoxy-amide,
8-(4-ethyl-phenyl)-5-oxo-2-{4-[1-(2,2,2-trifluoro-ethyl)-piperidin-4-yloxy]-phenylamino}-5,8-dihydro-pyrido[2,3-d]pyrimidine-6-carboxylic acid methoxy-amide,
2-(3-dimethylamino-phenylamino)-8-(4-ethyl-phenyl)-5-oxo-5,8-dihydro-pyrido[2,3-d]pyrimidine-6-carboxylic acid methoxy-amide,
8-(4-ethyl-phenyl)-2-(3-morpholin-4-yl-phenylamino)-5-oxo-5,8-dihydro-pyrido[2,3-d]pyrimidine-6-carboxylic acid methoxy-amide,
8-(4-ethyl-phenyl)-2-[3-(4-methyl-piperazin-1-yl)-phenylamino]-5-oxo-5,8-dihydro-pyrido[2,3-d]pyrimidine-6-carboxylic acid methoxy-amide,
8-(4-ethyl-phenyl)-2-[3-(1-ethyl-piperidin-4-yloxy)-phenylamino]-5-oxo-5,8-dihydro-pyrido[2,3-d]pyrimidine-6-carboxylic acid methoxy-amide,
2-[3,4-bis-(3-methoxy-propoxy)-phenylamino]-8-(4-ethyl-phenyl)-5-oxo-5,8-dihydro-pyrido[2,3-d]pyrimidine-6-carboxylic acid methoxy-amide,
8-indan-5-yl-2-[3-(4-methyl-piperazin-1-yl)-phenylamino]-5-oxo-5,8-dihydro-pyrido[2,3-d]pyrimidine-6-carboxylic acid methoxy-amide,
8-indan-5-yl-2-(3-morpholin-4-yl-phenylamino)-5-oxo-5,8-dihydro-pyrido[2,3-d]pyrimidine-6-carboxylic acid methoxy-amide, and
2-(3-dimethylamino-phenylamino)-8-indan-5-yl-5-oxo-5,8-dihydro-pyrido[2,3-d]pyrimidine-6-carboxylic acid methoxy-amide.

22. A pharmaceutical composition, comprising a compound of claim 1 and a pharmaceutically acceptable carrier.

23. A method of treating or ameliorating a c-fms kinase mediated disorder in a subject in need thereof comprising administering to the subject an effective amount of at least one compound of claim 1.

24. The method of claim 23, wherein the cardiovascular disease, inflammatory disease or cancer is selected from glomerulonephritis, immune nephritis, rheumatoid arthritis, inflammatory bowel disease, prosthesis failure, sarcoidosis, congestive obstructive pulmonary disease, idiopathic pulmonary fibrosis, asthma, pancreatitis, HIV infection, psoriasis, graft rejection, diabetes, diabetic retinopathy, diabetic nephropathy, obesity, cardiac hypertrophy, atherosclerosis, restenosis, age-related macular degeneration, tumor related angiogenesis, solid tumor cancers, blood related cancers, multiple sclerosis, schizophrenia or Alzheimer's dementia.

25. The method of claim 23, wherein the effective amount is from about 0.001 mg/kg to about 10 g.
